SEQUENCE LISTING (374 sequences)

SEQ ID NO: 1 - skipped

SEQ ID NO: 2 - protein, 5 aa, Synthetic construct
SYGIS

SEQ ID NO: 3 - protein, 5 aa, Synthetic construct
SYGIS

SEQ ID NO: 4 - protein, 5 aa, Synthetic construct
SYGIS

SEQ ID NO: 5 - protein, 5 aa, Synthetic construct
DYYIH

SEQ ID NO: 6 - protein, 5 aa, Synthetic construct
DYYIH

SEQ ID NO: 7 - protein, 17 aa, Synthetic construct
WIXXXXGXTXYAQXXXG

SEQ ID NO: 8 - protein, 17 aa, Synthetic construct
WISAYNGNTNYAQNLRG

SEQ ID NO: 9 - protein, 17 aa, Synthetic construct
WISAYNGNTNYAQNLRG

SEQ ID NO: 10 - protein, 17 aa, Synthetic construct
WIIAYNGNTNYAQKLQG

SEQ ID NO: 11 - protein, 17 aa, Synthetic construct
WINPNTGATKYAQKFQG

SEQ ID NO: 12 - protein, 17 aa, Synthetic construct
WINPNTGATKYAQKFQG

SEQ ID NO: 13 - protein, 15 aa, Synthetic construct
VSLVWFGELPRGFDY

SEQ ID NO: 14 - protein, 15 aa, Synthetic construct
VSLVWFGELPRGFDY

SEQ ID NO: 15 - protein, 8 aa, Synthetic construct
DTGFSFDY

SEQ ID NO: 16 - protein, 12 aa, Synthetic construct
EDNGNYGDVFDI

SEQ ID NO: 17 - protein, 12 aa, Synthetic construct
EDNGNYGDVFDI

SEQ ID NO: 18 - protein, 7 aa, Synthetic construct
TSGVGVG

SEQ ID NO: 19 - protein, 7 aa, Synthetic construct
TSGVGVG

SEQ ID NO: 20 - protein, 7 aa, Synthetic construct
TSGVGVG

SEQ ID NO: 21 - protein, 16 aa, Synthetic construct
LIYWNDDKXYSPSLKS

SEQ ID NO: 22 - protein, 16 aa, Synthetic construct
LIYWNDDKRYSPSLKS

SEQ ID NO: 23 - protein, 16 aa, Synthetic construct
LIYWNDDKQYSPSLKS

SEQ ID NO: 24 - skipped

SEQ ID NO: 25 - protein, 10 aa, Synthetic construct
DYGDYWFFDL

SEQ ID NO: 26 - protein, 10 aa, Synthetic construct
NPDYGDFFDY

SEQ ID NO: 27 - skipped

SEQ ID NO: 28 - protein, 5 aa, Synthetic construct
VYDMH

SEQ ID NO: 29 - protein, 5 aa, Synthetic construct
SHDMH

SEQ ID NO: 30 - protein, 5 aa, Synthetic construct
SYDMH

SEQ ID NO: 31 - protein, 5 aa, Synthetic construct
DYDMH

SEQ ID NO: 32 - protein, 5 aa, Synthetic construct
DYGMS

SEQ ID NO: 33 - protein, 5 aa, Synthetic construct
DYGMS

SEQ ID NO: 34 - protein, 5 aa, Synthetic construct
SYSMN

SEQ ID NO: 35 - protein, 5 aa, Synthetic construct
TYSMN

SEQ ID NO: 36 - protein, 5 aa, Synthetic construct
SYAMS

SEQ ID NO: 37 - protein, 5 aa, Synthetic construct
SYAMS

SEQ ID NO: 38 - protein, 5 aa, Synthetic construct
SYGMH

SEQ ID NO: 39 - protein, 5 aa, Synthetic construct
SYGMH

SEQ ID NO: 40 - protein, 5 aa, Synthetic construct
SYGMH

SEQ ID NO: 41 - protein, 5 aa, Synthetic construct
SYGMH

SEQ ID NO: 42 - protein, 5 aa, Synthetic construct
DFAMH

SEQ ID NO: 43 - skipped

SEQ ID NO: 44 - protein, 16 aa, Synthetic construct
GIDPDGDTYYPGSVKG

SEQ ID NO: 45 - protein, 16 aa, Synthetic construct
GIDPAGDTYYPDSVKG

SEQ ID NO: 46 - protein, 16 aa, Synthetic construct
GIDPDGDTYYPGSVKG

SEQ ID NO: 47 - protein, 16 aa, Synthetic construct
GIDPDGDTYYPGSLKG

SEQ ID NO: 48 - protein, 17 aa, Synthetic construct
GINWNGGSTGYADSVKG

SEQ ID NO: 49 - protein, 17 aa, Synthetic construct
TINWNGGNTGYADSVKG

SEQ ID NO: 50 - protein, 16 aa, Synthetic construct
SITSSSYIFYADSLKG

SEQ ID NO: 51 - protein, 17 aa, Synthetic construct
SITSSSSYMFYADSVMG

SEQ ID NO: 52 - protein, 17 aa, Synthetic construct
AISGSGGSTYYADSVKG

SEQ ID NO: 53 - protein, 17 aa, Synthetic construct
AISGSGGSTYYADSVKG

SEQ ID NO: 54 - protein, 17 aa, Synthetic construct
VISYYGSNKYYADSVKG

SEQ ID NO: 55 - protein, 17 aa, Synthetic construct
VMSYDGINGYYADSVKG

SEQ ID NO: 56 - protein, 17 aa, Synthetic construct
HITYDGSDKYYGDSVKG

SEQ ID NO: 57 - protein, 17 aa, Synthetic construct
IISYDGNNKYYADSVKG

SEQ ID NO: 58 - protein, 17 aa, Synthetic construct
GIGWNSGSIGYADSVKG

SEQ ID NO: 59 - protein, 13 aa, Synthetic construct
GEESNSSEDGFDI

SEQ ID NO: 60 - protein, 13 aa, Synthetic construct
GEASSSSEDAFDI

SEQ ID NO: 61 - protein, 13 aa, Synthetic construct
GEDNSSSEDAFDI

SEQ ID NO: 62 - protein, 13 aa, Synthetic construct
GEDSSSSEDAFDI

SEQ ID NO: 63 - protein, 11 aa, Synthetic construct
YSGSFRDAFDI

SEQ ID NO: 64 - protein, 13 aa, Synthetic construct
DEGAAGLGYYFDY

SEQ ID NO: 65 - protein, 10 aa, Synthetic construct
DSNWGEAFDI

SEQ ID NO: 66 - protein, 10 aa, Synthetic construct
DSNWGEAFDI

SEQ ID NO: 67 - protein, 11 aa, Synthetic construct
DGDYYYYYMDV

SEQ ID NO: 68 - protein, 16 aa, Synthetic construct
VERDMVRGHYYYYMDV

SEQ ID NO: 69 - protein, 6 aa, Synthetic construct
GITMDV

SEQ ID NO: 70 - protein, 10 aa, Synthetic construct
LVYADDAFDI

SEQ ID NO: 71 - protein, 12 aa, Synthetic construct
DGYSSGWWYFDY

SEQ ID NO: 72 - protein, 17 aa, Synthetic construct
ERGYSSSSGYYYYYMDV

SEQ ID NO: 73 - protein, 11 aa, Synthetic construct
APDYGDYYFDY

SEQ ID NO: 74 - protein, 7 aa, Synthetic construct
SXSXYWX

SEQ ID NO: 75 - protein, 7 aa, Synthetic construct
SSSSYWG

SEQ ID NO: 76 - protein, 7 aa, Synthetic construct
SSSSYWG

SEQ ID NO: 77 - protein, 7 aa, Synthetic construct
SGSYYWS

SEQ ID NO: 78 - protein, 16 aa, Synthetic construct
XIYYSGSTXSNPSLKS

SEQ ID NO: 79 - protein, 16 aa, Synthetic construct
SIYYSGSTYSNPSLKS

SEQ ID NO: 80 - protein, 16 aa, Synthetic construct
SIYYSGSTYSNPSLKS

SEQ ID NO: 81 - protein, 16 aa, Synthetic construct
YIYYSGSTSYNPSLKS

SEQ ID NO: 82 - skipped

SEQ ID NO: 83 - protein, 11 aa, Synthetic construct
QYISGTEYFQY

SEQ ID NO: 84 - protein, 11 aa, Synthetic construct
QYISGTEYFQY

SEQ ID NO: 85 - protein, 10 aa, Synthetic construct
AKWEGDYFDY

SEQ ID NO: 86 - protein, 11 aa, Synthetic construct
RASQXIXXXLX

SEQ ID NO: 87 - protein, 11 aa, Synthetic construct
RASQGISSWLA

SEQ ID NO: 88 - protein, 11 aa, Synthetic construct
RASQGIRNDLG

SEQ ID NO: 89 - protein, 11 aa, Synthetic construct
RASQGIRIDLG

SEQ ID NO: 90 - protein, 11 aa, Synthetic construct
RASQGIRNDLG

SEQ ID NO: 91 - protein, 11 aa, Synthetic construct
RASQGISSYLA

SEQ ID NO: 92 - protein, 11 aa, Synthetic construct
RASQSISSYLN

SEQ ID NO: 93 - protein, 11 aa, Synthetic construct
RASQSISSWLA

SEQ ID NO: 94 - protein, 11 aa, Synthetic construct
RASQSISIYLN

SEQ ID NO: 95 - protein, 11 aa, Synthetic construct
RASQSISSYLN

SEQ ID NO: 96 - protein, 11 aa, Synthetic construct
RASQSIYSYLN

SEQ ID NO: 97 - protein, 11 aa, Synthetic construct
RASQGIRNDLG

SEQ ID NO: 98 - protein, 7 aa, Synthetic construct
AXSSLXS

SEQ ID NO: 99 - protein, 7 aa, Synthetic construct
AASSLQS

SEQ ID NO: 100 - protein, 7 aa, Synthetic construct
AASSLPS

SEQ ID NO: 101 - protein, 7 aa, Synthetic construct
AASSLPS

SEQ ID NO: 102 - protein, 7 aa, Synthetic construct
AASSLQS

SEQ ID NO: 103 - protein, 7 aa, Synthetic construct
AASSLQS

SEQ ID NO: 104 - protein, 7 aa, Synthetic construct
AASSLQS

SEQ ID NO: 105 - protein, 7 aa, Synthetic construct
ATSSLQS

SEQ ID NO: 106 - protein, 7 aa, Synthetic construct
AASSLQS

SEQ ID NO: 107 - protein, 7 aa, Synthetic construct
AASSLQS

SEQ ID NO: 108 - protein, 7 aa, Synthetic construct
AASSLQS

SEQ ID NO: 109 - protein, 7 aa, Synthetic construct
AASSLQS

SEQ ID NO: 110 - skipped

SEQ ID NO: 111 - protein, 9 aa, Synthetic construct
QQANSFPWT

SEQ ID NO: 112 - protein, 9 aa, Synthetic construct
LQHNSYPWT

SEQ ID NO: 113 - protein, 9 aa, Synthetic construct
LQHNSYPWT

SEQ ID NO: 114 - protein, 9 aa, Synthetic construct
LQHNSYPLT

SEQ ID NO: 115 - protein, 9 aa, Synthetic construct
QQSFSTPLT

SEQ ID NO: 116 - protein, 9 aa, Synthetic construct
QQSFSTPLT

SEQ ID NO: 117 - protein, 9 aa, Synthetic construct
QQSYSTPIT

SEQ ID NO: 118 - protein, 9 aa, Synthetic construct
QQSYTTPLT

SEQ ID NO: 119 - protein, 9 aa, Synthetic construct
QQSYSTPLT

SEQ ID NO: 120 - protein, 10 aa, Synthetic construct
QQSYSTPPYT

SEQ ID NO: 121 - protein, 9 aa, Synthetic construct
LQDYNYPYT

SEQ ID NO: 122 - protein, 16 aa, Synthetic construct
RSSQSLXXSXXXXYLX

SEQ ID NO: 123 - protein, 16 aa, Synthetic construct
RSSQSLLHSNRYNYLD

SEQ ID NO: 124 - protein, 16 aa, Synthetic construct
RSSQSLVYSDGNTYLN

SEQ ID NO: 125 - protein, 16 aa, Synthetic construct
RSSQSLVYSDGNTYLN

SEQ ID NO: 126 - protein, 16 aa, Synthetic construct
RSSQSLVFSDGNTYLN

SEQ ID NO: 127 - protein, 16 aa, Synthetic construct
RSSQSLVYSDGNTYLN

SEQ ID NO: 128 - protein, 7 aa, Synthetic construct
XXSNRXS

SEQ ID NO: 129 - protein, 7 aa, Synthetic construct
LGSNRAS

SEQ ID NO: 130 - protein, 7 aa, Synthetic construct
KVSNRDS

SEQ ID NO: 131 - protein, 7 aa, Synthetic construct
EVSNRDS

SEQ ID NO: 132 - protein, 7 aa, Synthetic construct
KVSNRDS

SEQ ID NO: 133 - protein, 7 aa, Synthetic construct
EVSNRDS

SEQ ID NO: 134 - protein, 9 aa, Synthetic construct
MQXXXXPXT

SEQ ID NO: 135 - protein, 9 aa, Synthetic construct
MQTLQTPYT

SEQ ID NO: 136 - protein, 9 aa, Synthetic construct
MQGTHWPWT

SEQ ID NO: 137 - protein, 9 aa, Synthetic construct
MQGTHWPWT

SEQ ID NO: 138 - protein, 9 aa, Synthetic construct
MQGTHWPWT

SEQ ID NO: 139 - protein, 9 aa, Synthetic construct
MQGTHWPWT

SEQ ID NO: 140 - protein, 12 aa, Synthetic construct
RASQSVSXXXXA

SEQ ID NO: 141 - protein, 11 aa, Synthetic construct
RASQSVSSYLA

SEQ ID NO: 142 - protein, 11 aa, Synthetic construct
RASQSVSSYLA

SEQ ID NO: 143 - protein, 11 aa, Synthetic construct
RASQSVSNYLA

SEQ ID NO: 144 - protein, 12 aa, Synthetic construct
RASQSVSSSYLA

SEQ ID NO: 145 - protein, 12 aa, Synthetic construct
RASQSVSSSYLA

SEQ ID NO: 146 - protein, 12 aa, Synthetic construct
RASQSVSSSYLA

SEQ ID NO: 147 - protein, 12 aa, Synthetic construct
RASQSVSSSYLA

SEQ ID NO: 148 - protein, 12 aa, Synthetic construct
RASQSVSSSYLA

SEQ ID NO: 149 - protein, 12 aa, Synthetic construct
RASQSVSRSYLA

SEQ ID NO: 150 - protein, 7 aa, Synthetic construct
XASXRAT

SEQ ID NO: 151 - protein, 7 aa, Synthetic construct
DASNRAT

SEQ ID NO: 152 - protein, 7 aa, Synthetic construct
DASNRAT

SEQ ID NO: 153 - protein, 7 aa, Synthetic construct
DASNRAT

SEQ ID NO: 154 - protein, 7 aa, Synthetic construct
GASSRAT

SEQ ID NO: 155 - protein, 7 aa, Synthetic construct
GASSRAT

SEQ ID NO: 156 - protein, 7 aa, Synthetic construct
GASSRAT

SEQ ID NO: 157 - protein, 7 aa, Synthetic construct
GASSRAT

SEQ ID NO: 158 - protein, 7 aa, Synthetic construct
GASSRAT

SEQ ID NO: 159 - protein, 7 aa, Synthetic construct
GASTRAT

SEQ ID NO: 160 - skipped

SEQ ID NO: 161 - protein, 9 aa, Synthetic construct
QQRSNWPLT

SEQ ID NO: 162 - protein, 9 aa, Synthetic construct
QQRSNWPLT

SEQ ID NO: 163 - protein, 8 aa, Synthetic construct
QQRSNWPS

SEQ ID NO: 164 - protein, 9 aa, Synthetic construct
QQYGSSPPT

SEQ ID NO: 165 - protein, 8 aa, Synthetic construct
QQYGSSLT

SEQ ID NO: 166 - protein, 9 aa, Synthetic construct
QQYGSSPPT

SEQ ID NO: 167 - protein, 9 aa, Synthetic construct
QQYGSSPPT

SEQ ID NO: 168 - protein, 9 aa, Synthetic construct
QQYGSSPPT

SEQ ID NO: 169 - protein, 9 aa, Synthetic construct
QQGQVIPPT

SEQ ID NO: 170 - protein, 124 aa, Synthetic construct
QVQLVQSGAEVKKPGASVKVSCKASGYTFSSYGISWVRQAPGQGLEWMGWISAYNGNTNYAQNLRGRVAMTTDTSTTTAYMELRSLRSDDTAVYYCARVSLVWFGELPRGFDYWGQGTLVTVSS

SEQ ID NO: 171 - protein, 117 aa, Synthetic construct
QVQLVQSGAEVKKPGASVKVSCKASGYTFTSYGISWVRQAPGQGLEWMGWIIAYNGNTNYAQKLQGRITMTTNTSTNTAYMELRSLRSDDTAVYYCGRDTGFSFDYWGQGTLVTVSS

SEQ ID NO: 172 - protein, 124 aa, Synthetic construct; identical to SEQ ID NO: 170

SEQ ID NO: 173 - protein, 121 aa, Synthetic construct
QVQLVQSGAEVKKPGASVKVSCKASGYTFTDYYIHWVRQAPGPGLEWLGWINPNTGATKYAQKFQGRVTMTRDTSISTAYMELSRLRSDDTAVYYWAREDNGNYGDVFDIWGQGTMVTVSS

SEQ ID NO: 174 - protein, 121 aa, Synthetic construct
QVQLVQSGAEVKKPGASVKVSCKASGYTFTDYYIHWVRQAPGPGLEWLGWINPNTGATKYAQKFQGRVTMTRDTSISTAYMELSRLRSDDTAVYYCAREDNGNYGDVFDIWGQGTMVTVSS

SEQ ID NO: 175 - DNA, 372 bp, Synthetic construct
  source 1..372: mol_type = other DNA
caggttcagctggtgcagtctggagctgaggtgaagaagcctggggcctcagtgaaggtctcctgcaaggcttctggttacaccttttccagctatggtatcagctgggtgcgacaggcccctggacaagggcttgagtggatgggatggatcagcgcttacaatggtaacacaaactatgcacagaacctccggggcagagtcgccatgaccacagacacatccacgaccacagcctacatggagttgaggagcctgagatctgacgacacggccgtgtattactgtgcgagagtttcattagtatggtttggggaattacccaggggttttgactactggggccagggaaccctggtcaccgtctcctca

SEQ ID NO: 176 - DNA, 351 bp, Synthetic construct
  source 1..351: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcatcgcttacaatggtaacacaaactatgcacagaagctccagggcagaatcaccatgaccacaaacacatccacgaacacagcctacatggagctgaggagcctgagatctgacgacacggccgtgtattactgtgggagagatacggggttttcttttgactactggggccagggaaccctggtcaccgtctcctca

SEQ ID NO: 177 - DNA, 372 bp, Synthetic construct; identical to SEQ ID NO: 175
  source 1..372: mol_type = other DNA

SEQ ID NO: 178 - DNA, 363 bp, Synthetic construct
  source 1..363: mol_type = other DNA
caggtgcagctggtgcagtctggggctgaggtgaagaagcctggggcctcagtgaaggtctcctgcaaggcttctggatacaccttcaccgactactatatacactgggtgcgacaggcccctgggccagggcttgagtggttgggctggatcaaccctaacactggtgccacaaagtatgcacagaagtttcagggcagggtcaccatgaccagggacacgtccatcagcacagcctatatggagctgagcaggctgagatctgacgacacggccgtatattattgggcgagagaagataatgggaactacggggatgtttttgatatctggggacaagggacaatggtcaccgtctcttca

SEQ ID NO: 179 - DNA, 363 bp, Synthetic construct
  source 1..363: mol_type = other DNA
caggtgcagctggtgcagtctggggctgaggtgaagaagcctggggcctcagtgaaggtctcctgcaaggcttctggatacaccttcaccgactactatatacactgggtgcgacaggcccctgggccagggcttgagtggttgggctggatcaaccctaacactggtgccacaaagtatgcacagaagtttcagggcagggtcaccatgaccagggacacgtccatcagcacagcctatatggagctgagcaggctgagatctgacgacacggccgtatattattgtgcgagagaagataatgggaactacggggatgtttttgatatctggggacaagggacaatggtcaccgtctcttca

SEQ ID NO: 180 - protein, 108 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTFGGGTKVEIK

SEQ ID NO: 181 - protein, 107 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVDIK

SEQ ID NO: 182 - protein, 112 aa, Synthetic construct
DIVMTQSPLSLPVTPGEPASISCRSSQSLLHSNRYNYLDWYLQKPGQSPQLLIYLGSNRASGVPDRFSGSGSGTDFTLQISRVEAEDVGVYYCMQTLQTPYTFGQGTKLEIK

SEQ ID NO: 183 - protein, 108 aa, Synthetic construct; identical to SEQ ID NO: 180

SEQ ID NO: 184 - protein, 108 aa, Synthetic construct; identical to SEQ ID NO: 180

SEQ ID NO: 185 - DNA, 324 bp, Synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccactttcggcggagggaccaaggtggagatcaaa

SEQ ID NO: 186 - DNA, 321 bp, Synthetic construct
  source 1..321: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactcactttcggcggagggaccaaagtggatatcaaa

SEQ ID NO: 187 - DNA, 336 bp, Synthetic construct
  source 1..336: mol_type = other DNA
gatattgtgatgactcagtctccactctccctgcccgtcacccctggagagccggcctccatctcctgcaggtctagtcagagcctcctgcatagtaatagatacaactatttggattggtacctgcagaagccagggcagtctccacagctcctgatctatttgggttctaatcgggcctccggggtccctgacaggttcagtggcagtggatcaggcacagattttacactgcaaatcagcagagtggaggctgaggatgttggggtttattactgcatgcaaactctacaaactccgtacacttttggccaggggaccaagctggagatcaaa

SEQ ID NO: 188 - DNA, 324 bp, Synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccactttcggcggagggaccaaggtggaaatcaaa

SEQ ID NO: 189 - DNA, 324 bp, Synthetic construct; identical to SEQ ID NO: 188
  source 1..324: mol_type = other DNA

SEQ ID NO: 190 - protein, 120 aa, Synthetic construct
QITLKESGPTLVKPTQTLTLTCTFSGFSLSTSGVGVGWIRQPPGKALEWLALIYWNDDKQYSPSLKSRLTITKDTSKNQVVLTMTNMDPMDTATYYCAHNPDYGDFFDYWGQGTLVTVSS

SEQ ID NO: 191 - protein, 120 aa, Synthetic construct
QITLKESGPTLVKPTQTLTLTCTFSGFSLSTSGVGVGWIRQPPGKALEWLALIYWNDDKRYSPSLKSRLTITKDTSKNLVVLTMTNMDPVDTATYYCARDYGDYWFFDLWGRGTPVTVSS

SEQ ID NO: 192 - DNA, 360 bp, Synthetic construct
  source 1..360: mol_type = other DNA
cagatcaccttgaaggagtctggtcctacgctggtgaaacccacacagaccctcacgttgacctgcaccttctctgggttctcactcagcactagtggcgtgggtgtgggctggatccgtcagcccccaggaaaggccctggagtggcttgcactcatttattggaatgatgataaacagtacagcccttctctgaagagcaggctcaccatcaccaaggacacctccaaaaaccaggtggtccttacaatgaccaacatggaccctatggacacagccacatattactgtgcacacaaccctgactacggtgacttctttgactactggggccagggaaccctggtcaccgtctcctca

SEQ ID NO: 193 - DNA, 360 bp, Synthetic construct
  source 1..360: mol_type = other DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaatgatgataagcgctacagcccatctctgaagagcaggctcaccatcaccaaggacacctccaaaaacctggtggtccttacaatgaccaacatggaccctgtggacacagccacatattactgtgcacgtgactacggtgactactggttcttcgatctctggggccgtggcaccccggtcaccgtctcctca

SEQ ID NO: 194 - protein, 107 aa, Synthetic construct
DIQMTQSPSSLSASVGDRVTITCRASQSISSYLNWYQQKPGKAPKLLIYAASSLQSGVPSRFSGSGSGTDFILTISSLQPEDFATYYCQQSFSTPLTFGGGTKLEIK

SEQ ID NO: 195 - protein, 107 aa, Synthetic construct
DIQMTQSPSSLSASVGDRVTITCRASQGISSYLAWYQQKPGKAPKLLIYAASSLQSGVPSRFSGSGSGTDFTLTISSLQPEDFATYYCQQSFSTPLTFGGGTKLEIK

SEQ ID NO: 196 - DNA, 321 bp, Synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcattctcaccatcagcagtctgcaacctgaagattttgcaacttactactgtcaacagagtttcagtaccccgctcactttcggcggagggaccaagctggaaatcaaa

SEQ ID NO: 197 - DNA, 321 bp, Synthetic construct
  source 1..321: mol_type = other DNA
gacatccagatgacccagtctccatcctccctgtctgcatctgtaggagacagagtcaccatcacttgccgggccagtcagggcattagcagttatttagcctggtatcagcaaaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagtttcagtaccccgctcactttcggcggagggaccaagctggagatcaaa

SEQ ID NO: 198 - protein, 121 aa, Synthetic construct
EVQLVESGGGLVQPGGSLRLSCAASGFTLSVYDMHWVRQVSGKGLEWVSGIDPDGDTYYPGSVKGRFTISRENAKNSLYLQMNSLRVGDTAVYYCLRGEESNSSEDGFDIWGQGTMVTVSS

SEQ ID NO: 199 - protein, 121 aa, Synthetic construct
EVQLVESGGGLVQPGGSLRLSCAASGFTFSSHDMHWVRQVTGKGLEWVSGIDPAGDTYYPDSVKGRFTISRENAKNSLYLQMNSLRAGDTAVYYCTRGEASSSSEDAFDIWGQGTMVTVSS

SEQ ID NO: 200 - protein, 121 aa, Synthetic construct
EVQLVESGGGLVQPGGSLRLSCAASGFTLSSYDMHWVRQVIGKGLEWVSGIDPDGDTYYPGSVKGRFTISREDAKNSLYLQMNSLRAGDTAVYYCTRGEDNSSSEDAFDIWGQGTMVTVSS

SEQ ID NO: 201 - protein, 121 aa, Synthetic construct
EVQLVESGGGLVQPGGSLRLSCAASGFTISDYDMHWVRQTTGKGLEWVSGIDPDGDTYYPGSLKGRFTISRENAKNSLYLQMNSLRAGDTAVYYCTRGEDSSSSEDAFDIWGQGTMVTVSS

SEQ ID NO: 202 - protein, 120 aa, Synthetic construct
EVQLVESGGGVVRPGGSLRLSCAASGFSFDDYGMSWVRQVPGKGLEWVSGINWNGGSTGYADSVKGRFTISRDNAKNSLYLQMSSLRAEDTALYYCASYSGSFRDAFDIWGQGTMVTVSS

SEQ ID NO: 203 - protein, 122 aa, Synthetic construct
EVQLVESGGGVVRPGGSLRLSCAASGFTFDDYGMSWVRQAPGKGLEWVSTINWNGGNTGYADSVKGRFTISRDNAKNSLYLQMNSLRAEDTALYYCARDEGAAGLGYYFDYWGQGTLVTVSS

SEQ ID NO: 204 - protein, 118 aa, Synthetic construct
EVQLVESGGGLVKPGGSLRLSCAASGFTFSSYSMNWVRQAPGKGLEWVSSITSSSYIFYADSLKGRFTISRDNAKNSLFLQMNSLRAEDTAVYYCARDSNWGEAFDIWGQGTMVTVSS

SEQ ID NO: 205 - protein, 119 aa, Synthetic construct
EVQLVESGGGLVKPGGSLRLSCAASGFTFSTYSMNWVRQAPGKGLEWVSSITSSSSYMFYADSVMGRFTISRDNAKNSLYLQMNSLRAEDTADYYCARDSNWGEAFDIWGQGTMVTVSS

SEQ ID NO: 206 - protein, 120 aa, Synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGDYYYYYMDVWGKGTTVTVSS

SEQ ID NO: 207 - protein, 125 aa, Synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVERDMVRGHYYYYMDVWGKGTTVTVSS

SEQ ID NO: 208 - protein, 121 aa, Synthetic construct
QVQLVESGGGVVQPGRSLRLSCAASGFTFSSYGMHWVRQAPGKGLEWVSHITYDGSDKYYGDSVKGRFTISRDNSKNTLYLQMNSLRAEDTAVYYCAKDGYSSGWWYFDYWGQGTLVTVSS

SEQ ID NO: 209 - protein, 126 aa, Synthetic construct
QVQLVESGGGVVQPGRSLRLSCAASGFTFSSYGMHWVRQAPGKGLEWVAIISYDGNNKYYADSVKGRFTISRDNSKNTLYLQMNSLRDEDTAVYYCAKERGYSSSSGYYYYYMDVWGKGTTVTVSS

SEQ ID NO: 210 - protein, 119 aa, Synthetic construct
QVQLVESGGGVVQPGRSLRLSCAASGFPFSSYGMHWVRQAPGKGLEWVAVMSYDGINGYYADSVKGRFTISRDNSKNTLYLQMNSLRAEDTAVYYCARLVYADDAFDIWGQGTTVTVSS

SEQ ID NO: 211 - protein, 115 aa, Synthetic construct
QVQLVESGGGVVQPGRSLRLSCAASGFAFSSYGMHWVRQAPGKGLEWVAVISYYGSNKYYADSVKGRFTISRDNSKNTLYLQMNSLRAEDTAVYHCARGITMDVWGKGTTVTVSS

SEQ ID NO: 212 - protein, 120 aa, Synthetic construct
EVQLVESGGGLVQPGRSLRLSCAASGFTFDDFAMHWVRQGPGKGLEWVSGIGWNSGSIGYADSVKGRFTISRDNAKNSLYLQMNSLRAEDTALYYCAKAPDYGDYYFDYWGQGTLVTVSS

SEQ ID NO: 213 - DNA, 363 bp, Synthetic construct
  source 1..363: mol_type = other DNA
gaggtgcagctggtggagtcggggggaggcttggtacagcctggggggtccctgagactctcctgtgcagcctctggattcaccctcagtgtctacgacatgcactgggtccgccaagtttcaggaaaaggtctggagtgggtctcaggtattgatcctgatggtgacacatactatcccggctccgtgaagggccgattcaccatctccagagaaaatgccaagaactccttgtatcttcaaatgaacagcctgagagtcggggacacggctgtgtattattgtctaagaggggaggaaagcaactcgtccgaggatggttttgatatatggggacaagggacaatggtcaccgtctcctca

SEQ ID NO: 214 - DNA, 363 bp, Synthetic construct
  source 1..363: mol_type = other DNA
gaggtgcagctggtggagtctgggggaggcttggtacagcctggggggtccctgagactctcctgtgcagcctctggattcaccttcagtagccacgacatgcactgggtccgccaagttacaggaaaaggtctggagtgggtctcaggtattgatcctgctggtgacacatactatccagactccgtgaagggccgattcaccatctctagagaaaatgccaagaactccttgtatcttcaaatgaacagcctgagagccggggacacggctgtgtattactgtacaagaggagaggctagtagttcgtccgaggatgcttttgatatctggggacaagggacaatggtcactgtctcctca

SEQ ID NO: 215 - DNA, 363 bp, Synthetic construct
  source 1..363: mol_type = other DNA
gaggtgcagctggtggagtctgggggaggcttggtacagcctggggggtccctgagactctcctgtgcagcctctggattcaccctcagtagctacgacatgcactgggtccgccaagttataggaaaaggtctggagtgggtctcaggtattgatcctgatggtgacacatactatccaggctccgtgaagggccgattcaccatctccagagaagatgccaagaactccttgtatcttcaaatgaacagcctgagagccggggacacggctgtgtattactgtacaagaggagaggataacagctcgtccgaggatgcttttgatatctggggacaagggacaatggtcactgtctcctca

SEQ ID NO: 216 - DNA, 363 bp, Synthetic construct
  source 1..363: mol_type = other DNA
gaggtccagctggtggagtctgggggaggcttggtacagccgggggggtccctgagactctcctgtgcagcctctggattcaccatcagtgactacgacatgcactgggtccgccaaactacaggaaaaggtctagaatgggtctcaggtattgatcctgatggcgacacatattatccaggctccttgaaggggcgattcaccatctccagagaaaatgccaagaattctttgtatcttcaaatgaacagtctgagagccggggacacggctgtgtattactgtacaagaggagaggatagtagttcgtccgaggatgcttttgatatctggggacaagggacaatggtcaccgtctcctca

SEQ ID NO: 217 - DNA, 360 bp, Synthetic construct
  source 1..360: mol_type = other DNA
gaggtgcagctggtggagtctgggggaggtgtggtacggcctggggggtccctgagactctcctgtgcagcctctggattcagctttgatgattatggcatgagctgggtccgccaagttccagggaaggggctggagtgggtctctggtattaattggaatggtggtagtacaggttatgcagactctgtgaagggccgattcaccatctccagagacaacgccaagaactccctgtatctgcaaatgagcagtctgagagccgaggacacggccttgtattactgtgcgagctatagtgggagcttccgtgatgcttttgatatctggggacaagggacaatggtcaccgtctcttca

SEQ ID NO: 218 - DNA, 366 bp, Synthetic construct
  source 1..366: mol_type = other DNA
gaggtgcagctggtggagtctgggggaggtgtggtacggcctggggggtccctgagactctcctgtgcagcctctggattcacctttgatgattatggcatgagctgggtccgccaagctccagggaaggggctggagtgggtctctactattaattggaatggtggtaacacaggttatgcagactctgtgaagggccgattcaccatctccagagacaacgccaagaactccctgtatctgcaaatgaacagtctgagagccgaggacacggccttgtattactgtgcgagagatgaaggagcagctggacttggatactactttgactactggggccagggaaccctggtcaccgtctcctca

SEQ ID NO: 219 - DNA, 354 bp, Synthetic construct
  source 1..354: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattactagtagtagttacatattctacgcagactcattgaagggccgattcaccatctccagagacaacgccaagaactcactgtttctgcaaatgaacagcctgagagccgaggacacagctgtttattactgtgcgagagattctaactggggcgaggcttttgatatctggggacaagggacaatggtcaccgtctcttca

SEQ ID NO: 220 - DNA, 357 bp, Synthetic construct
  source 1..357: mol_type = other DNA
gaggtgcagctggtggagtctgggggaggcctggtcaagcctggggggtccctgagactctcctgtgcagcctctggtttcaccttcagtacttatagcatgaactgggtccgccaggctccagggaaggggctggagtgggtctcatccattactagtagtagtagttacatgttctacgcagactcagtgatgggccgattcaccatctccagagacaacgccaagaactcactgtacctacaaatgaacagcctgagagccgaggacacagctgattattactgtgcgagagattctaattggggggaggcttttgatatctggggacaagggacaatggtcaccgtctcttca

SEQ ID NO: 221 - DNA, 360 bp, Synthetic construct
  source 1..360: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagacggtgactactactactactacatggacgtctggggcaaagggaccacggtcaccgtctcctca

SEQ ID NO: 222 - DNA, 375 bp, Synthetic construct
  source 1..375: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagtggaaagggatatggttcggggccactactactactacatggacgtctggggcaaagggaccacggtcaccgtctcttca

SEQ ID NO: 223 - DNA, 363 bp, Synthetic construct
  source 1..363: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtgtcacatataacatatgatggaagtgataaatactatggagactccgtgaagggccgattcaccatctccagagacaattccaagaatacgctgtatctgcaaatgaacagcctgagagctgaggacacggctgtgtattactgtgcgaaagatgggtatagcagtggctggtggtactttgactactggggccagggaaccctggtcaccgtctcctca

SEQ ID NO: 224 - DNA, 378 bp, Synthetic construct
  source 1..378: mol_type = other DNA
caggtgcagctggtggagtccgggggaggcgtggtccagcctgggaggtccctgagactctcctgtgcagcctctggattcaccttcagtagctatggcatgcactgggtccgccaggctccaggcaaggggctggagtgggtggcaattatatcatatgatggaaataataaatactatgcagactccgtgaagggccgattcaccatctccagagacaattccaagaacacgctgtatctgcaaatgaacagcctgagagatgaggacacggctgtttattattgtgcgaaagagagggggtatagcagctcgtccggctactactactactacatggacgtctggggcaaagggaccacggtcaccgtctcctca

SEQ ID NO: 225 - DNA, 357 bp, Synthetic construct
  source 1..357: mol_type = other DNA
caagtgcaattggtggagtccggtggaggagtagtacagccgggaaggtcactgagactttcctgcgctgctagtggattcccatttagctcctacgggatgcactgggttcgccaagcgccaggtaagggactggaatgggtggctgtcatgagttacgatggaattaacggctattatgccgatagcgtcaaaggccggttcacgatctcaagagacaactctaagaacacgctctacttgcagatgaacagtctccgcgcagaagatacggccgtgtattactgtgcgagattggtgtatgccgatgatgcttttgatatctggggacaagggaccacggtcaccgtctcctca

SEQ ID NO: 226 - DNA, 345 bp, Synthetic construct
  source 1..345: mol_type = other DNA
caagtgcaattggtggagtccggtggaggagtagtacagccgggaaggtcactgagactttcctgcgctgctagtggattcgcctttagttcctacggcatgcactgggttcgccaagcgccaggtaagggactggaatgggtggctgtcatctcctactacggatctaataaatattatgccgatagcgtcaaaggccggttcacgatctcaagagacaactctaagaacacgctctacttgcagatgaacagtctccgcgcagaagatacggccgtgtatcactgtgcgagagggattactatggacgtctggggcaaagggaccacggtcaccgtctcctca

SEQ ID NO: 227 - DNA, 360 bp, Synthetic construct
  source 1..360: mol_type = other DNA
gaagtgcagctggtggagtctgggggaggcttggtacagcctggcaggtccctgagactctcctgtgcagcctctggattcacctttgatgattttgccatgcactgggtccggcaaggtccagggaagggcctggagtgggtctcaggtattggttggaatagtggtagcatcggctatgcggactctgtgaagggccgattcaccatctccagagacaacgccaagaactccctgtatctgcaaatgaacagtctgagagctgaggacacggccttatattactgtgcaaaagcccctgactacggtgactactactttgactactggggccagggaaccctggtcaccgtctcctca

SEQ ID NO: 228 - protein, 112 aa, Synthetic construct
DVVMTQSPLSLPVTLGQPASISCRSSQSLVYSDGNTYLNWFQQRPGQSPRRLIYKVSNRDSGVPDRFSGSGSGTDFTLKISKVEAEDVGIYYCMQGTHWPWTFGQGTKVDIK

SEQ ID NO: 229 - protein, 112 aa, Synthetic construct
DVVMTQSPLSLPVTLGQPASISCRSSQSLVYSDGNTYLNWFQQRPGQSPRRLIHEVSNRDSGVPDRFSGSGSGTDFTLKISRVEAEDVGVYYCMQGTHWPWTFGQGTKVDIK

SEQ ID NO: 230 - protein, 112 aa, Synthetic construct
DVVMTQSPLSLPVTLGQPASISCRSSQSLVFSDGNTYLNWFQQRPGQSPRRLIYKVSNRDSGVPDRFSGSGSGTDFTLKISRVEAEDVGVYYCMQGTHWPWTFGQGTKVDIK

SEQ ID NO: 231 - protein, 112 aa, Synthetic construct; identical to SEQ ID NO: 229

SEQ ID NO: 232 - protein, 107 aa, Synthetic construct
AIQMTQSPSSLSASVGDRVTITCRASQGIRNDLGWYQQKPGKAPKLLIYAASSLQSGVPSRFSGSGSGTDFTLTISSLQPEDFATYYCLQDYNYPYTFGQGTKLEIK

SEQ ID NO: 233 - protein, 107 aa, Synthetic construct
DIQMTQSPSTLSASVGDRVTITCRASQSISSWLAWYQQKPGKAPKLLIYATSSLQSGVPSRFSGSGSGTDFTLTISSLQPEDFATYYCQQSYSTPITFGQGTRLEIK

SEQ ID NO: 234 - protein, 107 aa, Synthetic construct
DIQMTQSPSSLSASVGDRVTITCRASQGIRNDLGWYQQKPGKAPKRLIYAASSLPSGVPSRFSGSGSGTEFTLTISSLQPEDFAIYYCLQHNSYPWTFGQGTKVEIK

SEQ ID NO: 235 - protein, 107 aa, Synthetic construct
DIQMTQSPSSLSASVGDRVTITCRASQGIRIDLGWFQQKPGKAPKRLIYAASSLPSGVPSRFSGSGSGTEFTLTISSLQPEDFATYYCLQHNSYPWTFGQGTKVEIK

SEQ ID NO: 236 - protein, 108 aa, Synthetic construct; identical to SEQ ID NO: 180

SEQ ID NO: 237 - protein, 107 aa, Synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPWTFGQGTKVDIK

SEQ ID NO: 238 - protein, 107 aa, Synthetic construct
DIQMTQSPSSLSASVGDRVTITCRASQGIRNDLGWYQQKPGKAPKRLIYAASSLQSGVPSRFSGSGSGTEFALTISSLQPEDFATYYCLQHNSYPLTFGGGTKLEIK

SEQ ID NO: 239 - protein, 108 aa, Synthetic construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 240 - protein, 107 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 241 - protein, 107 aa, Synthetic construct; identical to SEQ ID NO: 240

SEQ ID NO: 242 - protein, 107 aa, Synthetic construct
DIQMTQSPSSLSASVGDRVTITCRASQSISIYLNWYQQKPGTAPKLLIYAASSLQSGVPSRFSGSGSGTDFTLTISSLQPEDFAAYYCQQSYTTPLTFGGGTKVEIK

SEQ ID NO: 243 - DNA, 336 bp, Synthetic construct
  source 1..336: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcaaggtggaggctgaggatgttgggatttattactgcatgcaaggtacacactggccgtggacgttcggccaagggaccaaagtggatatcaaa

SEQ ID NO: 244 - DNA, 336 bp, Synthetic construct
  source 1..336: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaattcatgaggtttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaagtggatatcaaa

SEQ ID NO: 245 - DNA, 336 bp, Synthetic construct
  source 1..336: mol_type = other DNA
gatgttgtgatgactcagtctccactctccctgcccgtcacccttggacagccggcctccatctcctgcaggtctagtcaaagcctcgtattcagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaagtggatatcaaa

SEQ ID NO: 246 - DNA, 336 bp, Synthetic construct; identical to SEQ ID NO: 244
  source 1..336: mol_type = other DNA

SEQ ID NO: 247 - DNA, 321 bp, Synthetic construct
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgcaacttattactgtctacaagattacaattacccgtacacttttggccaggggaccaagctggagatcaaa

SEQ ID NO: 248 - DNA, 321 bp, Synthetic construct
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgctacatccagtttgcaaagtggggtcccatcaaggttcagtggcagtggatctgggacagatttcactctcaccatcagcagtctgcaacctgaagattttgcaacttactactgtcaacagagttacagtaccccgatcaccttcggccaagggacacgactggagattaaa

SEQ ID NO: 249 - DNA, 321 bp, Synthetic construct
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgccaagtggggtcccatcaaggttcagcggcagtggatctgggacagaattcactctcacaatcagcagcctgcagcctgaagattttgcaatttattactgtctacagcataatagttacccgtggacgttcggccaagggaccaaggtggaaatcaaa

SEQ ID NO: 250 - DNA, 321 bp, Synthetic construct
  source 1..321: mol_type = other DNA
gacatccagatgacccagtctccatcctccctgtccgcatctgtaggagacagagtcaccatcacttgccgggcaagtcagggcattagaattgatttaggctggtttcagcagaaaccagggaaagcccctaagcgcctcatctatgctgcatccagtttgccaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggagatcaaa

SEQ ID NO: 251 - DNA, 324 bp, Synthetic construct; identical to SEQ ID NO: 188
  source 1..324: mol_type = other DNA

SEQ ID NO: 252 - DNA, 321 bp, Synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgtggacgttcggccaagggaccaaagtggatatcaaa

SEQ ID NO: 253 - DNA, 321 bp, Synthetic construct
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcgctctcacaatcagcagcctgcagcctgaagattttgcaacttattactgtctacagcataatagttaccctctcactttcggcggagggaccaagctggagatcaaa

SEQ ID NO: 254 - DNA, 324 bp, Synthetic construct
  source 1..324: mol_type = other DNA
gaaattgtgctgactcagtctcccggcacactgtctcttagccctggcgaaagagccacactgagctgtagagccagccagagcgtgtccagaagctacctggcttggtatcagcagaagcccggacaggctcccagactgctgatcatcggagcctctacaagagccaccggcattcccgatagattcagcggctctggcagcggcaccgatttcaccctgacaatcagcagactggaacccgaggacttcgccgtgtactactgtcagcagggccaagtgatccctcctacctttggccagggcaccaaggtggaaatcaaa

SEQ ID NO: 255 - DNA, 321 bp, Synthetic construct
  source 1..321: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 256 - DNA, 321 bp, Synthetic construct; identical to SEQ ID NO: 255
  source 1..321: mol_type = other DNA

SEQ ID NO: 257 - DNA, 321 bp, Synthetic construct
  source 1..321: mol_type = other DNA
gacatccagatgacccagtctccatcctccctgtctgcatctgtaggagacagagtcaccatcacttgccgggcaagtcagagcattagcatctatttaaattggtatcagcagaaaccagggacagcccctaagctcctgatctatgctgcatccagtttgcaaagtggggtcccatcaaggttcagtggcagtggatctgggacagatttcactctcaccatcagcagtctgcaacctgaagattttgcagcttactactgtcaacagagttacactaccccgctcactttcggcggagggaccaaggtggaaatcaaa

SEQ ID NO: 258 - protein, 121 aa, Synthetic construct
QLQLQESGPGLVKPSETLSLTCTVSGGSISSSSSYWGWIRQPPGKGLEWIGSIYYSGSTYSNPSLKSRVTISVDTSKNQFSLKLSSVTAADTAVYYCARQYISGTEYFQYWGQGTLVTVSS

SEQ ID NO: 259 - protein, 121 aa, Synthetic construct; identical to SEQ ID NO: 258

SEQ ID NO: 260 - protein, 120 aa, Synthetic construct
QVQLQESGPGLVKPSETLSLTCTVSGGSVSSGSYYWSWIRQPPGKGLEWIGYIYYSGSTSYNPSLKSRVSISVDTSKNQFSLKLSSVTAADTAVFYCARAKWEGDYFDYWGQGTLVTVSS

SEQ ID NO: 261 - DNA, 363 bp, Synthetic construct
  source 1..363: mol_type = other DNA
cagctgcagctgcaggagtcgggcccaggactggtgaagccttcggagaccctgtccctcacctgcactgtctctggtggttccatcagcagtagtagttcctactggggctggatccgccagcccccagggaagggactggagtggattgggagtatctattatagtgggagcacctactccaacccgtccctcaagagtcgagtcaccatatccgtagacacgtccaagaatcagttctccctgaagctgagctctgtgaccgccgcagacacggctgtgtattactgtgcgagacagtatataagtggaactgaatacttccagtactggggccagggcaccctggtcaccgtctcctca

SEQ ID NO: 262 - DNA, 363 bp, Synthetic construct; identical to SEQ ID NO: 261
  source 1..363: mol_type = other DNA

SEQ ID NO: 263 - DNA, 360 bp, Synthetic construct
  source 1..360: mol_type = other DNA
caggtgcagctgcaggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccagctacaacccctccctcaagagtcgagtctccatatcagtagacacgtccaagaaccagttctccctgaagctgagctctgtgaccgctgcggacacggccgtgttttactgtgcgagagctaagtgggaaggggactactttgactactggggccagggaaccctggtcaccgtctcctca

SEQ ID NO: 264 - protein, 106 aa, Synthetic construct
EIVLTQSPATLSLSPGERATLSCRASQSVSNYLAWYQQKPGQAPRLLIYDASNRATGIPARFSGSGSGTDFTLTISSLEPEDFAVYYCQQRSNWPSFGQGTRLEIK

SEQ ID NO: 265 - protein, 107 aa, Synthetic construct [sequence omitted]

SEQ ID NO: 266 - protein, 108 aa, Synthetic construct
DIQMTQSPSSLSASVGDRVTITCRASQSIYSYLNWYQQKPGKAPNHLIYAASSLQSGVPSRFSGSGSGTDFTLTISSLHPEDFATYYCQQSYSTPPYTFGQGTKVEIK

SEQ ID NO: 267 - DNA, 318 bp, Synthetic construct
  source 1..318: mol_type = other DNA
gaaattgtgttgacacagtctccagccaccctgtctttgtctccaggggaaagagccaccctctcctgcagggccagtcagagtgttagcaactacttagcctggtaccaacagaaacctggccaggctcccaggctcctcatctatgatgcatccaacagggccactggcatcccagccagattcagtggcagtgggtctgggacagacttcactctcaccatcagcagcctggagcctgaagattttgcagtttattactgtcagcagcgtagcaactggccctccttcggccaagggacacgactggagattaaa

SEQ ID NO: 268 - DNA, 321 bp, Synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggaaatcaaa

SEQ ID NO: 269 - DNA, 324 bp, Synthetic construct
  source 1..324: mol_type = other DNA
gacatccagatgacccagtctccatcctccctgtctgcatctgtaggagacagagtcaccatcacttgccgggcaagtcagagcatttacagctatttaaattggtatcagcagaaaccagggaaagcccctaatcacctgatctatgctgcatccagtttgcaaagtggggtcccatcaaggttcagtggcagtggatctgggacagatttcactctcaccatcagcagtctgcaccctgaagattttgcaacttactactgtcaacagagttacagtacccctccgtacacttttggccaggggaccaaggtggaaatcaaa

SEQ ID NO: 270 - protein, 453 aa, Synthetic construct
QVQLVQSGAEVKKPGASVKVSCKASGYTFSSYGISWVRQAPGQGLEWMGWISAYNGNTNYAQNLRGRVAMTTDTSTTTAYMELRSLRSDDTAVYYCARVSLVWFGELPRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 271 - protein, 446 aa, Synthetic construct
QVQLVQSGAEVKKPGASVKVSCKASGYTFTSYGISWVRQAPGQGLEWMGWIIAYNGNTNYAQKLQGRITMTTNTSTNTAYMELRSLRSDDTAVYYCGRDTGFSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 272 - protein, 453 aa, Synthetic construct; identical to SEQ ID NO: 270

SEQ ID NO: 273 - protein, 450 aa, Synthetic construct
QVQLVQSGAEVKKPGASVKVSCKASGYTFTDYYIHWVRQAPGPGLEWLGWINPNTGATKYAQKFQGRVTMTRDTSISTAYMELSRLRSDDTAVYYWAREDNGNYGDVFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 274 - protein, 450 aa, Synthetic construct
QVQLVQSGAEVKKPGASVKVSCKASGYTFTDYYIHWVRQAPGPGLEWLGWINPNTGATKYAQKFQGRVTMTRDTSISTAYMELSRLRSDDTAVYYCAREDNGNYGDVFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 275 - DNA, 1359 bp, Synthetic construct
  source 1..1359: mol_type = other DNA
caggttcagctggtgcagtctggagctgaggtgaagaagcctggggcctcagtgaaggtctcctgcaaggcttctggttacaccttttccagctatggtatcagctgggtgcgacaggcccctggacaagggcttgagtggatgggatggatcagcgcttacaatggtaacacaaactatgcacagaacctccggggcagagtcgccatgaccacagacacatccacgaccacagcctacatggagttgaggagcctgagatctgacgacacggccgtgtattactgtgcgagagtttcattagtatggtttggggaattacccaggggttttgactactggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 276 - DNA, 1338 bp, Synthetic construct
  source 1..1338: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcatcgcttacaatggtaacacaaactatgcacagaagctccagggcagaatcaccatgaccacaaacacatccacgaacacagcctacatggagctgaggagcctgagatctgacgacacggccgtgtattactgtgggagagatacggggttttcttttgactactggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 277 - DNA, 1359 bp, Synthetic construct; identical to SEQ ID NO: 275
  source 1..1359: mol_type = other DNA

SEQ ID NO: 278 - DNA, 1350 bp, Synthetic construct
  source 1..1350: mol_type = other DNA
caggtgcagctggtgcagtctggggctgaggtgaagaagcctggggcctcagtgaaggtctcctgcaaggcttctggatacaccttcaccgactactatatacactgggtgcgacaggcccctgggccagggcttgagtggttgggctggatcaaccctaacactggtgccacaaagtatgcacagaagtttcagggcagggtcaccatgaccagggacacgtccatcagcacagcctatatggagctgagcaggctgagatctgacgacacggccgtatattattgggcgagagaagataatgggaactacggggatgtttttgatatctggggacaagggacaatggtcaccgtctcttcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 279 - DNA, 1350 bp, Synthetic construct
  source 1..1350: mol_type = other DNA
caggtgcagctggtgcagtctggggctgaggtgaagaagcctggggcctcagtgaaggtctcctgcaaggcttctggatacaccttcaccgactactatatacactgggtgcgacaggcccctgggccagggcttgagtggttgggctggatcaaccctaacactggtgccacaaagtatgcacagaagtttcagggcagggtcaccatgaccagggacacgtccatcagcacagcctatatggagctgagcaggctgagatctgacgacacggccgtatattattgtgcgagagaagataatgggaactacggggatgtttttgatatctggggacaagggacaatggtcaccgtctcttcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 280 - protein, 215 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 214 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 219 aa, Synthetic construct
DIVMTQSPLSLPVTPGEPASISCRSSQSLLHSNRYNYLDWYLQKPGQSPQLLIYLGSNRASGVPDRFSGSGSGTDFTLQISRVEAEDVGVYYCMQTLQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 215 aa, Synthetic construct; identical to SEQ ID NO: 280

SEQ ID NO: 284 - protein, 215 aa, Synthetic construct; identical to SEQ ID NO: 280

SEQ ID NO: 285 - DNA, 645 bp, Synthetic construct
  source 1..645: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccactttcggcggagggaccaaggtggagatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 286 - DNA, 642 bp, Synthetic construct
  source 1..642: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactcactttcggcggagggaccaaagtggatatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 287 - DNA, 657 bp, Synthetic construct
  source 1..657: mol_type = other DNA
gatattgtgatgactcagtctccactctccctgcccgtcacccctggagagccggcctccatctcctgcaggtctagtcagagcctcctgcatagtaatagatacaactatttggattggtacctgcagaagccagggcagtctccacagctcctgatctatttgggttctaatcgggcctccggggtccctgacaggttcagtggcagtggatcaggcacagattttacactgcaaatcagcagagtggaggctgaggatgttggggtttattactgcatgcaaactctacaaactccgtacacttttggccaggggaccaagctggagatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 288 - DNA, 645 bp, Synthetic construct
  source 1..645: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcccactttcggcggagggaccaaggtggaaatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 289 - DNA, 645 bp, Synthetic construct; identical to SEQ ID NO: 288
  source 1..645: mol_type = other DNA

SEQ ID NO: 290 - protein, 449 aa, Synthetic construct
QITLKESGPTLVKPTQTLTLTCTFSGFSLSTSGVGVGWIRQPPGKALEWLALIYWNDDKQYSPSLKSRLTITKDTSKNQVVLTMTNMDPMDTATYYCAHNPDYGDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 291 - protein, 449 aa, Synthetic construct
QITLKESGPTLVKPTQTLTLTCTFSGFSLSTSGVGVGWIRQPPGKALEWLALIYWNDDKRYSPSLKSRLTITKDTSKNLVVLTMTNMDPVDTATYYCARDYGDYWFFDLWGRGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 292 - DNA, 1347 bp, Synthetic construct
  source 1..1347: mol_type = other DNA
cagatcaccttgaaggagtctggtcctacgctggtgaaacccacacagaccctcacgttgacctgcaccttctctgggttctcactcagcactagtggcgtgggtgtgggctggatccgtcagcccccaggaaaggccctggagtggcttgcactcatttattggaatgatgataaacagtacagcccttctctgaagagcaggctcaccatcaccaaggacacctccaaaaaccaggtggtccttacaatgaccaacatggaccctatggacacagccacatattactgtgcacacaaccctgactacggtgacttctttgactactggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 293 - DNA, 1347 bp, Synthetic construct; identical to SEQ ID NO: 292
  source 1..1347: mol_type = other DNA

SEQ ID NO: 294 - protein, 214 aa, Synthetic construct
DIQMTQSPSSLSASVGDRVTITCRASQSISSYLNWYQQKPGKAPKLLIYAASSLQSGVPSRFSGSGSGTDFILTISSLQPEDFATYYCQQSFSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 214 aa, Synthetic construct
DIQMTQSPSSLSASVGDRVTITCRASQGISSYLAWYQQKPGKAPKLLIYAASSLQSGVPSRFSGSGSGTDFTLTISSLQPEDFATYYCQQSFSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - DNA, 642 bp, Synthetic construct
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcattctcaccatcagcagtctgcaacctgaagattttgcaacttactactgtcaacagagtttcagtaccccgctcactttcggcggagggaccaagctggaaatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 297 - DNA, 642 bp, Synthetic construct
  source 1..642: mol_type = other DNA
gacatccagatgacccagtctccatcctccctgtctgcatctgtaggagacagagtcaccatcacttgccgggccagtcagggcattagcagttatttagcctggtatcagcaaaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagtttcagtaccccgctcactttcggcggagggaccaagctggagatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 298 - protein, 450 aa, Synthetic construct
EVQLVESGGGLVQPGGSLRLSCAASGFTLSVYDMHWVRQVSGKGLEWVSGIDPDGDTYYPGSVKGRFTISRENAKNSLYLQMNSLRVGDTAVYYCLRGEESNSSEDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 299 - protein, 450 aa, Synthetic construct
EVQLVESGGGLVQPGGSLRLSCAASGFTFSSHDMHWVRQVTGKGLEWVSGIDPAGDTYYPDSVKGRFTISRENAKNSLYLQMNSLRAGDTAVYYCTRGEASSSSEDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 300 - protein, 450 aa, Synthetic construct
EVQLVESGGGLVQPGGSLRLSCAASGFTLSSYDMHWVRQVIGKGLEWVSGIDPDGDTYYPGSVKGRFTISREDAKNSLYLQMNSLRAGDTAVYYCTRGEDNSSSEDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 301 - protein, 450 aa, Synthetic construct
EVQLVESGGGLVQPGGSLRLSCAASGFTISDYDMHWVRQTTGKGLEWVSGIDPDGDTYYPGSLKGRFTISRENAKNSLYLQMNSLRAGDTAVYYCTRGEDSSSSEDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 302 - protein, 449 aa, Synthetic construct
EVQLVESGGGVVRPGGSLRLSCAASGFSFDDYGMSWVRQVPGKGLEWVSGINWNGGSTGYADSVKGRFTISRDNAKNSLYLQMSSLRAEDTALYYCASYSGSFRDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 303 - protein, 451 aa, Synthetic construct
EVQLVESGGGVVRPGGSLRLSCAASGFTFDDYGMSWVRQAPGKGLEWVSTINWNGGNTGYADSVKGRFTISRDNAKNSLYLQMNSLRAEDTALYYCARDEGAAGLGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 304 - protein, 447 aa, Synthetic construct
EVQLVESGGGLVKPGGSLRLSCAASGFTFSSYSMNWVRQAPGKGLEWVSSITSSSYIFYADSLKGRFTISRDNAKNSLFLQMNSLRAEDTAVYYCARDSNWGEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 305 - protein, 448 aa, Synthetic construct
EVQLVESGGGLVKPGGSLRLSCAASGFTFSTYSMNWVRQAPGKGLEWVSSITSSSSYMFYADSVMGRFTISRDNAKNSLYLQMNSLRAEDTADYYCARDSNWGEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 306 - protein, 449 aa, Synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGDYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 307 - protein, 454 aa, Synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVERDMVRGHYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 308 - protein, 450 aa, Synthetic construct
QVQLVESGGGVVQPGRSLRLSCAASGFTFSSYGMHWVRQAPGKGLEWVSHITYDGSDKYYGDSVKGRFTISRDNSKNTLYLQMNSLRAEDTAVYYCAKDGYSSGWWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 309 - protein, 455 aa, Synthetic construct
QVQLVESGGGVVQPGRSLRLSCAASGFTFSSYGMHWVRQAPGKGLEWVAIISYDGNNKYYADSVKGRFTISRDNSKNTLYLQMNSLRDEDTAVYYCAKERGYSSSSGYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 310 - protein, 448 aa, Synthetic construct
QVQLVESGGGVVQPGRSLRLSCAASGFPFSSYGMHWVRQAPGKGLEWVAVMSYDGINGYYADSVKGRFTISRDNSKNTLYLQMNSLRAEDTAVYYCARLVYADDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 311 - protein, 444 aa, Synthetic construct
QVQLVESGGGVVQPGRSLRLSCAASGFAFSSYGMHWVRQAPGKGLEWVAVISYYGSNKYYADSVKGRFTISRDNSKNTLYLQMNSLRAEDTAVYHCARGITMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 312 - protein, 444 aa, Synthetic construct; identical to SEQ ID NO: 311

SEQ ID NO: 313 - DNA, 1350 bp, Synthetic construct
  source 1..1350: mol_type = other DNA
gaggtgcagctggtggagtcggggggaggcttggtacagcctggggggtccctgagactctcctgtgcagcctctggattcaccctcagtgtctacgacatgcactgggtccgccaagtttcaggaaaaggtctggagtgggtctcaggtattgatcctgatggtgacacatactatcccggctccgtgaagggccgattcaccatctccagagaaaatgccaagaactccttgtatcttcaaatgaacagcctgagagtcggggacacggctgtgtattattgtctaagaggggaggaaagcaactcgtccgaggatggttttgatatatggggacaagggacaatggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 314 - DNA, 1350 bp, Synthetic construct
  source 1..1350: mol_type = other DNA
gaggtgcagctggtggagtctgggggaggcttggtacagcctggggggtccctgagactctcctgtgcagcctctggattcaccttcagtagccacgacatgcactgggtccgccaagttacaggaaaaggtctggagtgggtctcaggtattgatcctgctggtgacacatactatccagactccgtgaagggccgattcaccatctctagagaaaatgccaagaactccttgtatcttcaaatgaacagcctgagagccggggacacggctgtgtattactgtacaagaggagaggctagtagttcgtccgaggatgcttttgatatctggggacaagggacaatggtcactgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 315 - DNA, 1350 bp, Synthetic construct
  source 1..1350: mol_type = other DNA
gaggtgcagctggtggagtctgggggaggcttggtacagcctggggggtccctgagactctcctgtgcagcctctggattcaccctcagtagctacgacatgcactgggtccgccaagttataggaaaaggtctggagtgggtctcaggtattgatcctgatggtgacacatactatccaggctccgtgaagggccgattcaccatctccagagaagatgccaagaactccttgtatcttcaaatgaacagcctgagagccggggacacggctgtgtattactgtacaagaggagaggataacagctcgtccgaggatgcttttgatatctggggacaagggacaatggtcactgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 316 - DNA, 1350 bp, Synthetic construct
  source 1..1350: mol_type = other DNA
gaggtccagctggtggagtctgggggaggcttggtacagccgggggggtccctgagactctcctgtgcagcctctggattcaccatcagtgactacgacatgcactgggtccgccaaactacaggaaaaggtctagaatgggtctcaggtattgatcctgatggcgacacatattatccaggctccttgaaggggcgattcaccatctccagagaaaatgccaagaattctttgtatcttcaaatgaacagtctgagagccggggacacggctgtgtattactgtacaagaggagaggatagtagttcgtccgaggatgcttttgatatctggggacaagggacaatggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 317 - DNA, 1347 bp, Synthetic construct
  source 1..1347: mol_type = other DNA
gaggtgcagctggtggagtctgggggaggtgtggtacggcctggggggtccctgagactctcctgtgcagcctctggattcagctttgatgattatggcatgagctgggtccgccaagttccagggaaggggctggagtgggtctctggtattaattggaatggtggtagtacaggttatgcagactctgtgaagggccgattcaccatctccagagacaacgccaagaactccctgtatctgcaaatgagcagtctgagagccgaggacacggccttgtattactgtgcgagctatagtgggagcttccgtgatgcttttgatatctggggacaagggacaatggtcaccgtctcttcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 318 - DNA, 1353 bp, Synthetic construct
  source 1..1353: mol_type = other DNA
gaggtgcagctggtggagtctgggggaggtgtggtacggcctggggggtccctgagactctcctgtgcagcctctggattcacctttgatgattatggcatgagctgggtccgccaagctccagggaaggggctggagtgggtctctactattaattggaatggtggtaacacaggttatgcagactctgtgaagggccgattcaccatctccagagacaacgccaagaactccctgtatctgcaaatgaacagtctgagagccgaggacacggccttgtattactgtgcgagagatgaaggagcagctggacttggatactactttgactactggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 319 - DNA, 1341 bp, Synthetic construct
  source 1..1341: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattactagtagtagttacatattctacgcagactcattgaagggccgattcaccatctccagagacaacgccaagaactcactgtttctgcaaatgaacagcctgagagccgaggacacagctgtttattactgtgcgagagattctaactggggcgaggcttttgatatctggggacaagggacaatggtcaccgtctcttcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 320 - DNA, 1344 bp, Synthetic construct
  source 1..1344: mol_type = other DNA
gaggtgcagctggtggagtctgggggaggcctggtcaagcctggggggtccctgagactctcctgtgcagcctctggtttcaccttcagtacttatagcatgaactgggtccgccaggctccagggaaggggctggagtgggtctcatccattactagtagtagtagttacatgttctacgcagactcagtgatgggccgattcaccatctccagagacaacgccaagaactcactgtacctacaaatgaacagcctgagagccgaggacacagctgattattactgtgcgagagattctaattggggggaggcttttgatatctggggacaagggacaatggtcaccgtctcttcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 321 - DNA, 1347 bp, Synthetic construct
  source 1..1347: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagacggtgactactactactactacatggacgtctggggcaaagggaccacggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 322 - DNA, 1362 bp, Synthetic construct
  source 1..1362: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagtggaaagggatatggttcggggccactactactactacatggacgtctggggcaaagggaccacggtcaccgtctcttcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 323 - DNA, 1350 bp, Synthetic construct
  source 1..1350: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtgtcacatataacatatgatggaagtgataaatactatggagactccgtgaagggccgattcaccatctccagagacaattccaagaatacgctgtatctgcaaatgaacagcctgagagctgaggacacggctgtgtattactgtgcgaaagatgggtatagcagtggctggtggtactttgactactggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 324 - DNA, 1365 bp, Synthetic construct
  source 1..1365: mol_type = other DNA
caggtgcagctggtggagtccgggggaggcgtggtccagcctgggaggtccctgagactctcctgtgcagcctctggattcaccttcagtagctatggcatgcactgggtccgccaggctccaggcaaggggctggagtgggtggcaattatatcatatgatggaaataataaatactatgcagactccgtgaagggccgattcaccatctccagagacaattccaagaacacgctgtatctgcaaatgaacagcctgagagatgaggacacggctgtttattattgtgcgaaagagagggggtatagcagctcgtccggctactactactactacatggacgtctggggcaaagggaccacggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 325 - DNA, 1344 bp, Synthetic construct
  source 1..1344: mol_type = other DNA
caagtgcaattggtggagtccggtggaggagtagtacagccgggaaggtcactgagactttcctgcgctgctagtggattcccatttagctcctacgggatgcactgggttcgccaagcgccaggtaagggactggaatgggtggctgtcatgagttacgatggaattaacggctattatgccgatagcgtcaaaggccggttcacgatctcaagagacaactctaagaacacgctctacttgcagatgaacagtctccgcgcagaagatacggccgtgtattactgtgcgagattggtgtatgccgatgatgcttttgatatctggggacaagggaccacggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 326 - DNA, 1332 bp, Synthetic construct
  source 1..1332: mol_type = other DNA
caagtgcaattggtggagtccggtggaggagtagtacagccgggaaggtcactgagactttcctgcgctgctagtggattcgcctttagttcctacggcatgcactgggttcgccaagcgccaggtaagggactggaatgggtggctgtcatctcctactacggatctaataaatattatgccgatagcgtcaaaggccggttcacgatctcaagagacaactctaagaacacgctctacttgcagatgaacagtctccgcgcagaagatacggccgtgtatcactgtgcgagagggattactatggacgtctggggcaaagggaccacggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 327 - DNA, 1347 bp, Synthetic construct
  source 1..1347: mol_type = other DNA
gaagtgcagctggtggagtctgggggaggcttggtacagcctggcaggtccctgagactctcctgtgcagcctctggattcacctttgatgattttgccatgcactgggtccggcaaggtccagggaagggcctggagtgggtctcaggtattggttggaatagtggtagcatcggctatgcggactctgtgaagggccgattcaccatctccagagacaacgccaagaactccctgtatctgcaaatgaacagtctgagagctgaggacacggccttatattactgtgcaaaagcccctgactacggtgactactactttgactactggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 328 - protein, 219 aa, Synthetic construct
DVVMTQSPLSLPVTLGQPASISCRSSQSLVYSDGNTYLNWFQQRPGQSPRRLIYKVSNRDSGVPDRFSGSGSGTDFTLKISKVEAEDVGIYYCMQGTHWPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 219 aa, Synthetic construct
DVVMTQSPLSLPVTLGQPASISCRSSQSLVYSDGNTYLNWFQQRPGQSPRRLIHEVSNRDSGVPDRFSGSGSGTDFTLKISRVEAEDVGVYYCMQGTHWPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 219 aa, Synthetic construct
DVVMTQSPLSLPVTLGQPASISCRSSQSLVFSDGNTYLNWFQQRPGQSPRRLIYKVSNRDSGVPDRFSGSGSGTDFTLKISRVEAEDVGVYYCMQGTHWPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 219 aa, Synthetic construct; identical to SEQ ID NO: 329

SEQ ID NO: 332 - protein, 214 aa, Synthetic construct
AIQMTQSPSSLSASVGDRVTITCRASQGIRNDLGWYQQKPGKAPKLLIYAASSLQSGVPSRFSGSGSGTDFTLTISSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 214 aa, Synthetic construct
DIQMTQSPSTLSASVGDRVTITCRASQSISSWLAWYQQKPGKAPKLLIYATSSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 214 aa, Synthetic construct
DIQMTQSPSSLSASVGDRVTITCRASQGIRNDLGWYQQKPGKAPKRLIYAASSLPSGVPSRFSGSGSGTEFTLTISSLQPEDFAIYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 214 aa, Synthetic construct
DIQMTQSPSSLSASVGDRVTITCRASQGIRIDLGWFQQKPGKAPKRLIYAASSLPSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 336 - protein, 215 aa, Synthetic construct; identical to SEQ ID NO: 280

SEQ ID NO: 337 - protein, 214 aa, Synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 214 aa, Synthetic construct
DIQMTQSPSSLSASVGDRVTITCRASQGIRNDLGWYQQKPGKAPKRLIYAASSLQSGVPSRFSGSGSGTEFALTISSLQPEDFATYYCLQHNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 339 - protein, 215 aa, Synthetic construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 214 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - protein, 214 aa, Synthetic construct; identical to SEQ ID NO: 340

SEQ ID NO: 342 - protein, 214 aa, Synthetic construct
DIQMTQSPSSLSASVGDRVTITCRASQSISIYLNWYQQKPGTAPKLLIYAASSLQSGVPSRFSGSGSGTDFTLTISSLQPEDFAAYYCQQSYTTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - DNA, 657 bp, Synthetic construct
  source 1..657: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcaaggtggaggctgaggatgttgggatttattactgcatgcaaggtacacactggccgtggacgttcggccaagggaccaaagtggatatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 344 - DNA, 657 bp, Synthetic construct
  source 1..657: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaattcatgaggtttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaagtggatatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 345 - DNA, 657 bp, Synthetic construct
  source 1..657: mol_type = other DNA
gatgttgtgatgactcagtctccactctccctgcccgtcacccttggacagccggcctccatctcctgcaggtctagtcaaagcctcgtattcagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaagtggatatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 346 - DNA, 657 bp, Synthetic construct; identical to SEQ ID NO: 344
  source 1..657: mol_type = other DNA

SEQ ID NO: 347 - DNA, 642 bp, Synthetic construct
  source 1..642: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgcaacttattactgtctacaagattacaattacccgtacacttttggccaggggaccaagctggagatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 348 - DNA, 642 bp, Synthetic construct
  source 1..642: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgctacatccagtttgcaaagtggggtcccatcaaggttcagtggcagtggatctgggacagatttcactctcaccatcagcagtctgcaacctgaagattttgcaacttactactgtcaacagagttacagtaccccgatcaccttcggccaagggacacgactggagattaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 349 - DNA, 642 bp, Synthetic construct
  source 1..642: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgccaagtggggtcccatcaaggttcagcggcagtggatctgggacagaattcactctcacaatcagcagcctgcagcctgaagattttgcaatttattactgtctacagcataatagttacccgtggacgttcggccaagggaccaaggtggaaatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 350 - DNA, 642 bp, Synthetic construct
  source 1..642: mol_type = other DNA
gacatccagatgacccagtctccatcctccctgtccgcatctgtaggagacagagtcaccatcacttgccgggcaagtcagggcattagaattgatttaggctggtttcagcagaaaccagggaaagcccctaagcgcctcatctatgctgcatccagtttgccaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggagatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 351 - DNA, 645 bp, Synthetic construct; identical to SEQ ID NO: 288
  source 1..645: mol_type = other DNA

SEQ ID NO: 352 - DNA, 642 bp, Synthetic construct
  source 1..642: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgtggacgttcggccaagggaccaaagtggatatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 353 - DNA, 642 bp, Synthetic construct
  source 1..642: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcgctctcacaatcagcagcctgcagcctgaagattttgcaacttattactgtctacagcataatagttaccctctcactttcggcggagggaccaagctggagatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 354 - DNA, 645 bp, Synthetic construct
  source 1..645: mol_type = other DNA
gaaattgtgctgactcagtctcccggcacactgtctcttagccctggcgaaagagccacactgagctgtagagccagccagagcgtgtccagaagctacctggcttggtatcagcagaagcccggacaggctcccagactgctgatcatcggagcctctacaagagccaccggcattcccgatagattcagcggctctggcagcggcaccgatttcaccctgacaatcagcagactggaacccgaggacttcgccgtgtactactgtcagcagggccaagtgatccctcctacctttggccagggcaccaaggtggaaatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 355 - DNA, 642 bp, Synthetic construct
  source 1..642: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 356 - DNA, 642 bp, Synthetic construct; identical to SEQ ID NO: 355
  source 1..642: mol_type = other DNA

SEQ ID NO: 357 - DNA, 642 bp, Synthetic construct
  source 1..642: mol_type = other DNA
gacatccagatgacccagtctccatcctccctgtctgcatctgtaggagacagagtcaccatcacttgccgggcaagtcagagcattagcatctatttaaattggtatcagcagaaaccagggacagcccctaagctcctgatctatgctgcatccagtttgcaaagtggggtcccatcaaggttcagtggcagtggatctgggacagatttcactctcaccatcagcagtctgcaacctgaagattttgcagcttactactgtcaacagagttacactaccccgctcactttcggcggagggaccaaggtggaaatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 358 - protein, 450 aa, Synthetic construct
QLQLQESGPGLVKPSETLSLTCTVSGGSISSSSSYWGWIRQPPGKGLEWIGSIYYSGSTYSNPSLKSRVTISVDTSKNQFSLKLSSVTAADTAVYYCARQYISGTEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 359 - protein, 450 aa, Synthetic construct; identical to SEQ ID NO: 358

SEQ ID NO: 360 - protein, 449 aa, Synthetic construct
QVQLQESGPGLVKPSETLSLTCTVSGGSVSSGSYYWSWIRQPPGKGLEWIGYIYYSGSTSYNPSLKSRVSISVDTSKNQFSLKLSSVTAADTAVFYCARAKWEGDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 361 - DNA, 1350 bp, Synthetic construct
  source 1..1350: mol_type = other DNA
cagctgcagctgcaggagtcgggcccaggactggtgaagccttcggagaccctgtccctcacctgcactgtctctggtggttccatcagcagtagtagttcctactggggctggatccgccagcccccagggaagggactggagtggattgggagtatctattatagtgggagcacctactccaacccgtccctcaagagtcgagtcaccatatccgtagacacgtccaagaatcagttctccctgaagctgagctctgtgaccgccgcagacacggctgtgtattactgtgcgagacagtatataagtggaactgaatacttccagtactggggccagggcaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 362 - DNA, 1350 bp, Synthetic construct; identical to SEQ ID NO: 361
  source 1..1350: mol_type = other DNA

SEQ ID NO: 363 - DNA, 1347 bp, Synthetic construct
  source 1..1347: mol_type = other DNA
caggtgcagctgcaggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccagctacaacccctccctcaagagtcgagtctccatatcagtagacacgtccaagaaccagttctccctgaagctgagctctgtgaccgctgcggacacggccgtgttttactgtgcgagagctaagtgggaaggggactactttgactactggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacgcctcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 364 - protein, 213 aa, Synthetic construct
EIVLTQSPATLSLSPGERATLSCRASQSVSNYLAWYQQKPGQAPRLLIYDASNRATGIPARFSGSGSGTDFTLTISSLEPEDFAVYYCQQRSNWPSFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 365 - protein, 214 aa, Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 366 - protein, 215 aa, Synthetic construct
DIQMTQSPSSLSASVGDRVTITCRASQSIYSYLNWYQQKPGKAPNHLIYAASSLQSGVPSRFSGSGSGTDFTLTISSLHPEDFATYYCQQSYSTPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 367 - DNA, 639 bp, Synthetic construct
  source 1..639: mol_type = other DNA
gaaattgtgttgacacagtctccagccaccctgtctttgtctccaggggaaagagccaccctctcctgcagggccagtcagagtgttagcaactacttagcctggtaccaacagaaacctggccaggctcccaggctcctcatctatgatgcatccaacagggccactggcatcccagccagattcagtggcagtgggtctgggacagacttcactctcaccatcagcagcctggagcctgaagattttgcagtttattactgtcagcagcgtagcaactggccctccttcggccaagggacacgactggagattaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 368 - DNA, 642 bp, Synthetic construct
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggaaatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 369 - DNA, 645 bp, Synthetic construct
  source 1..645: mol_type = other DNA
gacatccagatgacccagtctccatcctccctgtctgcatctgtaggagacagagtcaccatcacttgccgggcaagtcagagcatttacagctatttaaattggtatcagcagaaaccagggaaagcccctaatcacctgatctatgctgcatccagtttgcaaagtggggtcccatcaaggttcagtggcagtggatctgggacagatttcactctcaccatcagcagtctgcaccctgaagattttgcaacttactactgtcaacagagttacagtacccctccgtacacttttggccaggggaccaaggtggaaatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 370 - protein, 346 aa, Homo sapiens
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 371 - protein, 346 aa, Homo sapiens
MLKKPLSAVTWLCIFIVAFVSHPAWLQKLSKHKTPAQPQLKAANCCEEVKELKAQVANPSSLLSELNKKQERDWVSVVMQVMEPESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 372 - protein, 350 aa, Homo sapiens
MLKKPLSAVTWLCIFIVAFVSHPAWLQKLSKHKTPAQPQLKAANCCEEVKELKAQVANGGPGGSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 373 - protein, 215 aa, Homo sapiens
YDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPED

SEQ ID NO: 374 - protein, 346 aa, Homo sapiens; identical to SEQ ID NO: 370

(19) United States
(12) Patent Application Publication
(10) Pub. No.: US 2025/0296996 A1
(43) Pub. Date: Sep. 25, 2025

(54) ANGIOPOIETIN-RELATED PROTEIN 7-SPECIFIC ANTIBODIES AND USES THEREOF

(71) Applicant: Broadwing Bio, Inc., South San Francisco, CA (US)

(72) Inventor: Andrew Peterson, South San Francisco, CA (US)

(21) Appl. No.: 18/860,128

(22) PCT Filed: Apr. 28, 2023
(86) PCT No.: PCT/US2023/020354

§ 371 Date: Oct. 25, 2024

Related U.S. Application Data
(60) Provisional application No. 63/336,747, filed on Apr. 29, 2022.

(51) Int. Cl. C07K 16/22; A61K 39/00; A61P 27/06

(52) U.S. Cl. CPC C07K 16/22; A61P 27/06; A61K 2039/505; C07K 2317/34; C07K 2317/35; C07K 2317/92

(57) ABSTRACT

The present disclosure relates to the treatment and/or prevention of glaucoma and other diseases affecting the optic nerve and retinal ganglion cells. In particular, the present disclosure provides novel therapeutic antibodies, and related compositions and methods, that target angiopoietin-related protein 7 (ANGPTL7) to reduce intraocular pressure (IOP) in order to prevent optic nerve damage and restore vision.

### CROSS REFERENCE TO RELATED APPLICATIONS

This application claims priority to and the benefit of U.S. Provisional Patent Application No. 63/336,747 filed Apr. 29, 2022, which is incorporated herein by reference in its entirety for all purposes.

### SEQUENCE LISTING

The text of the computer readable sequence listing filed herewith, titled “40185_601_SequenceListing,” created Mar. 30, 2023, having a file size of 410,410 bytes, is hereby incorporated by reference in its entirety.

### FIELD

Embodiments of the present disclosure relate to the treatment and/or prevention of glaucoma and other diseases affecting the optic nerve and retinal ganglion cells. In particular, the present disclosure provides novel therapeutic antibodies, and related compositions and methods, that target angiopoietin-related protein 7 (ANGPTL7) to reduce intraocular pressure (IOP) in order to prevent optic nerve damage and restore vision.

### BACKGROUND

Glaucoma is a group of optic neuropathies associated with characteristic structural changes at the optic nerve head that may lead to visual field loss and, ultimately, blindness. Blindness is most commonly defined as 20/200 or worse visual acuity on a Snellen eye chart or a visual field of less than 20 degrees. Legal blindness refers to the fulfillment of these criteria by the better-seeing eye. By 2020, approximately 79.6 million people worldwide will have glaucoma and more than 11 million will be bilaterally blind from glaucoma. More than 2 million Americans 40 years and older have glaucoma, and studies of the U.S. population estimate that more than one-half of these cases may be undiagnosed or untreated. Among black and Hispanic persons, glaucoma is the leading cause of irreversible blindness. Glaucoma accounts for more than 25% of cases of blindness in these groups, making it a more common cause of blindness than diabetic retinopathy (accounting for 7.3% and 14.3% of cases in blacks and Hispanics, respectively) and age-related macular degeneration (accounting for 4.4% and 14.3% of cases in blacks and Hispanics, respectively). Among Hispanics, glaucoma causes blindness more often than cataracts do (28.6% vs. 14.3%). In 2009, Medicare beneficiaries spent $748 million on glaucoma-related visits, testing, and procedures. Patients with glaucoma who are not blind may have functional limitations, leading to driving cessation and decreased ability to read.

The two most common forms of glaucoma are primary open-angle glaucoma (POAG) and primary angle-closure glaucoma (PACG), with the former approximately seven times more common than the latter in the United States and Europe. When POAG and PACG are left untreated, the typical disease course is chronic, progressive, and irreversible visual field loss, which may progress to tunnel vision and, ultimately, loss of central vision. Treatment that reduces intraocular pressure has been shown to improve outcomes in randomized clinical trials. The angle of the eye is the junction between the iris and cornea, where the trabecular meshwork drains aqueous humor from the anterior chamber of the eye. In POAG, the angle remains open as the trabecular meshwork is unblocked by iris tissue. Intraocular pressure is transmitted to the axons of retinal ganglion cells at the optic nerve as mechanical stress, leading to cell death. However, about 50% of patients with glaucoma have intraocular pressure within the so-called “normal” range of 10 to 21 mm Hg at diagnosis. Only after 30% of retinal ganglion cells have been lost are visual field defects present on perimetric testing. In PACG, the peripheral iris obstructs normal aqueous outflow. This can lead to increased intraocular pressure and optic nerve damage. Eyes that are at risk of PACG tend to be shorter with a shallower anterior chamber. Patients with PACG may experience acute or subacute events that occur after a sudden rise in intraocular pressure or from chronic PACG that is insidious in onset and largely asymptomatic.

### SUMMARY

Embodiments of the present disclosure include an antibody, or an antigen binding fragment thereof, which specifically binds human Angiopoietin-Like Protein 7 (ANGPTL7), optionally wherein said human ANGPTL7 is a polypeptide which comprises or consists of the amino acid sequence of any one of SEQ ID NOs: 370 to 374.

In some embodiments, the antibody, or an antigen binding fragment thereof, exhibits any one or more the following functional characteristics: increases outflow facility compared to a control when administered to the eye of a subject, optionally wherein the control is vehicle treatment, dexamethasone treatment, ANGPTL7 protein treatment, or ANGPTL7 protein with an isotype control antibody treatment; and/or binds to ANGPTL7 with a K_{D} of about 100 nM or lower; and/or binds to the same epitope on ANGPTL7 as an antibody comprising the VH and VL sequences of any one of the exemplary antibodies the sequences of which are provided in Table 11; and/or competes for binding to ANGPTL7 with an antibody comprising the VH and VL sequences of any one of the exemplary antibodies the sequences of which are provided in Table 11.

In some embodiments, the antibody, or an antigen binding fragment thereof, is monoclonal, optionally recombinant. In some embodiments, the antibody, or an antigen binding fragment thereof, is human, humanized, or chimeric.

In some embodiments, the antibody, or an antigen binding fragment thereof, is a full length antibody, a single chain antibody, a single chain variable fragment (scFv), a variable fragment (Fv), a fragment antigen-binding region (Fab), a Fab-C, a Fab′-SH, a (Fab′)2, a single-domain antibody (sdAb), a VHH antibody, a nanobody, a camelid-derived single-domain antibody, a shark TgNAR-derived single-domain antibody fragment (VNAR), a diabody, a triabody, an anticalin or an aptamer, optionally wherein the antibody is a full length antibody comprising an Fc region such as a human IgG1, IgG2, IgG3 or IgG4 region.

In some embodiments, the antibody, or an antigen binding fragment thereof, is conjugated to at least one additional moiety, optionally selected from: an antigen binding moiety, such as an antibody or antigen-binding fragment thereof, which is capable of specific binding to a target which is not human ANGPTL7, preferably wherein said target is expressed in the human eye; a therapeutic or cytotoxic moiety; a detection moiety; a purification moiety; a half-life extension moiety, optionally a polypeptide that is at least 20 amino acids in length and comprises any combination of G, A, S T, E, and P residue, which polypeptide is conjugated to the C- or N-terminus of the antibody.

In some embodiments, the antibody, or an antigen binding fragment thereof is a polypeptide comprising: one, two or all three HCDRs of any one of the exemplary antibodies the sequences of which are provided in Table 11, and optionally also one, two or all three of the corresponding LCDRs of the exemplary antibody; and/or a VH sequence having at least 90% identity to the VH sequence of any one of the exemplary antibodies the sequences of which are provided in Table 11, and optionally also a VL sequence having at least 90% identity to the corresponding VL sequence of the exemplary antibody, preferably wherein variation is not permitted in the HCDRs or LCDRs; and/or all six CDRs of any of the exemplary antibodies exemplary antibodies the sequences of which are provided in Table 11; and/or the VH and VL sequences of any one of the exemplary antibodies the sequences of which are provided in Table 11; and/or the full length heavy chain (VH+constant) sequence of any one of the exemplary antibodies the sequences of which are provided in Table 11, and optionally also the corresponding full length light chain (VL+constant) sequence of the exemplary antibody.

Embodiments of the present disclosure also include a polynucleotide encoding an antibody, or an antigen binding fragment thereof, of any of the preceding paragraphs, optionally wherein said polynucleotide comprises or consists of a nucleic acid sequence having at least 70%, 80%, 90% or 100% identity to a nucleic acid sequence of any one of the exemplary antibodies the sequences of which are provided in Table 11.

Embodiments of the present disclosure also include an expression vector comprising the polynucleotide of the preceding paragraph, which is optionally an adeno-associated virus (AAV) vector, a lentiviral (LV) vector, a herpes simplex virus (HSV) vector, or a retrovirus vector.

Embodiments of the present disclosure also include a pharmaceutical composition comprising an antibody, or an antigen binding fragment thereof, a polynucleotide, or a vector according to any one of the preceding paragraphs, and optionally: at least one pharmaceutically acceptable carrier, diluent or preservative; and/or at least one additional active ingredient. In some embodiments, the pharmaceutical composition is suitable for ocular administration to a subject, optionally by delivery using a conjunctival insert, a contact lens, a gel, a nanoparticle, a mucoadhesive polymer, an ointment, a solution, a suspension, eye drops, and/or an implant, preferably by injection into the vitreous fluid.

Embodiments of the present disclosure also include the antibody, or an antigen binding fragment thereof, the polynucleotide, the vector, or the compositions of any of the preceding paragraphs, for use as a medicament, optionally for use in a method of treating a disease of the eye in a subject. In some embodiments, the disease is characterized by increased intraocular pressure and/or reduced outflow facility in the eye of the subject. In some embodiments, the method comprises ocular administration of the antibody, preferably by injection into the vitreous fluid, and wherein said administration preferably relieves at least one symptom in the subject selected from eye pain, eye pressure, headaches, rainbow-colored halos around lights, low vision, blurred vision, narrowed vision, impaired peripheral vision, blind spots, nausea, vomiting, and red eyes. In some embodiments, the disease is glaucoma and/or a disease affecting the optic nerve or retinal ganglion cells, optionally wherein said glaucoma is primary or glucocorticoid-induced glaucoma.

Embodiments of the present disclosure include antibodies against Angiopoietin-Like Protein 7 (ANGPTL7) peptides, or an antigen-binding fragment thereof, comprising a heavy chain variable region (VH) comprising complementarity determining regions (CDRs) HCDR1, HCDR2, and HCDR3, and a light chain variable region (VL) comprising complementarity determining regions (CDRs) LCDR1, LCDR2, and LCDR3. In some embodiments, the HCDR1 comprises one of the following amino acid sequences: (a) X₁YX₂IX₃ (SEQ ID NO: 1), wherein X₁ is S or D; X₂ is G or Y; X₃ is S or H; (b) TSGVGVG (SEQ ID NO: 18); (c) X₁X₂X₃MX₄ (SEQ ID NO: 27), wherein X₁ is V, S, D, or T; X₂ is Y, H, or F; X₃ is D, G, S, or A; X₄ is H, S, or N; or (d) SX₁SX₂YWX₃ (SEQ ID NO: 74), wherein X₁ is S or G; X₂ is S or Y; X₃ is G or S. In some embodiments, the HCDR2 comprises one of the following amino acid sequences: (a) WIX₁X₂X₃X₄GXSTX₆YAQX₇X₈X₉G (SEQ ID NO: 7), wherein X₁ is S, 1, or N; X₂ is A or P; X₃ is Y or N; X₄ is N or T; X₅ is N or A; X₆ is N or K; X₇ is N or K; X₈ is L or F; X₉ is R or Q; (b) LIYWNDDKX₁YSPSLKS (SEQ ID NO: 21), wherein X₁ is R or Q; (c) X₁X₂X₃X₄X₅X₆X₇X₈X₉X₁₀X₁₁X₁₂X₁₃X₁₄X₁₅G (SEQ ID NO: 43), wherein X₁ is G, T, S, A, V, H, or T; X₂ is I or M; X₃ is D, N, T, S, or G; X₄ is P, W, S, G, or Y; X₅ is D, A, N, S, or Y; X₆ is G or S; X₇ is D, G, Y, S, I, or N; X₈ is T, S, N, I, Y, or D; X₉ is Y, T, F, M, K, G, or I; X₁₀ is Y, G, or F; X₁₁ is P, Y, or A; X₁₂ is G, D, or A; X₁₃ is S or D; X₁₄ is V, L, or S; X₁₅ is K or M; or (d) X₁IYYSGSTX₂SNPSLKS (SEQ ID NO: 78) wherein X₁ is S, or Y; X₂ is Y or S. In some embodiments, the HCDR3 comprises one of the following amino acid sequences: (a) SEQ ID NOs: 13-17; (b) X₁X₂X₃X₄X₅X₆FFDX₇ (SEQ ID NO: 24) wherein X₁ is S, D, or N; X₂ is Y or P; X₃ is G or D; X₄ is D or Y; X₅ is Y or G; X₆ is W or D; X₇ is L or Y; (c) SEQ ID NOs: 59-73; or (d) X₁X₂X₃X₄GX₅X₆X₇X₈X₉Y (SEQ ID NO: 82) wherein X₁ is Q or A; X₂ is Y or K; X₃ is I or W; X₄ is S or E; X₅ is T or D; X₆ is E or Y; X₇ is Y or F; X₈ is F or D; X₉ is Q or Y.

In accordance with the above embodiments, the LCDR1 of the anti-ANGPTL7 antibodies of the present disclosure includes an amino acid sequences of any of SEQ ID NOs: 87-97, SEQ ID NOs: 123-127, or SEQ ID NOs: 141-149; the LCDR2 comprises an amino acid sequence of any of SEQ ID NOs: 99-109, SEQ ID NOs: 129-133, or SEQ ID NOs 151-159; and the LCDR3 comprises an amino acid sequence of any of SEQ ID NOs: 111-121, SEQ ID NOs: 135-139, or SEQ ID NOs: 161-169.

In some embodiments, the present disclosure provides antibodies directed against ANGPTL7 peptides, or an antigen-binding fragment thereof, that include a VH comprising complementarity determining regions HCDR1, HCDR2, and HCDR3, and a VL comprising complementarity determining regions LCDR1, LCDR2, and LCDR3, wherein the LCDR1 comprises one of the following amino acid sequences: (a) RASQX₁IX₂X₃X₄LX₅ (SEQ ID NO: 86), wherein X₁ is G or S; X₂ is S, R, or Y; X₃ is S, N, or I; X₄ is W, D, or Y; X₅ is A, G, or N; (b) RSSQSLX₁X₂SX₃X₄X₅X₆YLX₇ (SEQ ID NO: 122), wherein X₁ is L or V; X₂ is H, Y, or F; X₃ is N or D; X₄ is R or G; X₅ is Y or N; X₆ is N or T; X₇ is D or N; or (c) RASQSVSX₁X₂X₃X₄A (SEQ ID NO: 140), wherein X₁ is S, N, or R; X₂ is Y or S; X₃ is L or Y; X₄ is A or L. In some embodiments, the LCDR2 comprises one of the following amino acid sequences: (a) AX₁SSLX₂S (SEQ ID NO: 98), wherein X₁ is A or T; X₂ is Q or P; (b) X₁X₂SNRX₃S (SEQ ID NO: 128), wherein X₁ is L, K, or E; X₂ is G or V; X₃ is A or D; or (c) X₁ASX₂RAT (SEQ ID NO: 150), wherein X₁ is D or G; X₂ is N, S, or T. In some embodiments, the LCDR3 comprises one of the following amino acid sequences: (a) X₁QX₂X₃X₄X₅PX₆X₇ (SEQ ID NO: 110), wherein X₁ is L or Q; X₂ is A, H, S, or D; X₃ is N, F, or Y; X₄ is S, T, or N; X₅ is F, Y, or T; X₆ is W, L, I, P, or Y; X₇ is T or Y; (b) MQX₁X₂X₃X₄PX₅T (SEQ ID NO: 134), wherein X₁ is T or G; X₂ is L or T; X₃ is Q or H; X₄ is T or W; X₅ is Y or W; or (c) QQX₁X₂X₃X₄X₅X₆T (SEQ ID NO: 160), wherein X₁ is R, Y, or G; X₂ is S, G, or Q; X₃ is N, S, or V; X₄ is W, S, or I; X₅ is P or L; X₆ is L, S, P, or T.

In accordance with the above embodiments, the HCDR1 of the anti-ANGPTL7 antibodies of the present disclosure includes an amino acid sequences of any of SEQ ID NOs: 2-6, SEQ ID NOs: 19-20, SEQ ID NOs: 28-42, or SEQ ID NOs: 75-77; the HCDR2 comprises an amino acid sequence of any of SEQ ID NOs: 8-12, SEQ ID NOs: 22-23, SEQ ID NOs 44-58, or SEQ ID NOs: 79-81; and the HCDR3 comprises an amino acid sequence of any of SEQ ID NOs: 13-17, SEQ ID NOs: 25-26, SEQ ID NOs: 59-73, or SEQ ID NOs: 83-85.

In some embodiments, the HCDR1 comprises the amino acid sequence of SEQ ID NO: 2; the HCDR2 comprises the amino acid sequence of SEQ ID NO: 8; and the HCDR3 comprises the amino acid sequence of SEQ ID NO: 13. In some embodiments, the HCDR1 comprises the amino acid sequence of SEQ ID NO: 3; the HCDR2 comprises the amino acid sequence of SEQ ID NO: 9; and the HCDR3 comprises the amino acid sequence of SEQ ID NO: 14. In some embodiments, the HCDR1 comprises the amino acid sequence of SEQ ID NO: 4; the HCDR2 comprises the amino acid sequence of SEQ ID NO: 10; and the HCDR3 comprises the amino acid sequence of SEQ ID NO: 15. In some embodiments, the HCDR1 comprises the amino acid sequence of SEQ ID NO. 5; the HCDR2 comprises the amino acid sequence of SEQ ID NO: 11; and the HCDR3 comprises the amino acid sequence of SEQ ID NO: 16. In some embodiments, the HCDR1 comprises the amino acid sequence of SEQ ID NO: 6; the HCDR2 comprises the amino acid sequence of SEQ ID NO: 12; and the HCDR3 comprises the amino acid sequence of SEQ ID NO: 17. In some embodiments, the HCDR1 comprises the amino acid sequence of SEQ ID NO: 7; the HCDR2 comprises the amino acid sequence of SEQ ID NO: 14; and the HCDR3 comprises the amino acid sequence of SEQ ID NO: 20. In some embodiments, the HCDR1 comprises the amino acid sequence of SEQ ID NO: 19; the HCDR2 comprises the amino acid sequence of SEQ ID NO: 22; and the HCDR3 comprises the amino acid sequence of SEQ ID NO: 25. In some embodiments, the HCDR1 comprises the amino acid sequence of SEQ ID NO: 20; the HCDR2 comprises the amino acid sequence of SEQ ID NO: 23; and the HCDR3 comprises the amino acid sequence of SEQ ID NO: 26. In some embodiments, the HCDR1 comprises the amino acid sequence of SEQ ID NO: 28; the HCDR2 comprises the amino acid sequence of SEQ ID NO: 44; and the HCDR3 comprises the amino acid sequence of SEQ ID NO: 59. In some embodiments, the HCDR1 comprises the amino acid sequence of SEQ ID NO: 29; the HCDR2 comprises the amino acid sequence of SEQ ID NO: 45; and the HCDR3 comprises the amino acid sequence of SEQ ID NO: 60. In some embodiments, the HCDR1 comprises the amino acid sequence of SEQ ID NO: 30; the HCDR2 comprises the amino acid sequence of SEQ ID NO: 46; and the HCDR3 comprises the amino acid sequence of SEQ ID NO: 61. In some embodiments, the HCDR1 comprises the amino acid sequence of SEQ ID NO: 31; the HCDR2 comprises the amino acid sequence of SEQ ID NO: 47; and the HCDR3 comprises the amino acid sequence of SEQ ID NO: 62. In some embodiments, the HCDR1 comprises the amino acid sequence of SEQ ID NO: 32, the HCDR2 comprises the amino acid sequence of SEQ ID NO: 48; and the HCDR3 comprises the amino acid sequence of SEQ ID NO: 63. In some embodiments, the HCDR1 comprises the amino acid sequence of SEQ ID NO: 33; the HCDR2 comprises the amino acid sequence of SEQ ID NO: 49; and the HCDR3 comprises the amino acid sequence of SEQ ID NO: 64. In some embodiments, the HCDR1 comprises the amino acid sequence of SEQ ID NO: 34, the HCDR2 comprises the amino acid sequence of SEQ ID NO: 50; and the HCDR3 comprises the amino acid sequence of SEQ ID NO: 65. In some embodiments, the HCDR1 comprises the amino acid sequence of SEQ ID NO: 35; the HCDR2 comprises the amino acid sequence of SEQ ID NO: 51; and the HCDR3 comprises the amino acid sequence of SEQ ID NO: 66. In some embodiments, the HCDR1 comprises the amino acid sequence of SEQ ID NO: 36; the HCDR2 comprises the amino acid sequence of SEQ ID NO: 52; and the HCDR3 comprises the amino acid sequence of SEQ ID NO: 67. In some embodiments, the HCDR1 comprises the amino acid sequence of SEQ ID NO: 37; the HCDR2 comprises the amino acid sequence of SEQ ID NO: 53; and the HCDR3 comprises the amino acid sequence of SEQ ID NO: 68. In some embodiments, the HCDR1 comprises the amino acid sequence of SEQ ID NO: 38; the HCDR2 comprises the amino acid sequence of SEQ ID NO: 54; and the HCDR3 comprises the amino acid sequence of SEQ ID NO: 69. In some embodiments, the HCDR1 comprises the amino acid sequence of SEQ ID NO: 39; the HCDR2 comprises the amino acid sequence of SEQ ID NO: 55; and the HCDR3 comprises the amino acid sequence of SEQ ID NO: 70. In some embodiments, the HCDR1 comprises the amino acid sequence of SEQ ID NO: 40; the HCDR2 comprises the amino acid sequence of SEQ ID NO: 56; and the HCDR3 comprises the amino acid sequence of SEQ ID NO: 71. In some embodiments, the HCDR1 comprises the amino acid sequence of SEQ ID NO: 41; the HCDR2 comprises the amino acid sequence of SEQ ID NO: 57; and the HCDR3 comprises the amino acid sequence of SEQ ID NO: 72. In some embodiments, the HCDR1 comprises the amino acid sequence of SEQ ID NO: 42; the HCDR2 comprises the amino acid sequence of SEQ ID NO: 58; and the HCDR3 comprises the amino acid sequence of SEQ ID NO: 73. In some embodiments, the HCDR1 comprises the amino acid sequence of SEQ ID NO: 75; the HCDR2 comprises the amino acid sequence of SEQ ID NO: 79; and the HCDR3 comprises the amino acid sequence of SEQ ID NO: 83. In some embodiments, the HCDR1 comprises the amino acid sequence of SEQ ID NO: 76; the HCDR2 comprises the amino acid sequence of SEQ ID NO: 80; and the HCDR3 comprises the amino acid sequence of SEQ ID NO: 84. In some embodiments, the HCDR1 comprises the amino acid sequence of SEQ ID NO: 77; the HCDR2 comprises the amino acid sequence of SEQ ID NO: 81; and the HCDR3 comprises the amino acid sequence of SEQ ID NO: 85. In some embodiments, the LCDR1 comprises the amino acid sequence of SEQ ID NO: 87; the LCDR2 comprises the amino acid sequence of SEQ ID NO: 99; and the LCDR3 comprises the amino acid sequence of SEQ ID NO: 111. In some embodiments, the LCDR1 comprises the amino acid sequence of SEQ ID NO: 88; the LCDR2 comprises the amino acid sequence of SEQ ID NO: 100; and the LCDR3 comprises the amino acid sequence of SEQ ID NO: 112. In some embodiments, the LCDR1 comprises the amino acid sequence of SEQ ID NO: 89; the LCDR2 comprises the amino acid sequence of SEQ ID NO: 101; and the LCDR3 comprises the amino acid sequence of SEQ ID NO: 113. In some embodiments, the LCDR1 comprises the amino acid sequence of SEQ ID NO: 90; the LCDR2 comprises the amino acid sequence of SEQ ID NO: 102; and the LCDR3 comprises the amino acid sequence of SEQ ID NO: 114. In some embodiments, the LCDR1 comprises the amino acid sequence of SEQ ID NO: 91; the LCDR2 comprises the amino acid sequence of SEQ ID NO. 103; and the LCDR3 comprises the amino acid sequence of SEQ ID NO: 115. In some embodiments, the LCDR1 comprises the amino acid sequence of SEQ ID NO: 92; the LCDR2 comprises the amino acid sequence of SEQ ID NO: 104; and the LCDR3 comprises the amino acid sequence of SEQ ID NO: 116. In some embodiments, the LCDR1 comprises the amino acid sequence of SEQ ID NO: 93; the LCDR2 comprises the amino acid sequence of SEQ ID NO: 105; and the LCDR3 comprises the amino acid sequence of SEQ ID NO: 117. In some embodiments, the LCDR1 comprises the amino acid sequence of SEQ ID NO: 94; the LCDR2 comprises the amino acid sequence of SEQ ID NO: 106; and the LCDR3 comprises the amino acid sequence of SEQ ID NO: 118. In some embodiments, the LCDR1 comprises the amino acid sequence of SEQ ID NO: 95; the LCDR2 comprises the amino acid sequence of SEQ ID NO: 107; and the LCDR3 comprises the amino acid sequence of SEQ ID NO: 119. In some embodiments, the LCDR1 comprises the amino acid sequence of SEQ ID NO: 96; the LCDR2 comprises the amino acid sequence of SEQ ID NO: 108; and the LCDR3 comprises the amino acid sequence of SEQ ID NO: 120. In some embodiments, the LCDR1 comprises the amino acid sequence of SEQ H) NO; 97; the LCDR2 comprises the amino acid sequence of SEQ ID NO: 109; and the LCDR3 comprises the amino acid sequence of SEQ ID NO: 121. In some embodiments, the LCDR1 comprises the amino acid sequence of SEQ ID NO: 123; the LCDR2 comprises the amino acid sequence of SEQ ID NO: 129; and the LCDR3 comprises the amino acid sequence of SEQ ID NO: 135. In some embodiments, the LCDR1 comprises the amino acid sequence of SEQ ID NO: 124; the LCDR2 comprises the amino acid sequence of SEQ ID NO: 130; and the LCDR3 comprises the amino acid sequence of SEQ ID NO: 136. In some embodiments, the LCDR1 comprises the amino acid sequence of SEQ ID NO: 125: the LCDR2 comprises the amino acid sequence of SEQ ID NO: 131; and the LCDR3 comprises the amino acid sequence of SEQ ID NO: 137. In some embodiments, the LCDR1 comprises the amino acid sequence of SEQ ID NO: 126; the LCDR2 comprises the amino acid sequence of SEQ ID NO: 132; and the LCDR3 comprises the amino acid sequence of SEQ ID NO: 138. In some embodiments, the LCDR1 comprises the amino acid sequence of SEQ ID NO: 127; the LCDR2 comprises the amino acid sequence of SEQ ID NO: 133; and the LCDR3 comprises the amino acid sequence of SEQ ID NO: 139. In some embodiments, the LCDR1 comprises the amino acid sequence of SEQ ID NO: 141; the LCDR2 comprises the amino acid sequence of SEQ ID NO: 151; and the LCDR3 comprises the amino acid sequence of SEQ ID NO: 161. In some embodiments, the LCDR1 comprises the amino acid sequence of SEQ ID NO: 142; the LCDR2 comprises the amino acid sequence of SEQ ID NO: 152; and the LCDR3 comprises the amino acid sequence of SEQ ID NO: 162. In some embodiments, the LCDR1 comprises the amino acid sequence of SEQ ID NO: 143; the LCDR2 comprises the amino acid sequence of SEQ ID NO: 153; and the LCDR3 comprises the amino acid sequence of SEQ ID NO: 163. In some embodiments, the LCDR1 comprises the amino acid sequence of SEQ ID NO: 144; the LCDR2 comprises the amino acid sequence of SEQ ID NO: 154; and the LCDR3 comprises the amino acid sequence of SEQ ID NO: 164. In some embodiments, the LCDR1 comprises the amino acid sequence of SEQ ID NO: 145; the LCDR2 comprises the amino acid sequence of SEQ ID NO: 155; and the LCDR3 comprises the amino acid sequence of SEQ ID NO: 165. In some embodiments, the LCDR1 comprises the amino acid sequence of SEQ ID NO: 146; the LCDR2 comprises the amino acid sequence of SEQ ID NO: 156; and the LCDR3 comprises the amino acid sequence of SEQ ID NO: 166. In some embodiments, the LCDR1 comprises the amino acid sequence of SEQ ID NO: 147; the LCDR2 comprises the amino acid sequence of SEQ ID NO: 157; and the LCDR3 comprises the amino acid sequence of SEQ ID NO: 167. In some embodiments, the LCDR1 comprises the amino acid sequence of SEQ ID NO: 148; the LCDR2 comprises the amino acid sequence of SEQ ID NO: 158; and the LCDR3 comprises the amino acid sequence of SEQ ID NO: 168. In some embodiments, the LCDR1 comprises the amino acid sequence of SEQ ID NO: 149; the LCDR2 comprises the amino acid sequence of SEQ ID NO: 159; and the LCDR3 comprises the amino acid sequence of SEQ ID NO: 169.

In some embodiments, the VH of the anti-ANGPTL7 antibodies of the present disclosure includes an amino acid sequence that is at least 90% identical to any of: (a) SEQ ID NOs: 170-174; (b) SEQ ID NOs: 190-191; (c) SEQ ID NOs: 198-212; or (d) SEQ ID NOs: 258-260. In some embodiments, the VL of the anti-ANGPTL7 antibodies of the present disclosure includes an amino acid sequence that is at least 90% identical to any of: (a) SEQ ID NOs: 180-184; (b) SEQ ID NOs: 194-195; (c) SEQ ID NOs: 228-242; or (d) SEQ ID NOs: 264-266. In some embodiments, the VH comprises an amino acid sequence that is at least 90% identical to SEQ ID NO: 170 and the VL comprises an amino acid sequence that is at least 90% identical to SEQ ID NO: 180. In some embodiments, the VH comprises an amino acid sequence that is at least 90% identical to SEQ ID NO: 171 and the VL comprises an amino acid sequence that is at least 90% identical to SEQ ID NO: 181. In some embodiments, the VH comprises an amino acid sequence that is at least 90% identical to SEQ ID NO: 172 and the VL comprises an amino acid sequence that is at least 90% identical to SEQ ID NO: 182. In some embodiments, the VH comprises an amino acid sequence that is at least 90% identical to SEQ ID NO: 173 and the VL comprises an amino acid sequence that is at least 90% identical to SEQ ID NO: 183. In some embodiments, the VH comprises an amino acid sequence that is at least 90% identical to SEQ ID NO: 174 and the VL comprises an amino acid sequence that is at least 90% identical to SEQ ID NO: 184. In some embodiments, the VH comprises an amino acid sequence that is at least 90% identical to SEQ ID NO: 190 and the VL comprises an amino acid sequence that is at least 90% identical to SEQ ID NO: 194. In some embodiments, the VH comprises an amino acid sequence that is at least 90% identical to SEQ ID NO: 191 and the VL comprises an amino acid sequence that is at least 90 identical to SEQ ID NO: 195. In some embodiments, the VH comprises an amino acid sequence that is at least 90% identical to SEQ ID NO: 198 and the VL comprises an amino acid sequence that is at least 90% identical to SEQ ID NO: 228. In some embodiments, the VH comprises an amino acid sequence that is at least 90% identical to SEQ ID NO: 199 and the VL comprises an amino acid sequence that is at least 90% identical to SEQ ID NO: 229. In some embodiments, the VH comprises an amino acid sequence that is at least 90% identical to SEQ ID NO: 200 and the VL comprises an amino acid sequence that is at least 90% identical to SEQ ID NO: 230. In some embodiments, the VH comprises an amino acid sequence that is at least 90% identical to SEQ ID NO: 201 and the VL comprises an amino acid sequence that is at least 90% identical to SEQ ID NO: 231. In some embodiments, the VH comprises an amino acid sequence that is at least 90% identical to SEQ ID NO: 202 and the VL comprises an amino acid sequence that is at least 90% identical to SEQ ID NO: 232. In some embodiments, the VH comprises an amino acid sequence that is at least 90% identical to SEQ ID NO: 203 and the VL comprises an amino acid sequence that is at least 90% identical to SEQ ID NO: 233. In some embodiments, the VH comprises an amino acid sequence that is at least 90% identical to SEQ ID NO: 204 and the VL comprises an amino acid sequence that is at least 90% identical to SEQ ID NO: 234. In some embodiments, the VH comprises an amino acid sequence that is at least 90% identical to SEQ ID NO: 205 and the VL comprises an amino acid sequence that is at least 90% identical to SEQ ID NO: 235. In some embodiments, the VH comprises an amino acid sequence that is at least 90% identical to SEQ ID NO: 206 and the VL comprises an amino acid sequence that is at least 90% identical to SEQ ID NO: 236. In some embodiments, the VH comprises an amino acid sequence that is at least 90% identical to SEQ ID NO: 207 and the VL comprises an amino acid sequence that is at least 90% identical to SEQ ID NO: 237. In some embodiments, the VH comprises an amino acid sequence that is at least 90% identical to SEQ ID NO: 208 and the VL comprises an amino acid sequence that is at least 90% identical to SEQ ID NO: 238. In some embodiments, the VH comprises an amino acid sequence that is at least 90% identical to SEQ ID NO: 209 and the VL comprises an amino acid sequence that is at least 90% identical to SEQ ID NO: 239. In some embodiments, the VH comprises an amino acid sequence that is at least 90% identical to SEQ ID NO: 210 and the VL comprises an amino acid sequence that is at least 90% identical to SEQ ID NO: 240. In some embodiments, the VH comprises an amino acid sequence that is at least 90% identical to SEQ ID NO: 211 and the VL comprises an amino acid sequence that is at least 90% identical to SEQ ID NO: 241. In some embodiments, the VH comprises an amino acid sequence that is at least 90% identical to SEQ ID NO: 212 and the VL comprises an amino acid sequence that is at least 90% identical to SEQ ID NO: 242. In some embodiments, the VH comprises an amino acid sequence that is at least 90% identical to SEQ ID NO: 258 and the VL comprises an amino acid sequence that is at least 90% identical to SEQ ID NO: 264. In some embodiments, the VH comprises an amino acid sequence that is at least 90% identical to SEQ ID NO: 259 and the VL comprises an amino acid sequence that is at least 90% identical to SEQ ID NO: 265. In some embodiments, the VH comprises an amino acid sequence that is at least 90% identical to SEQ ID NO: 260 and the VL comprises an amino acid sequence that is at least 90% identical to SEQ ID NO: 266.

In accordance with the above embodiments, the present disclosure provides anti-ANGPTL7 antibodies comprising various functional characteristics. In some embodiments, the anti-ANGPTL7 antibodies described herein bind ANGPTL7 (or a fragment thereof) and increase outflow facility compared to a control. In some embodiments, the control is selected from the group consisting of vehicle treatment, dexamethasone treatment, ANGPTL7 protein treatment, and ANGPTL7 protein with an isotype control antibody treatment. In some embodiments, the anti-ANGPTL7 antibody comprises: (a) a VH comprising an amino acid sequence that is at least 90% identical to SEQ ID NO: 210 and a VL comprising an amino acid sequence that is at least 90% identical to SEQ ID NO. 240; (b) a VH comprising an amino acid sequence that is at least 90% identical to SEQ ID NO: 200 and a VL comprising an amino acid sequence that is at least 90% identical to SEQ ID NO: 230; (c) a VH comprising an amino acid sequence that is at least 90% identical to SEQ ID NO: 258 and a VL comprising an amino acid sequence that is at least 90% identical to SEQ ID NO: 264; (d) a VH comprising an amino acid sequence that is at least 90% identical to SEQ ID NO: 207 and a VL comprising an amino acid sequence that is at least 90% identical to SEQ ID NO: 237; (e) a VH comprising an amino acid sequence that is at least 90% identical to SEQ ID NO: 204 and a VL comprising an amino acid sequence that is at least 90% identical to SEQ ID NO: 234; (f) a VH comprising an amino acid sequence that is at least 90% identical to SEQ ID NO: 260 and a VL comprising an amino acid sequence that is at least 90% identical to SEQ ID NO: 266; (g) a VH comprising an amino acid sequence that is at least 90% identical to SEQ ID NO: 205 and a VL comprising an amino acid sequence that is at least 90% identical to SEQ ID NO: 235; (h) a VH comprising an amino acid sequence that is at least 90% identical to SEQ ID NO: 206 and a VL comprising an amino acid sequence that is at least 90% identical to SEQ ID NO: 236 (i) a VH comprising an amino acid sequence that is at least 90% identical to SEQ ID NO: 208 and a VL comprising an amino acid sequence that is at least 90% identical to SEQ ID NO: 238; (j) a VH comprising an amino acid sequence that is at least 90% identical to SEQ ID NO: 191 and a VL comprising an amino acid sequence that is at least 90% identical to SEQ ID NO: 195; (k) a VH comprising an amino acid sequence that is at least 90% identical to SEQ ID NO: 203 and a VL comprising an amino acid sequence that is at least 90% identical to SEQ ID NO: 233; (l) a VH comprising an amino acid sequence that is at least 90% identical to SEQ ID NO: 212 and a VL comprising an amino acid sequence that is at least 90% identical to SEQ ID NO: 242; (m) a VH comprising an amino acid sequence that is at least 90% identical to SEQ ID NO: 198 and a VL comprising an amino acid sequence that is at least 90% identical to SEQ ID NO: 228; (n) a VH comprising an amino acid sequence that is at least 90% identical to SEQ ID NO: 190 and a VL comprising an amino acid sequence that is at least 90% identical to SEQ ID NO: 194; (o) a VH comprising an amino acid sequence that is at least 90% identical to SEQ ID NO: 202 and a VL comprising an amino acid sequence that is at least 90% identical to SEQ ID NO: 232; (p) a VH comprising an amino acid sequence that is at least 90% identical to SEQ ID NO: 211 and a VL comprising an amino acid sequence that is at least 90% identical to SEQ ID NO: 241; or (q) a VH comprising an amino acid sequence that is at least 90% identical to SEQ ID NO: 199 and a VL comprising an amino acid sequence that is at least 90% identical to SEQ ID NO: 229.

In some embodiments, the anti-ANGPTL7 antibody comprises: (a) a VH comprising an amino acid sequence that is at least 90% identical to SEQ ID NO: 210 and a VL comprising an amino acid sequence that is at least 90% identical to SEQ ID NO: 240; (b) a VH comprising an amino acid sequence that is at least 90% identical to SEQ ID NO: 200 and a VL comprising an amino acid sequence that is at least 90% identical to SEQ ID NO: 230; or (c) a VH comprising an amino acid sequence that is at least 90% identical to SEQ ID NO: 258 and a VL comprising an amino acid sequence that is at least 90% identical to SEQ ID NO: 264.

In some embodiments, the anti-ANGPTL7 antibody comprises: (a) a VH comprising an amino acid sequence that is at least 90% identical to SEQ ID NO: 207 and a VL comprising an amino acid sequence that is at least 90% identical to SEQ ID NO: 237; (b) a VH comprising an amino acid sequence that is at least 90% identical to SEQ ID NO: 204 and a VL comprising an amino acid sequence that is at least 90% identical to SEQ ID NO: 234; (c) a VH comprising an amino acid sequence that is at least 90% identical to SEQ ID NO: 260 and a VL comprising an amino acid sequence that is at least 90% identical to SEQ ID NO: 266; (d) a VH comprising an amino acid sequence that is at least 90% identical to SEQ ID NO: 205 and a VL comprising an amino acid sequence that is at least 90% identical to SEQ ID NO: 235; (e) a VH comprising an amino acid sequence that is at least 90% identical to SEQ ID NO: 206 and a VL comprising an amino acid sequence that is at least 90% identical to SEQ ID NO: 236 (f) a VH comprising an amino acid sequence that is at least 90% identical to SEQ ID NO: 208 and a VL comprising an amino acid sequence that is at least 90% identical to SEQ ID NO: 238; (g) a VH comprising an amino acid sequence that is at least 90% identical to SEQ ID NO: 191 and a VL comprising an amino acid sequence that is at least 90% identical to SEQ ID NO: 195; (h) a VH comprising an amino acid sequence that is at least 90% identical to SEQ ID NO: 203 and a VL comprising an amino acid sequence that is at least 90% identical to SEQ ID NO: 233; (i) a VH comprising an amino acid sequence that is at least 90% identical to SEQ ID NO: 212 and a VL comprising an amino acid sequence that is at least 90% identical to SEQ ID NO: 242; (j) a VH comprising an amino acid sequence that is at least 90% identical to SEQ ID NO: 198 and a VL comprising an amino acid sequence that is at least 90% identical to SEQ ID NO: 228; or (k) a VH comprising an amino acid sequence that is at least 90% identical to SEQ ID NO: 190 and a VL comprising an amino acid sequence that is at least 90% identical to SEQ ID NO: 194.

In some embodiments, the anti-ANGPTL7 antibody comprises: (a) a VH comprising an amino acid sequence that is at least 90% identical to SEQ ID NO: 202 and a VL comprising an amino acid sequence that is at least 90% identical to SEQ ID NO: 232; (b) a VH comprising an amino acid sequence that is at least 90% identical to SEQ ID NO: 211 and a VL comprising an amino acid sequence that is at least 90% identical to SEQ ID NO: 241; or (c) a VH comprising an amino acid sequence that is at least 90% identical to SEQ ID NO: 199 and a VL comprising an amino acid sequence that is at least 90% identical to SEQ ID NO: 229.

In accordance with the above embodiments, the anti-ANGPTL7 antibodies of the present disclosure can be a monoclonal antibody, a human antibody, a humanized antibody, and/or a chimeric antibody. In some embodiments, the antibody is a fragment selected from the group consisting of Fab, Fab-C, Fab′-SH, Fv, scFv, and (Fab′)₂ fragments. In some embodiments, the anti-ANGPTL7 antibody is a monospecific antibody. In some embodiments, the anti-ANGPTL7 antibody is a bispecific antibody. In some embodiments, the anti-ANGPTL7 antibody comprises two or more single-domain antibodies that form a bivalent antibody, a trivalent antibody, or a tetravalent antibody that recognizes different epitopes on the same or different antigens.

In some embodiments, the antibody comprises a detection moiety. In some embodiments, the antibody comprises a purification moiety. In some embodiments, the antibody comprises a half-life extension moiety. In some embodiments, the half-life extension moiety comprises a polypeptide that is at least 20 amino acids in length and comprises any combination of G, A, S T, E, and P residues. In some embodiments, the half-life extension polypeptide is attached to the C-terminus or N-terminus of the antibody.

The anti-ANGPTL7 antibodies of the present disclosure can be administered as part of a pharmaceutical composition in a therapeutically effective amount to treat an eye disease (e.g., glaucoma and/or a disease affecting the optic nerve or retinal ganglion cells). In some embodiments, the composition is suitable for ocular administration. In some embodiments, ocular administration comprises injection into vitreous fluid. In some embodiments, ocular administration comprises delivering the antibody using a conjunctival insert, a contact lens, a gel, a nanoparticle, a mucoadhesive polymer, an ointment, a solution, a suspension, eye drops, and/or an implant.

Embodiments of the present disclosure also include methods of treating glaucoma and/or a disease affecting the optic nerve and/or retinal ganglion cells. In accordance with these embodiments, the methods include administering a pharmaceutical composition comprising a therapeutically effective amount of an anti-ANGPTL7 antibody of the present disclosure. In some embodiments, the pharmaceutical composition is administered ocularly and treats at least one symptom associated with glaucoma and/or a disease affecting the optic nerve or retinal ganglion cells. In some embodiments, the at least one symptom associated with glaucoma and/or a disease affecting the optic nerve or retinal ganglion cells comprises eye pain, eye pressure, headaches, rainbow-colored halos around lights, low vision, blurred vision, narrowed vision, impaired peripheral vision, blind spots, nausea, vomiting, and red eyes. In some embodiments, administering the pharmaceutical composition attenuates intraocular pressure and/or increases outflow facility in the subject's eye. In some embodiments, the pharmaceutical composition is administered at a dose ranging from about 0.0001 mg/dose to about 100 mg/dose. In some embodiments, the pharmaceutical composition is administered at a dose ranging from about 0.0001 mg/ml to about 100 mg/ml.

Embodiments of the present disclosure also include a polynucleotide encoding any of the anti-ANGPTL7 antibodies of the present disclosure. In some embodiments, the polynucleotide comprises a sequence that is at least 70% identical to any of the following nucleic acid sequences: (a) SEQ ID NOs: 175-179; (b) SEQ ID NOs: 192-193; (c) SEQ ID NOs: 213-227; or (d) SEQ ID NOs: 261-263. In some embodiments, the polynucleotide comprises a sequence that is at least 70% identical to any of the following nucleic acid sequences: (a) SEQ ID NOs: 185-189; (b) SEQ ID NOs: 196-197; (c) SEQ ID NOs: 243-257; or (d) SEQ ID NOs: 267-269. In some embodiments, the polynucleotide comprises a sequence that is at least 80% identical to any of the following nucleic acid sequences: (a) SEQ ID NOs: 175-179; (b) SEQ ID NOs: 192-193; (c) SEQ ID NOs: 213-227; or (d) SEQ ID NOs: 261-263. In some embodiments, the polynucleotide comprises a sequence that is at least 80% identical to any of the following nucleic acid sequences: (a) SEQ ID NOs: 185-189; (b) SEQ ID NOs: 196-197; (c) SEQ ID NOs: 243-257; or (d) SEQ ID NOs: 267-269.

In some embodiments, the polynucleotide encoding an anti-ANGPTL7 antibody of the present disclosure comprises: (a) a nucleic acid sequence that is at least 70% identical to SEQ ID NO: 175 and a nucleic acid sequence that is at least 70% identical to SEQ ID NO: 185; (b) a nucleic acid sequence that is at least 70% identical to SEQ ID NO: 176 and a nucleic acid sequence that is at least 70% identical to SEQ ID NO: 186; (c) a nucleic acid sequence that is at least 70% identical to SEQ ID NO: 177 and a nucleic acid sequence that is at least 70% identical to SEQ ID NO: 187; (d) a nucleic acid sequence that is at least 70% identical to SEQ ID NO: 178 and a nucleic acid sequence that is at least 70% identical to SEQ ID NO: 188; or (e) a nucleic acid sequence that is at least 70% identical to SEQ ID NO: 179 and a nucleic acid sequence that is at least 70% identical to SEQ ID NO: 189.

In some embodiments, the polynucleotide encoding an anti-ANGPTL7 antibody of the present disclosure comprises: (a) a nucleic acid sequence that is at least 70% identical to SEQ ID NO: 192 and a nucleic acid sequence that is at least 70% identical to SEQ ID NO: 196; or (b) a nucleic acid sequence that is at least 70% identical to SEQ ID NO: 193 and a nucleic acid sequence that is at least 70% identical to SEQ ID NO: 197.

In some embodiments, the polynucleotide encoding an anti-ANGPTL7 antibody of the present disclosure comprises: (a) a nucleic acid sequence that is at least 70% identical to SEQ ID NO: 213 and a nucleic acid sequence that is at least 70% identical to SEQ ID NO: 243; (b) a nucleic acid sequence that is at least 70% identical to SEQ ID NO: 214 and a nucleic acid sequence that is at least 70% identical to SEQ ID NO: 244; (c) a nucleic acid sequence that is at least 70% identical to SEQ ID NO: 215 and a nucleic acid sequence that is at least 70% identical to SEQ ID NO: 245; (d) a nucleic acid sequence that is at least 70% identical to SEQ ID NO: 216 and a nucleic acid sequence that is at least 70% identical to SEQ ID NO: 246; (e) a nucleic acid sequence that is at least 70% identical to SEQ ID NO: 217 and a nucleic acid sequence that is at least 70% identical to SEQ ID NO: 247; (f) a nucleic acid sequence that is at least 70% identical to SEQ ID NO: 218 and a nucleic acid sequence that is at least 70% identical to SEQ ID NO: 248; (g) a nucleic acid sequence that is at least 70% identical to SEQ ID NO: 219 and a nucleic acid sequence that is at least 70% identical to SEQ ID NO: 249; (h) a nucleic acid sequence that is at least 70% identical to SEQ ID NO: 220 and a nucleic acid sequence that is at least 70% identical to SEQ ID NO: 250; (i) a nucleic acid sequence that is at least 70% identical to SEQ ID NO: 221 and a nucleic acid sequence that is at least 70% identical to SEQ ID NO: 251; (j) a nucleic acid sequence that is at least 70% identical to SEQ ID NO: 222 and a nucleic acid sequence that is at least 70% identical to SEQ ID NO: 252; or (k) a nucleic acid sequence that is at least 70% identical to SEQ ID NO: 223 and a nucleic acid sequence that is at least 70% identical to SEQ ID NO: 253; (l) a nucleic acid sequence that is at least 70% identical to SEQ ID NO: 224 and a nucleic acid sequence that is at least 70% identical to SEQ ID NO: 254; (m) a nucleic acid sequence that is at least 70% identical to SEQ ID NO: 225 and a nucleic acid sequence that is at least 70% identical to SEQ ID NO: 255; (n) a nucleic acid sequence that is at least 70% identical to SEQ ID NO: 226 and a nucleic acid sequence that is at least 70% identical to SEQ ID NO: 256; or (o) a nucleic acid sequence that is at least 70% identical to SEQ ID NO: 227 and a nucleic acid sequence that is at least 70% identical to SEQ ID NO: 257.

In some embodiments, the polynucleotide encoding an anti-ANGPTL7 antibody of the present disclosure comprises: (a) a nucleic acid sequence that is at least 70% identical to SEQ ID NO: 261 and a nucleic acid sequence that is at least 70% identical to SEQ ID NO: 267; (b) a nucleic acid sequence that is at least 70% identical to SEQ ID NO: 262 and a nucleic acid sequence that is at least 70% identical to SEQ ID NO: 268; or (c) a nucleic acid sequence that is at least 70% identical to SEQ ID NO: 263 and a nucleic acid sequence that is at least 70% identical to SEQ ID NO: 269.

In accordance with these embodiments, the present disclosure includes an expression vector comprising any of the polynucleotides encoding an anti-ANGPTL7 antibody of the present disclosure. In some embodiments, the expression vector is suitable for manufacturing an anti-ANGPTL7 antibody of the present disclosure for delivery of the antibody to a subject. In some embodiments, the expression vector is suitable for use in gene therapy (e.g., an expression vector for delivering a polynucleotide encoding an anti-ANGPTL7 antibody of the present disclosure to a subject). In some embodiments, the expression vector is an adeno-associated virus (AAV) vector, or comprises an AAV backbone. In some embodiments, the expression vector is a lentiviral vector (LV), or comprises an LV backbone. In some embodiments, the expression vector is a herpes simplex virus (HSV) vector, or a retrovirus vector.

In accordance with these embodiments, the present disclosure also provides a method of administering ocular gene therapy to a subject in need thereof comprising injecting a pharmaceutical composition comprising an effective amount of an expression vector described herein (e.g., an expression vector comprising a polynucleotides encoding an anti-ANGPTL7 antibody of the present disclosure). In accordance with these embodiments, the present disclosure also provides a method of treating glaucoma and/or a disease affecting the optic nerve or retinal ganglion cells comprising administering a pharmaceutical composition comprising an effective amount of an expression vector described herein (e.g., an expression vector comprising a polynucleotides encoding an anti-ANGPTL7 antibody of the present disclosure). In some embodiments, administering the pharmaceutical composition treats at least one symptom of glaucoma and/or a disease affecting the optic nerve or retinal ganglion cells.

In accordance with the above embodiments, the anti-ANGPTL7 antibodies of the present disclosure bind an epitope from an ANGPTL7 polypeptide having any amino acid sequence of SEQ ID NOs: 370-374, or a variant thereof. In some embodiments, the anti-ANGPTL7 antibodies of the present disclosure bind an epitope from an ANGPTL7 polypeptide with a K_{D} of about 100 nM or lower.

### BRIEF DESCRIPTION OF THE DRAWING(S)

FIG. 1: Representative data demonstrating the levels of ANGTPL7 gene expression relative to control following treatment with dexamethasone for 5 days.

FIG. 2: Representative data of treatment-induced gene changes from 3 days of ANGPTL7 (50 mg/ml) treatment in human TM and SC cells using an RNAseq panel.

FIGS. 3A-3C: Representative schematic diagram of 3D-HTM scaffolding technology (Glauconix) used as an ex-vivo human eye tissue model to assess the effects of ANGPTL7 on outflow facility (FIG. 3A). FIG. 3B includes representative data demonstrating outflow facility for 3D-HTM donor 2 treated with vehicle (DMSO), 500 nM Dexamethasone, 50 ng/mL of ANGPTL7. Samples were analyzed using One-way ANOVA (GraphPad Prism Software, Inc., La Jolla, CA), *P<0.05, **P<0.0.01, ***P<0.001 N=4 per treatment group. FIG. 3C includes representative data demonstrating outflow facility for 3D-HTM donor 3 treated with vehicle (DMSO), 500 nM Dexamethasone, 50 ng/mL of ANGPTL7. Samples were analyzed using One-way ANOVA (GraphPad Prism Software, Inc., La Jolla, CA), *P<0.05, **P<0.0.01, ***P<0.001 N=4 per treatment group.

FIGS. 4A-4C: FIG. 4A includes representative data demonstrating outflow facility for donor 1 treated with vehicle (DMSO), 500 nM Dexamethasone, or 25, 50, 150 ng/mL of ANGPTL7. Samples were analyzed using One-way ANOVA (GraphPad Prism Software, Inc., La Jolla, CA), *P<0.05, **P<0.0.01, ***P<0.001 N=3 per treatment group. FIG. 4B includes representative data demonstrating outflow facility for donor 2 treated with vehicle (DMSO), 500 nM Dexamethasone, or 25, 50, 150 ng/mL of ANGPTL7. Samples were analyzed using One-way ANOVA (GraphPad Prism Software, Inc., La Jolla, CA), *P<0.05, **P<0.0.01, ***P<0.001 N=3 per treatment group. FIG. 4C includes representative data demonstrating outflow facility for donor 3 treated with vehicle (DMSO), 500 nM Dexamethasone, or 25, 50, 150 ng/mL of ANGPTL7. Samples were analyzed using One-way ANOVA (GraphPad Prism Software, Inc., La Jolla, CA), *P<0.05, **P<0.0.01, ***P<0.001 N=3 per treatment group. (See FIG. 3A for schematic diagram of 3D-HTM scaffolding technology used to generate data in FIGS. 4A-4C.)

FIGS. 5A-5D: Representative ELISA results used to determine antigen positive serum titers of mice immunized with ANGPTL7, according to four different immunization protocols/cohorts (FIG. 5A—Cohort 1; FIG. 5B—Cohort 2; FIG. 5C—Cohort 3; FIG. 5D—Cohort 4).

FIGS. 6A-6I: Representative results of ANGPTL7 antibody cross-blocking experiments, including data from a representative heatmap analyzing the ability of the antibodies to block one another for binding to the antigen (FIG. 6A), and representative dendrograms, which progressively group antibodies with similar competition profiles. FIG. 6B includes data from a granular binning network. FIG. 6C: includes data from a combined binary dendrogram (color indicates bins in the Community binning network). FIG. 6D includes data from a community binning network. FIG. 6E includes binning data based on affinity for huANGPTL7-his (P62). FIG. 6F includes binning data based on antibody source (hybridoma or phage). FIG. 6G includes binning data based on fibrinogen domain (P60P) binding. FIG. 6H includes binning data based on rabbit ANGPTL7 (p66) binding. FIG. 6I includes binning data based on mouse ANGPTL7 binding.

FIG. 7: Representative results of the effects of anti-ANGPTL7 antibodies on conventional outflow facility using a 3D HTM/HSC Tissue Model (see FIG. 3A). Outflow facility of one donor cell line treated with vehicle (DMSO), 500 nM Dexamethasone, 50 ng/mL ANGPTL7 (1.1 nM) alone, 50 ng ANGPTL7+an isotype control Antibody (330) at 11.1 nM, and twenty different Anti-ANGPTL7 antibodies at 11.1 nM together with 50 ng/mL ANGPTL7. Samples were analyzed for effects against isotype control (330) using One-way ANOVA (GraphPad Prism Software, Inc., La Jolla, CA), *P<0.05, **P<0.0.01, ***P<0.001, ****P<0.0001 N=3 per treatment group.

FIGS. 8A-8C: Representative data of the effects of dexamethasone-induced ocular hypertension in ANGPTL7 knockout mice. FIG. 8A includes body weight data of mice post-implantation surgery; dexamethasone impaired weight gain in male mice compared to PBS-treated mice, thereby confirming the proper functioning of the dexamethasone osmotic pumps. FIG. 8B includes intraocular pressure (IOP) data of ANGPTL7 WT and ANGPTL7 KO mice dosed with PBS control or Dexamethasone (4 mg/kg/day); osmotic pumps were implanted on day 0. FIG. 8C includes representative data of the changes in TOP of ANGPTL7 WT and ANGPTL7 KO mice implanted with osmotic pumps containing PBS vehicle or dexamethasone (4 mg/kg/day) over 28 days. All TOP values were normalized to baseline (day 0) readings.

FIGS. 9A-9R: Representative data demonstrating the in vivo tolerability of anti-ANGPTL7 antibodies using single intravitreal (IVT) injections in New Zealand White Rabbits. The anti-ANGPTL7 antibodies indicated were injected at either a 0.5 mg dose (FIGS. 9C, 9D, 9G, 9H, 9K, 9L, 9O, 9P) or a 2.0 mg dose (FIGS. 9E, 9F, 9I, 9J, 9M, 9N, 9Q, 9R) in the right eye (OD), or with a corresponding vehicle dose in the left eye (OS). (FIGS. 9A and 9B include data from controls at 2 mg doses). Intraocular pressure (TOP) measurements (FIGS. 9A, 9C, 9E, 9G, 9I, 9K, 9M, 9O, 9Q) and changes in TOP (FIGS. 9B, 9D, 9F, 9H, 9J, 9L, 9N, 9P, 9R) were taken at the indicated time points over a 21-day period.

FIGS. 10A-10C: Representative pharmacokinetic (PK) data for ANGPTL7 antibody, ATX-P-424, including intraocular pressure (IOP) measurements at 0.5 mg or 2.0 mg (OU) doses compared to vehicle (FIG. 10A); changes in IOP compared to baseline (FIG. 10B); and total ocular examination scores (OE) (FIG. 10C).

FIGS. 11A-1 IC: Representative pharmacokinetic (PK) data for ANGPTL7 antibody, ATX-P-439, including intraocular pressure (IOP) measurements at 0.5 mg or 2.0 mg (OU) doses compared to vehicle (FIG. 11A); changes in IOP compared to baseline (FIG. 11B); and total ocular examination scores (OE) (FIG. 11C).

FIGS. 12A-12C: Representative pharmacokinetic (PK) data for ANGPTL7 antibody, ATX-P-448, including intraocular pressure (IOP) measurements at 0.5 mg or 2.0 mg (OU) doses compared to vehicle (FIG. 12A); changes in IOP compared to baseline (FIG. 12B); and total ocular examination scores (OE) (FIG. 12C).

FIGS. 13A-13: Representative data demonstrating the in vivo tolerability of anti-ANGPTL7 antibodies using single intravitreal (IVT) injections in African Green Monkeys. Data includes absolute IOP values at baseline (day 3) and on day 10, 6 hours post topical administration of saline and Latanoprost (FIG. 13A); changes in IOP 6 hours post topical administration of either saline (day 3) or Latanoprost (day 10) (FIG. 13B); and changes in IOP between vehicle and Latanoprost administration (FIG. 13C).

FIGS. 14A-14H: Representative IOP measurements (FIGS. 14A, 14C, 14E, and 14G) and changes in IOP (FIGS. 14B, 14D, 14F, and 14H) in African Green Monkeys dosed with 2 mg of the indicated anti-ANGPTL7 antibody compared to an isotype control (BTX-330) and a single vehicle eye.

FIG. 15: Representative clinical scores of ocular examinations (OE) across all the dosed groups in FIGS. 14A-14H.

### DETAILED DESCRIPTION

Embodiments of the present disclosure relate to the treatment and/or prevention of glaucoma and other ocular diseases affecting the optic nerve and retinal ganglion cells. In particular, the present disclosure provides novel therapeutic antibodies that target angiopoietin-related protein 7 (ANGPTL7) as a means for reducing intraocular pressure (IOP) and/or increasing outflow facility, thereby preventing optic nerve damage and/or restoring vision.

Angiopoietin-like proteins (ANGPTL) are a family of proteins that have structural similarity to angiopoietin proteins. Seven proteins have been initially grouped into this family (ANGPTL1-7), and more recently, another protein called ANGPTL8 has been identified. ANGPTL proteins have an amino-terminal coiled-coil domain as well as a carboxyl-terminal fibrinogen-like domain, except ANGPTL8, which lacks the later domain. ANGPTL proteins are not known to bind tyrosine kinase receptors such as Tie 1 and Tie 2, distinguishing them from angiopoietin proteins. ANGPTL proteins have been shown to play different physiological roles in metabolism, inflammation and cancer. Increasing evidence is connecting these proteins to obesity and insulin resistance. ANGPTL2, for example, has been shown to associate with adiposity and insulin resistance as well as the development of type 2 diabetes. ANGPTL3, 4, and 8 have been shown to play a major role in regulating lipid metabolism through their inhibition of lipoprotein lipase. Similarly, ANGPTL6 has been shown to be higher in subjects with metabolic syndrome and to positively associate with HDL level. The levels of ANGPTL8 has been shown to be higher in obese and diabetic subjects and to positively associate with insulin resistance and fasting blood glucose in non-diabetic subjects.

ANGPTL7, however, is a poorly studied member of the ANGPTL protein family that has been initially discovered in the stromal layer of the cornea. Levels of ANGPTL7 have been shown to be elevated in glaucoma and its overexpression increases the collagen expression level while, its induction by glucocorticoids caused the up-regulation of important glaucoma-related proteins including fibronectin, myocilin and MMP1. These data suggest that ANGPTL7 may coordinate the trabecular meshwork's extracellular matrix and its response to steroids. Additionally, ANGPTL7 has been associated with various cancers potentially through its interaction with the WNT/-beta-catenin signaling pathway. Currently, there are no therapeutic approaches that target ANGPTL7.

Glaucoma is a leading cause of worldwide irreversible vision loss, characterized by progressive optic neuropathy. The most common form of glaucoma is primary open-angle glaucoma (POAG), which is always accompanied by high intraocular pressure (IOP), the key risk factor for the pathogenesis of POAG. In some cases, prolonged use of dexamethasone (DEX) poses a high risk of elevated IOP and results in secondary glaucoma, which has many common characteristics with POAG. The pathogenesis of POAG can be deduced from the mechanisms underlying DEX-induced ocular hypertension. Understanding the DEX-induced molecular mechanisms may assist in developing therapies for glucocorticoid-induced glaucoma and POAG. Additionally, high IOP is caused by increased outflow resistance of aqueous humor (AH). Accumulating evidence suggests that actin cytoskeletal rearrangement of the trabecular meshwork TM forming cross-linked actin networks is a crucial contributor to this increased resistance. Previous studies have found that the concentration of angiopoietin-like 7 (ANGPTL7) is increased in glaucomatous AH and that overexpression of ANGPTL7 in the TM alters the components of the extracellular matrix (ECM). A recent study found that ANGPTL7 protein-altering variants exert a strong protective effect on glaucoma and suggested ANGPTL7 as a therapeutic target for glaucoma. Thus, ANGPTL7 may play a vital role in modulating TM's ECM and regulating IOP.

In light of this, experiments were performed to determine the role that ANGPTL7 may play in the etiology of glaucoma and other related diseases affecting the optic nerve and retinal ganglion cells, and concomitantly, to develop a therapeutic platform based on modulating ANGPTL7 activity using anti-ANGPTL7 antibodies.

### Definitions

To facilitate an understanding of the present technology, a number of terms and phrases are defined below. Additional definitions are set forth throughout the detailed description.

The use of the terms “a” and “an” and “the” and “at least one” and similar referents in the context of describing the embodiments of the present disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the various embodiments of the present disclosure, and does not pose a limitation on the scope of these embodiment unless otherwise claimed. No language in the specification should be construed as indicating any non-claimed element as essential to the practice of the various embodiments of the present disclosure.

As used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “one or more,” as used herein, refers to a number higher than one. For example, the term “one or more” encompasses any of the following: two or more, three or more, four or more, five or more, six or more, seven or more, eight or more, nine or more, ten or more, eleven or more, twelve or more, thirteen or more, fourteen or more, fifteen or more, twenty or more, fifty or more, 100 or more, or an even greater number.

The term “one or more but less than a higher number,” “two or more but less than a higher number,” “three or more but less than a higher number,” “four or more but less than a higher number,” “five or more but less than a higher number,” “six or more but less than a higher number,” “seven or more but less than a higher number,” “eight or more but less than a higher number,” “nine or more but less than a higher number,” “ten or more but less than a higher number,” “eleven or more but less than a higher number,” “twelve or more but less than a higher number,” “thirteen or more but less than a higher number,” “fourteen or more but less than a higher number,” or “fifteen or more but less than a higher number” is not limited to a higher number. For example, the higher number can be 10,000, 1,000, 100, 50, etc. For example, the higher number can be approximately 50 (e.g., 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10, 9, 8, 7, 6, 5, 4, 3 or 2).

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (C_{H1}, C_{H2}, and C_{H3}) regions, and each light chain contains one N-terminal variable (VL) region and one C-terminal constant (CL) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹¹ M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). Any antigen-binding fragment of the antibody described herein is within the scope of the present disclosure. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H1} domains, (ii) a F(ab′)2 fragment, which is a bivalent fragment comprising two Fab fragments linked by a disulfide bridge at the hinge region, (iii) a Fv fragment consisting of the VL and VH domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a domain antibody (dAb), which is an antibody single variable region domain (VH or VL) polypeptide that specifically binds antigen.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA: see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid.” The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands affects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a “gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “wild-type” when made in reference to a protein refers to a protein that has the characteristics of a naturally occurring protein. The term “naturally-occurring” as applied to an object refers to the fact that an object can be found in nature. For example, a polypeptide or polynucleotide sequence that is present in an organism (including viruses) that can be isolated from a source in nature, and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population, or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. The term “epitope” refers to a sequence of an antigen that is recognized by an antibody or an antigen receptor. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The antigen can be a protein or peptide of viral, bacterial, parasitic, fungal, protozoan, prion, cellular, or extracellular origin, which provokes an immune response in a mammal, preferably leading to protective immunity.

A “pharmaceutically acceptable carrier” as used herein generally refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “pharmaceutical formulation” as used herein generally refers to a preparation which is in such form as to permit the biological activity of an active ingredient (e.g., an anti-ANGPTL7 antibody, an antibody conjugate, a fusion protein, or a polymeric formulation) contained therein to be effective, and which contains no additional components which are unacceptably toxic to a subject to which the formulation would be administered.

As used herein, “treatment” (and grammatical variations thereof such as “treat” or “treating”) generally refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, anti-ANGPTL7 antibodies of the present disclosure or other compositions that include an anti-ANGPTL7 antibody of the present disclosure (e.g., an antibody conjugate, a fusion protein, or a polymeric formulation) are used to delay development of a disease or to slow the progression of a disease.

The term “half-life” as used herein generally refers to the time required for the concentration of a substance (e.g., an anti-ANGPTL7 antibody, an antibody conjugate, a fusion protein (e.g., a Fab fusion protein), or a polymeric formulation) to decrease by one-half, in vivo (e.g., in the eye (e.g., the vitreous)) or in vitro.

An “effective amount” of an agent, e.g., a pharmaceutical formulation, as used herein generally refers to an amount effective, at dosages and for periods of time necessary, to achieve the desired therapeutic or prophylactic result.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. A “subject” may be a “patient.”

### Anti-ANGPTL7 Antibodies

Embodiments of the present disclosure relate to the treatment and/or prevention of glaucoma and other ocular diseases affecting the optic nerve and retinal ganglion cells. In particular, the present disclosure provides novel therapeutic antibodies that target angiopoietin-related protein 7 (ANGPTL7) as a means for reducing intraocular pressure (IOP) and/or increasing outflow facility, thereby preventing optic nerve damage and/or restoring vision.

As described further herein, embodiments of the present disclosure include an antibody, or an antigen binding fragment thereof, which specifically binds human Angiopoietin-Like Protein 7 (ANGPTL7), optionally wherein said human ANGPTL7 is a polypeptide which comprises or consists of the amino acid sequence of any one of SEQ ID NOs: 370 to 374.

In some embodiments, the antibody, or an antigen binding fragment thereof, exhibits any one or more the following functional characteristics: increases outflow facility compared to a control when administered to the eye of a subject, optionally wherein the control is vehicle treatment, dexamethasone treatment, ANGPTL7 protein treatment, or ANGPTL7 protein with an isotype control antibody treatment; and/or binds to ANGPTL7 with a K_{D} of about 100 nM or lower; and/or binds to the same epitope on ANGPTL7 as an antibody comprising the VH and VL sequences of any one of the exemplary antibodies the sequences of which are provided in Table 11; and/or competes for binding to ANGPTL7 with an antibody comprising the VH and VL sequences of any one of the exemplary antibodies the sequences of which are provided in Table 11.

In some embodiments, the antibody, or an antigen binding fragment thereof, is monoclonal, optionally recombinant. In some embodiments, the antibody, or an antigen binding fragment thereof, is human, humanized, or chimeric.

In some embodiments, the antibody, or an antigen binding fragment thereof, is a full length antibody, a single chain antibody, a single chain variable fragment (scFv), a variable fragment (Fv), a fragment antigen-binding region (Fab), a Fab-C, a Fab′-SH, a (Fab′)2, a single-domain antibody (sdAb), a VHH antibody, a nanobody, a camelid-derived single-domain antibody, a shark IgNAR-derived single-domain antibody fragment (VNAR), a diabody, a triabody, an anticalin or an aptamer, optionally wherein the antibody is a full length antibody comprising an Fc region such as a human IgG1, IgG2, IgG3 or IgG4 region.

In some embodiments, the antibody, or an antigen binding fragment thereof, is conjugated to at least one additional moiety, optionally selected from: an antigen binding moiety, such as an antibody or antigen-binding fragment thereof, which is capable of specific binding to a target which is not human ANGPTL7, preferably wherein said target is expressed in the human eye; a therapeutic or cytotoxic moiety; a detection moiety; a purification moiety; a half-life extension moiety, optionally a polypeptide that is at least 20 amino acids in length and comprises any combination of G, A, S T, E, and P residue, which polypeptide is conjugated to the C- or N-terminus of the antibody.

In some embodiments, the antibody, or an antigen binding fragment thereof is a polypeptide comprising: one, two or all three HCDRs of any one of the exemplary antibodies the sequences of which are provided in Table 11, and optionally also one, two or all three of the corresponding LCDRs of the exemplary antibody; and/or a VH sequence having at least 90% identity to the VH sequence of any one of the exemplary antibodies the sequences of which are provided in Table 11, and optionally also a VL sequence having at least 90% identity to the corresponding VL sequence of the exemplary antibody, preferably wherein variation is not permitted in the HCDRs or LCDRs; and/or all six CDRs of any of the exemplary antibodies exemplary antibodies the sequences of which are provided in Table 11; and/or the VH and VL sequences of any one of the exemplary antibodies the sequences of which are provided in Table 11; and/or the full length heavy chain (VH+constant) sequence of any one of the exemplary antibodies the sequences of which are provided in Table 11, and optionally also the corresponding full length light chain (VL+constant) sequence of the exemplary antibody.

Embodiments of the present disclosure also include a polynucleotide encoding an antibody, or an antigen binding fragment thereof, of any of the preceding paragraphs, optionally wherein said polynucleotide comprises or consists of a nucleic acid sequence having at least 70%, 80%, 90% or 100% identity to a nucleic acid sequence of any one of the exemplary antibodies the sequences of which are provided in Table 11.

Embodiments of the present disclosure also include an expression vector comprising the polynucleotide of the preceding paragraph, which is optionally an adeno-associated virus (AAV) vector, a lentiviral (LV) vector, a herpes simplex virus (HSV) vector, or a retrovirus vector.

Embodiments of the present disclosure also include a pharmaceutical composition comprising an antibody, or an antigen binding fragment thereof, a polynucleotide, or a vector according to any one of the preceding paragraphs, and optionally: at least one pharmaceutically acceptable carrier, diluent or preservative; and/or at least one additional active ingredient. In some embodiments, the pharmaceutical composition is suitable for ocular administration to a subject, optionally by delivery using a conjunctival insert, a contact lens, a gel, a nanoparticle, a mucoadhesive polymer, an ointment, a solution, a suspension, eye drops, and/or an implant, preferably by injection into the vitreous fluid.

Embodiments of the present disclosure also include the antibody, or an antigen binding fragment thereof, the polynucleotide, the vector, or the compositions of any of the preceding paragraphs, for use as a medicament, optionally for use in a method of treating a disease of the eye in a subject. In some embodiments, the disease is characterized by increased intraocular pressure and/or reduced outflow facility in the eye of the subject. In some embodiments, the method comprises ocular administration of the antibody, preferably by injection into the vitreous fluid, and wherein said administration preferably relieves at least one symptom in the subject selected from eye pain, eye pressure, headaches, rainbow-colored halos around lights, low vision, blurred vision, narrowed vision, impaired peripheral vision, blind spots, nausea, vomiting, and red eyes. In some embodiments, the disease is glaucoma and/or a disease affecting the optic nerve or retinal ganglion cells, optionally wherein said glaucoma is primary or glucocorticoid-induced glaucoma.

As described further herein, anti-ANGPTL7 antibodies were generated, and their structural and functional properties were elucidated. Based on these data, embodiments of the present disclosure include anti-ANGPTL7 antibodies, or antigen-binding fragments thereof, that are comprised of a heavy chain variable region (VH) comprising complementarity determining regions (CDRs) HCDR1, HCDR2, and HCDR3, and a light chain variable region (VL) comprising complementarity determining regions (CDRs) LCDR1, LCDR2, and LCDR3. In some embodiments, the HCDR1 comprises one of the following amino acid sequences: (a) X₁YX₂IX₃ (SEQ ID NO: 1), wherein X₁ is S or D; X₂ is G or Y; X₃ is S or H; (b) TSGVGVG (SEQ ID NO: 18); (c) X₁X₂X₃MX₄ (SEQ ID NO: 27), wherein X₁ is V, S, D, or T; X₂ is Y, H, or F; X₃ is D, G, S, or A; X₄ is H, S, or N; or (d) SX₁SX₂YWX₃ (SEQ ID NO: 74), wherein X₁ is S or G; X₂ is S or Y; X₃ is G or S. In some embodiments, the HCDR2 comprises one of the following amino acid sequences: (a) WIX₁X₂X₃X₄GX₅TX₆YAQX₇X₈X₉G (SEQ ID NO: 7), wherein X₁ is S, I, or N; X₂ is A or P; X₃ is Y or N; X₄ is N or T; X₅ is N or A; X₆ is N or K; X₇ is N or K; X₈ is L or F; X₉ is R or Q; (b) LIYWNDDKX₁YSPSLKS (SEQ ID NO: 21), wherein X₁ is R or Q; (c) X₁X₂X₃X₄X₅X₆X₇X₈X₉X₁₀X₁X₁₂X₁₃X₁₄X₁₅G (SEQ ID NO: 43), wherein X₁ is G, T, S, A, V, H, or I; X₂ is I or M; X₃ is D, N, T, S, or G; X₄ is P, W, S, G, or Y; X₅ is D, A, N, S, or Y; X₆ is G or S; X₇ is D, G, Y, S, I, or N; X₈ is T, S, N, I, Y, or D; X₉ is Y, T, F, M, K, G, or I; X₁₀ is Y, G, or F; X₁₁ is P, Y, or A; X₁₂ is G, D, or A; X₁₃ is S or D; X₁₄ is V, L, or S; X₁₅ is K or M; or (d) X₁IYYSGSTX₂SNPSLKS (SEQ ID NO: 78) wherein X₁ is S, or Y; X₂ is Y or S. In some embodiments, the HCDR3 comprises one of the following amino acid sequences: (a) SEQ ID NOs: 13-17; (b) X₁X₂X₃X₄X₅X₆FFDX₇ (SEQ ID NO: 24) wherein X₁ is S, D, or N; X₂ is Y or P; X₃ is G or D; X₄ is D or Y; X₅ is Y or G; X₆ is W or D; X₇ is L or Y; (c) SEQ ID NOs: 59-73; or (d) X₁X₂X₃X₄GX₅X₆X₇X₈X₉Y (SEQ ID NO: 82) wherein X₁ is Q or A; X₂ is Y or K; X₃ is I or W; X₄ is S or E; X₅ is T or D; X₆ is E or Y; X₇ is Y or F; X₈ is F or D; X₉ is Q or Y.

In addition to the above HCDR1, HCDR2, and HCDR3 sequences, anti-ANGPTL7 antibodies of the present disclosure include an LCDR1 comprising an amino acid sequence of any of SEQ ID NOs: 87-97, SEQ ID NOs: 123-127, or SEQ ID NOs: 141-149; the LCDR2 comprises an amino acid sequence of any of SEQ ID NOs: 99-109, SEQ ID NOs: 129-133, or SEQ ID NOs 151-159; and the LCDR3 comprises an amino acid sequence of any of SEQ ID NOs: 111-121, SEQ ID NOs: 135-139, or SEQ ID NOs: 161-169.

In some embodiments, the present disclosure provides anti-ANGPTL7 antibodies, or antigen-binding fragments thereof, that include a VH comprising complementarity determining regions HCDR1, HCDR2, and HCDR3, and a VL comprising complementarity determining regions LCDR1, LCDR2, and LCDR3. In some embodiments, the LCDR1 comprises one of the following amino acid sequences: (a) RASQX₁IX₂X₃X₄LX₅ (SEQ ID NO: 86), wherein X₁ is G or S; X₂ is S, R, or Y; X₃ is S, N, or I; X₄ is W, D, or Y; X₅ is A, G, or N; (b) RSSQSLX₁X₂SX₃X₄X₅X₆YLX₇ (SEQ ID NO: 122), wherein X₁ is L or V; X₂ is H, Y, or F; X₃ is N or D; X₄ is R or G; X₅ is Y or N; X₆ is N or T; X₇ is D or N; or (c) RASQSVSX₁X₂X₃X₄A (SEQ ID NO: 140), wherein X₁ is S, N, or R; X₂ is Y or S; X₃ is L or Y; X₄ is A or L. In some embodiments, the LCDR2 comprises one of the following amino acid sequences: (a) AX₁SSLX₂S (SEQ ID NO: 98), wherein X₁ is A or T; X₂ is Q or P; (b) X₁X₂SNRX₃S (SEQ ID NO: 128), wherein X₁ is L, K, or E; X₂ is G or V; X₃ is A or D; or (c) X₁ASX₂RAT (SEQ ID NO: 150), wherein X₁ is D or G; X₂ is N, S, or T. In some embodiments, the LCDR3 comprises one of the following amino acid sequences: (a) X₁QX₂X₃X₄X₅PX₆X₇ (SEQ ID NO: 110), wherein X₁ is L or Q; X₂ is A, H, S, or D; X₃ is N, F, or Y; X₄ is S, T, or N; X₅ is F, Y, or T; X₆ is W, L, 1, P, or Y; X₇ is T or Y; (b) MQX₁X₂X₃X₄PX₅T (SEQ ID NO: 134), wherein X₁ is T or G; X₂ is L or T; X₃ is Q or H; X₄ is T or W; X₅ is Y or W; or (c) QQX₁X₂X₃X₄X₅X₆T (SEQ ID NO: 160), wherein X₁ is R, Y, or G; X₂ is S, G, or Q; X₃ is N, S, or V; X₄ is W, S, or I; X₅ is P or L; X₆ is L, S, P, or T.

In addition to the above LCDR1, LCDR2, and LCDR3 sequences, anti-ANGPTL7 antibodies of the present disclosure include an HCDR1 comprising an amino acid sequence of any of SEQ ID NOs: 2-6, SEQ ID NOs: 19-20, SEQ ID NOs: 2842, or SEQ ID NOs: 75-77; the HCDR2 comprises an amino acid sequence of any of SEQ ID NOs: 8-12, SEQ ID NOs: 22-23, SEQ ID NOs 44-58, or SEQ ID NOs: 79-81; and the HCDR3 comprises an amino acid sequence of any of SEQ ID NOs: 13-17, SEQ ID NOs: 25-26, SEQ ID NOs: 59-73, or SEQ ID NOs: 83-85.

In some embodiments, an anti-ANGPTL7 antibody comprises the HCDR1 of SEQ ID NO: 2, the HCDR2 of SEQ ID NO: 8, and the HCDR3 of SEQ ID NO: 13. In some embodiments, an anti-ANGPTL7 antibody comprises the HCDR1 of SEQ ID NO: 3, the HCDR2 of SEQ ID NO: 9, and the HCDR3 of SEQ ID NO: 14. In some embodiments, an anti-ANGPTL7 antibody comprises the HCDR1 of SEQ ID NO: 4, the HCDR2 of SEQ ID NO: 10, and the HCDR3 of SEQ ID NO: 15. In some embodiments, an anti-ANGPTL7 antibody comprises the HCDR1 of SEQ ID NO: 5, the HCDR2 of SEQ ID NO: 11, and the HCDR3 of SEQ ID NO: 16. In some embodiments, an anti-ANGPTL7 antibody comprises the HCDR1 of SEQ ID NO: 6, the HCDR2 of SEQ ID NO: 12, and the HCDR3 of SEQ ID NO: 17. In some embodiments, an anti-ANGPTL7 antibody comprises the HCDR1 of SEQ ID NO: 7, the HCDR2 comprises the amino acid sequence of SEQ ID NO: 14, and the HCDR3 of SEQ ID NO: 20. In some embodiments, an anti-ANGPTL7 antibody comprises the HCDR1 of SEQ ID NO: 19, the HCDR2 comprises the amino acid sequence of SEQ ID NO: 22, and the HCDR3 comprises the amino acid sequence of SEQ ID NO: 25. In some embodiments, an anti-ANGPTL7 antibody comprises the HCDR1 of SEQ ID NO: 20, the HCDR2 of SEQ ID NO: 23, and the HCDR3 of SEQ ID NO: 26. In some embodiments, an anti-ANGPTL7 antibody comprises the HCDR1 of SEQ ID NO: 28, the HCDR2 of SEQ ID NO: 44, and the HCDR3 of SEQ ID NO: 59. In some embodiments, an anti-ANGPTL7 antibody comprises the HCDR1 of SEQ ID NO: 29, the HCDR2 of SEQ ID NO: 45, and the HCDR3 of SEQ ID NO: 60. In some embodiments, an anti-ANGPTL7 antibody comprises the HCDR1 of SEQ ID NO: 30, the HCDR2 of SEQ ID NO: 46, and the HCDR3 of SEQ ID NO: 61. In some embodiments, an anti-ANGPTL7 antibody comprises the HCDR1 of SEQ ID NO: 31, the HCDR2 of SEQ ID NO: 47, and the HCDR3 of SEQ ID NO: 62. In some embodiments, an anti-ANGPTL7 antibody comprises the HCDR1 of SEQ ID NO: 32, the HCDR2 of SEQ ID NO: 48, and the HCDR3 of SEQ ID NO: 63. In some embodiments, an anti-ANGPTL7 antibody comprises the HCDR1 of SEQ ID NO: 33, the HCDR2 of SEQ ID NO: 49, and the HCDR3 of SEQ ID NO: 64. In some embodiments, an anti-ANGPTL7 antibody comprises the HCDR1 of SEQ ID NO: 34, the HCDR2 of SEQ ID NO: 50, and the HCDR3 of SEQ ID NO: 65. In some embodiments, an anti-ANGPTL7 antibody comprises the HCDR1 of SEQ ID NO: 35, the HCDR2 of SEQ ID NO: 51, and the HCDR3 of SEQ ID NO: 66. In some embodiments, an anti-ANGPTL7 antibody comprises the HCDR1 of SEQ ID NO: 36, the HCDR2 of SEQ ID NO: 52, and the HCDR3 of SEQ ID NO: 67. In some embodiments, an anti-ANGPTL7 antibody comprises the HCDR1 of SEQ ID NO: 37, the HCDR2 of SEQ ID NO: 53, and the HCDR3 of SEQ ID NO: 68. In some embodiments, an anti-ANGPTL7 antibody comprises the HCDR1 of SEQ ID NO: 38, the HCDR2 of SEQ ID NO: 54, and the HCDR3 of SEQ ID NO: 69. In some embodiments, an anti-ANGPTL7 antibody comprises the HCDR1 of SEQ ID NO: 39, the HCDR2 of SEQ ID NO: 55, and the HCDR3 of SEQ ID NO: 70. In some embodiments, an anti-ANGPTL7 antibody comprises the HCDR1 of SEQ ID NO: 40, the HCDR2 of SEQ ID NO: 56, and the HCDR3 of SEQ ID NO: 71. In some embodiments, an anti-ANGPTL7 antibody comprises the HCDR1 of SEQ ID NO: 41, the HCDR2 of SEQ ID NO: 57, and the HCDR3 of SEQ ID NO: 72. In some embodiments, an anti-ANGPTL7 antibody comprises the HCDR1 of SEQ ID NO: 42, the HCDR2 of SEQ ID NO: 58, and the HCDR3 of SEQ ID NO: 73. In some embodiments, an anti-ANGPTL7 antibody comprises the HCDR1 of SEQ ID NO: 75, the HCDR2 of SEQ ID NO: 79, and the HCDR3 of SEQ ID NO: 83. In some embodiments, an anti-ANGPTL7 antibody comprises the HCDR1 of SEQ ID NO: 76, the HCDR2 of SEQ ID NO: 80, and the HCDR3 of SEQ ID NO: 84. In some embodiments, an anti-ANGPTL7 antibody comprises the HCDR1 of SEQ ID NO: 77, the HCDR2 of SEQ ID NO: 81 and the HCDR3 of SEQ ID NO: 85.

In some embodiments, an anti-ANGPTL7 antibody comprises the LCDR1 of SEQ ID NO: 87, the LCDR2 of SEQ ID NO: 99, and the LCDR3 of SEQ ID NO: 111. In some embodiments, an anti-ANGPTL7 antibody comprises the LCDR1 of SEQ ID NO: 88, the LCDR2 of SEQ ID NO: 100, and the LCDR3 of SEQ ID NO: 112. In some embodiments, an anti-ANGPTL7 antibody comprises the LCDR1 of SEQ ID NO: 89, the LCDR2 of SEQ ID NO: 101, and the LCDR3 of SEQ ID NO: 113. In some embodiments, an anti-ANGPTL7 antibody comprises the LCDR1 of SEQ ID NO: 90, the LCDR2 of SEQ ID NO: 102, and the LCDR3 of SEQ ID NO: 114. In some embodiments, an anti-ANGPTL7 antibody comprises the LCDR1 of SEQ ID NO: 91, the LCDR2 of SEQ ID NO: 103, and the LCDR3 of SEQ ID NO: 115. In some embodiments, an anti-ANGPTL7 antibody comprises the LCDR1 of SEQ ID NO: 92, the LCDR2 of SEQ ID NO: 104, and the LCDR3 of SEQ ID NO: 116. In some embodiments, an anti-ANGPTL7 antibody comprises the LCDR1 of SEQ ID NO: 93, the LCDR2 of SEQ ID NO: 105, and the LCDR3 of SEQ ID NO: 117. In some embodiments, an anti-ANGPTL7 antibody comprises the LCDR1 of SEQ ID NO: 94, the LCDR2 of SEQ ID NO: 106, and the LCDR3 of SEQ ID NO: 118. In some embodiments, an anti-ANGPTL7 antibody comprises the LCDR1 of SEQ ID NO: 95, the LCDR2 of SEQ ID NO: 107, and the LCDR3 of SEQ ID NO: 119. In some embodiments, an anti-ANGPTL7 antibody comprises the LCDR1 of SEQ ID NO: 96, the LCDR2 of SEQ ID NO: 108, and the LCDR3 of SEQ ID NO: 120. In some embodiments, an anti-ANGPTL7 antibody comprises the LCDR1 of SEQ ID NO: 97, the LCDR2 of SEQ ID NO: 109, and the LCDR3 of SEQ ID NO: 121. In some embodiments, an anti-ANGPTL7 antibody comprises the LCDR1 of SEQ ID NO: 123, the LCDR2 of SEQ ID NO: 129, and the LCDR3 of SEQ ID NO: 135. In some embodiments, an anti-ANGPTL7 antibody comprises the LCDR1 of SEQ ID NO: 124, the LCDR2 of SEQ ID NO: 130, and the LCDR3 of SEQ ID NO: 136. In some embodiments, an anti-ANGPTL7 antibody comprises the LCDR1 of SEQ ID NO: 125, the HCDR2 of SEQ ID NO: 131, and the LCDR3 of SEQ ID NO: 137. In some embodiments, an anti-ANGPTL7 antibody comprises the LCDR1 of SEQ ID NO: 126, the LCDR2 of SEQ ID NO: 132, and the LCDR3 of SEQ ID NO: 138. In some embodiments, an anti-ANGPTL7 antibody comprises the LCDR1 of SEQ ID NO: 127, the LCDR2 of SEQ ID NO: 133, and the LCDR3 of SEQ ID NO: 139. In some embodiments, an anti-ANGPTL7 antibody comprises the LCDR1 of SEQ ID NO: 141, the LCDR2 of SEQ ID NO: 151, and the LCDR3 of SEQ ID NO: 161. In some embodiments, an anti-ANGPTL7 antibody comprises the LCDR1 of SEQ ID NO: 142, the LCDR2 of SEQ ID NO: 152, and the LCDR3 of SEQ ID NO: 162. In some embodiments, an anti-ANGPTL7 antibody comprises the LCDR1 of SEQ ID NO: 143, the LCDR2 of SEQ ID NO: 153, and the LCDR3 of SEQ ID NO: 163. In some embodiments, an anti-ANGPTL7 antibody comprises the LCDR1 of SEQ ID NO: 144, the LCDR2 of SEQ ID NO: 154, and the LCDR3 of SEQ ID NO: 164. In some embodiments, an anti-ANGPTL7 antibody comprises the LCDR1 of SEQ ID NO: 145, the LCDR2 of SEQ ID NO: 155, and the LCDR3 SEQ ID NO: 165. In some embodiments, an anti-ANGPTL7 antibody comprises the LCDR1 of SEQ ID NO: 146, the LCDR2 of SEQ ID NO: 156, and the LCDR3 of SEQ ID NO: 166. In some embodiments, an anti-ANGPTL7 antibody comprises the LCDR1 of SEQ ID NO: 147, the LCDR2 of SEQ ID NO: 157, and the LCDR3 of SEQ ID NO: 167. In some embodiments, an anti-ANGPTL7 antibody comprises the LCDR1 of SEQ ID NO: 148, the LCDR2 of SEQ ID NO: 158, and the LCDR3 of SEQ ID NO: 168. In some embodiments, an anti-ANGPTL7 antibody comprises the LCDR1 of SEQ ID NO: 149, the LCDR2 of SEQ ID NO: 159, and the LCDR3 of SEQ ID NO: 169. 1001051 In some embodiments, the VH of the anti-ANGPTL7 antibodies of the present disclosure includes an amino acid sequence that is at least 90% identical (e.g., at least 91%, at least 92%, at least 93%, at least 94%, at least 95%, at least 96%, at least 97%, at least 98%, at least 99%, or 100% identical) to any of: (a) SEQ ID NOs: 170-174; (b) SEQ ID NOs: 190-191; (c) SEQ ID NOs: 198-212; or (d) SEQ ID NOs: 258-260. In some embodiments, the VL of the anti-ANGPTL7 antibodies of the present disclosure includes an amino acid sequence that is at least 90% identical (e.g., at least 91%, at least 92%, at least 93%, at least 94%, at least 95%, at least 96%, at least 97%, at least 98%, at least 99%, or 100% identical) to any of: (a) SEQ ID NOs: 180-184; (b) SEQ ID NOs: 194-195; (c) SEQ ID NOs: 228-242; or (d) SEQ ID NOs: 264-266.

In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 170 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 180. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 171 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 181. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 172 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 182. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 173 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 183. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 174 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 184. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 190 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 194. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 191 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 195. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 198 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 228. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 199 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 229. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 200 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 230. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 201 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 231. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 202 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 232. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 203 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 233. In some embodiments, the VH comprises an amino acid sequence that is at least 90% identical (e.g., at least 91%, at least 92%, at least 93%, at least 94%, at least 950%, at least 96%, at least 97%, at least 98%, at least 99%, or 100% identical) to SEQ ID NO: 204 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 234. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 205 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 235. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 206 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 236. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 207 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 237. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 208 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 238. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 209 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 239. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 210 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 240. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 211 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 241. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 212 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 242. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 258 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 264. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 259 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 265. In some embodiments, the VH comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 260 and the VL comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 266.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

As would be recognized by one of ordinary skill in the art based on the present disclosure, one or more amino acids of the aforementioned anti-ANGPTL7 antibodies, or antigen fragments thereof, can be replaced or substituted with a different amino acid. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg). Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine. Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure. Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into the anti-ANGPTL7 antibodies, or antigen-binding fragments thereof (e.g., insertion into the heavy and/or light chain variable region amino acid sequence). Any number of suitable amino acids can be inserted into the amino acid sequence of the antibody or antigen-binding fragment thereof. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the antibody or antigen-binding fragment thereof. For example, 1-10 amino acids (e.g., 1, 2, 3, 4, 5, 6, 7, 8, 9, or 10 amino acids) may be inserted into the amino acid sequence of the monoclonal antibody or antigen-binding fragment thereof. In this respect, the amino acid(s) can be inserted into an antibody or antigen-binding fragment thereof in any suitable location. Preferably, the amino acid(s) are inserted into a CDR (e.g., CDR1, CDR2, or CDR3) of the antibody or antigen-binding fragment thereof.

The amino acid sequences of the anti-ANGPTL7 antibodies, or antigen-binding fragments thereof, are not limited to the specific amino acid sequences described herein. Indeed, an anti-ANGPTL7 antibody or antigen-binding fragment thereof can comprise any heavy chain polypeptide or light chain polypeptide that competes with the anti-ANGPTL7 antibodies or antigen-binding fragments thereof for conformational binding to ANGPTL7. Antibody competition can be assayed using routine peptide competition assays such as, for example, ELISA, Western blot, or immunohistochemistry methods (see, e.g., U.S. Pat. Nos. 4,828,981 and 8,568,992; and Braitbard et al., Proteome Sci., 4: 12 (2006)).

An anti-ANGPTL7 antibody of the present disclosure may be a whole antibody, or an antigen-binding fragment of a whole antibody. As defined herein, antigen-binding antibody fragments encompassed by the present disclosure include, but are not limited to, F(ab′)₂, Fab′, Fab, Fv, scFv, dsFv, dAb, and single chain binding polypeptides. Antibody fragments and their therapeutic utility are further described in, e.g., Nelson, A. L., MAbs. 2010 January-February; 2(1): 77-83; Joosten et al., Microbial Cell Factories volume 2, Article number: 1 (2003); and Bates A, Power C A., Antibodies (Basel). 2019; 8(2):28; doi:10.3390/antib8020028). In some embodiments, the anti-ANGPTL7 antigen-binding fragment is a single-chain variable fragment (scFv), which is an engineered antibody generated by the fusion of the heavy (VH) and light chains (VL) of immunoglobulins through a short polypeptide linker. Single chain variable domain (Fv) fragments (scFv) are used in the art in a variety of clinical and therapeutic applications, primarily due to their improved pharmacokinetic properties as compared to the parent monoclonal antibodies and the relative ease of producing them in large quantities at low cost (Monnier et al., Antibodies 2013, 2(2), 193-208; doi.org/10.3390/antib2020193; Safdari et al., Mol Med. 2016; 22: 258-270; and Lu, R, Hwang, Y., Liu, I. et al. Development of therapeutic antibodies for the treatment of diseases. J Biomed Sci 27, 1 (2020). https://doi.org/10.1186/s12929-019-0592-z).

An anti-ANGPTL7 antibody of the present disclosure may be a diabody. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003). An anti-ANGPTL7 antibody of the present disclosure may be a single-domain antibody (also referred to as a nanobody). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). Antibody fragments can be made by various techniques, including but not limited to proteolytic digestion of an intact antibody as well as production by recombinant host cells (e.g., E. coli or phage), as described herein.

In other embodiments, the anti-ANGPTL7 antibody is a whole antibody. As defined herein, a whole antibody comprises two identical copies of a heavy (H) chain polypeptide and two identical copies of a light (L) chain polypeptide. Each of the heavy chains contains one N-terminal variable (VH) region and three C-terminal constant (C_{H1}, C_{H2}, and C_{H3}) regions, and each light chain contains one N-terminal variable (VL) region and one C-terminal constant (CL). The heavy chain C-terminal constant region contains the fragment crystallizable (Fc) domain, which determines antibody class and is responsible for humoral and cellular effector functions. Antibodies are divided into five major classes (or “isotypes”), IgG, IgM, IgA, IgD and IgE, which differ in their function in the immune system. IgGs are the most abundant immunoglobulins in the blood, representing 60% of total serum antibodies in humans. IgG antibodies may be subclassified as IgG1, IgG2, IgG3, and IgG4, named in order of their abundance in serum (IgG1 being the most abundant) (Vidarsson et al., Frontiers in Immunology. 5: 520 (2014)). A whole anti-ANGPTL7 monoclonal antibody described herein may be of any suitable class and/or subclass. In some embodiments, the monoclonal antibody is of class IgG (e.g., IgG1, IgG2, IgG3, or IgG4). For example, the monoclonal antibody may be an IgG1 antibody.

As discussed above, the Fc domain mediates several effector functions of antibodies, such as binding to receptors on target cells and complement fixation (triggering effector functions that eliminate the antigen). In some embodiments, the Fc domain may be modified or engineered to alter its effector functions. For example, Fc domains may be modified to improve antibody-dependent cellular cytotoxicity (ADCC) and antibody-dependent cellular phagocytosis (ADCP), and to control serum half-life. In some embodiments, the Fc domain of the anti-ANGPTL7 antibody may be engineered to modulate affinity for an Fc receptor, such as Fcγ receptors (FcγRs) and the neonatal Fc receptor (FcRn). Indeed, optimization of the interactions between antibodies and FcγRs has emerged as a promising approach for enhancing the activity of therapeutic antibodies for the treatment of various diseases (Mimoto et al., Curr. Pharm. Biotechnol. 17, 1298-1314 (2016); Lazar et al., Proc. Natl Acad. Sci. USA 103, 4005-4010 (2006); Richards et al., Mol. Cancer Ther. 7, 2517-2527 (2008); Nordstrom et al., Breast Cancer Res. 13, R123 (2011); and Kang, T. H., Jung, S. T., Exp Mol Med 51, 1-9 (2019)). The Fc domain also may be modified to improve serum half-life, e.g., by engineering IgG Fc for higher FcRn binding (Zalevsky et al., Nat. Biotechnol. 28, 157-159 (2010); and Dall'Acqua et al., J. Immunol. 169, 5171-5180 (2002)). In other embodiments, the Fc domain may be modified to create monovalency or antibody bispecificity for improving therapeutic potency. For example, an Fc domain may be generated that does not form a homodimer but remains as a soluble monomer, mFc, that exhibits high affinity for FcγRI but no detectable binding to FcγRIIIa. In other embodiments, a heterodimeric Fc domain may be generated to obtain bispecific properties for antigen binding to circumvent homodimer formation. Engineered Fc domains may be generated by inducing point mutations or by modifying glycosylation of the Fc domain (Saunders, K. O., Front Immunol. 2019; 10:1296; Kelley, R. F., Meng, Y. G., Liu et al., J Biol Chem. 2014; 289:3571-90; Monnet et al., MAbs. 2014; 6:422-36; Li et al., Proc Natl Acad Sci USA. 2017; 114:3485-90; and Lin et al., Proc Natl Acad Sci USA. 2015; 112:10611-6; Kang and Jung, supra).

### Multispecific Anti-ANGPTL7 Antibodies

As described above, the anti-ANGPTL7 antibodies of the present disclosure can be a monoclonal antibody, a human antibody, a humanized antibody, and/or a chimeric antibody. In some embodiments, the antibody is a fragment selected from the group consisting of Fab, Fab-C, Fab′-SH, Fv, scFv, and (Fab′)₂ fragments. In some embodiments, the anti-ANGPTL7 antibody is a monospecific antibody. In some embodiments, the anti-ANGPTL7 antibody is a bispecific antibody. In some embodiments, the anti-ANGPTL7 antibody comprises two or more single-domain antibodies that form a bivalent antibody, a trivalent antibody, or a tetravalent antibody that recognizes different epitopes on the same or different antigens.

In some embodiments, an anti-ANGPTL7 antibody provided herein is a chimeric antibody. Certain chimeric antibodies are described, for example, in U.S. Pat. No. 4,816,567; and Morrison et al., Proc. Natl. Acad. Sci. USA. 81:6851-6855 (1984). In one example, a chimeric antibody comprises a non-human variable region (e.g., a variable domain derived from a mouse, rat, hamster, rabbit, or non-human primate, such as a monkey) and a human constant domain. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, for example, CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, for example, in Almagro and Fransson, Front. Biosci. 13:1619-1633 (2008), and are further described, for example, in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer. 83:252-260 (2000) (describing the “guided selection” approach to FR shuffling).

In accordance with the above embodiments, an anti-ANGPTL7 antibody of the present disclosure can be made into bivalent, trivalent, or tetravalent formats. For example, an anti-ANGPTL7 antibody of the present disclosure can be a bivalent, bispecific antibody with heteromeric heavy chains (e.g., Triomab, knobs-into-holes (KIH), Duobody, etc). An anti-ANGPTL7 antibody of the present disclosure can be a tetravalent multispecific antibody comprised of IgGs with other binding domains fused to either the N- or C-termini of either the heavy or light chains (e.g., dual variable domain [DVD], IgG-scFv fusion, Mabtyrin (IgG with non-antibody binding scaffold “centyrin” fused to C-terminal end of heavy chains). An anti-ANGPTL7 antibody of the present disclosure can be comprised of IgGs to which additional antigen combining sites have been added within the structure (e.g., two-in-one antibodies, MAT “Modular Antibody Technology” platform from F-Star). An anti-ANGPTL7 antibody of the present disclosure can be an engineered antibody fragment linked by short peptide linkers which can be made into bivalent, trivalent, or tetravalent formats addressing two to three targets (e.g., bispecific T-cell engager (BiTE), Nanobody platform, dual-affinity re-targeting (DART) antibodies, “tandem antibody” structures (TandAbs)). And an anti-ANGPTL7 antibody of the present disclosure can be comprised of chemically coupled IgGs.

In some embodiments, an anti-ANGPTL7 antibody of the present disclosure is a multispecific antibody, such as a bispecific antibody, which have binding specificities for at least two different antigens. In some embodiments, the anti-ANGPTL7 antibodies of the present disclosure, or antigen-binding fragments thereof, can be used to form one arm (e.g., antigen-binding portion) of a bispecific antibody, whereas the other arm of the bispecific antibody can be specific for a different antigen. In some embodiments, the other antigen includes, but is not limited to, interleukin-1 beta (IL-1β), interleukin-6 (IL-6); interleukin-6 receptor (IL-6R); interleukin-13 (IL-13); IL-13 receptor (IL-13R); PDGF (e.g., PDGF-BB); angiopoietin; angiopoietin 2 (Ang2); Tie2; SIP; integrins αvβ3, αvβ5, and α5β1; betacellulin; apelin/APJ; erythropoietin; complement factor D; TNFα; HtrA1; a VEGF receptor (e.g., VEGFR1, VEGFR2, VEGFR3, membrane-bound VEGF-receptor (mbVEGFR), or soluble VEGF receptor (sVEGFR)); ST-2 receptor; and proteins genetically linked to age-related macular degeneration (AMD) risk, such as complement pathway components C2, factor B, factor H, CFHR3, C3b, C5, C5a, and C3a; HtrA1; ARMS2; TIMP3; HLA; interleukin-8 (IL-8); CX3CR1; TLR3; TLR4; CETP; LIPC; COL10A1; and TNFRSF10A.

In some embodiments, a bispecific antibody of the present disclosure includes an anti-ANGPTL7 antibody, or an antigen-binding fragment thereof, and an anti-VEGF antibody, or an antigen-binding fragment thereof. Such bispecific antibodies can be used to target different mechanisms, and thus provide additional therapeutic benefits. For example, the anti-ANGPTL7 arm can be any of the anti-ANGPTL7 antibodies of the present disclosure, and the anti-VEGF arm can be any VEGF antagonist, including but not limited to, anti-VEGF antibodies (e.g., bevacizumab, sevacizumab, and ranibizumab), anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), anti-VEGF arms of VEGF bispecific antibodies (e.g., MP-0250, vanucizumab (VEGF-ANG2)), including anti-VEGF, anti-VEGFR1, and anti-VEGFR2 arms.

### Functional Characteristics of Anti-ANGPTL7 Antibodies

In accordance with the above embodiments, the present disclosure provides anti-ANGPTL7 antibodies comprising various functional characteristics. In some embodiments, the anti-ANGPTL7 antibodies described herein bind an antigen on ANGPTL7 (SEQ ID NO: 370), or a variant or isoform thereof, via interaction with its antigenic determinants (epitopes). In some embodiments, the anti-ANGPTL7 antibodies described herein bind an antigen/epitope from a human ANGPTL7 fibrinogen domain (e.g., ATX-P-60; SEQ ID NO: 373), an antigen/epitope from a full length human ANGPTL7 monomeric variant L59P_L84P (ATX-P-62; SEQ ID NO: 371), an antigen/epitope from a full length human ANGPTL7 monomeric variant L59 GGPGG (ATX-P-63; SEQ ID NO: 372), an antigen/epitope from a human wild type ANGPTL7 multimer (PExt-1; SEQ ID NO: 374).

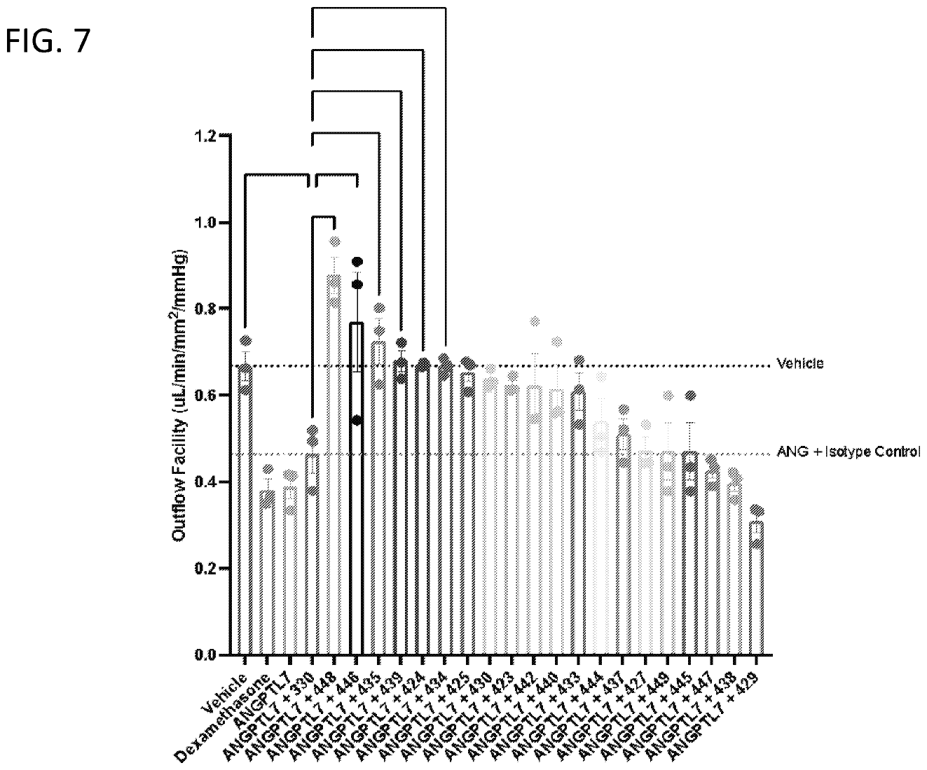

In some embodiments, binding of an anti-ANGPTL7 antibody to an ANGPTL7 polypeptide reduces intraocular pressure and/or increases outflow facility (see, e.g., Example 9 and FIG. 7). In some embodiments, the anti-ANGPTL7 antibody binds an epitope on human ANGPTL7 with a K_{D} of about 100 nM or lower (see, e.g., Example 10). In some embodiments, the anti-ANGPTL7 antibodies described herein bind ANGPTL7 (or a fragment thereof) and increase outflow facility compared to a control. In some embodiments, the control is selected from the group consisting of vehicle treatment, dexamethasone treatment, ANGPTL7 protein treatment, and treatment with an ANGPTL7 protein/polypeptide and an isotype control antibody (see, e.g., FIG. 7). In some embodiments, the anti-ANGPTL7 antibody comprises a VH comprising an amino acid sequence that is at least 90% identical (e.g., at least 91%, at least 920, at least 93%, at least 94%, at least 95%, at least 96%, at least 97%, at least 98%, at least 99%, or 100% identical) to SEQ ID NO: 210 and a VL comprising an amino acid sequence that is at least 90% identical (e.g., at least 91%, at least 92%, at least 93%, at least 94%, at least 95%, at least %%, at least 97%, at least 98%, at least 99%, or 100% identical) to SEQ ID NO: 240. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 200 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 230. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 258 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 264. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 207 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 237. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 204 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 234. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 260 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 266. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 205 and a VL comprising an amino acid sequence that is at least 90% identical (e.g., at least 91%, at least 92%, at least 93%, at least 94%, at least 95%, at least 96%, at least 97%, at least 98%, at least 99%, or 1009% identical) to SEQ ID NO: 235. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 206 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 236. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 208 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 238. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 191 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 195. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 203 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 233. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 212 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 242. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 198 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 228. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 190 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 194. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 202 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 232. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 211 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 241. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 199 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 229.

In some embodiments, the anti-ANGPTL7 antibodies described herein bind ANGPTL7 (or a fragment thereof) and increase outflow facility compared to a control. In some embodiments, the control is any of vehicle treatment, dexamethasone treatment, ANGPTL7 protein treatment, and/or treatment with an ANGPTL7 protein/polypeptide and an isotype control antibody. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 210 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 240. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 200 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 230. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 258 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 264.

In some embodiments, the anti-ANGPTL7 antibodies described herein bind ANGPTL7 (or a fragment thereof) and increase outflow facility compared to a control. In some embodiments, the control is any of dexamethasone treatment, ANGPTL7 protein treatment, and/or treatment with an ANGPTL7 protein/polypeptide and an isotype control antibody. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 207 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 237. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 204 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 234. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 260 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 266. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 205 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 235. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 206 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 236. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 208 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 238. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 191 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 195. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 203 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 233. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 212 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 242. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 198 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 228. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 190 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 194.

In some embodiments, the anti-ANGPTL7 antibodies described herein bind ANGPTL7 (or a fragment thereof) and increase outflow facility compared to a control. In some embodiments, the control is any of dexamethasone treatment and/or ANGPTL7 protein treatment. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 999%, or 100% identical) to SEQ ID NO: 202 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 232. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 211 and a VL comprising an amino acid sequence that is at least 90% identical (e.g., at least 91%, at least 92%, at least 93%, at least 94%, at least 95%, at least %%, at least 97%, at least 98%, at least 99%, or 100% identical) to SEQ ID NO: 241. In some embodiments, the anti-ANGPTL7 antibody comprises a VH comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 199 and a VL comprising an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 229.

### Polypeptides and Expression Vectors

Embodiments of the present disclosure also include a polynucleotide encoding any of the anti-ANGPTL7 antibodies of the present disclosure. In some embodiments, the polynucleotide comprises a sequence that is at least 70% identical (e.g., at least 70%, at least 75%, at least 80%, at least 85%, at least 90%, at least 95%, or 100% identical) to any of the following nucleic acid sequences: (a) SEQ ID NOs: 175-179; (b) SEQ ID NOs: 192-193; (c) SEQ ID NOs: 213-227; or (d) SEQ ID NOs: 261-263. In some embodiments, the polynucleotide comprises a sequence that is at least 70% identical (e.g., at least 70%, at least 75%, at least 80%, at least 85%, at least 90%, at least 95%, or 100% identical) to any of the following nucleic acid sequences: (a) SEQ ID NOs: 185-189; (b) SEQ ID NOs: 196-197; (c) SEQ ID NOs: 243-257; or (d) SEQ ID NOs: 267-269. In some embodiments, the polynucleotide comprises a sequence that is at least 80% identical (e.g., at least 80%, at least 85%, at least 90%, at least 95%, or 100% identical) to any of the following nucleic acid sequences: (a) SEQ ID NOs: 175-179; (b) SEQ ID NOs: 192-193; (c) SEQ ID NOs: 213-227; or (d) SEQ ID NOs: 261-263. In some embodiments, the polynucleotide comprises a sequence that is at least 80% identical (e.g., at least 80%, at least 85%, at least 90%, at least 95%, or 100% identical) to any of the following nucleic acid sequences: (a) SEQ ID NOs: 185-189; (b) SEQ ID NOs: 196-197; (c) SEQ ID NOs: 243-257; or (d) SEQ ID NOs: 267-269. In some embodiments, the polynucleotide comprises a sequence that is at least 85% identical (e.g., at least 85%, at least 90%, at least 95%, or 100% identical) to any of the following nucleic acid sequences: (a) SEQ ID NOs: 175-179; (b) SEQ ID NOs: 192-193; (c) SEQ ID NOs: 213-227; or (d) SEQ ID NOs: 261-263. In some embodiments, the polynucleotide comprises a sequence that is at least 85% identical (e.g., at least 85%, at least 90%, at least 95%, or 100% identical) to any of the following nucleic acid sequences: (a) SEQ ID NOs: 185-189; (b) SEQ ID NOs: 196-197; (c) SEQ ID NOs: 243-257; or (d) SEQ ID NOs: 267-269. In some embodiments, the polynucleotide comprises a sequence that is at least 90% identical (e.g., at least 90%, at least 95%, or 100% identical) to any of the following nucleic acid sequences: (a) SEQ ID NOs: 175-179; (b) SEQ ID NOs: 192-193; (c) SEQ ID NOs: 213-227; or (d) SEQ ID NOs: 261-263. In some embodiments, the polynucleotide comprises a sequence that is at least 90% identical (e.g., at least 90%, at least 95%, or 100% identical) to any of the following nucleic acid sequences: (a) SEQ ID NOs: 185-189; (b) SEQ ID NOs: 196-197; (c) SEQ ID NOs: 243-257; or (d) SEQ ID NOs: 267-269. In some embodiments, the polynucleotide comprises a sequence that is at least 95% identical (e.g., at least 95%, or 100% identical) to any of the following nucleic acid sequences: (a) SEQ ID NOs: 175-179; (b) SEQ ID NOs: 192-193; (c) SEQ ID NOs: 213-227; or (d) SEQ ID NOs: 261-263. In some embodiments, the polynucleotide comprises a sequence that is at least 95% identical (e.g., at least 95%, or 100% identical) to any of the following nucleic acid sequences: (a) SEQ ID NOs: 185-189; (b) SEQ ID NOs: 196-197; (c) SEQ ID NOs: 243-257; or (d) SEQ ID NOs: 267-269.

In some embodiments, the polynucleotide encoding an anti-ANGPTL7 antibody of the present disclosure comprises: (a) a nucleic acid sequence that is at least 70% identical (e.g., at least 70%, at least 75%, at least 80%, at least 85%, at least 90%, at least 95%, or 100% identical) to SEQ ID NO: 175 and a nucleic acid sequence that is at least 70% identical (e.g., at least 70%, at least 75%, at least 80%, at least 85%, at least 90%, at least 95%, or 100% identical) to SEQ ID NO: 185; (b) a nucleic acid sequence that is at least 70% identical (e.g., at least 70%, at least 75%, at least 80%, at least 85%, at least 90%, at least 95%, or 100% identical) to SEQ ID NO: 176 and a nucleic acid sequence that is at least 70% identical (e.g., at least 70%, at least 75%, at least 80%, at least 85%, at least 90%, at least 95%, or 100% identical) to SEQ ID NO: 186; (c) a nucleic acid sequence that is at least 70% identical (e.g., at least 70%, at least 75%, at least 80%, at least 85%, at least 90%, at least 95%, or 100% identical) to SEQ ID NO: 177 and a nucleic acid sequence that is at least 70% identical (e.g., at least 70%, at least 75%, at least 80%, at least 85%, at least 90%, at least 95%, or 100% identical) to SEQ ID NO: 187; (d) a nucleic acid sequence that is at least 70% identical (e.g., at least 70%, at least 75%, at least 80%, at least 85%, at least 90%, at least 95%, or 100% identical) to SEQ ID NO: 178 and a nucleic acid sequence that is at least 70% identical (e.g., at least 70%, at least 75%, at least 80%, at least 85%, at least 90%, at least 95%, or 100% identical) to SEQ ID NO: 188; (e) a nucleic acid sequence that is at least 70% identical (e.g., at least 70%, at least 75%, at least 80%, at least 85%, at least 90%, at least 95%, or 100% identical) to SEQ ID NO: 179 and a nucleic acid sequence that is at least 70% identical (e.g., at least 70%, at least 75%, at least 80%, at least 85%, at least 90%, at least 95%, or 100% identical) to SEQ ID NO: 189.

In some embodiments, the polynucleotide encoding an anti-ANGPTL7 antibody of the present disclosure comprises: (a) a nucleic acid sequence that is at least 70% identical (e.g., at least 70%, at least 75%, at least 80%, at least 85%, at least 90%, at least 95%, or 100% identical) to SEQ ID NO: 213 and a nucleic acid sequence that is at least 70% identical (e.g., at least 70%, at least 75%, at least 80%, at least 85%, at least 90%, at least 95%, or 100% identical) to SEQ ID NO: 243; (b) a nucleic acid sequence that is at least 70% identical (e.g., at least 70%, at least 75%, at least 80%, at least 85%, at least 90%, at least 95%, or 100% identical) to SEQ ID NO: 214 and a nucleic acid sequence that is at least 70% identical (e.g., at least 70%, at least 75%, at least 80%, at least 85%, at least 90%, at least 95%, or 100% identical) to SEQ ID NO: 244; (c) a nucleic acid sequence that is at least 70% identical (e.g., at least 70%, at least 75%, at least 80%, at least 85%, at least 90%, at least 95%, or 100% identical) to SEQ ID NO: 215 and a nucleic acid sequence that is at least 70% identical (e.g., at least 70%, at least 75%, at least 80%, at least 85%, at least 90%, at least 95%, or 100% identical) to SEQ ID NO: 245; (d) a nucleic acid sequence that is at least 70% identical (e.g., at least 70%, at least 75%, at least 80%, at least 85%, at least 90%, at least 95%, or 100% identical) to SEQ ID NO: 216 and a nucleic acid sequence that is at least 70% identical (e.g., at least 70%, at least 75%, at least 80%, at least 85%, at least 90%, at least 95%, or 100% identical) to SEQ ID NO: 246; (e) a nucleic acid sequence that is at least 70% identical (e.g., at least 70%, at least 75%, at least 80%, at least 85%, at least 90%, at least 95%, or 100% identical) to SEQ ID NO: 217 and a nucleic acid sequence that is at least 70% identical (e.g., at least 70%, at least 75%, at least 80%, at least 85%, at least 90%, at least 95%, or 100% identical) to SEQ ID NO: 247; (f) a nucleic acid sequence that is at least 70% identical (e.g., at least 70%, at least 75%, at least 80%, at least 85%, at least 90%, at least 95%, or 100% identical) to SEQ ID NO: 218 and a nucleic acid sequence that is at least 70% identical (e.g., at least 70%, at least 75%, at least 80%, at least 85%, at least 90%, at least 95%, or 100% identical) to SEQ ID NO: 248; (g) a nucleic acid sequence that is at least 70% identical (e.g., at least 70%, at least 75%, at least 80%, at least 85%, at least 90%, at least 95%, or 100% identical) to SEQ ID NO: 219 and a nucleic acid sequence that is at least 70% identical (e.g., at least 70%, at least 75%, at least 80%, at least 85%, at least 90%, at least 95%, or 100% identical) to SEQ ID NO: 249; (h) a nucleic acid sequence that is at least 70% identical (e.g., at least 70%, at least 75%, at least 80%, at least 85%, at least 90%, at least 95%, or 100% identical) to SEQ ID NO: 220 and a nucleic acid sequence that is at least 70% identical (e.g., at least 70%, at least 75%, at least 80%, at least 85%, at least 90%, at least 95%, or 100% identical) to SEQ ID NO: 250; (i) a nucleic acid sequence that is at least 70% identical (e.g., at least 70%, at least 75%, at least 80%, at least 85%, at least 90%, at least 95%, or 100% identical) to SEQ ID NO: 221 and a nucleic acid sequence that is at least 70% identical (e.g., at least 70%, at least 75%, at least 80%, at least 85%, at least 90%, at least 95%, or 100% identical) to SEQ ID NO: 251; or (j) a nucleic acid sequence that is at least 70% identical (e.g., at least 70%, at least 75%, at least 80%, at least 85%, at least 90%, at least 95%, or 100% identical) to SEQ ID NO: 222 and a nucleic acid sequence that is at least 70% identical (e.g., at least 70%, at least 75%, at least 80%, at least 85%, at least 90%, at least 95%, or 100% identical) to SEQ ID NO: 252; (k) a nucleic acid sequence that is at least 70% identical (e.g., at least 70%, at least 75%, at least 80%, at least 85%, at least 90%, at least 95%, or 100% identical) to SEQ ID NO: 223 and a nucleic acid sequence that is at least 70% identical (e.g., at least 70%, at least 75%, at least 80%, at least 85%, at least 90%, at least 95%, or 100% identical) to SEQ ID NO: 253; (l) a nucleic acid sequence that is at least 70% identical (e.g., at least 70%, at least 75%, at least 80%, at least 85%, at least 90%, at least 95%, or 100% identical) to SEQ ID NO: 224 and a nucleic acid sequence that is at least 70% identical (e.g., at least 70%, at least 75%, at least 80%, at least 85%, at least 90%, at least 95%, or 100% identical) to SEQ ID NO: 254; or (m) a nucleic acid sequence that is at least 70% identical (e.g., at least 70%, at least 75%, at least 80%, at least 85%, at least 90%, at least 95%, or 100% identical) to SEQ ID NO: 225 and a nucleic acid sequence that is at least 70% identical (e.g., at least 70%, at least 75%, at least 80%, at least 85%, at least 90%, at least 95%, or 100% identical) to SEQ ID NO: 255; (n) a nucleic acid sequence that is at least 70% identical (e.g., at least 70%, at least 75%, at least 80%, at least 85%, at least 90%, at least 95%, or 100% identical) to SEQ ID NO: 226 and a nucleic acid sequence that is at least 70% identical (e.g., at least 70%, at least 75%, at least 80%, at least 85%, at least 90%, at least 95%, or 100% identical) to SEQ ID NO: 256; or (o) a nucleic acid sequence that is at least 70% identical (e.g., at least 70%, at least 75%, at least 80%, at least 85%, at least 90%, at least 95%, or 100% identical) to SEQ ID NO: 227 and a nucleic acid sequence that is at least 70% identical (e.g., at least 70%, at least 75%, at least 80%, at least 85%, at least 90%, at least 95%, or 100% identical) to SEQ ID NO: 257.

In some embodiments, the polynucleotide encoding an anti-ANGPTL7 antibody of the present disclosure comprises: (a) a nucleic acid sequence that is at least 70% identical (e.g., at least 70%, at least 75%, at least 80%, at least 85%, at least 90%, at least 95%, or 100% identical) to SEQ ID NO: 261 and a nucleic acid sequence that is at least 70% identical (e.g., at least 70%, at least 75%, at least 80%, at least 85%, at least 90%, at least 95%, or 100% identical) to SEQ ID NO: 267; (b) a nucleic acid sequence that is at least 70% identical (e.g., at least 70%, at least 75%, at least 80%, at least 85%, at least 90%, at least 95%, or 100% identical) to SEQ ID NO: 262 and a nucleic acid sequence that is at least 70% identical (e.g., at least 70%, at least 75%, at least 80%, at least 85%, at least 90%, at least 95%, or 100% identical) to SEQ ID NO: 268; (c) a nucleic acid sequence that is at least 70% identical (e.g., at least 70%, at least 75%, at least 80%, at least 85%, at least 90%, at least 95%, or 100% identical) to SEQ ID NO: 263 and a nucleic acid sequence that is at least 70% identical (e.g., at least 70%, at least 75%, at least 80%, at least 85%, at least 90%, at least 95%, or 100% identical) to SEQ ID NO: 269.

In accordance with these embodiments, the present disclosure includes an expression vector comprising any of the polynucleotides encoding an anti-ANGPTL7 antibody of the present disclosure. In some embodiments, the expression vector is suitable for manufacturing an anti-ANGPTL7 antibody of the present disclosure for delivery of the antibody to a subject. In certain embodiments, the nucleic acid sequence is in the form of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 4th edition, Cold Spring Harbor Press, Cold Spring Harbor, N.Y. (2012), and Ausubel et al., Current Protocols in Molecular Biology, Greene Publishing Associates and John Wiley & Sons, New York, N.Y. (1994)).

In addition to the nucleic acid encoding an anti-ANGPTL7 antibody or antigen-binding fragment thereof, the vector desirably comprises expression control sequences, such as promoters, enhancers, polyadenylation signals, transcription terminators, internal ribosome entry sites (IRES), and the like, that provide for the expression of the antibody-encoding nucleic sequence in a host cell. Exemplary expression control sequences are known in the art and described in, for example, Goeddel, Gene Expression Technology: Methods in Enzymology, Vol. 185, Academic Press, San Diego, Calif. (1990).

A vector comprising a nucleic acid sequence encoding an anti-ANGPTL7 antibody or antigen-binding fragment thereof may be introduced into a host cell that is capable of expressing the polypeptides encoded thereby, including any suitable prokaryotic or eukaryotic cell. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102). Suitable eukaryotic cells are known in the art and include, for example, yeast cells, insect cells, and mammalian cells. Examples of suitable yeast cells include those from the genera Hansenula, Kluyveromyces, Pichia, Rhinosporidium, Saccharomyces, and Schizosaccharomyces. Suitable insect cells include Sf-9 and HIS cells (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants also are suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of such cells are well known in the art (see, e.g., Ausubel et al., eds., Short Protocols in Molecular Biology, 5th ed., John Wiley & Sons, Inc., Hoboken, N.J. (2002)). Preferably, the mammalian cell is a human cell.

In some embodiments, the vector can include means for attaching a detection moiety to an anti-ANGPTL7 antibody of the present disclosure. In some embodiments, the vector can include means for attaching a purification moiety to an anti-ANGPTL7 antibody of the present disclosure. Exemplary detection and/or purification moieties/tags that can be coupled to an anti-ANGPTL7 antibody of the present disclosure includes, but is not limited to, hemagglutinin (HA), c-Myc, V5, DYKDDDDK, His tag (e.g., 6×-HIS), Glutathione S-Transferase (GST), Maltose Binding Protein (MBP), a fluorophore (e.g., Green Fluorescent Protein (GFP), Red Fluorescent Protein (RFP), mCherry, a chromophore, and/or a luminescent peptide (e.g., luciferase).

In some embodiments, the expression vector is suitable for use in gene therapy (e.g., an expression vector for delivering a polynucleotide encoding an anti-ANGPTL7 antibody of the present disclosure to a subject). In some embodiments, the expression vector is a herpes simplex virus (HSV) vector, or a retrovirus vector. In some embodiments, the expression vector is an adeno-associated virus (AAV) vector, or comprises an AAV backbone. For example, AAV vectors have been designed, produced and used to mediate gene delivery in human subjects, including for therapeutic purposes. Typically, AAV vectors for use in gene transfer comprise a replication defective AAV genome lacking functional Rep and Cap coding viral sequences. Such replication defective AAV vectors more preferably lack most or all of the Rep and Cap coding sequences, and essentially retain one or two AAV ITR sequences and a packaging sequence. The defective genome is packaged in a viral particle, to form a defective, recombined AAV virus, also termed “AAV vector.” Methods of producing such AAV vectors have been disclosed in the literature, including using packaging cells, auxiliary viruses or plasmids, and/or baculovirus systems (Samulski et al., (1989) J. Virology 63, 3822: Xiao et al., (1998) J. Virology 72, 2224; Inoue et al., (1998) J. Virol. 72, 7024; WO98/22607; WO2005/072364). Methods of producing pseudotyped AAV vectors have also been reported (e.g., WO00/28004), as well as various modifications or formulations of AAV vectors, to reduce their immunogenicity upon in vivo administration (see e.g., WO01/23001; WOOO/73316; WO04/112727; WO05/005610; WO99/06562). AAV vectors may be prepared or derived from various serotypes of AAVs, which may be even mixed together or with other types of viruses to produce chimeric (e.g., pseudotyped) AAV viruses. Examples of tAAVs are human AAV4 vectors, human AAV7 vectors, human AAV9 vectors, human AAV10 vectors, or bovine AAV vectors. The AAV vector may be derived from a single AAV serotype or comprise sequences or components originating from at least two distinct AAV serotypes (pseudotyped AAV vector), e.g., an AAV vector comprising an AAV genome derived from one AAV serotype (for example AAV9), and a capsid derived at least in part from a distinct AAV serotype. An AAV vector, as used herein, is a vector which comprises at least one component part derivable from an adeno-associated virus. Preferably, that component part is involved in the biological mechanisms by which the vector infects or transduces target cells and expresses an anti-ANGPTL7 antibody of the present disclosure (e.g., ocular delivery/expression).

In other embodiments, the expression vector is a lentiviral vector (LV), or comprises an LV backbone. Lentiviruses are part of a larger group of retroviruses. A detailed list of lentiviruses may be found in Coffin et al (1997) “Retroviruses” Cold Spring Harbour Laboratory Press Eds: J M Coffin, SM Hughes, HE Varmus pp 758-763). For example, lentiviruses can be divided into primate and non-primate groups. Examples of primate lentiviruses include but are not limited to: the human immunodeficiency virus (HIV), the causative agent of human auto immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV). In one embodiment, the lentiviral vector is derived from HIV-1, HIV-2, SIV, FIV, BIV, EIAV, CAEV or Visna lentivirus. The lentivirus family differs from retroviruses in that lentiviruses have the capability to infect both dividing and non-dividing cells (Lewis et al (1992) EMBO J 11 (8); 3053-3058 and Lewis and Emermasn (1994) J Virol 68 (1):510-516). In contrast, other retroviruses, such as MLV, are unable to infect non-dividing or slowly dividing cells such as those that make up, for example, muscle, brain, lung and liver tissue. A lentiviral vector, as used herein, is a vector which comprises at least one component part derivable from a lentivirus. Preferably, that component part is involved in the biological mechanisms by which the vector infects or transduces target cells and expresses an anti-ANGPTL7 antibody of the present disclosure (e.g., ocular delivery/expression).

Additional compositions and methods for ocular gene therapy can be found in, e.g., Bordet, T., and Behar-Cohen, F., “Ocular gene therapies in clinical practice: viral vectors and nonviral alternatives,” Drug Discovery Today, Volume 24, Issue 8, August 2019, Pages 1685-1693). In some embodiments, gene therapy platforms, methods, and compositions that can be used to deliver an anti-ANGPTL7 antibody of the present disclosure to a subject (e.g., ocular delivery) includes the platforms, methods, and compositions disclosed in US20220025396, US20220011308, US20210371877, US20210363192, US20190078099, US20190038724, and U.S. Pat. No. 10,494,646B2, which are incorporated herein by reference. In other embodiments, gene therapy platforms, methods, and compositions that can be used to deliver an anti-ANGPTL7 antibody of the present disclosure to a subject (e.g., ocular delivery) includes the platforms, methods, and compositions based on HMR59 (Hemera Biosciences), which through its protein product soluble CD59, blocks the membrane attack complex that forms during the terminal step in the complement cascade. HMR59 is designed to be administered as a single intraocular injection.

In accordance with these embodiments, the present disclosure also provides a method of administering ocular gene therapy to a subject in need thereof comprising injecting a pharmaceutical composition comprising an effective amount of an expression vector described herein (e.g., an expression vector comprising a polynucleotides encoding an anti-ANGPTL7 antibody of the present disclosure). As described further below, the present disclosure also provides a method of treating glaucoma and other ocular diseases affecting the optic nerve and retinal ganglion cells. In some embodiments, the method comprises administering a pharmaceutical composition comprising an effective amount of an expression vector described herein (e.g., an expression vector comprising a polynucleotides encoding an anti-ANGPTL7 antibody of the present disclosure). In some embodiments, administering the pharmaceutical composition treats at least one symptom of glaucoma or other ocular diseases affecting the optic nerve and retinal ganglion cells.

### Compositions and Methods of Treatment

The anti-ANGPTL7 antibodies of the present disclosure can be administered as part of a pharmaceutical composition in a therapeutically effective amount to treat an eye disease (e.g., glaucoma). In some embodiments, the composition is suitable for ocular administration. In some embodiments, ocular administration comprises injection into vitreous fluid. In some embodiments, ocular administration comprises delivering the antibody using a conjunctival insert, a contact lens, a gel, a nanoparticle, a mucoadhesive polymer, an ointment, a solution, a suspension, eye drops, and/or an implant (e.g., Susvimo™). Recent methods and formulations for ocular administration can be found in, e.g., Souto, E. B., et al. “Advanced Formulation Approaches for Ocular Drug Delivery: State-Of-The-Art and Recent Patents,” Pharmaceutics, 2019 September; 11(9): 460).

In accordance with these embodiments, the methods include administering a pharmaceutical composition comprising a therapeutically effective amount of an anti-ANGPTL7 antibody of the present disclosure. In some embodiments, the pharmaceutical composition is administered ocularly and treats at least one symptom of glaucoma or other ocular diseases affecting the optic nerve and retinal ganglion cells. In some embodiments, the at least one symptom comprises eye pain, eye pressure, headaches, rainbow-colored halos around lights, low vision, blurred vision, narrowed vision, impaired peripheral vision, blind spots, nausea, vomiting, and red eyes. In some embodiments, administering the pharmaceutical composition reduces or attenuates intraocular pressure and/or increases or enhances outflow facility in the subject's eye.

In some embodiments, the pharmaceutical composition comprising a therapeutically effective amount of an anti-ANGPTL7 antibody of the present disclosure is administered at a dose ranging from about 0.0001 mg/dose to about 100 mg/dose. In some embodiments, the anti-ANGPTL7 antibody is administered at a dose ranging from about 0.001 mg/dose to about 100 mg/dose. In some embodiments, the anti-ANGPTL7 antibody is administered at a dose ranging from about 0.01 mg/dose to about 100 mg/dose. In some embodiments, the anti-ANGPTL7 antibody is administered at a dose ranging from about 0.1 mg/dose to about 100 mg/dose. In some embodiments, the anti-ANGPTL7 antibody is administered at a dose ranging from about 1.0 mg/dose to about 100 mg/dose. In some embodiments, the anti-ANGPTL7 antibody is administered at a dose ranging from about 10 mg/dose to about 100 mg/dose. In some embodiments, the anti-ANGPTL7 antibody is administered at a dose ranging from about 0.0001 mg/dose to about 10 mg/dose. In some embodiments, the anti-ANGPTL7 antibody is administered at a dose ranging from about 0.0001 mg/dose to about 1.0 mg/dose. In some embodiments, the anti-ANGPTL7 antibody is administered at a dose ranging from about 0.0001 mg/dose to about 0.1 mg/dose. In some embodiments, the anti-ANGPTL7 antibody is administered at a dose ranging from about 0.0001 mg/dose to about 0.001 mg/dose. In some embodiments, the anti-ANGPTL7 antibody is administered at a dose ranging from about 0.01 mg/dose to about 10 mg/dose. In some embodiments, the anti-ANGPTL7 antibody is administered at a dose ranging from about 0.001 mg/dose to about 1.0 mg/dose. In some embodiments, the anti-ANGPTL7 antibody is administered at a dose ranging from about 0.1 mg/dose to about 10 mg/dose.

In some embodiments, the pharmaceutical composition comprising a therapeutically effective amount of an anti-ANGPTL7 antibody of the present disclosure is administered at a dose ranging from about 0.0001 mg/ml to about 100 mg/ml. In some embodiments, the anti-ANGPTL7 antibody is administered at a dose ranging from about 0.001 mg/ml to about 100 mg/ml. In some embodiments, the anti-ANGPTL7 antibody is administered at a dose ranging from about 0.01 mg/ml to about 100 mg/ml. In some embodiments, the anti-ANGPTL7 antibody is administered at a dose ranging from about 0.1 mg/ml to about 100 mg/ml. In some embodiments, the anti-ANGPTL7 antibody is administered at a dose ranging from about 1.0 mg/ml to about 100 mg/ml. In some embodiments, the anti-ANGPTL7 antibody is administered at a dose ranging from about 10 mg/ml to about 100 mg/ml. In some embodiments, the anti-ANGPTL7 antibody is administered at a dose ranging from about 0.0001 mg/ml to about 10 mg/ml. In some embodiments, the anti-ANGPTL7 antibody is administered at a dose ranging from about 0.0001 mg/ml to about 1.0 mg/ml. In some embodiments, the anti-ANGPTL7 antibody is administered at a dose ranging from about 0.0001 mg/ml to about 0.1 mg/ml. In some embodiments, the anti-ANGPTL7 antibody is administered at a dose ranging from about 0.0001 mg/ml to about 0.01 mg/ml. In some embodiments, the anti-ANGPTL7 antibody is administered at a dose ranging from about 0.0001 mg/ml to about 0.001 mg/ml. In some embodiments, the anti-ANGPTL7 antibody is administered at a dose ranging from about 0.01 mg/ml to about 10 mg/ml. In some embodiments, the anti-ANGPTL7 antibody is administered at a dose ranging from about 0.001 mg/ml to about 1.0 mg/ml. In some embodiments, the anti-ANGPTL7 antibody is administered at a dose ranging from about 0.1 mg/ml to about 10 mg/ml.

As used herein, the terms “treatment,” “treating,” and the like refer to obtaining a desired pharmacologic and/or physiologic effect. In some embodiments, the effect is therapeutic, i.e., the effect partially or completely cures a disease and/or adverse symptom attributable to the disease. To this end, the methods of the present disclosure comprise administering a “therapeutically effective amount” of an anti-ANGPTL7 antibody, or composition comprising an anti-ANGPTL7 antibody. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the monoclonal antibody to elicit a desired response in the individual. For example, a therapeutically effective amount of an anti-ANGPTL7 antibody of the present disclosure is an amount that treats at least one symptom of glaucoma or other ocular diseases affecting the optic nerve and retinal ganglion cells in a subject. In some embodiments, the pharmacologic and/or physiologic effect may be prophylactic, i.e., the effect completely or partially prevents a disease or symptom thereof. In this respect, the methods of the present disclosure comprise administering a “prophylactically effective amount” of an anti-ANGPTL7 antibody or composition comprising an anti-ANGPTL7 antibody. A “prophylactically effective amount” refers to an amount effective, at dosages and for periods of time necessary, to achieve a desired prophylactic result (e.g., prevention of glaucoma or at least one symptom associated with glaucoma).

A typical dose of a therapeutically effective amount of an anti-ANGPTL7 antibody of the present disclosure can range from, for example, about 0.0001 mg/dose to about 100 mg/dose for each eye to be treated. In some embodiments, a therapeutically effective amount of an anti-ANGPTL7 antibody of the present disclosure can range from about 0.001 mg/dose to about 100 mg/dose, from about 0.01 mg/dose to about 100 mg/dose, from about 0.05 mg/dose to about 50 mg/dose, from about 0.1 mg/dose to about 10 mg/dose, from about 0.5 mg/dose to about 5 mg/dose, and from about 1 mg/dose to about 10 mg/dose. In some embodiments, a therapeutically effective concentration of an anti-ANGPTL7 antibody of the present disclosure can range from, for example, about 0.0001 mg to about 100 mg of the antibody per milliliter of solution. In some embodiments, a therapeutically effective concentration of an anti-ANGPTL7 antibody of the present disclosure can range from about 0.001 mg/ml to about 100 mg/ml, from about 0.01 mg/ml to about 100 mg/ml, from about 0.1 mg/ml to about 100 mg/ml, from about 1.0 mg/ml and about 100 mg/ml, from about 0.001 mg/ml and about 50 mg/ml, from about 0.01 mg/ml and about 50 mg/ml, from about 0.1 mg/ml and about 50 mg/ml, from about 0.1 mg/ml and about 25 mg/ml, from about 0.1 mg/ml and about 10 mg/ml, and from about 1.0 mg/ml and about 10 mg/ml. In some embodiments, a therapeutically effective dose of an anti-ANGPTL7 antibody of the present disclosure can be, exactly or approximately, 0.1 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 2.0 mg, 3.0 mg, 4.0 mg, 5.0 mg, 10.0 mg, 15.0 mg, 20.0 mg, or 25.0 mg, or can fall within a range delimited by any two of the foregoing values. For example, in certain embodiments, a sustained release formulation, (e.g., an ocular implant) can be, exactly or approximately, 0.1 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 2.0 mg, 3.0 mg, 4.0 mg, 5.0 mg, 10.0 mg, 15.0 mg, 20.0 mg, or 25.0 mg of an anti-ANGPTL7 antibody, or an amount that falls within a range delimited by any two of the foregoing values.

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the present disclosure. The desired dosage can be delivered by a single bolus administration of the composition, by multiple bolus administrations of the composition, or by continuous infusion administration of the composition. The composition comprising an anti-ANGPTL7 antibody, or antigen-binding fragment thereof, can be administered to a mammal using standard administration techniques, including ocular, oral, intravenous, intraperitoneal, subcutaneous, pulmonary, transdermal, intramuscular, intranasal, buccal, sublingual, or suppository administration. The composition preferably is suitable for ocular administration.

In accordance with the compositions and methods of treatment described herein, embodiments of the present disclosure include anti-ANGPTL7 antibodies that have enhanced half-life (e.g., after ocular administration), such that the anti-ANGPTL7 antibody can be administered less often to a subject. In some embodiments, the antibody comprises a half-life extension moiety. In some embodiments, the half-life extension moiety comprises a polypeptide that can be coupled to an anti-ANGPTL7 antibody of the present disclosure by any means known in the art (e.g., generation of a fusion protein). In some embodiments, the polypeptide that can be coupled to an anti-ANGPTL7 antibody of the present disclosure is at least 20 amino acids in length and comprises any combination of G, A, S T, E, and P residues. In some embodiments, the half-life extension polypeptide is attached to the C-terminus or N-terminus of the antibody. In some embodiments, this is referred to as “XTENylation,” as described further in U.S. Pat. Nos. 8,933,197, 7,846,445, 7,855,279, 8,492,530, 9,938,331, 8,673,860, 9,371,369, 9,926,351, 10,961,287, 10,172,953, and 10,953,073.

The present disclosure also provides a composition comprising any of the anti-ANGPTL7 antibodies or antigen-binding fragments thereof described herein. The composition desirably is a pharmaceutically acceptable (e.g., physiologically acceptable) composition, which comprises a carrier, preferably a pharmaceutically acceptable (e.g., physiologically acceptable) carrier, and the anti-ANGPTL7 antibody or antigen-binding fragment thereof. Any suitable carrier can be used within the context of the present disclosure, and such carriers are well known in the art. For example, the composition may contain preservatives, such as, for example, methylparaben, propylparaben, sodium benzoate, and benzalkonium chloride. A mixture of two or more preservatives optionally may be used. In addition, buffering agents may be included in the composition. Suitable buffering agents include, for example, citric acid, sodium citrate, phosphoric acid, potassium phosphate, and various other acids and salts. A mixture of two or more buffering agents optionally may be used. Methods for preparing compositions for pharmaceutical use are known to those skilled in the art and are described in, for example, Remington: The Science and Practice of Pharmacy, Lippincott Williams & Wilkins; 21st ed. (May 1, 2005).

Once administered to a mammal (e.g., a human), the biological activity of the anti-ANGPTL7 antibody, or antigen-binding fragment thereof, can be measured by any suitable method known in the art. For example, the biological activity can be assessed by determining the stability of the anti-ANGPTL7 antibody. The biological activity of the anti-ANGPTL7 antibody also can be assessed by determining its binding affinity to ANGPTL7 peptides and/or by assessing its binding affinity to peptides with which it may cross-react. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 femtomolar (fM) to about 1 millimolar (mM) (e.g., from about 1 picomolar (pM) to about 1 nanomolar (nM), or from about 1 nM to about 1 micromolar (μM)). In some embodiments, the affinity of an anti-ANGPTL7 antibody may be from about 1 nm to about 20 nm, and desirably from about 5 nm to about 10 nm. Antibody affinity for an antigen or epitope of interest can be measured using any art-recognized assay. Such methods include, for example, fluorescence activated cell sorting (FACS), separable beads (e.g., magnetic beads), antigen panning, and/or ELISA (see, e.g., Janeway et al. (eds.), Immunobiology, 5th ed., Garland Publishing, New York, N.Y., 2001).

In some embodiments, an anti-ANGPTL7 antibody, or composition comprising an anti-ANGPTL7 antibody, may be administered alone or in combination with other drugs/therapeutic agents. For example, the anti-ANGPTL7 antibody can be administered in combination with other agents for the treatment or prevention of glaucoma or other ocular disease affecting the optic nerve or retinal ganglion cells, as disclosed herein. For example, anti-ANGPTL7 antibodies of the present disclosure, or antibody conjugates, fusion proteins, or polymeric formulations thereof, can be used either alone or in combination with other agents in a therapy. For instance, an anti-ANGPTL7 antibody may be co-administered with at least one additional therapeutic agent. In certain embodiments, an additional therapeutic agent is another antibody, a chemotherapeutic agent, a cytotoxic agent, an anti-angiogenic agent, an immunosuppressive agent, a prodrug, a cytokine, a cytokine antagonist, cytotoxic radiotherapy, a corticosteroid, an anti-emetic, a cancer vaccine, an analgesic, a growth-inhibitory agent, or combinations thereof.

In certain embodiments, an anti-ANGPTL7 antibody of the present disclosure is administered with a drug/therapeutic agent that treats/prevents glaucoma or other ocular disease that affects the optic nerve or retinal ganglion cells. Any suitable glaucoma therapeutic agent can be administered as an additional therapeutic agent in combination with an anti-ANGPTL7 antibody of the present disclosure, or an antibody conjugate, fusion protein, and/or polymeric formulation thereof, for treatment of an ocular disorder (e.g., glaucoma or other disease associated with or affecting the optic nerve or retinal ganglion cells). For example, in some embodiments, an anti-ANGPTL7 antibody of the present disclosure can be administered with an agent that lowers intraocular pressure by promoting the drainage of fluid from the eye, including but not limited to, prostaglandins (e.g., Xalatan (latanoprost), Travatan Z (travoprost), Zioptan (tafluprost), and Lumigan (bimatoprost)), Rho kinase inhibitors (e.g., Rhopressa (netarsudil)), nitric oxides (e.g., Vyzulta (latanoprostene bunod), and miotic or cholinergic agents (e.g., Isopto Carpine (pilocarpine)). In some embodiments, an anti-ANGPTL7 antibody of the present disclosure can be administered with an agent that lowers intraocular pressure by reducing the amount of fluid produced in the eye, including but not limited to, alpha-adrenergic agonists (e.g., Iopidine (apraclonidine) and Alphagan P or Qoliana (brimonidine)), beta blockers (e.g., Betoptic (betaxolol) and Betimol, Istalol, or Timoptic (timolol)), and carbonic anhydrase inhibitors (e.g., Trusopt (dorzolamide) and Azopt (brinzolamide)). Additionally, one or more of these therapeutic agents can be administered with an anti-ANGPTL7 antibody of the present disclosure, in conjunction with other treatments, such as laser treatment (e.g., trabeculoplasty) and surgery (e.g., glaucoma implant surgery, and minimally invasive glaucoma surgery (MIGS)).

In some embodiments, an anti-ANGPTL7 antibody, antibody conjugate, fusion protein, or polymeric formulation of the present disclosure is administered simultaneously with an additional therapeutic agent. In some embodiments, an anti-ANGPTL7 antibody, antibody conjugate, fusion protein, or polymeric formulation of the present disclosure is administered before or after an additional therapeutic agent. In some embodiments, the additional therapeutic agent(s) binds to a second biological molecule selected from the group consisting of VEGF, IL-1β; IL-6; IL-6R; IL-13; IL-13R; PDGF; angiopoietin; Ang2; Tie2; SIP; integrins αvβ3, αvβ5, and α5β1; betacellulin; apelin/APJ; erythropoietin; complement factor D; TNFα; HtrA1; a VEGF receptor; ST-2 receptor; and proteins genetically linked to AMD risk, such as complement pathway components C2, factor B, factor H, CFHR3, C3b, C5, C5a, and C3a; HtrA1; ARMS2; TIMP3; HLA; interleukin-8 (IL-8); CX3CR1; TLR3; TLR4; CETP; LTPC; COL10A1; and TNFRSF10A. In some embodiments, the additional therapeutic agent is an antibody or antigen-binding fragment thereof. In some embodiments according to (or as applied to) any of the embodiments above, the ocular disorder is an intraocular neovascular disease selected from the group consisting of proliferative retinopathies, choroidal neovascularization (CNV), glaucoma, diseases affecting the optic nerve, diseases affecting the retinal ganglion cells, diabetic and other ischemia-related retinopathies, diabetic macular edema, pathological myopia, von Hippel-Lindau disease, histoplasmosis of the eye, retinal vein occlusion (RVO), including CRVO and BRVO, corneal neovascularization, retinal neovascularization, and retinopathy of prematurity (ROP).

In some embodiments, an anti-ANGPTL7 antibody of the present disclosure, or an antibody conjugate, fusion protein, and/or polymeric formulation thereof, may be administered in combination with at least one additional therapeutic agent for treatment of an ocular disorder, for example, an ocular disorder described herein (e.g., glaucoma or other disease associated with or affecting the optic nerve or retinal ganglion cells). Exemplary additional therapeutic agents for combination therapy for treatment of ocular disorders include, without limitation, anti-angiogenic agents, such as VEGF antagonists, including, for example, anti-VEGF antibodies (e.g., the anti-VEGF Fab LUCENTIS® (ranibizumab)), soluble receptor fusion proteins (e.g., the recombinant soluble receptor fusion protein EYLEA® (aflibercept, also known as VEGF Trap Eye; Regeneron/Aventis)), aptamers (e.g., the anti-VEGF pegylated aptamer MACUGEN® (pegaptanib sodium: NeXstar Pharmaceuticals/OSI Pharmaceuticals)), and VEGFR tyrosine kinase inhibitors (e.g., 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1-ylpropoxy)quinazoline (AZD2171), vatalanib (PTK787), semaxaminib (SU5416; SUGEN), and SUTENT® (sunitinib)); Tryptophanyl-tRNA synthetase (TrpRS); squalamine; RETAANE® (anecortave acetate for depot suspension; Alcon, Inc.); Combretastatin A4 Prodrug (CA4P); MIFEPREX® (mifepristone-ru486); subtenon triamcinolone acetonide; intravitreal crystalline triamcinolone acetonide; matrix metalloproteinase inhibitors (e.g., Prinomastat (AG3340; Pfizer)); fluocinolone acetonide (including fluocinolone intraocular implant; Bausch & Lomb/Control Delivery Systems); linomide; inhibitors of integrin 03 function; angiostatin, and combinations thereof.

Further examples of additional therapeutic agents that can be used in combination with an anti-ANGPTL7 antibody of the present disclosure, or an antibody conjugate, fusion protein, and/or polymeric formulation thereof, for treatment of an ocular disorder (e.g., glaucoma or other disease associated with or affecting the optic nerve or retinal ganglion cells), include, but are not limited to, VISUDYNE® (verteporfin; a light-activated drug that is typically used in conjunction with photodynamic therapy with a non-thermal laser), PKC412, Endovion (NS 3728; NeuroSearch A/S), neurotrophic factors (e.g., glial derived neurotrophic factor (GDNF) and ciliary neurotrophic factor (CNTF)), diltiazem, dorzolamide, PHOTOTROP®, 9-cis-retinal, eye medication (e.g., phospholine iodide, echothiophate, or carbonic anhydrase inhibitors), veovastat (AE-941; AEterna Laboratories, Inc.), Sirna-027 (AGF-745; Sirna Therapeutics, Inc.), neurotrophins (including, by way of example only, NT-4/5, Genentech), Cand5 (Acuity Pharmaceuticals), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celitech Group plc), ATX-S10 (Hamamatsu Photonics), TGF-beta 2 (Genzyme/Celtrix), tyrosine kinase inhibitors (e.g., those from Allergan, SUGEN, or Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), cyclosporin A, therapeutic agents used in photodynamic therapy (e.g., VISUDYNE®; receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; and motexafin lutetium, Pharmacyclics, Inc.), antisense oligonucleotides (including, by way of example, products tested by Novagali Pharma SA and ISIS-13650, Isis Pharmaceuticals), and combinations thereof.

An anti-ANGPTL7 antibody of the present disclosure, or an antibody conjugate, fusion protein, and/or polymeric formulation thereof, may be administered in combination with a therapy or surgical procedure for treatment of an ocular disorder (e.g., glaucoma or other disease associated with or affecting the optic nerve or retinal ganglion cells), including, for example, laser photocoagulation (e.g., panretinal photocoagulation (PRP)), drusen lasering, macular hole surgery, macular translocation surgery, implantable miniature telescopes, PHI-motion angiography (also known as micro-laser therapy and feeder vessel treatment), proton beam therapy, microstimulation therapy, retinal detachment and vitreous surgery, scleral buckle, submacular surgery, transpupillary thermotherapy, photosystem I therapy, use of RNA interference (RNAi), extracorporeal rheopheresis (also known as membrane differential filtration and rheotherapy), microchip implantation, stem cell therapy, gene replacement therapy, ribozyme gene therapy (including gene therapy for hypoxia response element, Oxford Biomedica; Lentipak, Genetix; and PDEF gene therapy, GenVec), photoreceptor/retinal cells transplantation (including transplantable retinal epithelial cells, Diacrin, Inc.; retinal cell transplant, Cell Genesys, Inc.), acupuncture, and combinations thereof.

In some embodiments, an anti-ANGPTL7 antibody of the present disclosure, or an antibody conjugate, fusion protein, and/or polymeric formulation thereof, can be administered in combination with an anti-angiogenic agent for treatment of an ocular disorder (e.g., glaucoma or other disease associated with or affecting the optic nerve or retinal ganglion cells). Any suitable anti-angiogenic agent can be used in combination with an antibody of the present disclosure, including, but not limited to, those listed by Carmeliet et al. Nature 407:249-257, 2000. In some embodiments, the anti-angiogenic agent is a VEGF antagonist, including, but not limited to, an anti-VEGF antibody (e.g., the anti-VEGF Fab LUCENTIS® (ranibizumab), RTH-258 (formerly ESBA-1008, an anti-VEGF single-chain antibody fragment; Novartis), or a bispecific anti-VEGF antibody (e.g., an anti-VEGF/anti-angiopoietin 2 bispecific antibody such as RG-7716; Roche)), a soluble recombinant receptor fusion protein (e.g., EYLEA® (aflibercept)), a VEGF variant, a soluble VEGFR fragment, an aptamer capable of blocking VEGF (e.g., pegaptanib) or VEGFR, a neutralizing anti-VEGFR antibody, a small molecule inhibitor of VEGFR tyrosine kinases, an anti-VEGF DARPin® (e.g., abicipar pegol), a small interfering RNAs which inhibits expression of VEGF or VEGFR, a VEGFR tyrosine kinase inhibitor (e.g., 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1-ylpropoxy)quinazoline (AZD2171), vatalanib (PTK787), semaxaminib (SU5416; SUGEN), and SUTENT® (sunitinib)), and combinations thereof.

Other suitable anti-angiogenic agents that may be administered in combination with an antibody of the present disclosure, or an antibody conjugate, fusion protein, and/or polymeric formulation thereof, for treatment of an ocular disorder (e.g., glaucoma or other disease associated with or affecting the optic nerve or retinal ganglion cells) include corticosteroids, angiostatic steroids, anecortave acetate, angiostatin, endostatin, tyrosine kinase inhibitors, matrix metalloproteinase (MMP) inhibitors, insulin-like growth factor-binding protein 3 (IGFBP3), stromal derived factor (SDF-1) antagonists (e.g., anti-SDF-1 antibodies), pigment epithelium-derived factor (PEDF), gamma-secretase, Delta-like ligand 4, integrin antagonists, hypoxia-inducible factor (HIF)-1α antagonists, protein kinase CK2 antagonists, agents that inhibit stem cell (e.g., endothelial progenitor cell) homing to the site of neovascularization (e.g., an anti-vascular endothelial cadherin (CD-144) antibody and/or an anti-SDF-1 antibody), and combinations thereof.

In some embodiments, an anti-ANGPTL7 antibody of the present disclosure, or an antibody conjugate, fusion protein, and/or polymeric formulation thereof, can be administered in combination with an agent that has activity against neovascularization for treatment of an ocular disorder (e.g., glaucoma or other disease associated with or affecting the optic nerve or retinal ganglion cells), such as an anti-inflammatory drug, a mammalian target of rapamycin (mTOR) inhibitor (e.g., rapamycin, AFINITOR® (everolimus), and TORISEL® (temsirolimus)), cyclosporine, a tumor necrosis factor (TNF) antagonist (e.g., an anti-TNFα antibody or antigen-binding fragment thereof (e.g., infliximab, adalimumab, certolizumab pegol, and golimumab) or a soluble receptor fusion protein (e.g., etanercept)), an anti-complement agent, a nonsteroidal anti-inflammatory agent (NSAID), or combinations thereof.

In addition to therapeutic uses, an anti-ANGPTL7 antibody or antigen-binding fragment, described herein can be used in diagnostic or research applications. Research applications include, for example, methods that utilize the anti-ANGPTL7 antibody and a label to detect an ANGPTL7 polypeptide or protein in a sample (e.g., in a human body fluid or in a cell or tissue extract). The anti-ANGPTL7 antibody or antigen-binding fragment thereof may be employed in any suitable assay for measuring ANGPTL7 in a sample for diagnostic and/or research purposes. Such assays include, but are not limited to, sandwich immunoassays, enzyme immunoassays (EIA), enzyme-linked immunosorbent assays (ELISA), lateral flow assays, competitive inhibition immunoassays (e.g., forward and reverse), competitive binding assays, Forster resonance energy transfer (FRET), one-step antibody detection assays, single molecule detection assays, radioimmunoassays (RIA), and FACS. Such methods are disclosed in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792; and Adamczyk et al., Anal. Chim. Acta, 579(1): 61-67 (2006).

The anti-ANGPTL7 antibody or antigen-binding fragment thereof can be provided in a kit, e.g., a packaged combination of reagents in predetermined amounts with instructions for performing an assay using the antibody (e.g., an assay that detects ANGPTL7). As such, the disclosure provides a kit comprising the antibody or antigen-binding fragment described herein and instructions for use thereof. The instructions can be in paper form or computer-readable form, such as a disk, CD, DVD, etc. Alternatively or additionally, the kit can comprise a calibrator or control, and/or at least one container (e.g., tube, microtiter plates, or strips) for conducting an assay, and/or a buffer, such as an assay buffer or a wash buffer. Ideally, the kit comprises all components, i.e., reagents, standards, buffers, diluents, etc., which are necessary to perform the assay. Other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

The following examples further illustrate the various embodiments of the present disclosure but should not be construed as in any way limiting its scope.

### EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

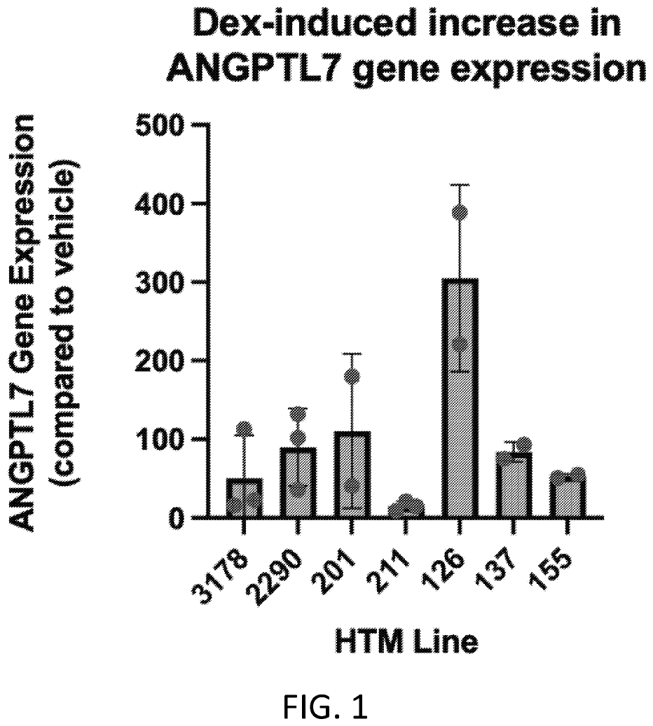

This example describes dexamethasone-induced changes in gene expression in primary human trabecular meshwork cells. Primary trabecular meshwork cells isolated from post-mortem human donors were treated with dexamethasone (DEX). Changes in ANGTPL7 gene expression relative to vehicle were quantified using qPCR, as shown in FIG. 1. Using these data, strong DEX responders were selected for future studies, such as RNAseq.

In particular, primary human trabecular meshwork cells were passed in DMEM with 10% fetal bovine serum (FBS) and 1% penicillin/streptomycin. Cells were plated in 96-well plates at 20,000 cells/well. Media was changed 3 times a week. After 1 week, cells were further differentiated using DMEM with 1% FBS and 1% penicillin/streptomycin. Cells were incubated with low serum for a minimum of 1 additional week, with media changes 3 times a week.

For dexamethasone (DEX) treatment, media were removed and replaced with media containing 100 nM DEX or 0.1% ethanol (EtOH, vehicle). Media was refreshed after 2-3 days, and cells were harvested for qPCR on day 5 using the Cells to Ct kit. qPCR was run using an ANGPTL7 TaqMan assay from Life Technologies on a Quantstudio6 qPCR machine. Gene expression was normalized to the EtOH control.

### Example 2

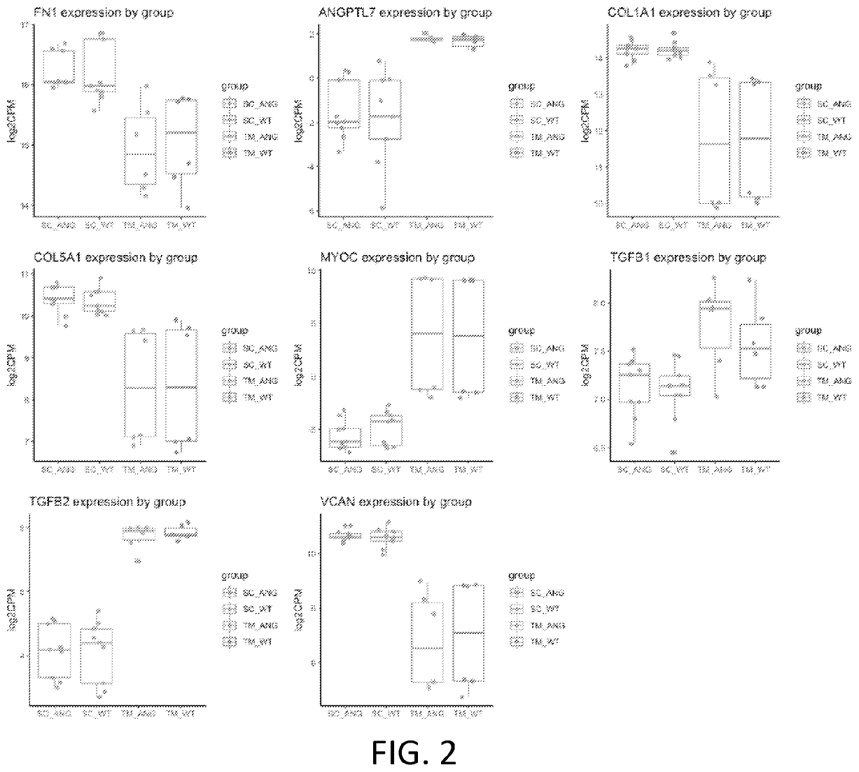

This example describes RNAseq of ANGPTL7 treatment-induced changes in gene expression of human trabecular meshwork (TM) and Schlemm's Canal (SC) cells (FIG. 2). Briefly, primary human trabecular meshwork and Schlemm's canal cells were passed in DMEM with 10% fetal bovine serum (PBS) with 1% penicillin/streptomycin. Cells were seeded at 200 k cells/well in 6-well formats. Cells were differentiated in low serum for a week with media changes 3 times a week. For ANGPTL7 treatment, media were removed and replaced with media containing 50 ng/mL of ANGPTL7 protein made from BIORTUS. Treatment media was refreshed daily for three days. After three days, cells were spun down and frozen into a pellet and then stored into a −80 C freezer until shipment to SeqMatic for processing.

### Example 3

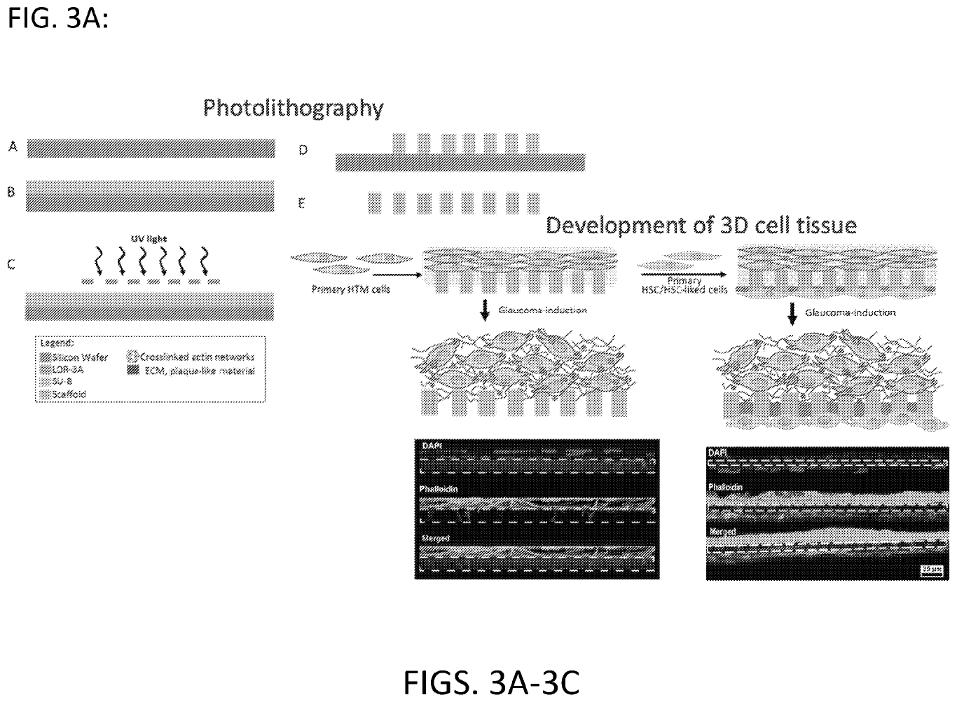

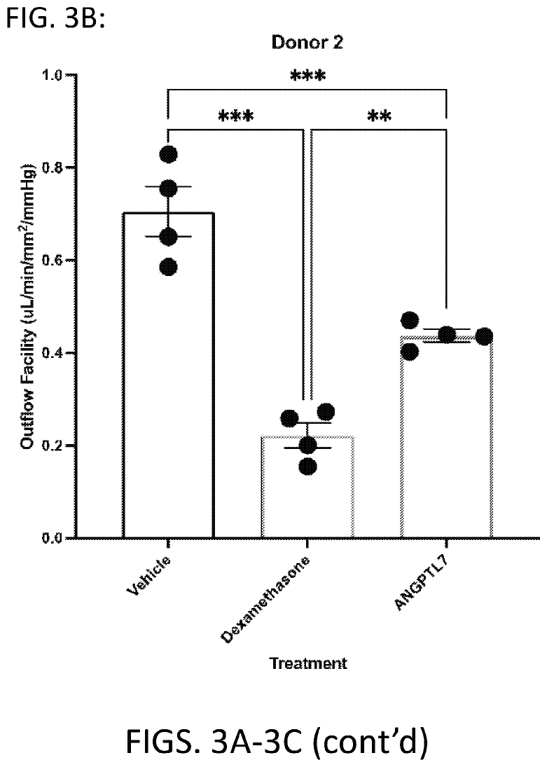

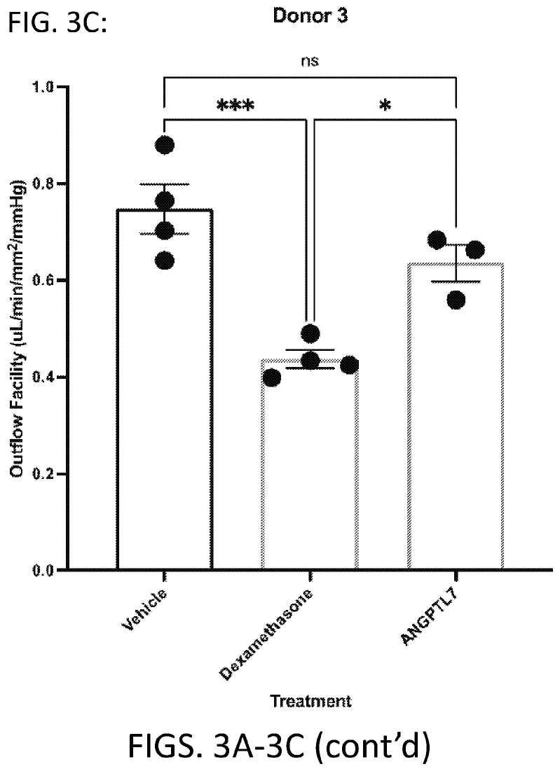

Experiments were conducted to determine the effects of ANGPTL7 on outflow facility in a bioengineered ex vivo human eye tissue model. Glauconix developed a proprietary technology allowing multi-layer growth of bioengineered 3D human trabecular meshwork (HTM) cells co-cultured with human Schlemms' canal (HSC) cells, thereby mimicking the architecture of the tissues responsible for pressure build-up in the human eye (FIG. 3A). This allows endpoint analyses relevant to IOP-modulation. This scaffolding of cells is used to compare treatment effects of vehicle (DMSO), dexamethasone (a steroid known to elevate IOP), and human ANGPTL7 protein on outflow facility.

Briefly, HTM cells were thawed and allowed to grow for about 7 days (media changes every 2 days). The HTM cells were seeded onto scaffolds and HSC cells were thawed on the same day; the HTM cells were allowed to grow for 7 days on the scaffolds with media changes every 2 days. HSCs were co-cultured on the back side of the HTM scaffolds and grown for 10-12 days with media changes every 2 days. HTM/HSC constructs were serum starved the in a 1% medium the day before treatment. The first day of treatment was called Day 0. On Day 3, supernatants were collected, re-treated, and this process was repeated on Days 6 and 9. Perfusion was done on Day 10.

FIG. 3B includes representative data demonstrating outflow facility for 3D-HTM donor 2 treated with vehicle (DMSO), 500 nM Dexamethasone, 50 ng/mL of ANGPTL7. Samples were analyzed using One-way ANOVA (GraphPad Prism Software, Inc., La Jolla, CA), *P<0.05, **P<0.0.01, ***P<0.001 N=4 per treatment group. FIG. 3C includes representative data demonstrating outflow facility for 3D-HTM donor 3 treated with vehicle (DMSO), 500 nM Dexamethasone, 50 ng/mL of ANGPTL7. Samples were analyzed using One-way ANOVA (GraphPad Prism Software, Inc., La Jolla, CA), *P<0.05, **P<0.0.01, ***P<0.001 N=4 per treatment group. These data demonstrate that ANGPTL7 treatment reduced outflow facility for both donors, similar to DEX treatment.

### Example 4

Experiments were conducted to determine the effects of ANGPTL7 on outflow facility using a bioengineered ex vivo human eye tissue model. Glauconix developed a proprietary technology allowing multi-layer growth of bioengineered 3D human trabecular meshwork (HTM) cells co-cultured with human Schlemms' canal (HSC) cells, thereby mimicking the architecture of the tissues responsible for pressure build-up in the human eye (FIG. 3A; see also exemplary protocol in Example 3). This allows endpoint analyses relevant to IOP-modulation.

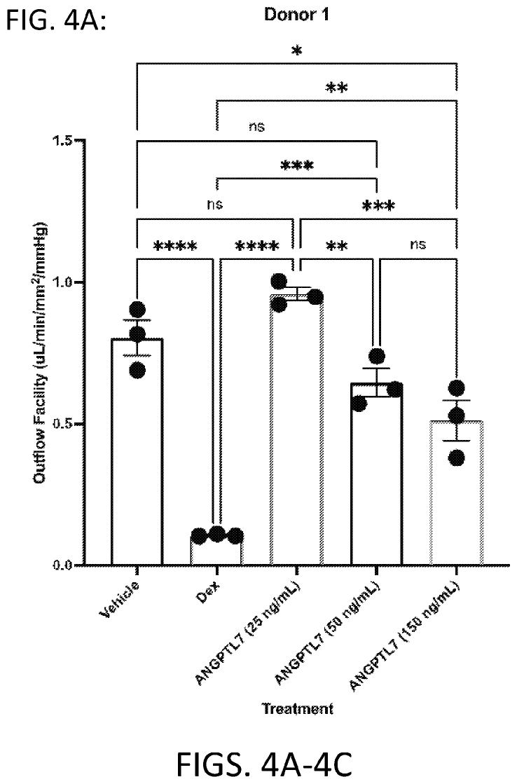

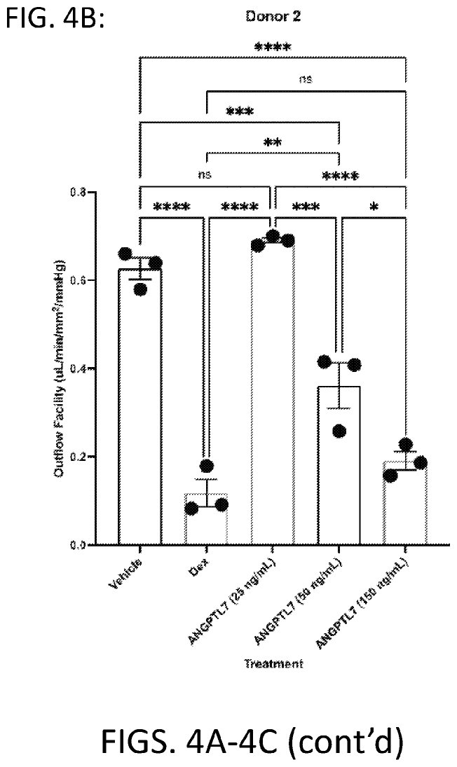

This experiment compared three different doses (25, 50, and 150 μg/ml) of ANGPTL7 protein on outflow facility in three different donor HTM cell lines (FIGS. 4A-4C). In this example, experiments were focused on the reproducibility of ANGPTL7 effects on outflow facility (from SOW1) and dose response effects in three different HTM donors.

### Example 5

Generation of recombinant ANGPTL7 proteins. Recombinant protein preparations were undertaken to produce specific proteins which could be used as immunogens/antigens, screening reagents, and/or control reagents. All efforts were made in service of the goal of generating a panel of anti-ANGPTL7 antibodies with the desired properties. In this particular example, outsourced material produced by Biortus was used for the human ANGPTL7 multimer, while protein engineering work was used to produce mutant human monomers. Truncated but non-engineered wild type fibrinogen domain only human ANGPTL7 monomer was also produced.

Proteins with the sequence corresponding to human, African green monkey, and rabbit ANGPTL7 were produced. The human and African green monkey sequences were engineered to favor the production of monomeric material, while the rabbit protein was a mixture of monomer, trimer, and hexamer. When finished, all protein preparations exceeded 90% purity by analytical methods and were less than <1 endotoxin unit per milliliter.

Beginning with in silico analysis, appropriate wild type amino acid sequences of human, rabbit, and African green monkey ANGPTL7 were located and extracted from publicly available databases. The sequences were then further analyzed and modified manually using Geneious Prime software in accordance with the experimental plan. Specifically, in regard to the production of monomeric ANGPTL7, the mechanism of multimerization was examined closely. Structurally, ANGPTL7 consists of two domains, a small N-terminal coiled coil domain and a larger C-terminal fibrinogen like domain. Given the similarities to fibrinogen protein at the domain level, previously published works, and the well documented biological function of coiled coil domains, the N-terminal region was identified as the area on which to focus in order to engineer monomeric mutants. Coiled coil domains follow a well-defined heptad repeat of hydrophobic and charged amino acids. This repeat, in conjugation with the property of approximately seven amino acids per two turns of an alpha helix, and the aqueous environment of either the extra or intracellular space, favor packing of the hydrophobic side chains into the interior of the coil while the charged amino acids face the aqueous environment. Dimerization, trimerization or other higher order multimers result as each linear alpha helix is dependent on others to reach the energetically favorable “packed” state. Approaches which disrupt linearity and/or the hydrophobic and charged heptad could lead to monomers. Two strategies were designed upon this theoretical framework. Constructs with substitutions of proline for leucine at both amino acid position 59 and 84 or with leucine at position 59 replaced with glycine-glycine-proline-glycine-glycine were built in silico and added to the panel of sequences designed for the project. The replacement of leucine at position 59 with five amino acids proved most successful. A version In addition, a truncated form of human ANGPTL7 corresponding to only the C-terminal Fibrinogen like domain sequence was added to the panel of sequences.

All Sequences were examined for liabilities and modified to add further amino acid sequence which encoded for “tags” to facilitate purification, lower immunogenicity, or simplify analytical screening. Examples of protein tags used in this work include the HIS tag and the Avi tag. Tags were separated from each other and from the ANGPTL7 sequences with short linker sequences. All tags were added to the C-terminal end of the sequences. Additionally, the native signal peptide was removed and replaced with a signal peptide conducive to recombinant expression.

After all modifications were complete, the amino acid sequences were back translated into DNA sequences and optimized for the codon biases found in the human genome. These optimized DNA sequences were sent to Integrated DNA Technologies and produced as DNA fragment(s) with DNA overhangs added to the 5′ and 3′ ends. Utilizing the DNA overhangs and the Gibson cloning method, these DNA fragments were assembled into the expected sequence and cloned into a mammalian expression plasmid driven by a CMV promoter. The plasmid was propagated in E. coli with appropriate antibiotic selection and prepared, at a scale useful for recombinant expression, utilizing commercially available preparation kits purchased from Qiagen. The sequence of the plasmid and the expression gene was then confirmed using Sanger sequencing.

Using polyethylenimine, the sequence verified plasmid was transfected into human embryonic kidney cells adapted for recombinant expression. One day after transfection, the cells were supplemented with chemicals and nutrients designed to increase recombinant protein expression. These supplements include sodium propionate, valproic acid, glucose, glutamine, and a variety of yeast lysates. Five days after transfection, the expressing cell cultures were harvested. As the recombinant protein is secreted into the growth medium, the cells, cell fragments, and cell debris, were removed via centrifugation and filtration through a membrane with pores no greater than 0.22 microns. The clarified culture medium, conditioned with the recombinant protein, was now ready for purification. Using a FPLC (Fast pressure liquid chromatography) and the appropriate commercially available pre-packed affinity chromatography column for the C-terminal tag(s) the protein of interest was purified from the cell culture medium and immobilized on the chromatography column. For HIS tagged proteins, a Ni-NTA agarose column was used. Multiple column washes followed, each specific for the type of column/tag/chromatography. The protein of interest was eluted from the column with 300 mM of imidazole for HIS tagged proteins.

Protein quality and quantity were assessed using a combination of SDS-PAGE gel electrophoresis, spectrophotometry, and analytical-SEC (size exclusion chromatography). Most proteins for this project required further purification. To further polish the proteins, the FPLC and a second chromatography column were employed. Separation by size, accomplished with a size exclusion column, enabled increased protein purities up to >90%. SEC also enabled buffer exchange out of the affinity chromatography elution buffer and into the final buffer of choice (PBS). The protein sample then moved onto quality control. If, at any point during purification, protein concentration needed to be increased, Amicon Ultra molecular weight cutoff (MWCO) filtration units were used to concentrate protein by separating protein from buffer. MWCO was chosen to ensure compatibility with the size of the protein of interest. Before moving to final quality control, all final samples were concentrated to >1 milligram per milliliter.

Endotoxin contamination in the final protein sample was assayed using Charles River's Endosafe PTS system. Spectrophotometry determined the final protein concentration. Three micrograms of the final sample were injected onto an analytical SEC column (YMC Diol 300) to determine its final purity. Additionally, sometimes SDS-PAGE electrophoresis was performed to determine final quality. Once all final metrics passed, the protein was sterilized in a biosafety cabinet using a sterile 0.22 micron filter. This was followed by sterile aliquoting and flash freezing in liquid nitrogen before storage at −80° C.

### Example 6

Recovery of ANGPTL7 antibody sequences from immunized mice. ANGPTL7 Immunization: Four cohorts of Alloy Therapeutic transgenic humanized mice, ATX-GK, were immunized with various human ANGPTL7 antigens using a standard 5-week RIMMS protocol: 10 μg subcutaneous dosing of antigen emulsified in complete Freund's adjuvant followed by 5 weekly subcutaneous dosing of antigen emulsified in incomplete Freund's adjuvant.

Cohort 1: Three ATX-GK mice immunized with human ANGPTL7 fibrinogen domain (ATX-P-60; SEQ ID NO: 373). Cohort 2: Three ATX-GK mice immunized with full length human ANGPTL7 monomeric variant L59P_L84P (ATX-P-62; SEQ ID NO: 371). Cohort 3: Three ATX-GK mice immunized with full length human ANGPTL7 monomeric variant L59 GGPGG (ATX-P-63; SEQ ID NO: 372). Cohort 4: Three ATX-GK mice immunized with human wild type ANGPTL7 multimer (PExt-1; SEQ ID NO: 374).

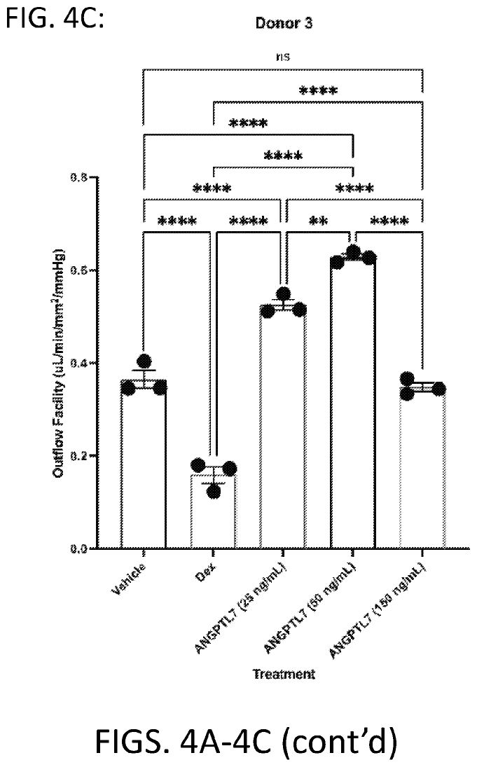

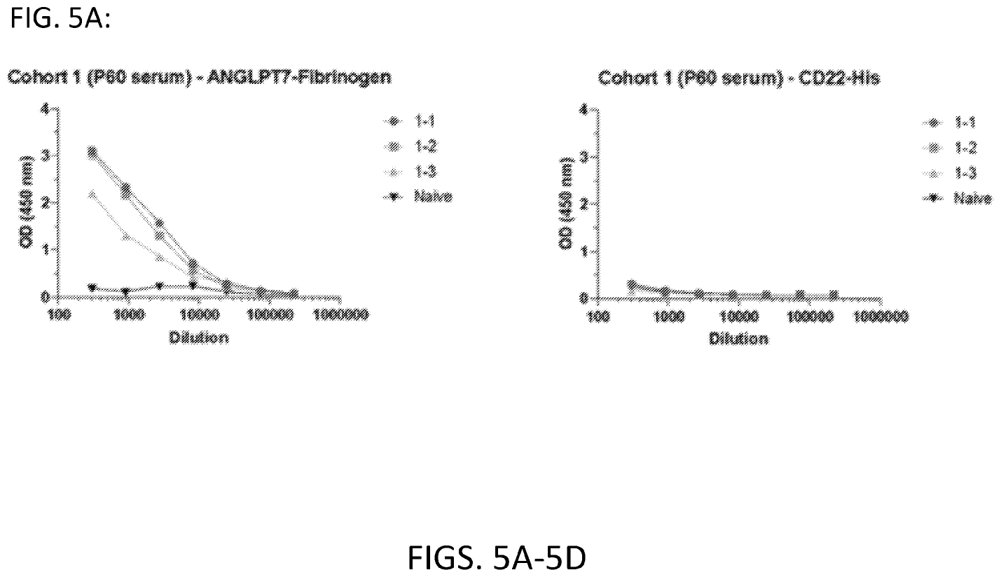

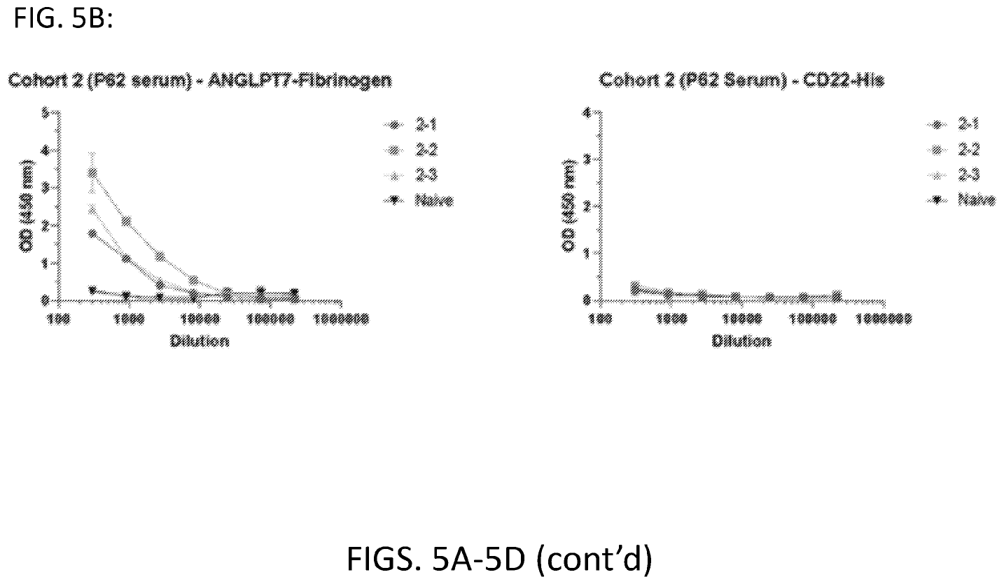

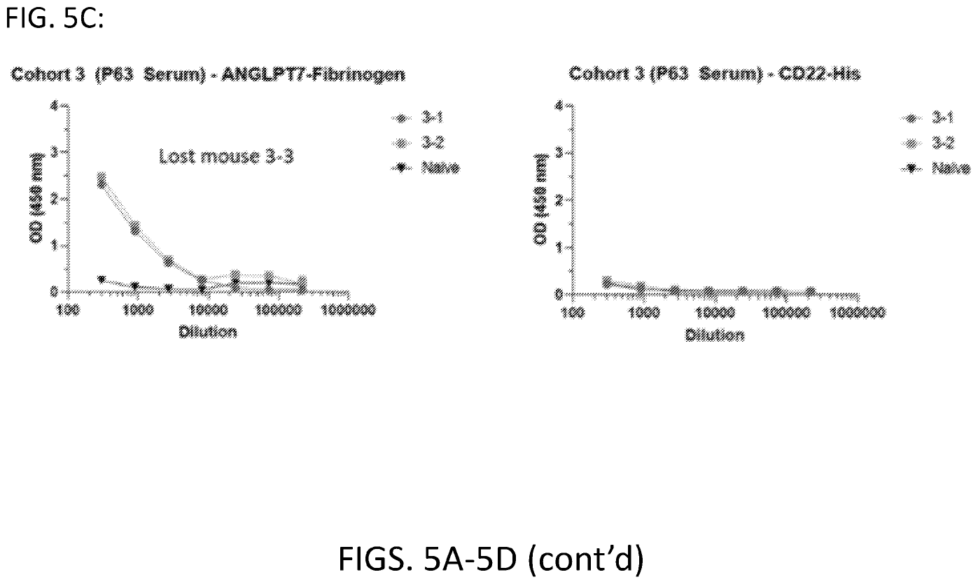

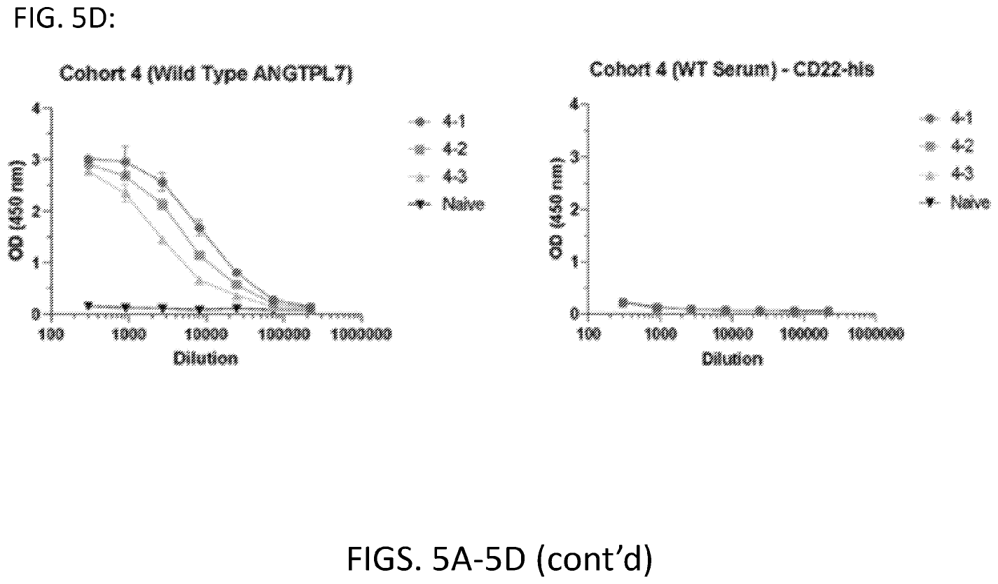

Sample bleeds were taken at week four and tested for antigen positive serum titer and purification tag negative serum titer by ELISA. ELISA plates were coated with either 1 ug/ml of ANGPTL7 immunogen or an irrelevant protein with the same purification tag as the immunogen. Antigen coated plates were incubated with seven 10-fold serial dilutions of sera starting at 1:300. Antibodies bound to antigen were detected by anti-mouse IgG HRP secondary antibody and one step TMB solution. The absorbance signal at 450 nm was measured with an ELISA microplate reader (FIGS. 5A-5D).

Hybridoma: Immune tissues from high titer mice were harvested and preserved for antibody discovery. Hybridoma cell lines producing ANGPTL7 antibodies were produced by fusion of single B Cells from spleen and lymph nodes of titer positive mice with myeloma cells. Twenty 96 well plates of hybridoma fusions were generated and expanded. Hybridomas expressing ANGPTL7 specific antibodies were detected by antigen binding by ELISA. Affinity of antibodies in the hybridoma supernatants were measured by SPR using the Octet instrument. ANGPTL7 antibodies in hybridoma supernatant were loaded on a biosensor. Response was measured as a nm shift in the interference pattern and was proportional to the number of antibodies bound to the surface of the biosensor. The binding interaction of ANGPTL7 to the immobilized antibodies was measured as association (kon). Following analyte association, the biosensor was dipped into PBS without ANGPTL7, and the bound antigen was allowed to dissociate from the antibody (kdis). KD (M), or affinity of the antibodies for ANGPTL7 was measured as kdis/kon.

Heavy and light chains from validated hybridomas were sequenced. RNA was isolated from ANGPTL7 antibody secreting hybridomas and heavy and light chain variable regions were cloned by reverse transcription using gene specific primers followed by PCR amplification with variable chain gene specific primers. PCR products were sequenced by standard Sanger sequencing methods.

Phage Display Variable heavy and light chains were amplified from the spleen of high titer immunized mice by reverse transcription using gene specific primers followed by PCR amplification with variable chain gene specific primers. Variable regions were cloned into a phage display vector designed to express Fabs on phage g3p protein. Libraries of phage expressing unique Fabs were amplified and purified. Phage were allowed to bind to biotinylated ANGPTL7 antigens captured on streptavidin magnetic beads. Phage remaining bound to antigen beads after several stringent washes was eluted using a basic triethylamine solution and neutralized with Tris buffer pH 8.0. Eluted phages were reinfected into TG1 bacterial cells, amplified by co-infection with M13 helper phage, and purified by PEG precipitation. Purified phages expressing Fabs were selected for antigen binding as described. Phage from the second round were diluted and infected into TG1 cells. Polyclonal pools of phage output from two rounds of panning were tested by ELISA to confirm that the pools contained ANGPTL7-specific phage. Variable heavy and light chain regions were sequenced from single infected bacterial colonies using a rolling circle amplification and standard Sanger sequencing.

Antibody Sequencing. Unique variable heavy and light chain pairs from hybridoma and phage display campaigns were cloned into vectors designed to express full length antibodies as IgGs in HEK293 cells under the control of a CMV promoter. Antibody expression vectors were complexed with polyethylenimine and transfected into HEK293 cultures. After 5 days of shaking at 37° C. in 293 cell culture media, antibodies were captured on agarose-based protein A resin. After several stringent washes, antibodies were eluted in glycine solution, pH 3, neutralized with Hepes, pH 9, and buffer exchanged into PBS.

### Example 7

Human ANGPTL7 monoclonal antibody differential scanning fluorimetry (DSF). Development of effective monoclonal antibodies depends not only on their biological activity but also on their physicochemical properties, such as homogeneity and stability. mAb stability can be affected by their formulation. Among the many techniques used to study the stability of mAbs, differential scanning fluorimetry (DSF) offers both excellent throughput and minimal material consumption. DSF measures the temperature of the protein unfolding transition (Tm) based on the change in fluorescence intensity of an environmentally sensitive dye.

Experiments were conducted to assess the thermal stability of the human ANGPTL7 monoclonal antibodies (“ATX” antibodies) of the present disclosure by determining the melting temperature. Thermal stability was assessed via differential scanning fluorimetry (DSF) utilizing the Protein Thermal Shift (PTS) assay from Applied Biosystems. The assay was performed according to the manufacturer's instruction. Briefly, the antibody to be evaluated was prepared in triplicate by mixing with Protein Thermal Shift dye and buffer; a real-time melt experiment from 25° C. to 95° C. was run on QuantStudio 3. Data was analyzed by using Protein Thermal Shift Software and the melting temperature (Tm) was calculated from the melt curve (Table 1).

TABLE 1
DSF analysis of human anti-ANGPTL7 monoclonal antibodies.
Protein Thermal Shift Assay Results

| Sample ID | Tm D - Mean | Tm 2 - Mean |
|---|---|---|
| ATX-P-423 | 62.50 | |
| ATX-P-424 | 62.69 | 73.14 |
| ATX-P-425 | 62.97 | 71.94 |
| ATX-P-427 | 62.78 | 67.87 |
| ATX-P-428 | 62.62 | 79.24 |
| ATX-P-429 | 62.64 | 80.49 |
| ATX-P-430 | 62.59 | |
| ATX-P-431 | 60.77 | |
| ATX-P-432 | 62.82 | |
| ATX-P-433 | 62.68 | 72.49 |
| ATX-P-434 | 62.82 | 75.29 |
| ATX-P-435 | 62.68 | 78.38 |
| ATX-P-436 | 62.33 | 80.83 |
| ATX-P-437 | 62.42 | |
| ATX-P-438 | 62.63 | 81.09 |
| ATX-P-439 | 62.77 | 83.42 |
| ATX-P-440 | 62.58 | 79.68 |
| ATX-P-442 | 62.44 | 85.10 |
| ATX-P-443 | 62.45 | |
| ATX-P-444 | 62.10 | |
| ATX-P-445 | 62.61 | |
| ATX-P-446 | 62.10 | |
| ATX-P-447 | 62.31 | |
| ATX-P-448 | 62.33 | 72.19 |
| ATX-P-449 | 62.24 | 80.41 |

### Example 8

ANGPTL7 Antibody Cross-Blocking. High-throughput epitope binning experiments were conducted on real-time label-free biosensors (Carterra LSA) to sort large panels of mAbs into bins based on their ability to block one another for binding to the antigen. In a pairwise epitope binning analysis, antigen and antibody 2 (analyte antibody) are sequentially applied to the sensor chip (HC200M) covalently pre-loaded with antibody 1 (ligand antibody). An increase in response upon exposure to the analyte antibody indicates non-competition between the two antibodies, whereas a lack of change in the signal indicates competition. Antibodies having the same blocking profiles towards others in the test set are grouped into one bin. Community network plots are used to explore clustering of mAbs that share similar but not necessarily identical competition profiles.

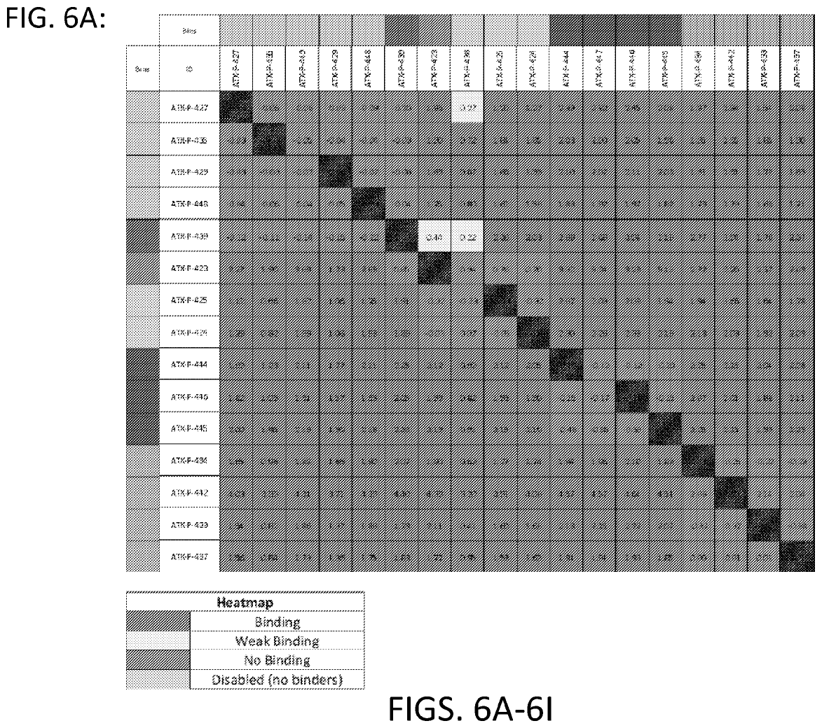

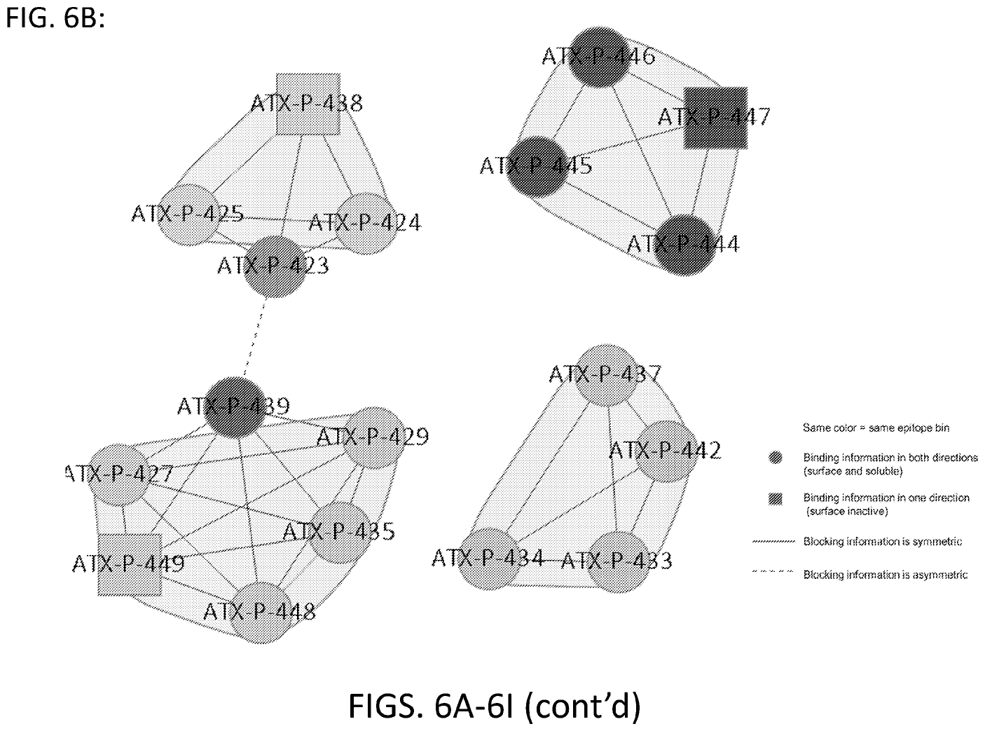

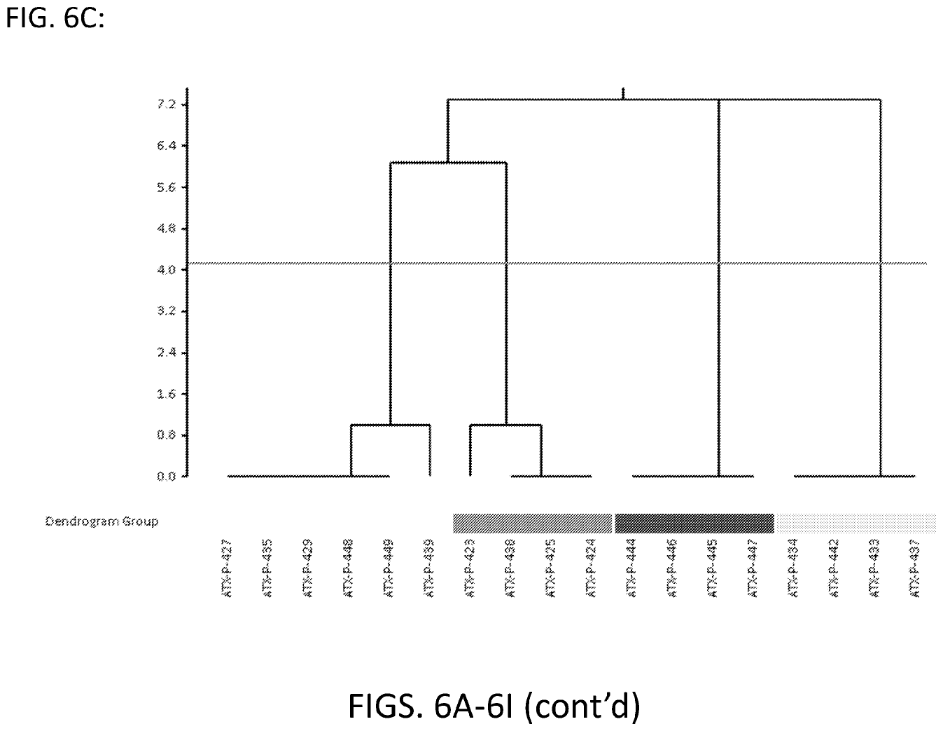

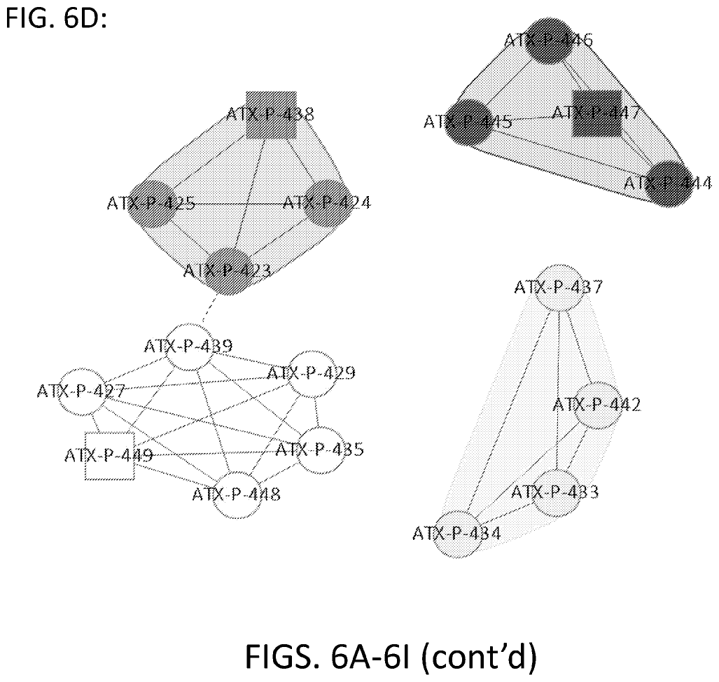

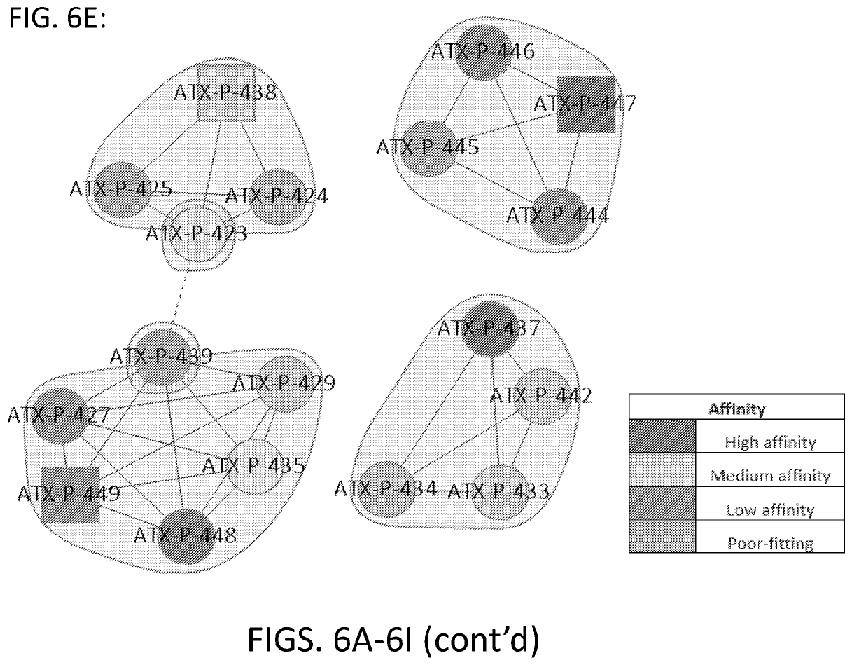

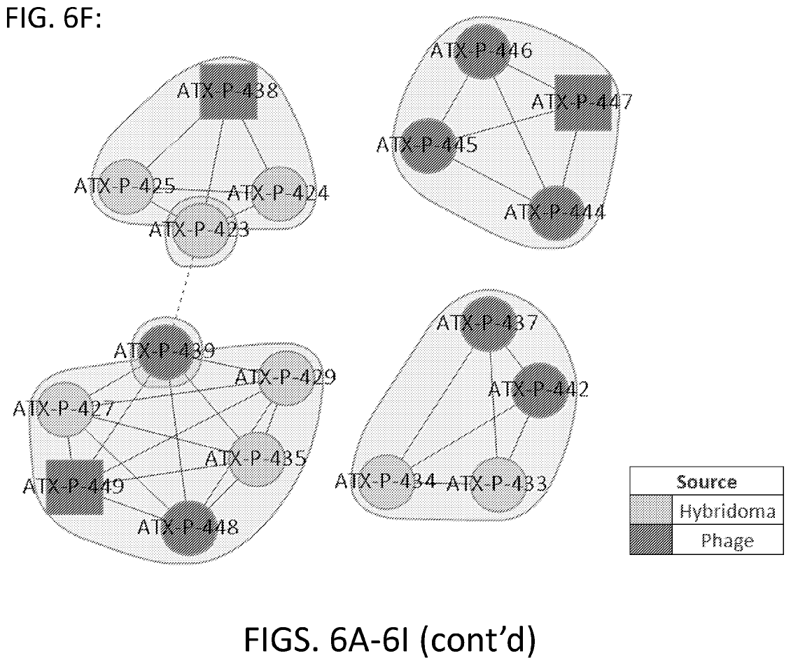

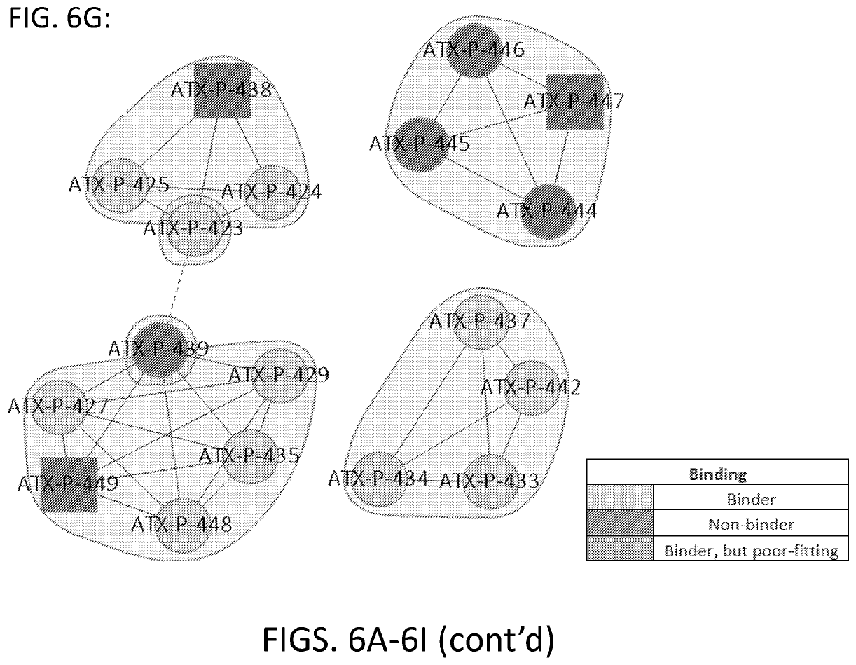

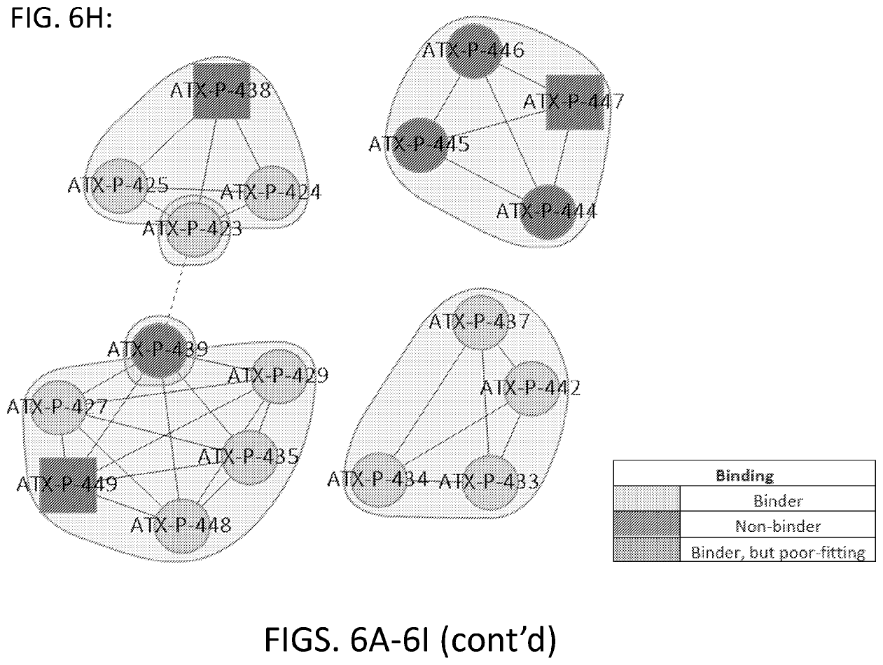

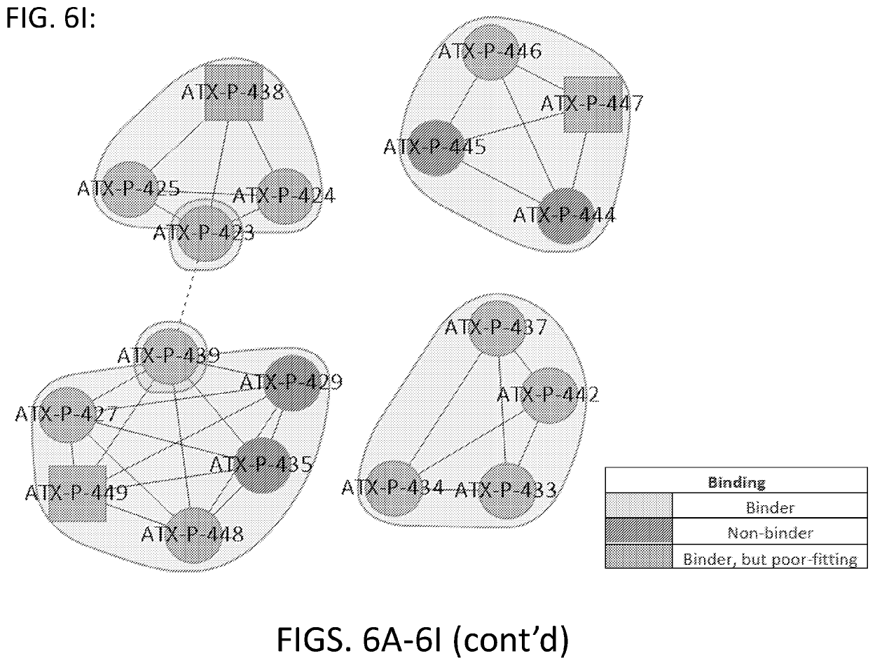

Rather than relying strictly on the sandwiching/blocking assignments in the heat map (FIG. 6A), as the Bin network plots do, hierarchical clustering was applied to the sorted heat map to generate various dendrograms and network plots, which progressively group mAbs based on various binning parameters. (FIGS. 6B-6I). FIG. 6B includes data from a granular binning network. FIG. 6C: includes data from a combined binary dendrogram (color indicates bins in the Community binning network). FIG. 6D includes data from a community binning network. FIG. 6E includes binning data based on affinity for huANGPTL7-his (P62). FIG. 6F includes binning data based on antibody source (hybridoma or phage). FIG. 6G includes binning data based on fibrinogen domain (P60P) binding. FIG. 6H includes binning data based on rabbit ANGPTL7 (p66) binding. FIG. 6I includes binning data based on mouse ANGPTL7 binding.

### Example 9

ANGPTL7 Functional Evaluation. Experiments were conducted to test the effects of anti-ANGPTL7 antibodies on conventional outflow facility using a 3D HTM/HSC Tissue Model (see FIG. 3A/Example 3). The purpose of this study is to screen antibodies that bind ANGPTL7 to determine whether they are able to block ANGPTL7's depression of outflow facility in an organoid model of the aqueous outflow tract. Antibodies that are capable of increasing outflow facility by blocking ANGPTL7's activity are drug candidates for further study.

The studies were carried out using a bioengineered ex vivo human outflow tract model described above. Glauconix developed a proprietary technology allowing multi-layer growth of bioengineered 3D human trabecular meshwork (HTM) cells co-cultured with human Schlemms' canal (HSC) cells, thereby mimicking the architecture of the tissues responsible for pressure build-up in the human eye. This allows endpoint analyses relevant to IOP-modulation. Previous experiments described above showed both dexamethasone and ANGPTL7 significantly decreased outflow facility in the 3D human tissue model. In this experiment, the effects of 20 anti-ANGPTL7 antibodies on outflow facility with ANGPTL7 treatment were investigated.

As show in FIG. 7, outflow facility of one donor cell line treated with vehicle (DMSO), 500 nM Dexamethasone, 50 ng/mL ANGPTL7 (1.1 nM) alone, 50 ng ANGPTL7+an isotype control Antibody (330) at 11.1 nM, and 20 different Anti-ANGPTL7 antibodies at 11.1 nM together with 50 ng/mL ANGPTL7. Samples were analyzed for effects against isotype control (330) using One-way ANOVA (GraphPad Prism Software, Inc., La Jolla, CA), *P<0.05, **P<0.0.01, ***P<0.001, ****P<0.0001 N=3 per treatment group. These data clearly demonstrate that the efficacy of the anti-ANGPTL7 antibodies of the present disclosure to restore outflow facility.

### Example 10

ANGPTL7 Antibody Kinetics. Experiments were conducted to determine the reactivity and dissociation constants (K_{D}) of anti-ANGPTL7 antibodies with the human fibrinogen domain of the ANGPTL7 protein. Binding experiments were performed on Octet HTX (ForteBio) in 1×PBS, pH 7.4, 0.1 mg/ml BSA, 0.05% Sodium Azide, and 0.02 Tween 20. Antibodies at a concentration of 50 nM were loaded on pre-hydrated AHC biosensors for 5 min. The loaded sensors were dipped into monovalent P60 (huANGPTL7-Fibrinogen-his) at a concentration of 200 nM for 5 min association, followed by 10 min dissociation. Results were analyzed with ForteBio Data Analysis 11.1 and fit to a 1:1 binding model to determine the monovalent K_{D}. Responses (nm) at the end of association were also determined (Table 2).

TABLE 2
Reactivity of anti-ANGPTL7 antibodies with the
human fibrinogen domain of the ANGPTL7 protein.

| | AHC > Antibody > P60 (huANGPTL7-Fibrinogen-his) @ 200 nM | | | | |
|---|---|---|---|---|---|
| Ab ID | Response | KD (M) | kon(1/Ms) | kdis(1/s) | Full R\hat{ }2 |
| ATX-P-423 | 0.69 | 1.83E−08 | 1.96E+05 | 3.60E−03 | 0.99 |
| ATX-P-424 | 0.41 | 1.06E−07 | 1.11E+05 | 1.18E−02 | 0.99 |
| ATX-P-425 | 0.36 | 3.05E−07 | 7.04E+04 | 2.14E−02 | 1.00 |
| ATX-P-427 | 0.50 | 6.51E−08 | 1.55E+05 | 1.01E−02 | 0.96 |
| ATX-P-428 | 0.02 | N.B. | N.B. | N.B. | 0.42 |
| ATX-P-429 | 0.52 | 7.53E−08 | 4.23E+04 | 3.18E−03 | 1.00 |
| ATX-P-430 | 0.13 | N.B. | N.B. | N.B. | 0.99 |
| ATX-P-431 | 0.11 | N.B. | N.B. | N.B. | 0.90 |
| ATX-P-432 | 0.10 | N.B. | N.B | N.B. | 0.95 |
| ATX-P-433 | 0.66 | 1.09E−08 | 2.23E+05 | 2.43E−03 | 0.96 |
| ATX-P-434 | 0.53 | 4.63E−10 | 2.58E+05 | 1.20E−04 | 0.99 |
| ATX-P-435 | 0.45 | 7.70E−08 | 2.52E+04 | 1.94E−03 | 1.00 |
| ATX-P-436 | 0.14 | N.B. | N.B. | N.B. | 0.99 |
| ATX-P-437 | 0.60 | 6.58E−10 | 2.13E+05 | 1.40E−04 | 1.00 |
| ATX-P-438 | 0.08 | N.B. | N.B | N.B. | 0.99 |
| ATX-P-439 | 0.28 | P.F | P.F | P.F | 0.75 |
| ATX-P-440 | 0.39 | 5.58E−08 | 4.65E+04 | 2.60E−03 | 0.97 |
| ATX-P-442 | 0.69 | 4.62E−09 | 3.41E+05 | 1.58E−03 | 0.96 |
| ATX-P-443 | 0.01 | N.B. | N.B. | N.B. | 0.41 |
| ATX-P-444 | 0.14 | N.B. | N.B | N.B. | 1.00 |
| ATX-P-445 | 0.12 | N.B. | N.B. | N.B. | 0.99 |
| ATX-P-446 | 0.09 | N.B. | N.B. | N.B. | 0.90 |
| ATX-P-447 | 0.08 | N.B. | N.B. | N.B. | 0.94 |
| ATX-P-448 | 0.54 | 4.69E−09 | 9.47E+04 | 4.44E−04 | 0.97 |
| ATX-P-449 | 0.08 | N.B. | N.B. | N.B. | 0.67 |

Experiments were also conducted to determine the reactivity and dissociation constants (K_{D}) of anti-ANGPTL7 antibodies with the human ANGPTL7-his protein. Binding experiments were performed on Octet HTX (ForteBio) in 1×PBS, pH 7.4, 0.1 mg/mi BSA, 0.05% Sodium Azide, and 0.02% Tween 20. To measure monovalent binding kinetics, antibodies at a concentration of 50 nM were loaded on pre-hydrated AHC biosensors for 5 min. The loaded sensors were dipped into monovalent P62 (huANGPTL7-his) at a range of concentration from 0.343-250 nM (a serial 3-fold dilution) for 5 min association, followed by 10 min dissociation. Results were analyzed with ForteBio Data Analysis 11.1 and fit globally to a 1:1 binding model to determine the monovalent K_{D} (Table 3).

TABLE 3
Reactivity of anti-ANGPTL7 antibodies with the human ANGPTL7-his protein.

| | AHC > Antibody > P62 (huANGPTL7-his) @ 250 nM, 3X dilution | | | |
|---|---|---|---|---|
| Ab ID | KD (M) | kon(1/Ms) | kdis(1/s) or (koff) | Full R\hat{ }2 |
| ATX-P-423 | 3.58E−08 | 1.48E+05 | 5.30E−03 | 1.00 |
| ATX-P-424 | 1.64E−07 | 5.30E+04 | 8.68E−03 | 0.99 |
| ATX-P-425 | 2.19E−07 | 6.50E+04 | 1.42E−02 | 0.98 |
| ATX-P-427 | 3.97E−07 | 6.60E+04 | 2.62E−02 | 1.00 |
| ATX-P-428 | N.B. | N.B. | N.B. | 0.00 |
| ATX-P-429 | 9.27E−08 | 3.75E+04 | 3.48E−03 | 0.99 |
| ATX-P-430 | 6.56E−08 | 1.33E+05 | 8.73E−03 | 0.92 |
| ATX-P-431 | N.B. | N.B. | N.B. | 0.00 |
| ATX-P-432 | N.B. | N.B. | N.B. | 0.00 |
| ATX-P-433 | 1.30E−09 | 4.31E+05 | 5.60E−04 | 0.99 |
| ATX-P-434 | 6.71E−10 | 2.62E+05 | 1.76E−04 | 1.00 |
| ATX-P-435 | 3.51E−08 | 3.89E+04 | 1.37E−03 | 0.98 |
| ATX-P-436 | N.B. | N.B. | N.B. | 0.00 |
| ATX-P-437 | Very low KD | Very low KD | Slow Koff | 1.00 |
| ATX-P-438 | 7.81E−08 | 1.28E+05 | 1.00E−02 | 0.98 |
| ATX-P-439 | 4.11E−10 | 1.02E+06 | 4.18E−04 | 1.00 |
| ATX-P-440 | 8.40E−08 | 3.96E+04 | 3.32E−03 | 0.98 |
| ATX-P-442 | 1.21E−09 | 4.68E+05 | 5.68E−04 | 0.99 |
| ATX-P-443 | N.B. | N.B. | N.B. | 0.00 |
| ATX-P-444 | 2.17E−10 | 3.57E+05 | 7.72E−05 | 1.00 |
| ATX-P-445 | 4.24E−10 | 6.49E+05 | 2.75E−04 | 0.99 |
| ATX-P-446 | 1.57E−10 | 4.00E+05 | 6.28E−05 | 1.00 |
| ATX-P-447 | Low KD | Low KD | Slow, drifting | 0.99 |
| ATX-P-448 | 9.83E−11 | 3.27E+05 | 3.22E−05 | 1.00 |
| ATX-P-449 | Very W.B. | Very W.B. | Very W.B. | 0.00 |

Experiments were also conducted to determine the reactivity and dissociation constants (K_{D}) of anti-ANGPTL7 antibodies with the human ANGPTL7 GGPGG_sub-his protein. Binding experiments were performed on Octet HTX (ForteBo) in 1×PBS, pH 7.4, 0.1 mg/mi BSA, 0.05% Sodium Azide, and 0.02% Tween 20. Antibodies at a concentration of 50 nM were loaded on pre-hydrated AHC biosensors for 5 min. The loaded sensors were dipped into monovalent P63 (huANGTPL7-GGPGG_sub-his) at a concentration of 100 nM for 5 min of association, followed by 10 min of dissociation. Results were analyzed with ForteBio Data Analysis 11.1 and fit to a 1:1 binding model to determine the monovalent K_{D}. Responses (nm) at the end of association were also determined (Table 4).

TABLE 4
Reactivity of anti-ANGPTL7 antibodies with
the human ANGPTL7 GGPGG_sub-his protein.

| | AHC > Antibody > P63 (huANGTPL7-GGPGG_sub-his) @ 100 nM | | | | |
|---|---|---|---|---|---|
| Ab ID | Response | KD (M) | kon(1/Ms) | kdis(1/s) | Full R\hat{ }2 |
| ATX-P-423 | 0.73 | 2.82E−08 | 1.29E+05 | 3.63E−03 | 0.98 |
| ATX-P-424 | 0.43 | 5.45E−08 | 9.62E+04 | 5.24E−03 | 0.96 |
| ATX-P-425 | 0.35 | 1.31E−07 | 8.04E+04 | 1.05E−02 | 0.97 |
| ATX-P-427 | 0.45 | P.F. | P.F. | P.F. | 0.89 |
| ATX-P-428 | 0.03 | N.B. | N.B. | N.B. | 0.75 |
| ATX-P-429 | 0.46 | 8.31E−08 | 4.06E+04 | 3.37E−03 | 1.00 |
| ATX-P-430 | 0.14 | N.B. | N.B. | N.B. | 0.97 |
| ATX-P-431 | 0.11 | N.B. | N.B. | N.B. | 0.91 |
| ATX-P-432 | 0.09 | N.B. | N.B. | N.B. | 0.95 |
| ATX-P-433 | 0.71 | 1.89E−08 | 1.34E+05 | 2.53E−03 | 0.96 |
| ATX-P-434 | 0.70 | 1.50E−09 | 1.40E+05 | 2.10E−04 | 0.99 |
| ATX-P-435 | 0.36 | 6.48E−08 | 3.07E+04 | 1.99E−03 | 0.99 |
| ATX-P-436 | 0.13 | N.B. | N.B. | N.B. | 0.98 |
| ATX-P-437 | 0.73 | 1.37E−09 | 1.29E+05 | 1.77E−04 | 0.99 |
| ATX-P-438 | 0.19 | N.B. | N.B. | N.B. | 0.95 |
| ATX-P-439 | 0.31 | P.F. | P.F. | P.F. | 0.80 |
| ATX-P-440 | 0.38 | 6.37E−08 | 4.33E+04 | 2.76E−03 | 0.97 |
| ATX-P-442 | 0.82 | 9.67E−09 | 1.85E+05 | 1.79E−03 | 0.97 |
| ATX-P-443 | 0.02 | N.B. | N.B. | N.B. | 0.80 |
| ATX-P-444 | 0.56 | 7.07E−10 | 4.76E+05 | 3.37E−04 | 0.96 |
| ATX-P-445 | 0.64 | P.F. | P.F. | P.F. | 0.90 |
| ATX-P-446 | 0.56 | 6.48E−10 | 4.97E+05 | 3.22E−04 | 0.94 |
| ATX-P-447 | 0.71 | 1.32E−09 | 4.65B+05 | 6.15E−04 | 0.93 |
| ATX-P-448 | 0.53 | 6.02E−09 | 8.34E+04 | 5.02E−04 | 0.97 |
| ATX-P-449 | 0.15 | N.B. | N.B. | N.B. | 0.84 |

Experiments were also conducted to determine the reactivity and dissociation constants (K_{D}) of anti-ANGPTL7 antibodies with the mouse ANGPTL7 protein. Binding experiments were performed on Octet HTX (ForteBio) in 1×PBS, pH 7.4, 0.1 mg/mi BSA, 0.05% Sodium Azide, and 0.02% Tween 20. Antibodies at a concentration of 50 nM were loaded on pre-hydrated AHC biosensors for 5 mA. The loaded sensors were dipped into monovalent muANGPTL7-his (R&D Systems) at a concentration of 200 nM for 5 min of association, followed by 10 min of dissociation. Results were analyzed with ForteBio Data Analysis 11.1 and fit to a 1:1 binding model to determine the monovalent K_{D}. Responses (nm) at the end of association were also determined (Table 5).

TABLE 5
Reactivity of anti-ANGPTL7 antibodies with the mouse ANGPTL7 protein.

| | AHC > Antibody > muANGPTL7-his (R&D Systems) @ 200 nM | | | | |
|---|---|---|---|---|---|
| Ab ID | Response | KD (M) | kon(1/Ms) | kdis(1/s) | Full R\hat{ }2 |
| ATX-P-423 | 0.14 | N.B. | N.B. | N.B. | 0.89 |
| ATX-P-424 | 0.14 | N.B. | N.B. | N.B. | 0.91 |
| ATX-P-425 | 0.20 | P.F./L.R. | P.F./L.R. | P.F./L.R. | 0.85 |
| ATX-P-427 | 0.48 | 1.03E−08 | 1.20E+05 | 1.23E−03 | 0.90 |
| ATX-P-428 | 0.04 | N.B. | N.B. | N.B. | 0.90 |
| ATX-P-429 | 0.00 | N.B. | N.B. | N.B. | 0.70 |
| ATX-P-430 | 0.01 | N.B. | N.B. | N.B. | 0.47 |
| ATX-P-431 | 0.04 | N.B. | N.B. | N.B. | 0.94 |
| ATX-P-432 | 0.03 | N.B. | N.B. | N.B. | 0.85 |
| ATX-P-433 | 0.17 | P.F./L.R. | P.F./L.R. | P.F./L.R. | 0.89 |
| ATX-P-434 | 0.54 | 7.28E−09 | 1.41E+05 | 1.03E−03 | 0.92 |
| ATX-P-435 | 0.01 | N.B. | N.B. | N.B. | 0.76 |
| ATX-P-436 | 0.01 | N.B. | N.B. | N.B. | 0.85 |
| ATX-P-437 | 0.02 | N.B. | N.B. | N.B. | 0.96 |
| ATX-P-438 | 0.01 | N.B. | N.B. | N.B. | 0.89 |
| ATX-P-439 | 0.53 | P.F. | P.F. | P.F. | 0.89 |
| ATX-P-440 | 0.21 | 5.53E−08 | 1.66E+04 | 9.15E−04 | 0.99 |
| ATX-P-442 | 0.32 | P.F. | P.F. | P.F. | 0.84 |
| ATX-P-443 | 0.02 | N.B. | N.B. | N.B. | 0.86 |
| ATX-P-444 | 0.01 | N.B. | N.B. | N.B. | 0.45 |
| ATX-P-445 | 0.01 | N.B. | N.B. | N.B. | 0.62 |
| ATX-P-446 | 0.05 | N.B. | N.B. | N.B. | 0.99 |
| ATX-P-447 | 0.12 | N.B. | N.B. | N.B. | 0.94 |
| ATX-P-448 | 0.69 | Low KD | Low KD | Slow Koff | 0.99 |
| ATX-P-449 | 0.75 | 8.57E−10 | 8.16E+04 | 7.00E−05 | 0.99 |

Experiments were also conducted to determine the reactivity and dissociation constants (KA) of anti-ANGPTL7 antibodies with the rabbit ANGPTL7 protein. Binding experiments were performed on Octet HTX (ForteBio) in Rx PBS, pH 7.4, 0.1 mg/mi BSA, 0.05% Sodium Azide, and 0.02% Tween 20. Antibodies at a concentration of 50 nM were loaded on pre-hydrated AHC biosensors for 5 min. The loaded sensors were dipped into monovalent P66 (rabbit ANGPTL7-his) at a concentration of 200 nM for 5 min of association, followed by 10 min of dissociation. Results were analyzed with ForteBio Data Analysis 11.1 and fit to a 1:1 binding model to determine the monovalent KD. Responses (nm) at the end of association were also determined (Table 6).

TABLE 6
Reactivity of anti-ANGPTL7 antibodies
with the rabbit ANGPTL7 protein.

| | AHC > Antibody > P66 (rabbit ANGPTL7-his) @ 200 nM | | | | |
|---|---|---|---|---|---|
| Ab ID | Response | KD (M) | kon(1/Ms) | kdis(1/8) | Full R\hat{ }2 |
| ATX-P-423 | 1.02 | 6.69E−09 | 8.29E+04 | 5.54E−04 | 0.99 |
| ATX-P-424 | 0.93 | 4.65E−09 | 5.24E+04 | 2.44E−04 | 0.99 |
| ATX-P-425 | 0.95 | 2.38E−09 | 4.12E+04 | 9.79E−05 | 1.00 |
| ATX-P-427 | 0.93 | 3.21E−09 | 4.16E+04 | 1.33E−04 | 1.00 |
| ATX-P-428 | 0.04 | N.B. | N.B. | N.B. | 0.81 |
| ATX-P-429 | 0.04 | N.B. | N.B. | N.B. | 0.92 |
| ATX-P-430 | 0.21 | N.B. | N.B. | N.B. | 0.96 |
| ATX-P-431 | 0.11 | N.B. | N.B. | N.B. | 0.86 |
| ATX-P-432 | 0.09 | N.B. | N.B. | N.B. | 0.70 |
| ATX-P-433 | 0.90 | 7.02E−09 | 1.04E+05 | 7.27E−04 | 0.99 |
| ATX-P-434 | 0.81 | Low KD | Low KD | Slow Koff | 0.99 |
| ATX-P-435 | 0.01 | N.B. | N.B. | N.B. | 0.67 |
| ATX-P-436 | 0.02 | N.B. | N.B. | N.B. | 0.70 |
| ATX-P-437 | 0.80 | 7.86E−10 | 1.27E+05 | 1.00E−04 | 0.99 |
| ATX-P-438 | 0.42 | 7.16E−09 | 6.79E+04 | 4.86E−04 | 0.98 |
| ATX-P-439 | 0.92 | 2.95E−09 | 1.01E+05 | 2.98E−04 | 0.98 |
| ATX-P-440 | 0.57 | 6.27E−09 | 5.07E+04 | 3.18E−04 | 0.98 |
| ATX-P-442 | 0.95 | 3.38E−09 | 1.44E+05 | 4.87E−04 | 0.99 |
| ATX-P-443 | 0.03 | N.B. | N.B. | N.B. | 0.85 |
| ATX-P-444 | 0.30 | N.B. | N.B. | N.B. | 0.96 |
| ATX-P-445 | 0.26 | N.B. | N.B. | N.B. | 0.96 |
| ATX-P-446 | 0.41 | 3.19E−09 | 1.08E+05 | 3.46E−04 | 0.98 |
| ATX-P-447 | 0.65 | 3.33E−09 | 8.31E+04 | 2.77E−04 | 0.99 |
| ATX-P-448 | 0.75 | 9.49E−10 | 4.77E+04 | 4.52E−05 | 0.99 |
| ATX-P-449 | 0.74 | 6.26E−10 | 6.53E+04 | 4.08E−05 | 0.99 |

Experiments were also conducted to determine the reactivity and dissociation constants (K_{D}) of anti-ANGPTL7 antibodies with the human ANGPTL7 multimer protein. Binding experiments were performed on Octet HTX (ForteBio) in 1×PBS, pH 7.4, 0.1 mg/ml BSA, 0.05% Sodium Azide, and 0.02% Tween 20. Antibodies at a concentration of 50 nM were loaded on pre-hydrated AHC biosensors for 5 mi. The loaded sensors were dipped into Pext-01 (huANGTPL7-FLAG) at a concentration of 100 nM for 5 min of association, followed by 10 min of dissociation. Results were analyzed with ForteBio Data Analysis 11.1 and fit to a 1:1 binding model to determine the apparent KD. Responses (nm) at the end of association were also determined (Table 7).

TABLE 7
Reactivity of anti-ANGPTL7 antibodies with
the human ANGPTL7 multimer protein.

| | AHC > Antibody > Pext-01 (huANGTPL7-FLAG multimer) @ 100 nM | | | | |
|---|---|---|---|---|---|
| Ab ID | Response | KD (M) | kon(1/Ms) | kdis(1/s) | Full R\hat{ }2 |
| ATX-P-423 | 0.87 | Low KD | Low KD | Slow Koff | 0.99 |
| ATX-P-424 | 0.76 | Low KD | Low KD | Slow Koff | 1.00 |
| ATX-P-425 | 0.83 | Low KD | Low KD | Slow Koff | 1.00 |
| ATX-P-427 | 0.82 | Low KD | Low KD | Slow Koff | 1.00 |
| ATX-P-428 | 0.05 | N.B. | N.B. | N.B. | 0.95 |
| ATX-P-429 | 0.58 | Low KD | Low KD | Slow Koff | 1.00 |
| ATX-P-430 | 0.20 | N.B. | N.B. | N.B. | 0.98 |
| ATX-P-431 | 0.21 | N.B. | N.B. | N.B. | 1.00 |
| ATX-P-432 | 0.22 | N.B. | N.B. | N.B. | 1.00 |
| ATX-P-433 | 0.72 | Low KD | Low KD | Slow Koff | 0.99 |
| ATX-P-434 | 0.68 | Low KD | Low KD | Slow Koff | 0.98 |
| ATX-P-435 | 0.45 | Low KD | Low KD | Slow Koff | 0.99 |
| ATX-P-436 | 0.16 | N.B. | N.B. | N.B. | 0.97 |
| ATX-P-437 | 0.71 | Low KD | Low KD | Slow Koff | 0.98 |
| ATX-P-438 | 0.60 | 6.53E−08 | 3.71E+04 | 2.42E−03 | 0.99 |
| ATX-P-439 | 0.87 | 1.28E−08 | 7.98E+04 | 1.02E−03 | 1.00 |
| ATX-P-440 | 0.61 | Low KD | Low KD | Slow Koff | 1.00 |
| ATX-P-442 | 0.79 | Low KD | Low KD | Slow Koff | 0.98 |
| ATX-P-443 | 0.03 | N.B. | N.B. | N.B. | 0.92 |
| ATX-P-444 | 0.70 | Low KD | Low KD | Slow Koff | 0.97 |
| ATX-P-445 | 0.80 | Low KD | Low KD | Slow Koff | 0.97 |
| ATX-P-446 | 0.75 | Low KD | Low KD | Slow Koff | 0.97 |
| ATX-P-447 | 0.91 | Low KD | Low KD | Slow Koff | 0.96 |
| ATX-P-448 | 0.58 | Low KD | Low KD | Slow Koff | 0.99 |
| ATX-P-449 | 0.65 | Low KD | Low KD | Slow Koff | 0.98 |

### Example 11

ANGPTL7 is a member of the ANGPTL family though its function is poorly characterized. Previous studies have shown that loss-of-function variants in the ANGPTL7 gene are associated with protection from glaucoma and reduced intraocular pressure (IOP). Therefore, experiments were conducted to investigate the role of ANGPTL7 in IOP homeostasis and its potential as a target for the development of therapeutics.

Briefly, an ANGPTL7 knockout model was generated by Lexicon therapeutics in 129SvEv-C57BL/6 mixed genetic background. ANGPTL7 WT (N=14) and ANGPTL7 KO (N=17) mice were implanted with osmotic pumps filled with PBS or dexamethasone-cyclodextrin solution (4 mg/kg/day) for 28 days. Body weight and IOP measurements were taken weekly. Alzet osmotic pumps (Model 1004) were sterile-filled with PBS or 45.45 mg/mL of dexamethasone-cyclodextrin solution to provide a dose of 4 mgs/kg/day for the average 30 g mouse. The pumps were then incubated in 0.9% normal saline at 37° C. for 48 hours before implantation. On Day 0, body weight and IOP measurements were taken and then used to randomize the mice into the following 4 treatment groups: Angptl7 KO+PBS, N=8; Angplt7 KO+Dexamethasone, N=9; Angptl7 WT+PBS, N=5; and Angptl7 WT+Dexamethasone, N=14. PBS or dexamethasone pumps were subcutaneously implanted into the mouse. IOP and body weight were taken weekly on days 7, 14, 21, and 28. On day 28, final body weight and IOP measurements were taken. OS/OD were enucleated and snap frozen in liquid nitrogen. Mice were euthanized following humane IACUC procedures.

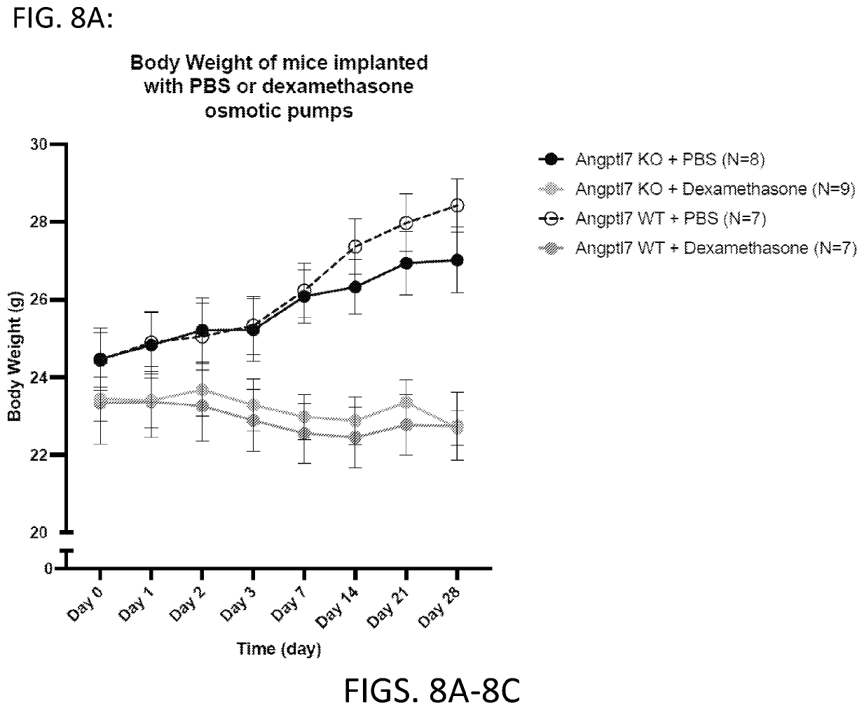

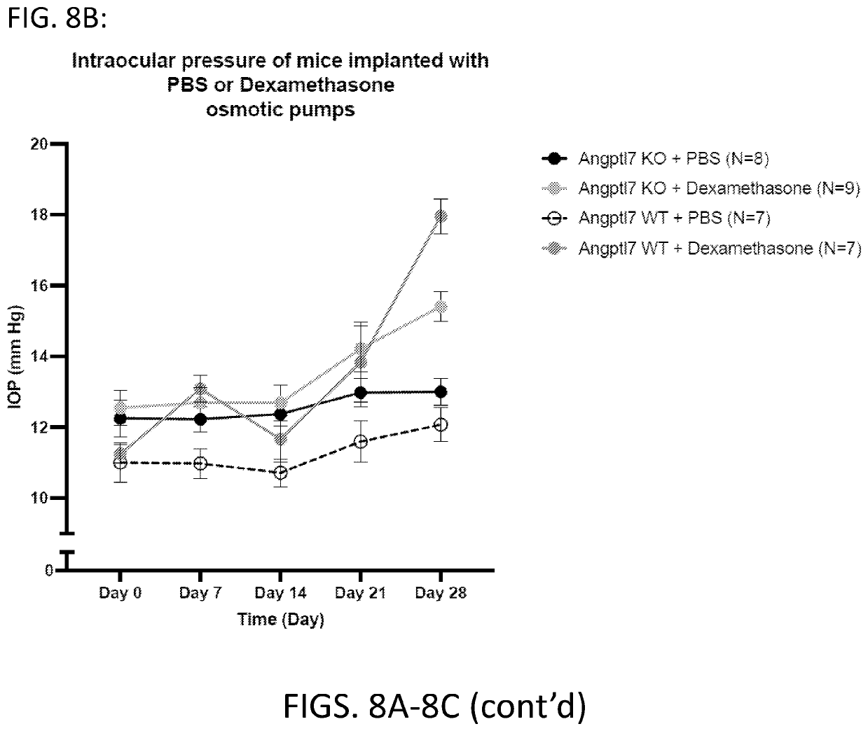

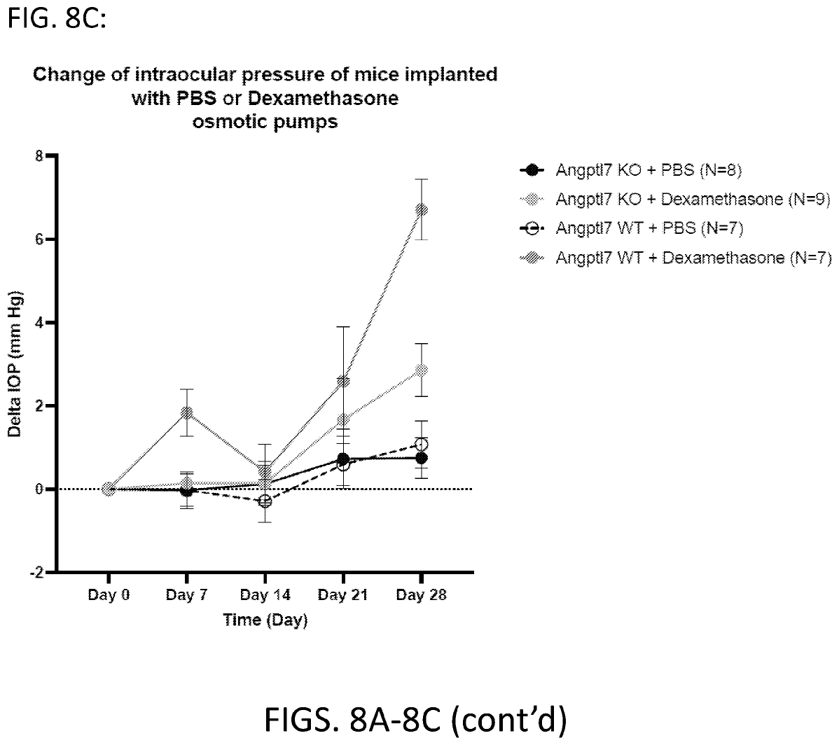

Representative results of the effects of dexamethasone-induced ocular hypertension in ANGPTL7 knockout mice are provided in FIGS. 8A-8C. FIG. 8A includes body weight data of mice post-implantation surgery; dexamethasone impaired weight gain in male mice compared to PBS-treated mice, thereby confirming the proper functioning of the dexamethasone osmotic pumps. FIG. 8B includes intraocular pressure (IOP) data of ANGPTL7 WT and ANGPTL7 KO mice dosed with PBS control or Dexamethasone (4 mg/kg/day); osmotic pumps were implanted on day 0. FIG. 8C includes representative data of the changes in IOP of ANGPTL7 WT and ANGPTL7 KO mice implanted with osmotic pumps containing PBS vehicle or dexamethasone (4 mg/kg/day) over 28 days. All IOP values were normalized to baseline (day 0) readings.

### Example 12

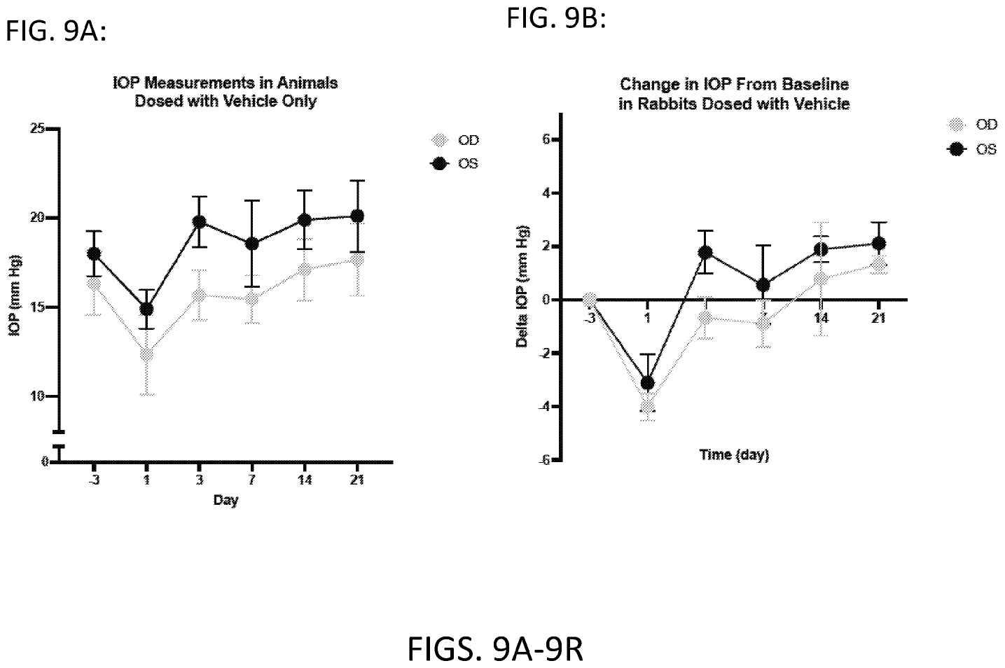

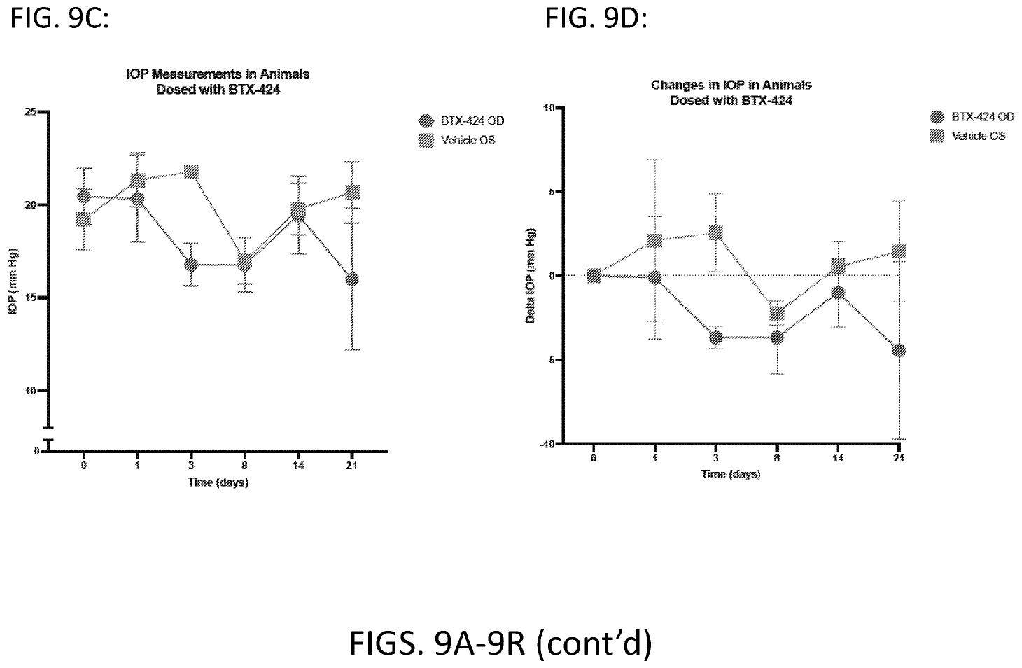

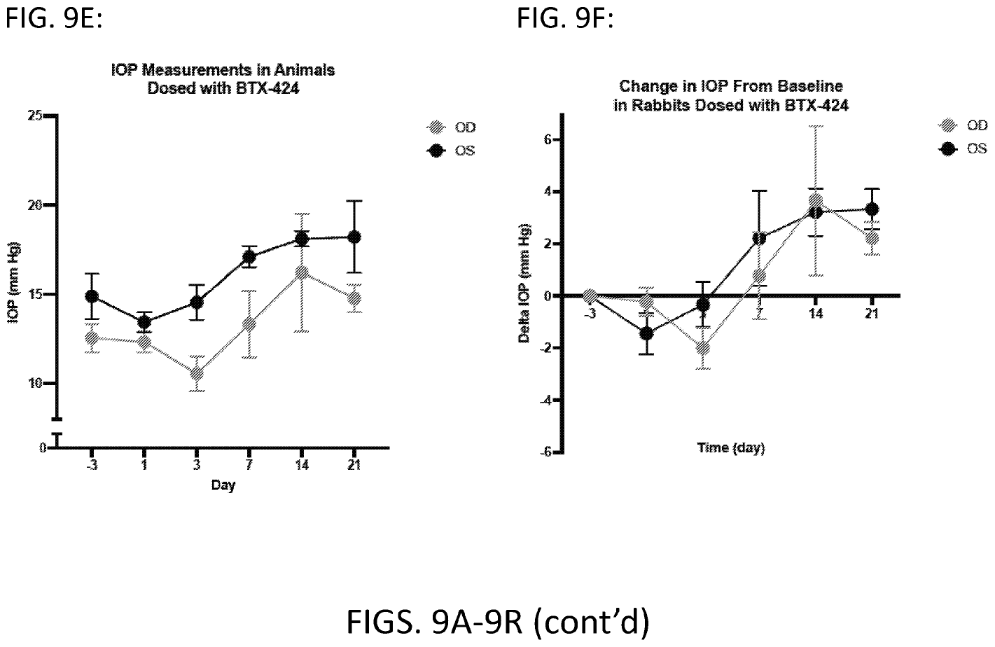

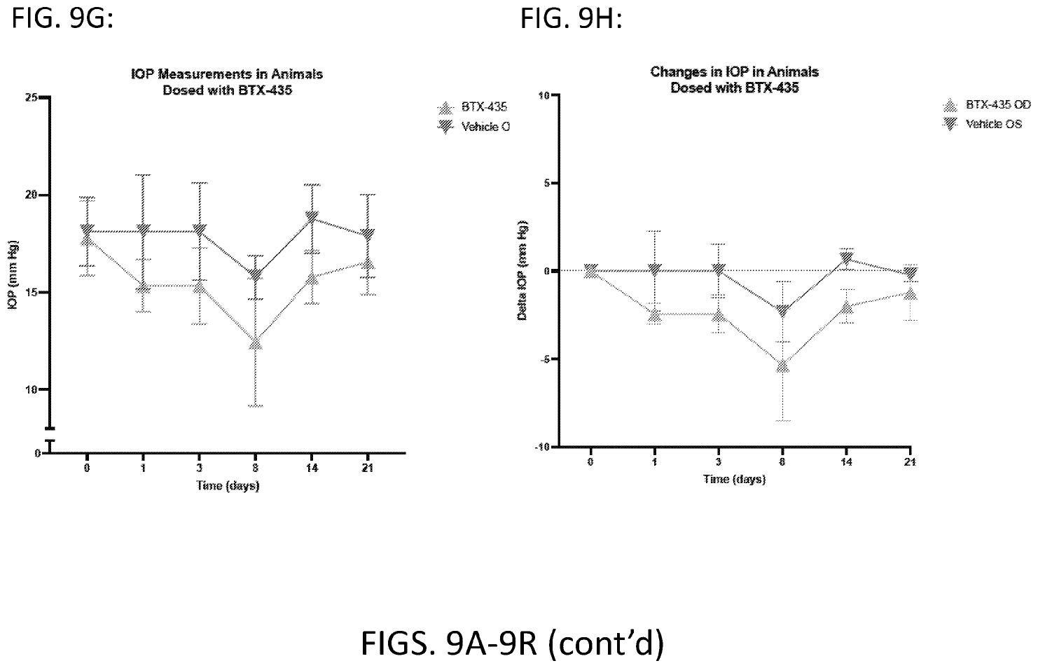

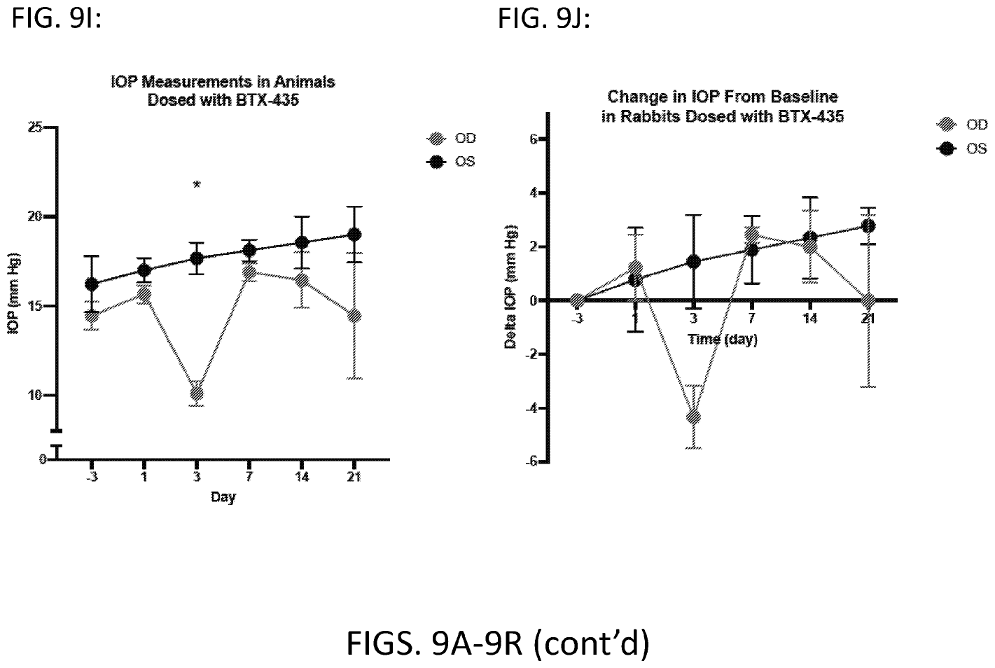

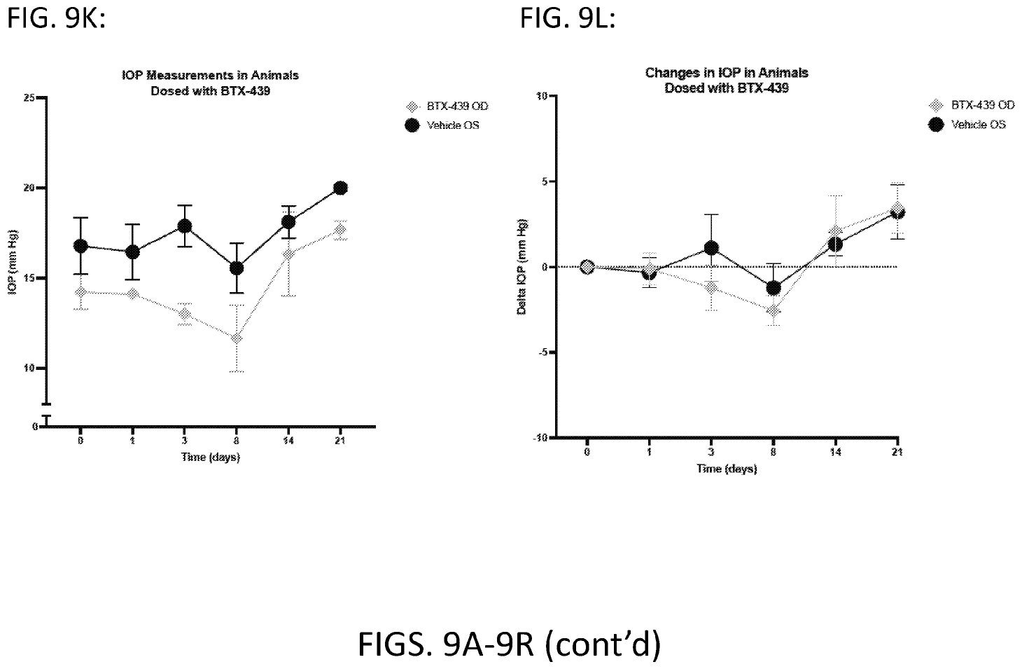

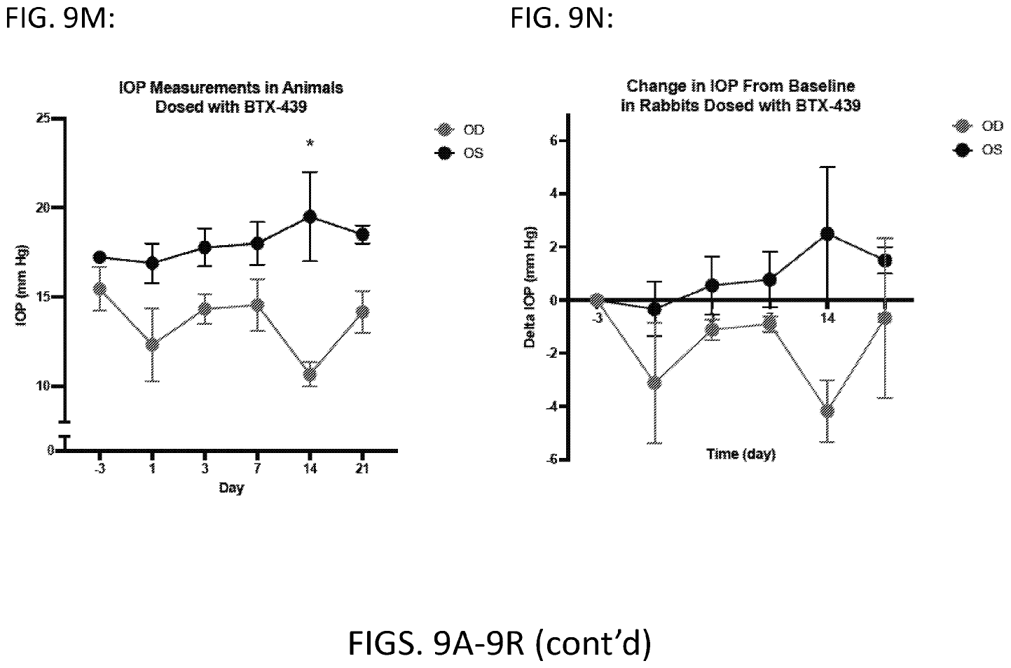

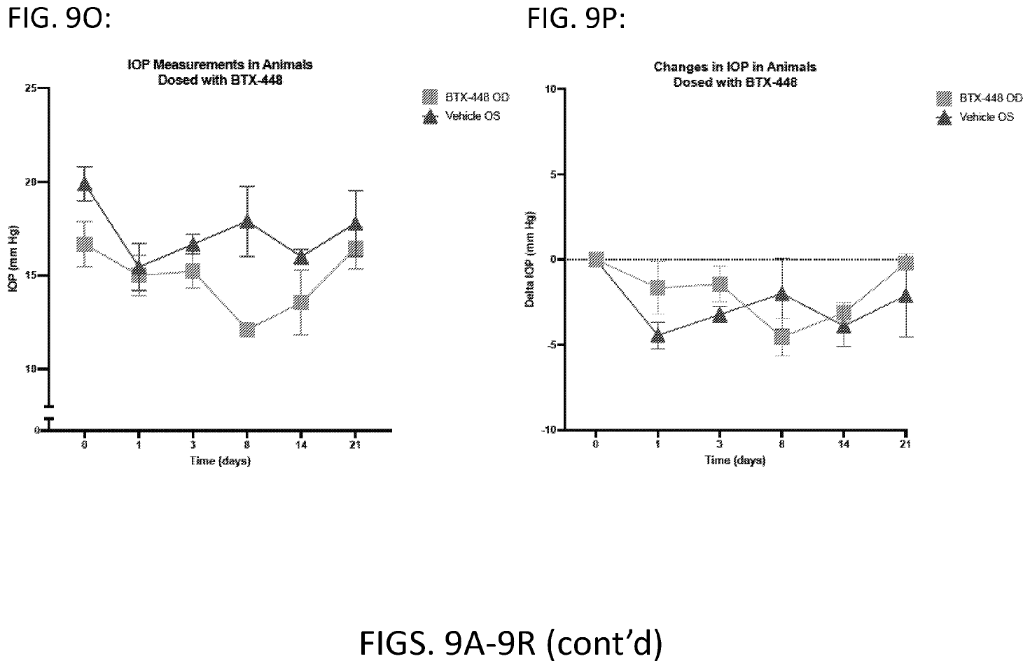

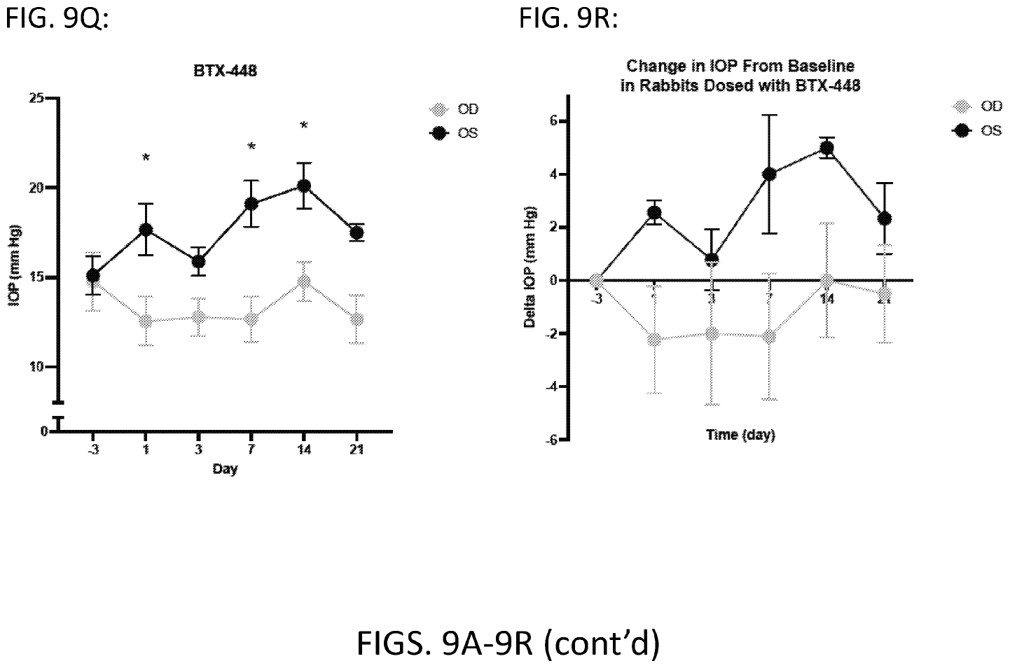

In Vivo Tolerability. Experiments were conducted to test the in vivo tolerability of anti-ANGPTL7 antibodies using single intravitreal (IVT) injections in New Zealand White Rabbits. Representative data is provided in FIGS. 9A-9R. The anti-ANGPTL7 antibodies indicated (“BTX” labels are interchangeable with “ATX-P” labels) were injected at either a 0.5 mg dose (FIGS. 9C, 9D, 9G, 9H, 9K, 9L, 9O, 9P) or a 2.0 mg dose (FIGS. 9E, 9F, 9I, 9J, 9M, 9N, 9Q, 9R) in the right eye (OD), or with a corresponding vehicle dose in the left eye (OS). (FIGS. 9A and 9B include data from controls at 2 mg doses). Intraocular pressure (IOP) measurements (FIGS. 9A, 9C, 9E, 9G, 9I, 9K, 9M, 9O, 9Q) and changes in IOP (FIGS. 9B, 9D, 9F, 9H, 9J, 9L, 9N, 9P, 9R) were taken at the indicated time points over a 21-day period.

Briefly, animals were dosed at Day 0, and IOP was normalized to Day −3 (N=3 per treatment group; p<0.05). Animals were dosed on Day 0. Experimental eyes (OD) were compared to vehicle eyes (OS) using two-way ANOVA. For the intravitreal injections (Day 0), rabbits underwent dilation with 1% tropicamide HCl, and were given buprenorphine (0.01-0.05 mg/kg) SC. Rabbits were sedated using a ketamine/xylazine cocktail (4-10/20-50 mg/kg) IM and the eyes were aseptically prepared using topical 5% betadine solution, followed by rinsing with sterile eye wash, and application of one drop of 0.5% proparacaine HCl. The conjunctiva were gently grasped with Colibri forceps, and the injection was made using a 27-30 G needle, 2-3 mm posterior to the superior limbus (through the pars plana), with the needle pointing slightly posteriorly to avoid contact with the lens. After dispensing the syringe contents, the needle was slowly withdrawn. Following the injection procedure, 1 drop of antibiotic ophthalmic solution was applied topically to the ocular surface. For each injection group, baseline ocular examinations and IOP measurements were obtained 24 hrs post-dose and at Days 3, 8, 14, and 21. Terminal blood collections for PK analysis (see below) were obtained on Day 21.

Intraocular pressure (IOP) was measured in both eyes of all animals at the timepoints indicated. The measurements were taken using a Tonovet probe (iCare Tonometer, Espoo, Finland) without use of topical anesthetic. The tip of the Tonovet probe was directed to gently contact the central cornea. Six consecutive measurements were obtained. After the sixth measurement, the average IOP shown was recorded. Animals did not require tranquilization for the procedure.

### Example 13

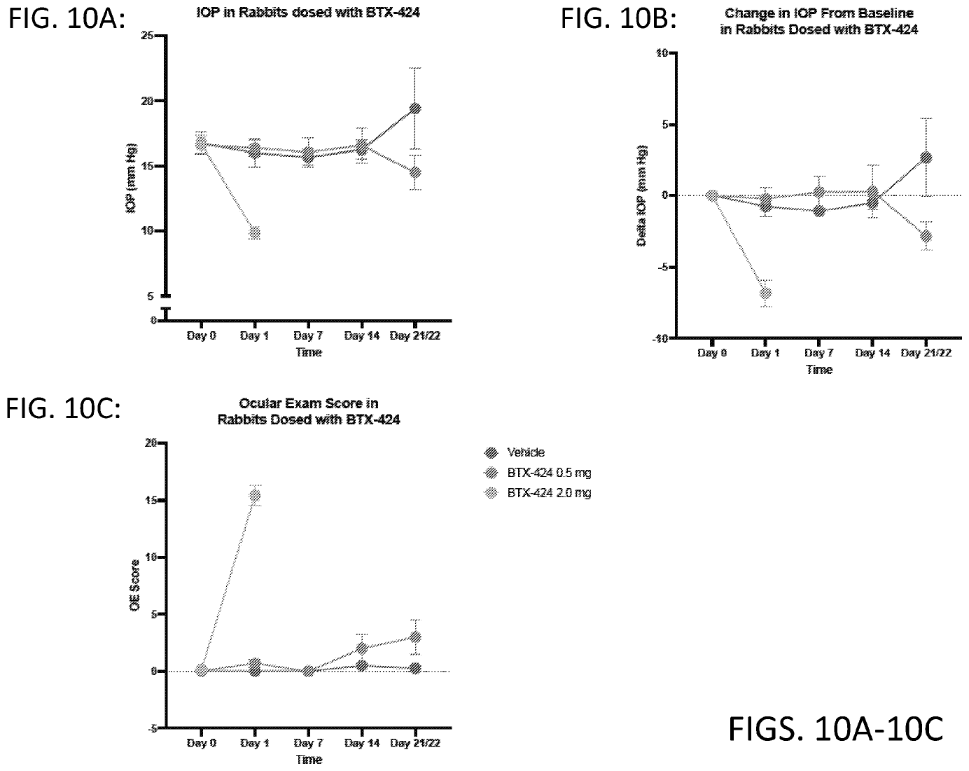

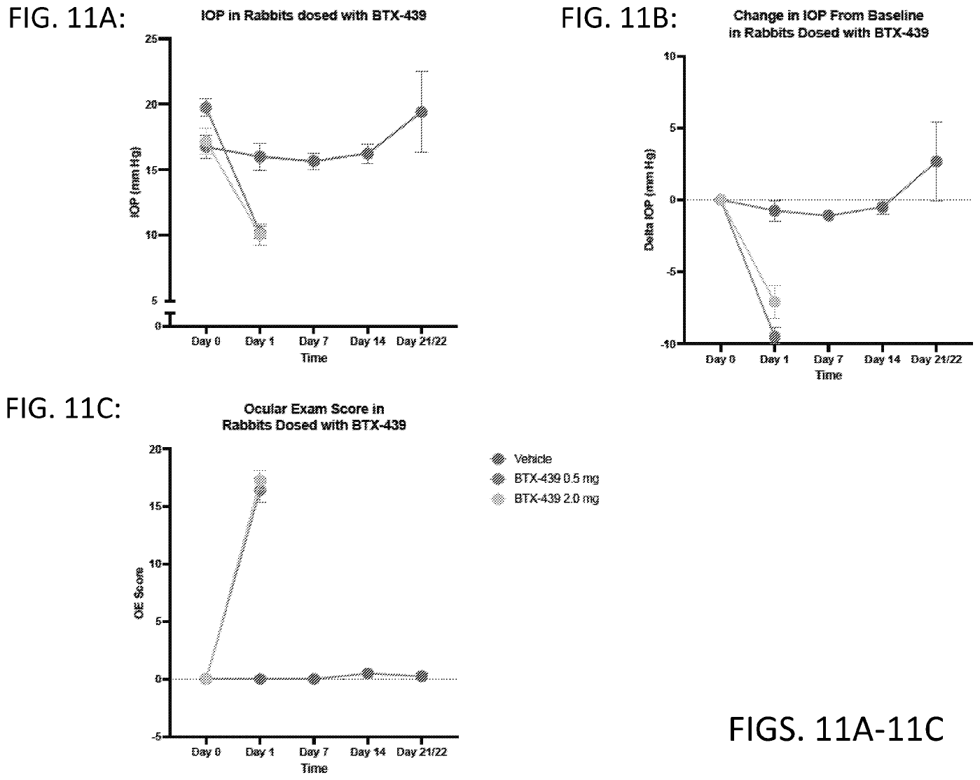

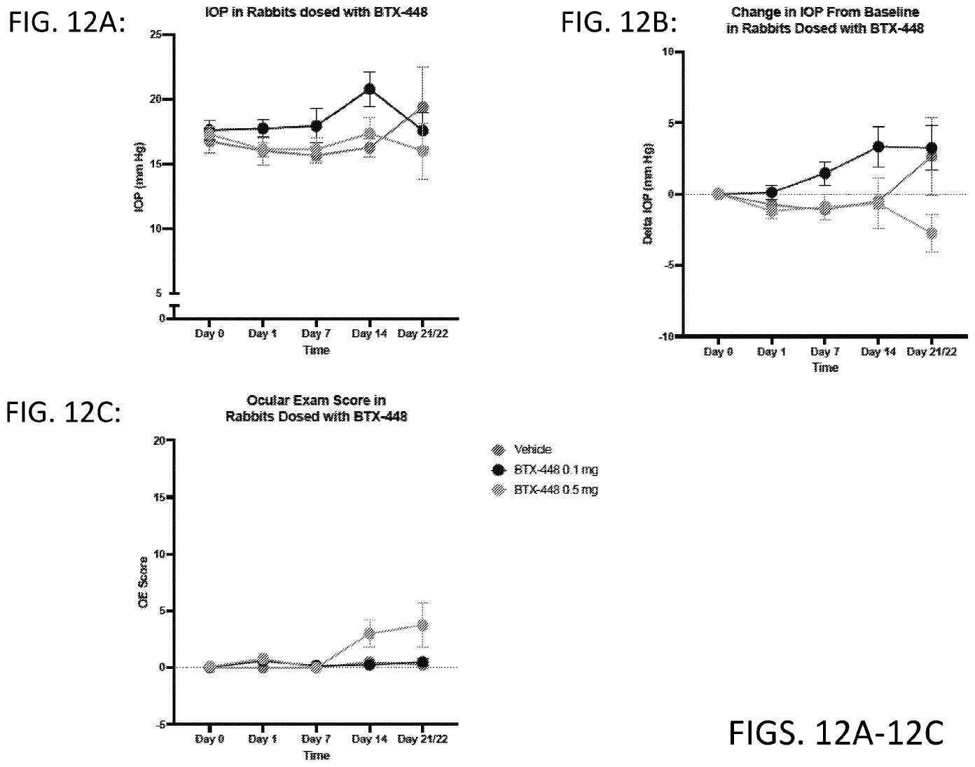

Pharmacokinetic (PK) Evaluation. Previous studies (described above) demonstrated that both 0.5 mg and 2.0 mg doses of several novel anti-ANGPTL7 antibodies were well tolerated in rabbits over a 21-day period. Thus, experiments were conducted to evaluate the pharmacokinetics (PK) of several of the well tolerated anti-ANGPTL7 antibodies. New Zealand White Rabbits received a single bilateral (OU) injection of each of ATX-P-424 (FIGS. 10A-10C), ATX-P-439 (FIGS. 11A-11C), and ATX-P-448 (FIGS. 12A-12C). (“BTX” labels are interchangeable with “ATX-P” labels.) Representative pharmacokinetic (PK) data for the ANGPTL7 antibody indicated included intraocular pressure (IOP) measurements dosed at 0.5 mg or 2.0 mg (OU) compared to vehicle (FIGS. 10A, 11A, and 12A); changes in IOP compared to baseline (FIGS. 10B, 11B, and 12B); and total ocular examination score (OE) (FIGS. 10C, 11C, and 12C).

Briefly, at least 1 mL of whole blood was drawn from the marginal ear vein or via cardiac puncture (terminal bleed only) and placed into plastic red top tubes (no anti-coagulant or with serum separator gel) for serum acquisition at the timepoints indicated. Alternatively, the central auricular artery was utilized if the vein is inaccessible or suboptimal for reuse for further collections. A thin layer of lidocaine 5% ointment was applied topically to the skin overlying the vessel prior to needle insertion. Oil of wintergreen was also applied to facilitate vasodilation as needed. After collection, the tubes were gently mixed by inverting 3-5 times. Blood samples were stored at room temperature for between 30 and 60 minutes prior to processing. The whole blood samples were centrifuged at 4° C. for 10 minutes at 2,000×g in a refrigerated centrifuge. Immediately after centrifugation, the clear serum was aliquoted into four (4) 200 μL samples and stored frozen at −80° C. until shipment for analysis. Additionally, a veterinary ophthalmologist performed complete ocular examinations using a slit lamp biomicroscope and indirect ophthalmoscope to evaluate ocular surface morphology and anterior and posterior segment inflammation on all animals prior to dosing to serve as a baseline and at the timepoints indicated. All animals received normal ocular examinations to be considered for this study. The modified Hackett and McDonald ocular grading system, with additional scoring parameters for the ocular posterior segment were used for scoring.

For analysis of ATX-P-424 at 0.5 mg, animals were dosed in two different cohorts (N=6 and N=4) staggered by one day. Animals were dosed on Day 0 and euthanized (N=2 per time point) at 24 h, and Day 7, 14, and 21. Number of eyes per measurement included N=20 on day 0 and 1, N=12 eyes on day 7, N=8 on day 14, and N=4 on day 21. For analysis of ATX-P-424 at 2.0 mg, on day 0, rabbits (N=6) exhibited OE scores of above 20 and were euthanized. For analysis of ATX-P-439, measurements included N=20 eyes at day 0 and 1. Animals dosed with ATX-P-439 at both doses (N=6 per group) exhibited OE scores of above 20 after 24 hours and were euthanized. No further animals were dosed from the second cohort. For analysis of ATX-P-448, animals were dosed in two different cohorts N=6 and N=4 staggered by one day. Animals were dosed on Day 0 and euthanized (N=2 per time point) at 24 h, and Day 7, 14, and 21. Number of eyes per measurement included N=20 on day 0 and 1, N=12 eyes on day 7, N=8 eyes on day 14, and N=4 eyes on day 21.

### Example 14

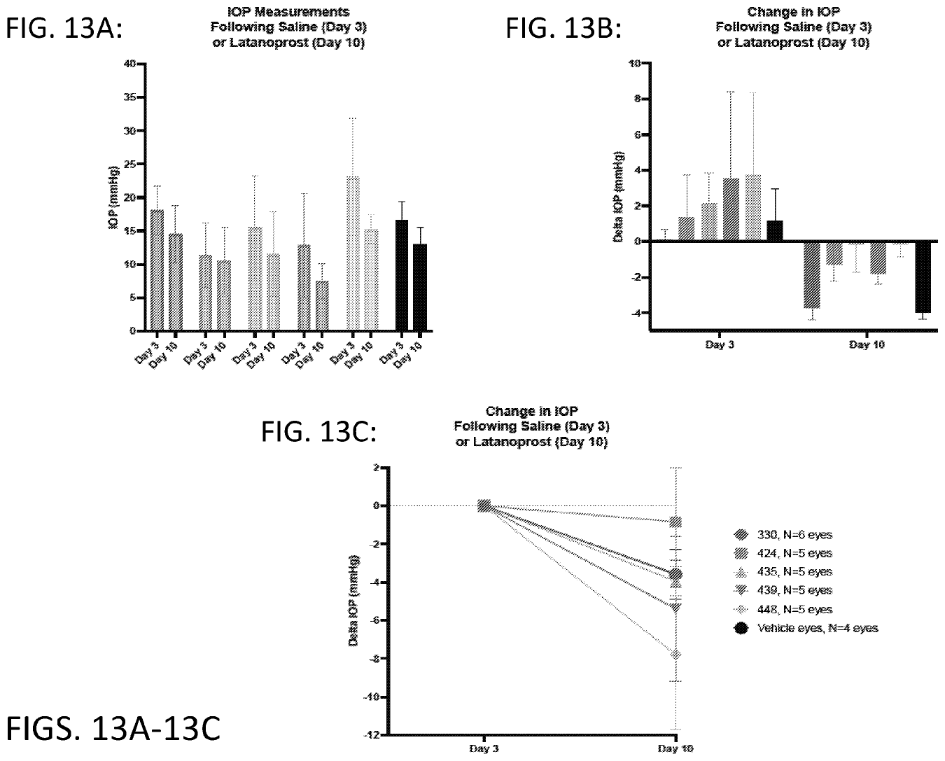

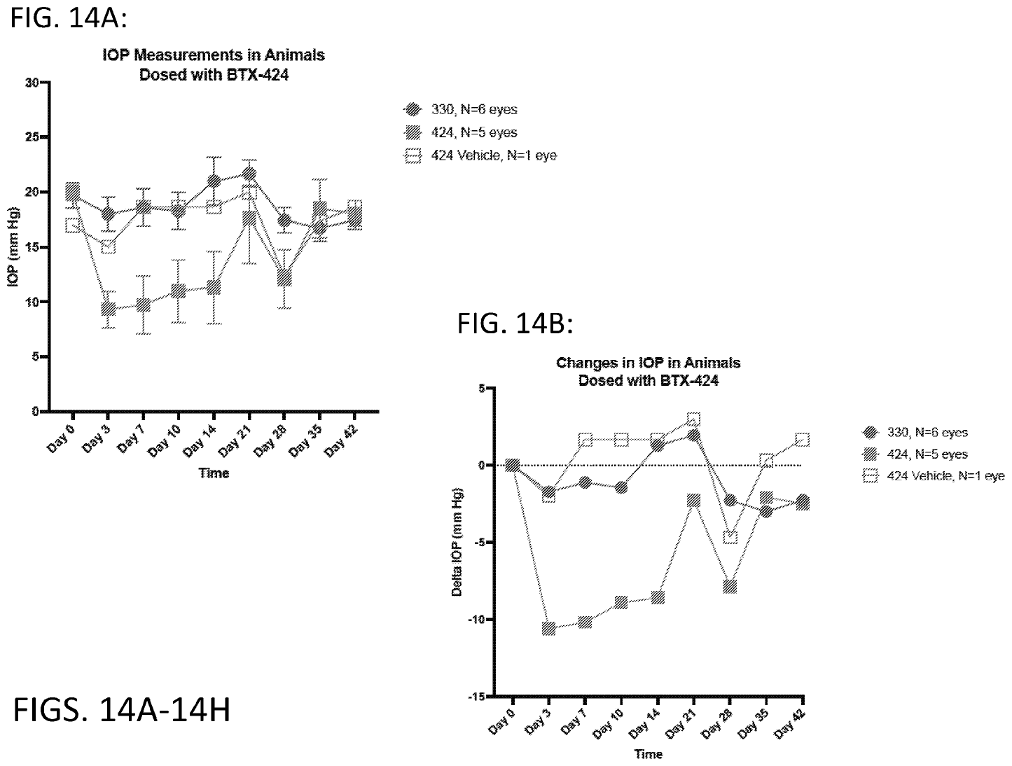

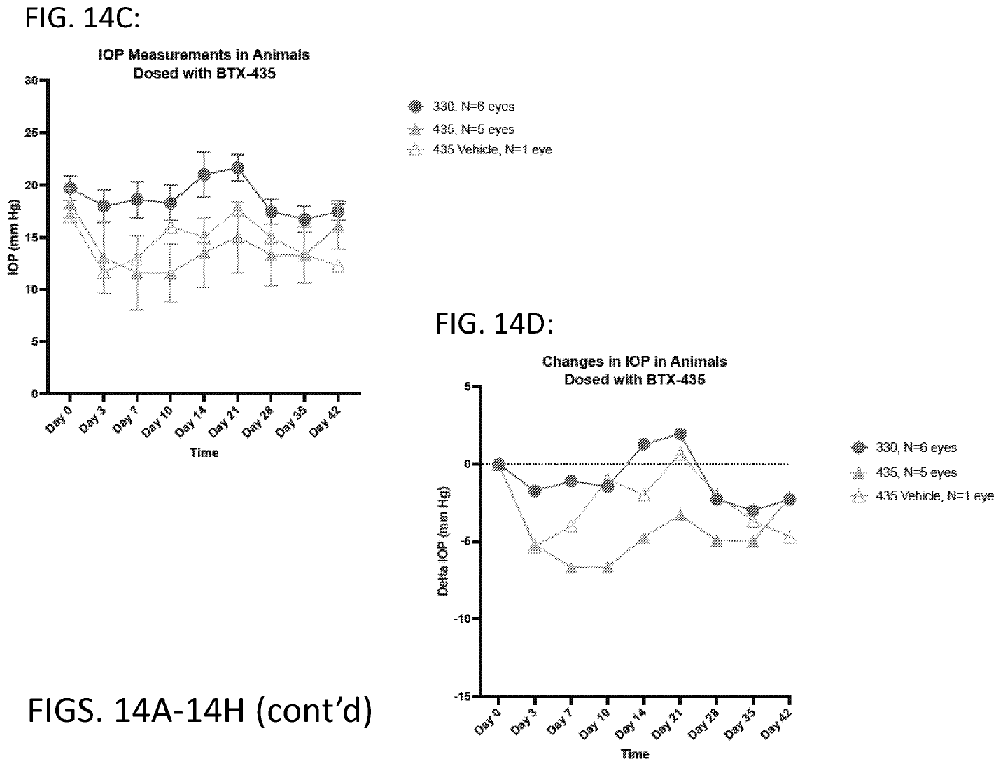

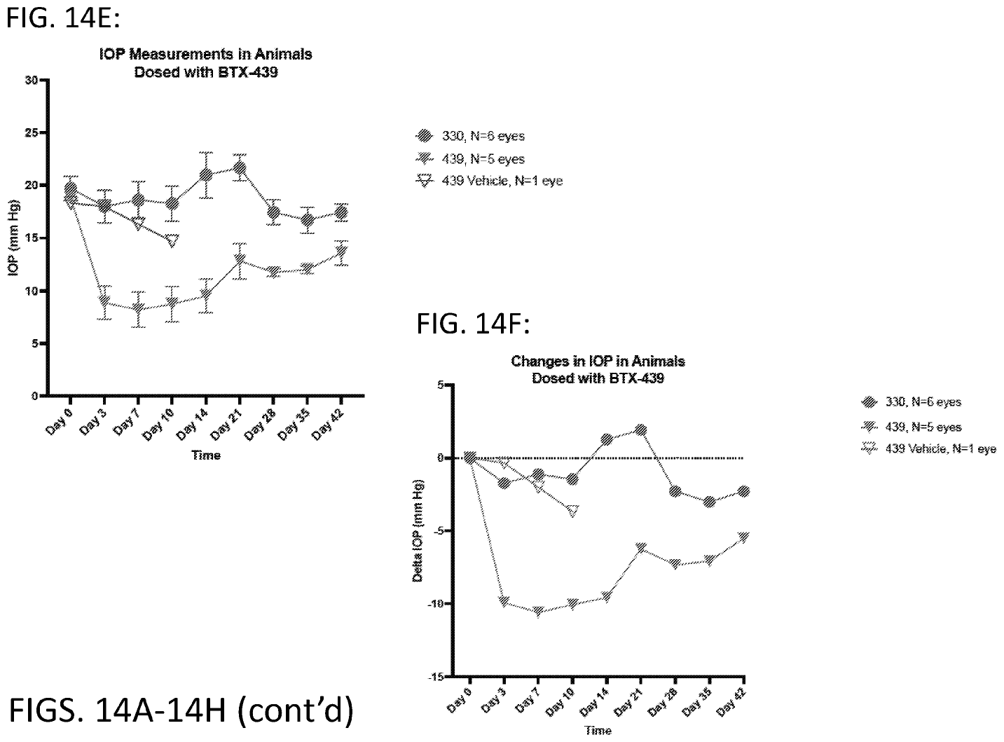

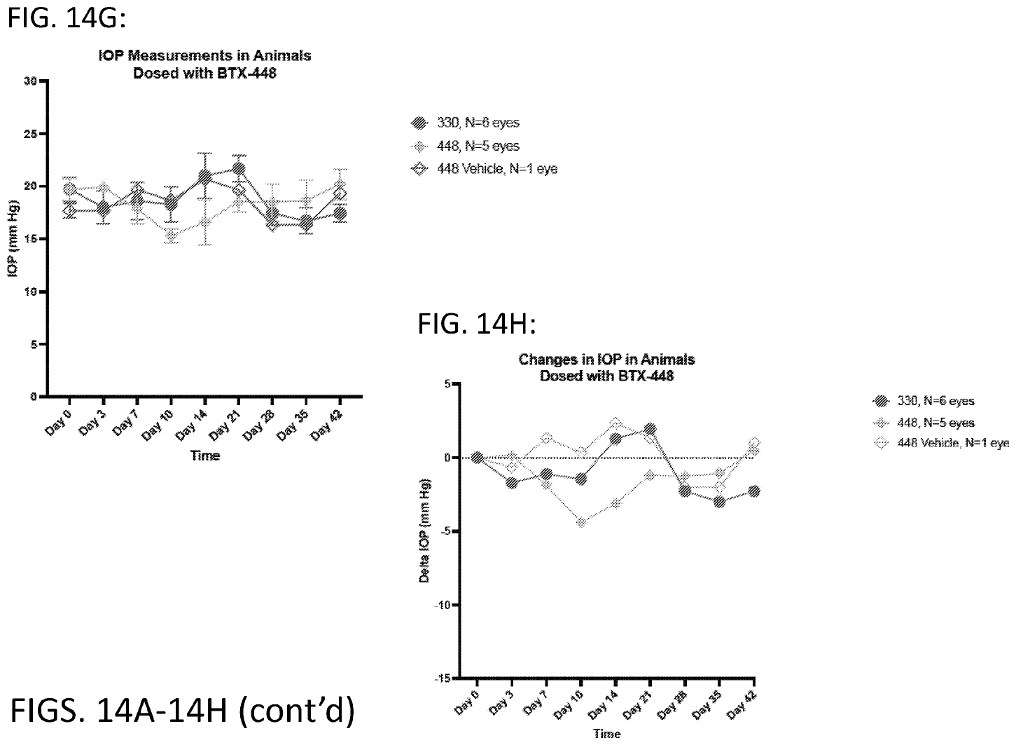

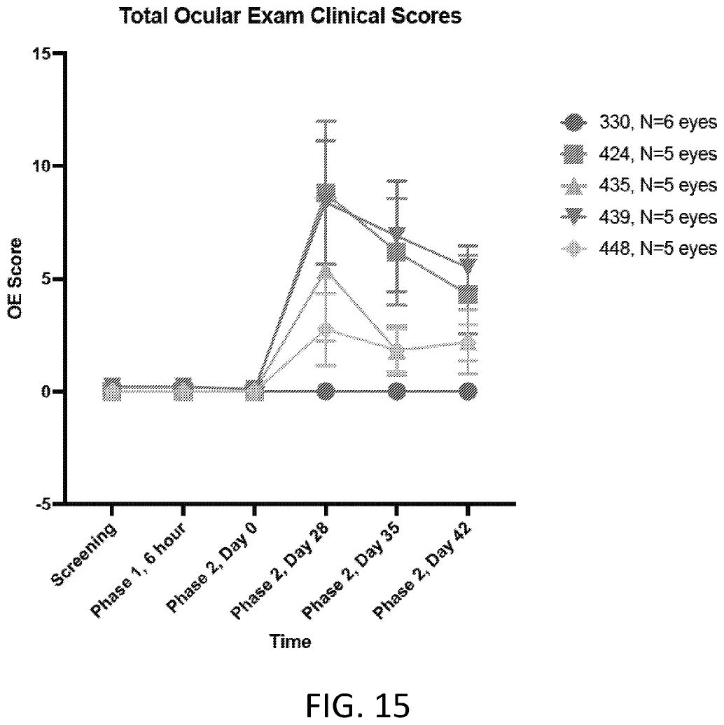

In Vivo Tolerability (Non-Human Primates). Experiments were also conducted to test the in vivo tolerability of anti-ANGPTL7 antibodies using single intravitreal (IVT) injections in Non-human primates (African Green Monkeys). FIGS. 13A-13C include representative data of absolute TOP values at baseline (day 3) and on day 10, 6 hours post topical administration of saline and Latanoprost (FIG. 13A); changes in TOP 6 hours post topical administration of either saline (day 3) or Latanoprost (day 10) (FIG. 13B); and changes in IOP between vehicle and Latanoprost administration (FIG. 13C). FIGS. 14A-14H include representative IOP measurements (FIGS. 14A, 14C, 14E, and 14G) and changes in IOP (FIGS. 14B, 14D, 14F, and 14H) in African Green Monkeys dosed with 2 mg of the indicated anti-ANGPTL7 antibody compared to an isotype control (BTX-330) and a single vehicle eye. And FIG. 15 includes representative clinical scores of ocular examinations (OE) across all the dosed groups.

Briefly, monkeys underwent prescreening to identify animals with basal IOP values greater than 16 mmHg and below 26 mmHg. For prescreening monkeys were sedated with ketamine/xylazine (8 mg/kg and 1.6 mg/kg, respectively) between 8:00-10:00 AM for IOP measures using a rebound tonometer (Tonovet®). In the event animals were not fully sedated additional ketamine/xylazine was administered to effect. Any animal requiring greater than 25% over target dose for IOP measurements was preferentially targeted for exclusion. Measurement of IOP were performed 5-10 minutes after initial sedation (T=0). Animals were placed in a supine position one minute prior to IOP measurement (OS will be analyzed immediately prior to OD) and prone at all other times while sedated. The sedation, body positioning and IOP measurement procedure was repeated in identical fashion 6 hours after initial prescreen assessments (T=6 hour). Any monkey demonstrating a greater than 5 mmHg drop in IOP between T=0 and T=6 hour (TOP sensitivity to sedation) was excluded from the study. Following completion of T=6 hour IOP measurements monkeys underwent ophthalmic screening to assess ocular and general health by slit lamp biomicroscopy, fundoscopy, and color fundus photography (CFP). Monkeys with normal exam findings were enrolled into the study and randomized to treatment groups based on prescreen T=0 IOP.

A minimum of 7 days after completion of screening activities and at least 7 days prior to initiation of Phase 2, animals were sedated and IOP assessed using the same methods employed for screening. Immediately after completion of T=0 IOP measures, one eye was dosed with 1 gtt (˜35 μL) of latanoprost 0.005% ophthalmic solution (Xalatan® or equivalent) and the other with 0.9% saline. Dosing was performed between 8:00 AM and 10:00 AM. Follow up IOP assessments were performed at T=6 hour to determine IOP lowering response for each animal. The absolute change in IOP between T=0 and T=6 h during screening and Phase 1 was calculated for each eye and subsequently the percent difference in ΔIOP between sedation alone and latanoprost (% ΔΔIOP) was determined using the formula:

$$= 100 - ( \frac{Δ IOP ( screening )}{Δ IOP ( phase 1 )} \times 100 )$$

Animals were subsequently ranked based on average % ΔΔIOP in response to timolol. Animals demonstrating minimal impact of sedation on IOP and response to timolol administration compared with sedation alone (“timolol responders”) were enrolled in the study. In the event less than 15 animals were identified for recruitment based on these criteria up to 8 additional animals were screened in identical fashion to complete study enrolment.

For IVT dosing, topical proparacaine 0.5% was administered, allowing 30 seconds to take effect, and an eye speculum placed, then the ocular surface was rinsed with 5% Betadine solution followed by sterile 0.9% saline. IVT injections were performed in both eyes (OU) according to the treatment assignment using a 31-gauge 5/16-inch needle/syringe (Ulticare VetRx U-100, or equivalent) inserted inferotemporally at the level of the or a serrata ˜2 mm posterior to the limbus. Following injection, a topical antibiotic ophthalmic ointment (neomycin, polymyxin, bacitracin or equivalent) was administered.

At the indicated time points, intraocular pressure (IOP) measurements were collected using a TonoVet (iCare, Finland) tonometer set to the dog (d) calibration setting. The TonoVet rebound tonometer was well tolerated and required no additional local analgesia. All TOP measures were performed while animals were sedated with ketamine and xylazine. Animals were sedated 5-10 minutes prior to each scheduled measure and placed in a supine position one minute prior to each IOP measurement (OS will be analyzed immediately prior to OD) and prone at all other times while sedated. After completion of each IOP assessment animals were shifted to a prone position and maintained in this position until recovered from sedation. Three measures were taken from each eye at each time point and the mean value employed for IOP analysis.

Additionally, at the designated time points, both eyes (OU) were examined by slit lamp biomicroscopy. Scoring was applied to qualitative clinical ophthalmic findings using a nonhuman primate ophthalmic scoring system and summary score derived from exam components. At the designated time points, bilateral color fundus images of the retina were captured with 50° of view centered on the fovea using a Topcon TRC-50EX retinal camera with Canon 6D digital imaging hardware and New Vision Fundus Image Analysis System software. At designated time points, blood samples (0.5-1 mL) were collected and transferred to K₂EDTA lavender top Vacutainers, gently inverted several times and retained on ice until CBC with differential analysis on an Abaxis VetScan HM5 hematology analyzer. Whole blood (3 mL) was collected via the femoral or saphenous vein at the designated time points. Blood was transferred to vacutainer tubes (in the absence of anticoagulant) and incubated at room temperature for approximately 1 hour before centrifugation 4000 rpm for 10 minutes at 4° C. and isolation of serum aliquots (˜0.5 mL×2 aliquots per time point). Aliquots were stored and shipped below −70° C. to a designated laboratory for NAb analysis. At the designated timepoints, topical proparacaine 0.5% was administered, allowing 30 seconds to take effect, an eye speculum placed, then the ocular surface rinsed with 5% Betadine solution followed by a sterile 0.9% saline rinse. Aqueous humor (50 μL) was sampled with a 0.3 mL insulin syringe with a 31-gauge needle advance into the anterior chamber ˜2 mm anterior to the temporal limbus. Aqueous samples were transferred to cryotubes, flash frozen and stored and shipped below −70° C. to the Sponsor, or to a Sponsor designated laboratory for analysis. Subjects were additionally assessed at cage-side twice daily for general wellbeing and evidence of ocular pathology. Data generated from protocol-defined endpoints were collated, summarized, and analyzed. Specified statistical analyses were performed where data meets required assumptions. If data failed to meet assumptions for defined statistical methods alternative methods were employed where possible. P values≤0.05 will be considered statistically significant.

### Sequences

The various amino acid sequences and nucleic acid sequences referenced herein are provided below.

TABLE 8
Anti-ANGPTL7 antibodies (CDR sequences)

| | | | |
|---|---|---|---|
| SEQ ID | Antibody | | |
| NO: | Name: | Descriptor: | Sequence: |

VH Family: IGHV1

| | | | |
|---|---|---|---|
| 1 | — | HCDR1 | X₁YX₂IX₃ |
| | | Consensus | (X₁ is S or D; X₂ is G or Y; X₃ is S or H) |
| 2 | ATX-P-428 | HCDR1 | SYGIS |
| 3 | ATX-P-429 | HCDR1 | SYGIS |
| 4 | ATX-P-443 | HCDR1 | SYGIS |
| 5 | ATX-P-431 | HCDR1 | DYYIH |
| 6 | ATX-P-432 | HCDR1 | DYYIH |
| 7 | — | HCDR2 | WIX₁X₂X₃X₄GX₅TX₆YAQX₇X₈X₉G |
| | | Consensus | (X₁ is S, I, or N; X₂ is A or P; |
| | | | X₃ is Y or N; X₄ is N or T; X₅ is N |
| | | | or A; X₆ is N or K; X₆ is N or K; |
| | | | X₈ is L or F; X₉ is R or Q) |
| 8 | ATX-P-428 | HCDR2 | WISAYNGNTNYAQNLRG |
| 9 | ATX-P-429 | HCDR2 | WISAYNGNTNYAQNLRG |
| 10 | ATX-P-443 | HCDR2 | WIIAYNGNTNYAQKLQG |
| 11 | ATX-P-431 | HCDR2 | WINPNTGATKYAQKFQG |
| 12 | ATX-P-432 | HCDR2 | WINPNTGATKYAQKFQG |
| 13 | ATX-P-428 | HCDR3 | VSLVWFGELPRGFDY |
| 14 | ATX-P-429 | HCDR3 | VSLVWFGELPRGFDY |
| 15 | ATX-P-443 | HCDR3 | DTGFSFDY |
| 16 | ATX-P-431 | HCDR3 | EDNGNYGDVEDI |
| 17 | ATX-P-432 | HCDR3 | EDNGNYGDVEDI |

VH Family: IGHV2

| | | | |
|---|---|---|---|
| 18 | — | HCDR1 | TSGVGVG |
| | | Consensus | |
| 19 | ATX-P-442 | HCDR1 | TSGVGVG |
| 20 | ATX-P-437 | HCDR1 | TSGVGVG |
| 21 | — | HCDR2 | LIYWNDDKX₁YSPSLKS |
| | | Consensus | (X₁ is R or Q) |
| 22 | ATX-P-442 | HCDR2 | LIYWNDDKRYSPSLKS |
| 23 | ATX-P-437 | HCDR2 | LIYWNDDKQYSPSLKS |
| 24 | — | HCDR3 | X₁X₂X₃X₄X₅X₆FFDX₇ |
| | | Consensus | (X₁ is S, D, or N; X₂ is Y or P; X₃ |
| | | | is G or D; X₄ is D or Y; X₅ is Y |
| | | | or G; X₆ is W or D; X₇ is L or Y) |
| 25 | ATX-P-442 | HCDR3 | DYGDYWFFDL |
| 26 | ATX-P-437 | HCDR3 | NPDYGDFFDY |

VH Family: IGHV3

| | | | |
|---|---|---|---|
| 27 | — | HCDR1 | X₁IX₂X₃MX₄ |
| | | Consensus | (X₁ is V, S, D, or T; X₂ is Y, H, or F; |
| | | | X₃ is D, G, S, or A; X₄ is H, |
| | | | S, or N) |
| 28 | ATX-P-444 | HCDR1 | VYDMH |
| 29 | ATX-P-445 | HCDR1 | SHDMH |
| 30 | ATX-P-446 | HCDR1 | SYDMH |
| 31 | ATX-P-447 | HCDR1 | DYDMH |
| 32 | ATX-P-427 | HCDR1 | DYGMS |
| 33 | ATX-P-440 | HCDR1 | DYGMS |
| 34 | ATX-P-424 | HCDR1 | SYSMN |
| 35 | ATX-P-425 | HCDR1 | TYSMN |
| 36 | ATX-P-430 | HCDR1 | SYAMS |
| 37 | ATX-P-439 | HCDR1 | SYAMS |
| 38 | ATX-P-449 | HCDR1 | SYGMH |
| 39 | ATX-P-448 | HCDR1 | SYGMH |
| 40 | ATX-P-423 | HCDR1 | SYGMH |
| 41 | ATX-P-438 | HCDR1 | SYGMH |
| 42 | ATX-P-433 | HCDR | DFAMH |
| 43 | — | HCDR2 | X₁X₂X₃X₄X₅X₆X₇X₈X₉X₁₀X₁X₁₂X₁₃X₁₄X₁₅G |
| | | Consensus | (X₁ is G, T, S, A, V. H, or I; |
| | | | X₂ is I or M; X₃ is D, N, T, S, or G; |
| | | | X₄ is P, W, S, G, or Y; X₅ is D, A, N, S, or Y; |
| | | | X₆ is G or S; X₇ is D, G, Y, S, I, or N; |
| | | | X₈ is T, S, N, I, Y, or D; |
| | | | X₉ is Y, T, F, M, K, G, or I; |
| | | | X₁₀ is Y, G, or F; X₁₁ is P, Y, or A; |
| | | | X₁₂ is G, D, or A; X₁₃ is S or D; |
| | | | X₁₄ is V, L, or S; X is is K or M) |
| 44 | ATX-P-444 | HCDR2 | GIDPDGDTYYPGSVKG |
| 45 | ATX-P-445 | HCDR2 | GIDPAGDTYYPDSVKG |
| 46 | ATX-P-446 | HCDR2 | GIDPDGDTYYPGSVKG |
| 47 | ATX-P-447 | HCDR2 | GIDPDGDTYYPGSLKG |
| 48 | ATX-P-427 | HCDR2 | GINWNGGSTGYADSVKG |
| 49 | ATX-P-440 | HCDR2 | TINWNGGNTGYADSVKG |
| 50 | ATX-P-424 | HCDR2 | SITSSSYIFYADSLKG |
| 51 | ATX-P-425 | HCDR2 | SITSSSSYMFYADSVMG |
| 52 | ATX-P-430 | HCDR2 | AISGSGGSTYYADSVKG |
| 53 | ATX-P-439 | HCDR2 | AISGSGGSTYYADSVKG |
| 54 | ATX-P-449 | HCDR2 | VISYYGSNKYYADSVKG |
| 55 | ATX-P-448 | HCDR2 | VMSYDGINGYYADSVKG |
| 56 | ATX-P-423 | HCDR2 | HITYDGSDKYYGDSVKG |
| 57 | ATX-P-438 | HCDR2 | IISYDGNNKYYADSVKG |
| 58 | ATX-P-433 | HCDR2 | GIGWNSGSIGYADSVKG |
| 59 | ATX-P-444 | HCDR3 | GEESNSSEDGEDI |
| 60 | ATX-P-445 | HCDR3 | GEASSSSEDAFDI |
| 61 | ATX-P-446 | HCDR3 | GEDNSSSEDAFDI |
| 62 | ATX-P-447 | HCDR3 | GEDSSSSEDAFDI |
| 63 | ATX-P-427 | HCDR3 | YSGSFRDAFDI |
| 64 | ATX-P-440 | HCDR3 | DEGAAGLGYYFDY |
| 65 | ATX-P-424 | HCDR3 | DSNWGEAFDI |
| 66 | ATX-P-425 | HCDR3 | DSNWGEAFDI |
| 67 | ATX-P-430 | HCDR3 | DGDYYYYYMDV |
| 68 | ATX-P-439 | HCDR3 | VERDMVRGHYYYYMDV |
| 69 | ATX-P-449 | HCDR3 | GITMDV |
| 70 | ATX-P-448 | HCDR3 | LVYADDAFDI |
| 71 | ATX-P-423 | HCDR3 | DGYSSGWWYFDY |
| 72 | ATX-P-438 | HCDR3 | ERGYSSSSGYYYYYMDV |
| 73 | ATX-P-433 | HCDR3 | APDYGDYYFDY |

VH Family: IGHV4

| | | | |
|---|---|---|---|
| 74 | — | HCDR1 | SX₁SX₂YWX₃ |
| | | Consensus | (X₁ is S or G; X₂ is S or Y; X₃ is G or S) |
| 75 | ATX-P-434 | HCDR1 | SSSSYWG |
| 76 | ATX-P-435 | HCDR1 | SSSSYWG |
| 77 | ATX-P-436 | HCDR1 | SGSYYWS |
| 78 | — | HCDR2 | X₁IYYSGSTX₂SNPSLKS |
| | | Consensus | (X₁ is S, or Y; X₂ is Y or S) |
| 79 | ATX-P-434 | HCDR2 | SIYYSGSTYSNPSLKS |
| 80 | ATX-P-435 | HCDR2 | SIYYSGSTYSNPSLKS |
| 81 | ATX-P-436 | HCDR2 | YIYYSGSTSYNPSLKS |
| 82 | — | HCDR3 | X₁X₂X₃X₄GX₅X₆X₇X₈X₉Y |
| | | Consensus | (X₁ is Q or A; X₂ is Y or K; X₃ is I or |
| | | | W; X₄ is S or E; X₅ is T or |
| | | | D; X₆ is E or Y; X₇ is Y or F; |
| | | | X₈ is F or D; X, is Q or Y) |
| 83 | ATX-P-434 | HCDR3 | QYISGTEYFQY |
| 84 | ATX-P-435 | HCDR3 | QYISGTEYFQY |
| 85 | ATX-P-436 | HCDR3 | AKWEGDYFDY |

VH Family: IGKV1

| | | | |
|---|---|---|---|
| 86 | — | LCDR1 | RASQX₁IX₂X₃X₄LX₅ |
| | | Consensus | (X₁ is G or S; X₂ is S, R, or Y; |
| | | | X₃ is S, N, or I; X₄ is W, D, or Y; |
| | | | X₅ is A, G, or N) |
| 87 | ATX-P-439 | LCDR1 | RASQGISSWLA |
| 88 | ATX-P-424 | LCDR1 | RASQGIRNDLG |
| 89 | ATX-P-425 | LCDR1 | RASQGIRIDLG |
| 90 | ATX-P-423 | LCDR1 | RASQGIRNDLG |
| 91 | ATX-P-442 | LCDR1 | RASQGISSYLA |
| 92 | ATX-P-437 | LCDR1 | RASQSISSYLN |
| 93 | ATX-P-440 | LCDR1 | RASQSISSWLA |
| 94 | ATX-P-433 | LCDR1 | RASQSISIYLN |
| 95 | ATX-P-436 | LCDR1 | RASQSISSYLN |
| 96 | ATX-P-434 | LCDR1 | RASQSIYSYLN |
| 97 | ATX-P-427 | LCDR1 | RASQGIRNDLG |
| 98 | — | LCDR2 | AX₁SSLX₂S |
| | | Consensus | (X₁ is A or T; X₂ is Q or P) |
| 99 | ATX-P-439 | LCDR2 | AASSLQS |
| 100 | ATX-P-424 | LCDR2 | AASSLPS |
| 101 | ATX-P-425 | LCDR2 | AASSLPS |
| 102 | ATX-P-423 | LCDR2 | AASSLQS |
| 103 | ATX-P-442 | LCDR2 | AASSLQS |
| 104 | ATX-P-437 | LCDR2 | AASSLQS |
| 105 | ATX-P-440 | LCDR2 | ATSSLQS |
| 106 | ATX-P-433 | LCDR2 | AASSLQS |
| 107 | ATX-P-436 | LCDR2 | AASSLQS |
| 108 | ATX-P-434 | LCDR2 | AASSLQS |
| 109 | ATX-P-427 | LCDR2 | AASSLQS |
| 110 | — | LCDR3 | X₁QX₂X₃X₄X₅PX₆X₇ |
| | | Consensus | (X₁ is L or Q; X₂ is A, H, S, or D; |
| | | | X₃ is N, F, or Y; X₄ is S, T, or |
| | | | N; X₅ is F, Y, or T; X₆ is W, L, I, |
| | | | P, or Y; X, is T or Y) |
| 111 | ATX-P-439 | LCDR3 | QQANSFPWT |
| 112 | ATX-P-424 | LCDR3 | LQHNSYPWT |
| 113 | ATX-P-425 | LCDR3 | LQHNSYPWT |
| 114 | ATX-P-423 | LCDR3 | LQHNSYPLT |
| 115 | ATX-P-442 | LCDR3 | QQSFSTPLT |
| 116 | ATX-P-437 | LCDR3 | QQSFSTPLT |
| 117 | ATX-P-440 | LCDR3 | QQSYSTPIT |
| 118 | ATX-P-433 | LCDR3 | QQSYTTPLT |
| 119 | ATX-P-436 | LCDR3 | QQSYSTPLT |
| 120 | ATX-P-434 | LCDR3 | QQSYSTPPYT |
| 121 | ATX-P-427 | LCDR3 | LQDYNYPYT |

VH Family: IGKV2

| | | | |
|---|---|---|---|
| 122 | — | LCDR1 | RSSQSLX₁X₂SX₃X₄X₅X₆YLX₇ |
| | | Consensus | (X₁ is L or V; X₂ is H, Y, or F; |
| | | | X₃ is N or D; X₄ is R or G; X₅ is Y |
| | | | or N; X₆ is N or T; X, is D or N) |
| 123 | ATX-P-429 | LCDR1 | RSSQSLLHSNRYNYLD |
| 124 | ATX-P-444 | LCDR1 | RSSQSL VYSDGNTYLN |
| 125 | ATX-P-445 | LCDR1 | RSSQSLVYSDGNTYLN |
| 126 | ATX-P-446 | LCDR1 | RSSQSLVFSDGNTYLN |
| 127 | ATX-P-447 | LCDR1 | RSSQSLVYSDGNTYLN |
| 128 | — | LCDR2 | X₁X₂SNRX₃S |
| | | Consensus | (X₁ is L, K, or E; X₂ is G or V; X₃ is A or D) |
| 129 | ATX-P-429 | LCDR2 | LGSNRAS |
| 130 | ATX-P-444 | LCDR2 | KVSNRDS |
| 131 | ATX-P-445 | LCDR2 | EVSNRDS |
| 132 | ATX-P-446 | LCDR2 | KVSNRDS |
| 133 | ATX-P-447 | LCDR2 | EVSNRDS |
| 134 | — | LCDR3 | MQX₁X₂X₃X₄PX₅T |
| | | Consensus | (X₁ is T or G; X₂ is L or T; |
| | | | X₃ is Q or H; X₄ is T or W; |
| | | | X₅ is Y or W) |
| 135 | ATX-P-429 | LCDR3 | MQTLQTPYT |
| 136 | ATX-P-444 | LCDR3 | MQGTHWPWT |
| 137 | ATX-P-445 | LCDR3 | MQGTHWPWT |
| 138 | ATX-P-446 | LCDR3 | MQGTHWPWT |
| 139 | ATX-P-447 | LCDR3 | MQGTHWPWT |

VH Family: IGKV3

| | | | |
|---|---|---|---|
| 140 | — | LCDR1 | RASQSVSX₁X₂X₃X₄A |
| | | Consensus | (X₁ is S, N, or R; X₂ is Y or S; |
| | | | X₃ is L or Y; X₄ is A or L) |
| 141 | ATX-P-449 | LCDR1 | RASQSVSSYLA |
| 1.42 | ATX-P-448 | LCDR1 | RASQSVSSYLA |
| 143 | ATX-P-435 | LCDR1 | RASQSVSNYLA |
| 144 | ATX-P-428 | LCDR1 | RASQSVSSSYLA |
| 145 | ATX-P-443 | LCDR1 | RASQSVSSSYLA |
| 146 | ATX-P-431 | LCDR1 | RASQSVSSSYLA |
| 147 | ATX-P-432 | LCDR1 | RASQSVSSSYLA |
| 148 | ATX-P-430 | LCDR1 | RASQSVSSSYLA |
| 149 | ATX-P-438 | LCDR1 | RASQSVSRSYLA |
| 150 | — | LCDR2 | X₁ASX₂RAT |
| | | Consensus | (X₁ is D or G; X₂ is N, S, or T) |
| 151 | ATX-P-449 | LCDR2 | DASNRAT |
| 152 | ATX-P-448 | LCDR2 | DASNRAT |
| 153 | ATX-P-435 | LCDR2 | DASNRAT |
| 154 | ATX-P-428 | LCDR2 | GASSRAT |
| 155 | ATX-P-443 | LCDR2 | GASSRAT |
| 156 | ATX-P-431 | LCDR2 | GASSRAT |
| 157 | ATX-P-432 | LCDR2 | GASSRAT |
| 158 | ATX-P-430 | LCDR2 | GASSRAT |
| 159 | ATX-P-438 | LCDR2 | GASTRAT |
| 160 | — | LCDR3 | QQX₁X₂X₃X₄X₅X₆T |
| | | Consensus | (X) is R, Y, or G; X₂ is S, G, or Q; |
| | | | X₃ is N, S, or V; X₄ is W, S, or I; |
| | | | X₃ is P or L; X₆ is L, S, P, or T) |
| 161 | ATX-P-449 | LCDR3 | QQRSNWPLT |
| 162 | ATX-P-448 | LCDR3 | QQRSNWPLT |
| 163 | ATX-P-435 | LCDR3 | QQRSNWPS |
| 164 | ATX-P-428 | LCDR3 | QQYGSSPPT |
| 165 | ATX-P-443 | LCDR3 | QQYGSSLT |
| 166 | ATX-P-431 | LCDR3 | QQYGSSPPT |
| 167 | ATX-P-432 | LCDR3 | QQYGSSPPT |
| 168 | ATX-P-430 | LCDR3 | QQYGSSPPT |
| 169 | ATX-P-438 | LCDR3 | QQGQVIPPT |

TABLE 9
Anti-ANGPTL7 antibodies (VH and VL sequences)

| SEQ ID | Antibody | | |
|---|---|---|---|
| NO: | Name: | Descriptor: | Sequence: |
| 170 | ATX-P-428 | VH | QVQLVQSGAEVKKPGASVKVSCKASGYTFSSYGISWVRQAPGQG |
| | | | LEWMGWISAYNGNTNYAQNLRGRVAMTTDTSTTTAYMELRSLR |
| | | | SDDTAVYYCARVSLVWFGELPRGFDYWGQGTLVTVSS |
| 171 | ATX-P-443 | VH | QVQLVQSGAEVKKPGASVKVSCKASGYTFTSYGISWVRQAPGQ |
| | | | GLEWMGWIIAYNGNTNYAQKLQGRITMTINTSTNTAYMELRSL |
| | | | RSDDTAVYYCGRDTGFSFDYWGQGTLVTVSS |
| 172 | ATX-P-429 | VH | QVQLVQSGAEVKKPGASVKVSCKASGYTFSSYGISWVRQAPGQG |
| | | | LEWMGWISAYNGNTNYAQNLRGRVAMTTDTSTTTAYMELRSLR |
| | | | SDDTAVYYCARVSLVWFGELPRGFDYWGQGTLVTVSS |
| 173 | ATX-P-431 | VH | QVQLVQSGAEVKKPGASVKVSCKASGYTFTDYYIHWVRQAPGP |
| | | | GLEWLGWINPNTGATKYAQKFQGRVTMTRDTSISTAYMELSRLR |
| | | | SDDTAVYYWAREDNGNYGDVFDIWGQGTMVTVSS |
| 174 | ATX-P-432 | VH | QVQLVQSGAEVKKPGASVKVSCKASGYTFTDYYIHWVRQAPGP |
| | | | GLEWLGWINPNTGATKYAQKFQGRVTMTRDTSISTAYMELSRLR |
| | | | SDDTAVYYCAREDNGNYGDVFDIWGQGTMVTVSS |
| 175 | ATX-P-428 | VH-nucleotide | CAGGTTCAGCTGGTGCAGTCTGGAGCTGAGGTGAAGAAGCCT |
| | | | GGGGCCTCAGTGAAGGTCTCCTGCAAGGCTTCTGGTTACACCT |
| | | | TTTCCAGCTATGGTATCAGCTGGGTGCGACAGGCCCCTGGACA |
| | | | AGGGCTTGAGTGGATGGGATGGATCAGCGCTTACAATGGTAA |
| | | | CACAAACTATGCACAGAACCTCCGGGGCAGAGTCGCCATGAC |
| | | | CACAGACACATCCACGACCACAGCCTACATGGAGTTGAGGAG |
| | | | CCTGAGATCTGACGACACGGCCGTGTATTACTGTGCGAGAGTT |
| | | | TCATTAGTATGGTTTGGGGAATTACCCAGGGGTTTTGACTACT |
| | | | GGGGCCAGGGAACCCTGGTCACCGTCTCCTCA |
| 176 | ATX-P-443 | VH-nucleotide | CAGGTTCAGCTGGTGCAGTCTGGAGCTGAGGTGAAGAAGCCT |
| | | | GGGGCCTCAGTGAAGGTCTCCTGCAAGGCTTCTGGTTACACCT |
| | | | TTACCAGCTATGGTATCAGCTGGGTGCGACAGGCCCCTGGACA |
| | | | AGGGCTTGAGTGGATGGGATGGATCATCGCTTACAATGGTAAC |
| | | | ACAAACTATGCACAGAAGCTCCAGGGCAGAATCACCATGACC |
| | | | ACAAACACATCCACGAACACAGCCTACATGGAGCTGAGGAGC |
| | | | CTGAGATCTGACGACACGGCCGTGTATTACTGTGGGAGAGATA |
| | | | CGGGGTTTTCTTTTGACTACTGGGGCCAGGGAACCCTGGTCAC |
| | | | CGTCTCCTCA |
| 177 | ATX-P-429 | VH-nucleotide | CAGGTTCAGCTGGTGCAGTCTGGAGCTGAGGTGAAGAAGCCT |
| | | | GGGGCCTCAGTGAAGGTCTCCTGCAAGGCTTCTGGTTACACCT |
| | | | TTTCCAGCTATGGTATCAGCTGGGTGCGACAGGCCCCTGGACA |
| | | | AGGGCTTGAGTGGATGGGATGGATCAGCGCTTACAATGGTAA |
| | | | CACAAACTATGCACAGAACCTCCGGGGCAGAGTCGCCATGAC |
| | | | CACAGACACATCCACGACCACAGCCTACATGGAGTTGAGGAG |
| | | | CCTGAGATCTGACGACACGGCCGTGTATTACTGTGCGAGAGTT |
| | | | TCATTAGTATGGTTTGGGGAATTACCCAGGGGTTTTGACTACT |
| | | | GGGGCCAGGGAACCCTGGTCACCGTCTCCTCA |
| 178 | ATX-P-431 | VH-nucleotide | CAGGTGCAGCTGGTGCAGTCTGGGGCTGAGGTGAAGAAGCCT |
| | | | GGGGCCTCAGTGAAGGTCTCCTGCAAGGCTTCTGGATACACCT |
| | | | TCACCGACTACTATATACACTGGGTGCGACAGGCCCCTGGGCC |
| | | | AGGGCTTGAGTGGTTGGGCTGGATCAACCCTAACACTGGTGCC |
| | | | ACAAAGTATGCACAGAAGTTTCAGGGCAGGGTCACCATGACC |
| | | | AGGGACACGTCCATCAGCACAGCCTATATGGAGCTGAGCAGG |
| | | | CTGAGATCTGACGACACGGCCGTATATTATTGGGCGAGAGAA |
| | | | GATAATGGGAACTACGGGGATGTTTTTGATATCTGGGGACAAG |
| | | | GGACAATGGTCACCGTCTCTTCA |
| 179 | ATX-P-432 | VH-nucleotide | CAGGTGCAGCTGGTGCAGTCTGGGGCTGAGGTGAAGAAGCCT |
| | | | GGGGCCTCAGTGAAGGTCTCCTGCAAGGCTTCTGGATACACCT |
| | | | TCACCGACTACTATATACACTGGGTGCGACAGGCCCCTGGGCC |
| | | | AGGGCTTGAGTGGTTGGGCTGGATCAACCCTAACACTGGTGCC |
| | | | ACAAAGTATGCACAGAAGTTTCAGGGCAGGGTCACCATGACC |
| | | | AGGGACACGTCCATCAGCACAGCCTATATGGAGCTGAGCAGG |
| | | | CTGAGATCTGACGACACGGCCGTATATTATTGTGCGAGAGAAG |
| | | | ATAATGGGAACTACGGGGATGTTTTTGATATCTGGGGACAAGG |
| | | | GACAATGGTCACCGTCTCTTCA |
| 180 | ATX-P-428 | VL | EIVLTQSPGTLSLSPGERATLSCRASQSVSSSYLAWYQQKPGQAP |
| | | | RLLIYGASSRATGIPDRFSGSGSGTDFTLTISRLEPEDFAVYYCQQ |
| | | | YGSSPPTFGGGTKVEIK |
| 181 | ATX-P-443 | VL | EIVLTQSPGTLSLSPGERATLSCRASQSVSSSYLAWYQQKPGQAP |
| | | | RLLIYGASSRATGIPDRESGSGSGTDFTLTISRLEPEDFAVYYCQQ |
| | | | YGSSLTFGGGTKVDIK |
| 182 | ATX-P-429 | VL | DIVMTQSPLSLPVTPGEPASISCRSSQSLLHSNRYNYLDWYLQKPG |
| | | | QSPQLLIYLGSNRASGVPDRFSGSGSGTDFTLQISRVEAEDVGVY |
| | | | YCMQTLQTPYTFGQGTKLEIK |
| 183 | ATX-P-431 | VL | EIVLTQSPGTLSLSPGERATLSCRASQSVSSSYLAWYQQKPGQAP |
| | | | RLLIYGASSRATGIPDRFSGSGSGTDFTLTISRLEPEDFAVYYCQQ |
| | | | YGSSPPTFGGGTKVEIK |
| 184 | ATX-P-432 | VL | EIVLTQSPGTLSLSPGERATLSCRASQSVSSSYLAWYQQKPGQAP |
| | | | RLLIYGASSRATGIPDRESGSGSGTDFTLTISRLEPEDFAVYYCQQ |
| | | | YGSSPPTFGGGTKVEIK |
| 185 | ATX-P-428 | VL-nucleotide | GAAATTGTGTTGACGCAGTCTCCAGGCACCCTGTCTTTGTCTCC |
| | | | AGGGGAAAGAGCCACCCTCTCCTGCAGGGCCAGTCAGAGTGT |
| | | | TAGCAGCAGCTACTTAGCCTGGTACCAGCAGAAACCTGGCCA |
| | | | GGCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCACTCTCACCATCAGCAGACTGGAGCCTGAAGATTTTGCAG |
| | | | TGTATTACTGTCAGCAGTATGGTAGCTCACCTCCCACTTTCGGC |
| | | | GGAGGGACCAAGGTGGAGATCAAA |
| 186 | ATX-P-443 | VL-nucleotide | GAAATTGTGTTGACGCAGTCTCCAGGCACCCTGTCTTTGTCTCC |
| | | | AGGGGAAAGAGCCACCCTCTCCTGCAGGGCCAGTCAGAGTGT |
| | | | TAGCAGCAGCTACTTAGCCTGGTACCAGCAGAAACCTGGCCA |
| | | | GGCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCACTCTCACCATCAGCAGACTGGAGCCTGAAGATTTTGCAG |
| | | | TGTATTACTGTCAGCAGTATGGTAGCTCACTCACTTTCGGCGG |
| | | | AGGGACCAAAGTGGATATCAAA |
| 187 | ATX-P-429 | VL-nucleotide | GATATTGTGATGACTCAGTCTCCACTCTCCCTGCCCGTCACCCC |
| | | | TGGAGAGCCGGCCTCCATCTCCTGCAGGTCTAGTCAGAGCCTC |
| | | | CTGCATAGTAATAGATACAACTATTTGGATTGGTACCTGCAGA |
| | | | AGCCAGGGCAGTCTCCACAGCTCCTGATCTATTTGGGTTCTAA |
| | | | TCGGGCCTCCGGGGTCCCTGACAGGTTCAGTGGCAGTGGATCA |
| | | | GGCACAGATTTTACACTGCAAATCAGCAGAGTGGAGGCTGAG |
| | | | GATGTTGGGGTTTATTACTGCATGCAAACTCTACAAACTCCGT |
| | | | ACACTTTTGGCCAGGGGACCAAGCTGGAGATCAAA |
| 188 | ATX-P-431 | VL-nucleotide | GAAATTGTGTTGACGCAGTCTCCAGGCACCCTGTCTTTGTCTCC |
| | | | AGGGGAAAGAGCCACCCTCTCCTGCAGGGCCAGTCAGAGTGT |
| | | | TAGCAGCAGCTACTTAGCCTGGTACCAGCAGAAACCTGGCCA |
| | | | GGCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCACTCTCACCATCAGCAGACTGGAGCCTGAAGATTTTGCAG |
| | | | TGTATTACTGTCAGCAGTATGGTAGCTCACCTCCCACTTTCGGC |
| | | | GGAGGGACCAAGGTGGAAATCAAA |
| 189 | ATX-P-432 | VL-nucleotide | GAAATTGTGTTGACGCAGTCTCCAGGCACCCTGTCTTTGTCTCC |
| | | | AGGGGAAAGAGCCACCCTCTCCTGCAGGGCCAGTCAGAGTGT |
| | | | TAGCAGCAGCTACTTAGCCTGGTACCAGCAGAAACCTGGCCA |
| | | | GGCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCACTCTCACCATCAGCAGACTGGAGCCTGAAGATTTTGCAG |
| | | | TGTATTACTGTCAGCAGTATGGTAGCTCACCTCCCACTTTCGGC |
| | | | GGAGGGACCAAGGTGGAAATCAAA |
| 190 | ATX-P-437 | VH | QITLKESGPTLVKPTQTLTLTCTFSGFSLSTSGVGVGWIRQPPGKA |
| | | | LEWLALIYWNDDKQYSPSLKSRLTITKDTSKNQVVLTMTNMDP |
| | | | MDTATYYCAHNPDYGDFFDYWGQGTLVTVSS |
| 191 | ATX-P-442 | VH | QITLKESGPTLVKPTQTLTLTCTFSGFSLSTSGVGVGWIRQPPGKA |
| | | | LEWLALIYWNDDKRYSPSLKSRLTITKDTSKNLVVLTMTNMDPV |
| | | | DTATYYCARDYGDYWFFDLWGRGTPVTVSS |
| 192 | ATX-P-437 | VH-nucleotide | CAGATCACCTTGAAGGAGTCTGGTCCTACGCTGGTGAAACCCA |
| | | | CACAGACCCTCACGTTGACCTGCACCTTCTCTGGGTTCTCACTC |
| | | | AGCACTAGTGGCGTGGGTGTGGGCTGGATCCGTCAGCCCCCAG |
| | | | GAAAGGCCCTGGAGTGGCTTGCACTCATTTATTGGAATGATGA |
| | | | TAAACAGTACAGCCCTTCTCTGAAGAGCAGGCTCACCATCACC |
| | | | AAGGACACCTCCAAAAACCAGGTGGTCCTTACAATGACCAAC |
| | | | ATGGACCCTATGGACACAGCCACATATTACTGTGCACACAACC |
| | | | CTGACTACGGTGACTTCTTTGACTACTGGGGCCAGGGAACCCT |
| | | | GGTCACCGTCTCCTCA |
| 193 | ATX-P-442 | VH-nucleotide | CAGATCACCTTGAAGGAGTCTGGTCCTACGCTGGTGAAACCCA |
| | | | CACAGACCCTCACGCTGACCTGCACCTTCTCTGGGTTCTCACTC |
| | | | AGCACTAGTGGAGTGGGTGTGGGCTGGATCCGTCAGCCCCCA |
| | | | GGAAAGGCCCTGGAGTGGCTTGCACTCATTTATTGGAATGATG |
| | | | ATAAGCGCTACAGCCCATCTCTGAAGAGCAGGCTCACCATCAC |
| | | | CAAGGACACCTCCAAAAACCTGGTGGTCCTTACAATGACCAAC |
| | | | ATGGACCCTGTGGACACAGCCACATATTACTGTGCACGTGACT |
| | | | ACGGTGACTACTGGTTCTTCGATCTCTGGGGCCGTGGCACCCC |
| | | | GGTCACCGTCTCCTCA |
| 194 | ATX-P-437 | VL | DIQMTQSPSSLSASVGDRVTITCRASQSISSYLNWYQQKPGKAPK |
| | | | LLIYAASSLQSGVPSRFSGSGSGTDFILTISSLQPEDFATYYCQQ |
| | | | SFSTPLTFGGGTKLEIK |
| 195 | ATX-P-442 | VL | DIQMTQSPSSLSASVGDRVTITCRASQGISSYLAWYQQKPGKAPK |
| | | | LLIYAASSLQSGVPSRFSGSGSGTDFTLTISSLQPEDFATYYCQQ |
| | | | SFSTPLTFGGGTKLEIK |
| 196 | ATX-P-437 | VL-nucleotide | GACATCCAGATGACCCAGTCTCCATCCTCCCTGTCTGCATCTGT |
| | | | AGGAGACAGAGTCACCATCACTTGCCGGGCAAGTCAGAGCAT |
| | | | TAGCAGCTATTTAAATTGGTATCAGCAGAAACCAGGGAAAGC |
| | | | CCCTAAGCTCCTGATCTATGCTGCATCCAGTTTGCAAAGTGGG |
| | | | GTCCCATCAAGGTTCAGTGGCAGTGGATCTGGGACAGATTTCA |
| | | | TTCTCACCATCAGCAGTCTGCAACCTGAAGATTTTGCAACTTA |
| | | | CTACTGTCAACAGAGTTTCAGTACCCCGCTCACTTTCGGCGGA |
| | | | GGGACCAAGCTGGAAATCAAA |
| 197 | ATX-P-442 | VL-nucleotide | GACATCCAGATGACCCAGTCTCCATCCTCCCTGTCTGCATCTGT |
| | | | AGGAGACAGAGTCACCATCACTTGCCGGGCCAGTCAGGGCAT |
| | | | TAGCAGTTATTTAGCCTGGTATCAGCAAAAACCAGGGAAAGC |
| | | | CCCTAAGCTCCTGATCTATGCTGCATCCAGTTTGCAAAGTGGG |
| | | | GTCCCATCAAGGTTCAGTGGCAGTGGATCTGGGACAGATTTCA |
| | | | CTCTCACCATCAGCAGTCTGCAACCTGAAGATTTTGCAACTTA |
| | | | CTACTGTCAACAGAGTTTCAGTACCCCGCTCACTTTCGGCGGA |
| | | | GGGACCAAGCTGGAGATCAAA |
| 198 | ATX-P-444 | VH | EVQLVESGGGLVQPGGSLRLSCAASGFTLSVYDMHWVRQVSGK |
| | | | GLEWVSGIDPDGDTYYPGSVKGRFTISRENAKNSLYLQMNSLRV |
| | | | GDTAVYYCLRGEESNSSEDGFDIWGQGTMVTVSS |
| 199 | ATX-P-445 | VH | EVQLVESGGGLVQPGGSLRLSCAASGFTFSSHDMHWVRQVTGK |
| | | | GLEWVSGIDPAGDTYYPDSVKGRFTISRENAKNSLYLQMNSLRA |
| | | | GDTAVYYCTRGEASSSSEDAFDIWGQGTMVTVSS |
| 200 | ATX-P-446 | VH | EVQLVESGGGLVQPGGSLRLSCAASGFTLSSYDMHWVRQVIGKG |
| | | | LEWVSGIDPDGDTYYPGSVKGRFTISREDAKNSLYLQMNSLRAG |
| | | | DTAVYYCTRGEDNSSSEDAFDIWGQGTMVTVSS |
| 201 | ATX-P-447 | VH | EVQLVESGGGLVQPGGSLRLSCAASGFTISDYDMHWVRQTTGKG |
| | | | LEWVSGIDPDGDTYYPGSLKGRFTISRENAKNSLYLQMNSLRAG |
| | | | DTAVYYCTRGEDSSSSEDAFDIWGQGTMVTVSS* |
| 202 | ATX-P-427 | VH | EVQLVESGGGVVRPGGSLRLSCAASGFSFDDYGMSWVRQVPGK |
| | | | GLEWVSGINWNGGSTGYADSVKGRFTISRDNAKNSLYLQMSSLR |
| | | | AEDTALYYCASYSGSFRDAFDIWGQGTMVTVSS |
| 203 | ATX-P-440 | VH | EVQLVESGGGVVRPGGSLRLSCAASGFTEDDYGMSWVRQAPGK |
| | | | GLEWVSTINWNGGNTGYADSVKGRFTISRDNAKNSLYLQMNSL |
| | | | RAEDTALYYCARDEGAAGLGYYFDYWGQGILVTVSS |
| 204 | ATX-P-424 | VH | EVQLVESGGGLVKPGGSLRLSCAASGFTFSSYSMNWVRQAPGKG |
| | | | LEWVSSITSSSYIFYADSLKGRFTISRDNAKNSLFLQMNSLRAEDT |
| | | | AVYYCARDSNWGEAFDIWGQGTMVTVSS |
| 205 | ATX-P-425 | VH | EVQLVESGGGLVKPGGSLRLSCAASGFTFSTYSMNWVRQAPGKG |
| | | | LEWVSSITSSSSYMFYADSVMGRFTISRDNAKNSLYLQMNSLRAE |
| | | | DTADYYCARDSNWGEAFDIWGQGTMVTVSS |
| 206 | ATX-P-430 | VH | EVQLVESGGGLVQPGGSLRLSCAASGFTFSSYAMSWVRQAPGKG |
| | | | LEWVSAISGSGGSTYYADSVKGRFTISRDNSKNTLYLQMNSLRAE |
| | | | DTAVYYCAKDGDYYYYYMDVWGKGTTVTVSS |
| 207 | ATX-P-439 | VH | EVQLVESGGGLVQPGGSLRLSCAASGFTFSSYAMSWVRQAPGKG |
| | | | LEWVSAISGSGGSTYYADSVKGRFTISRDNSKNTLYLQMNSLRAE |
| | | | DTAVYYCAKVERDMVRGHYYYYMDVWGKGTTVTVSS |
| 208 | ATX-P-423 | VH | QVQLVESGGGVVQPGRSLRLSCAASGFTFSSYGMHWVRQAPGK |
| | | | GLEWVSHITYDGSDKYYGDSVKGRFTISRDNSKNTLYLQMNSLR |
| | | | AEDTAVYYCAKDGYSSGWWYFDYWGQGTLVTVSS |
| 209 | ATX-P-438 | VH | QVQLVESGGGVVQPGRSLRLSCAASGFTFSSYGMHWVRQAPGK |
| | | | GLEWVAIISYDGNNKYYADSVKGRFTISRDNSKNTLYLQMNSLR |
| | | | DEDTAVYYCAKERGYSSSSGYYYYYMDVWGKGTTVTVSS |
| 210 | ATX-P-448 | VH | QVQLVESGGGVVQPGRSLRLSCAASGFPFSSYGMHWVRQAPGK. |
| | | | GLEWVAVMSYDGINGYYADSVKGRFTISRDNSKNTLYLQMNSL |
| | | | RAEDTAVYYCARLVYADDAFDIWGQGTTVTVSS |
| 211 | ATX-P-449 | VH | QVQLVESGGGVVQPGRSLRLSCAASGFAFSSYGMHWVRQAPGK |
| | | | GLEWVAVISYYGSNKYYADSVKGRFTISRDNSKNTLYLQMNSLR |
| | | | AEDTAVYHCARGITMDVWGKGTTVTVSS |
| 212 | ATX-P-433 | VH | EVQLVESGGGLVQPGRSLRLSCAASGFTFDDFAMHWVRQGPGK |
| | | | GLEWVSGIGWNSGSIGYADSVKGRFTISRDNAKNSLYLQMNSLR |
| | | | AEDTALYYCAKAPDYGDYYFDYWGQGTLVTVSS |
| 213 | ATX-P-444 | VH-nucleotide | GAGGTGCAGCTGGTGGAGTCGGGGGGAGGCTTGGTACAGCCT |
| | | | GGGGGGTCCCTGAGACTCTCCTGTGCAGCCTCTGGATTCACCC |
| | | | TCAGTGTCTACGACATGCACTGGGTCCGCCAAGTTTCAGGAAA |
| | | | AGGTCTGGAGTGGGTCTCAGGTATTGATCCTGATGGTGACACA |
| | | | TACTATCCCGGCTCCGTGAAGGGCCGATTCACCATCTCCAGAG |
| | | | AAAATGCCAAGAACTCCTTGTATCTTCAAATGAACAGCCTGAG |
| | | | AGTCGGGGACACGGCTGTGTATTATTGTCTAAGAGGGGAGGA |
| | | | AAGCAACTCGTCCGAGGATGGTTTTGATATATGGGGACAAGG |
| | | | GACAATGGTCACCGTCTCCTCA |
| 214 | ATX-P-445 | VH-nucleotide | GAGGTGCAGCTGGTGGAGTCTGGGGGAGGCTTGGTACAGCCT |
| | | | GGGGGGTCCCTGAGACTCTCCTGTGCAGCCTCTGGATTCACCT |
| | | | TCAGTAGCCACGACATGCACTGGGTCCGCCAAGTTACAGGAA |
| | | | AAGGTCTGGAGTGGGTCTCAGGTATTGATCCTGCTGGTGACAC |
| | | | ATACTATCCAGACTCCGTGAAGGGCCGATTCACCATCTCTAGA |
| | | | GAAAATGCCAAGAACTCCTTGTATCTTCAAATGAACAGCCTGA |
| | | | GAGCCGGGGACACGGCTGTGTATTACTGTACAAGAGGAGAGG |
| | | | CTAGTAGTTCGTCCGAGGATGCTTTTGATATCTGGGGACAAGG |
| | | | GACAATGGTCACTGTCTCCTCA |
| 215 | ATX-P-446 | VH-nucleotide | GAGGTGCAGCTGGTGGAGTCTGGGGGAGGCTTGGTACAGCCT |
| | | | GGGGGGTCCCTGAGACTCTCCTGTGCAGCCTCTGGATTCACCC |
| | | | TCAGTAGCTACGACATGCACTGGGTCCGCCAAGTTATAGGAAA |
| | | | AGGTCTGGAGTGGGTCTCAGGTATTGATCCTGATGGTGACACA |
| | | | TACTATCCAGGCTCCGTGAAGGGCCGATTCACCATCTCCAGAG |
| | | | AAGATGCCAAGAACTCCTTGTATCTTCAAATGAACAGCCTGAG |
| | | | AGCCGGGGACACGGCTGTGTATTACTGTACAAGAGGAGAGGA |
| | | | TAACAGCTCGTCCGAGGATGCTTTTGATATCTGGGGACAAGGG |
| | | | ACAATGGTCACTGTCTCCTCA |
| 216 | ATX-P-447 | VH--nucleotide | GAGGTCCAGCTGGTGGAGTCTGGGGGAGGCTTGGTACAGCCG |
| | | | GGGGGGTCCCTGAGACTCTCCTGTGCAGCCTCTGGATTCACCA |
| | | | TCAGTGACTACGACATGCACTGGGTCCGCCAAACTACAGGAA |
| | | | AAGGTCTAGAATGGGTCTCAGGTATTGATCCTGATGGCGACAC |
| | | | ATATTATCCAGGCTCCTTGAAGGGGCGATTCACCATCTCCAGA |
| | | | GAAAATGCCAAGAATTCTTTGTATCTTCAAATGAACAGTCTGA |
| | | | GAGCCGGGGACACGGCTGTGTATTACTGTACAAGAGGAGAGG |
| | | | ATAGTAGTTCGTCCGAGGATGCTTTTGATATCTGGGGACAAGG |
| | | | GACAATGGTCACCGTCTCCTCA |
| 217 | ATX-P-427 | VH-nucleotide | GAGGTGCAGCTGGTGGAGTCTGGGGGAGGTGTGGTACGGCCT |
| | | | GGGGGGTCCCTGAGACTCTCCTGTGCAGCCTCTGGATTCAGCT |
| | | | TTGATGATTATGGCATGAGCTGGGTCCGCCAAGTTCCAGGGAA |
| | | | GGGGCTGGAGTGGGTCTCTGGTATTAATTGGAATGGTGGTAGT |
| | | | ACAGGTTATGCAGACTCTGTGAAGGGCCGATTCACCATCTCCA |
| | | | GAGACAACGCCAAGAACTCCCTGTATCTGCAAATGAGCAGTCT |
| | | | GAGAGCCGAGGACACGGCCTTGTATTACTGTGCGAGCTATAGT |
| | | | GGGAGCTTCCGTGATGCTTTTGATATCTGGGGACAAGGGACAA |
| | | | TGGTCACCGTCTCTTCA |
| 218 | ATX-P-440 | VH-nucleotide | GAGGTGCAGCTGGTGGAGTCTGGGGGAGGTGTGGTACGGCCT |
| | | | GGGGGGTCCCTGAGACTCTCCTGTGCAGCCTCTGGATTCACCT |
| | | | TTGATGATTATGGCATGAGCTGGGTCCGCCAAGCTCCAGGGAA |
| | | | GGGGCTGGAGTGGGTCTCTACTATTAATTGGAATGGTGGTAAC |
| | | | ACAGGTTATGCAGACTCTGTGAAGGGCCGATTCACCATCTCCA |
| | | | GAGACAACGCCAAGAACTCCCTGTATCTGCAAATGAACAGTCT |
| | | | GAGAGCCGAGGACACGGCCTTGTATTACTGTGCGAGAGATGA |
| | | | AGGAGCAGCTGGACTTGGATACTACTTTGACTACTGGGGCCAG |
| | | | GGAACCCTGGTCACCGTCTCCTCA |
| 219 | ATX-P-424 | VH--nucleotide | GAGGTGCAGCTGGTGGAGTCTGGGGGAGGCCTGGTCAAGCCT |
| | | | GGGGGGTCCCTGAGACTCTCCTGTGCAGCCTCTGGATTCACCT |
| | | | TCAGTAGCTATAGCATGAACTGGGTCCGCCAGGCTCCAGGGA |
| | | | AGGGGCTGGAGTGGGTCTCATCCATTACTAGTAGTAGTTACAT |
| | | | ATTCTACGCAGACTCATTGAAGGGCCGATTCACCATCTCCAGA |
| | | | GACAACGCCAAGAACTCACTGITTCTGCAAATGAACAGCCTGA |
| | | | GAGCCGAGGACACAGCTGTTTATTACTGTGCGAGAGATTCTAA |
| | | | CTGGGGCGAGGCTTTTGATATCTGGGGACAAGGGACAATGGT |
| | | | CACCGTCTCTTCA |
| 220 | ATX-P-425 | VH-nucleotide | GAGGTGCAGCTGGTGGAGTCTGGGGGAGGCCTGGTCAAGCCT |
| | | | GGGGGGTCCCTGAGACTCTCCTGTGCAGCCTCTGGTTTCACCT |
| | | | TCAGTACTTATAGCATGAACTGGGTCCGCCAGGCTCCAGGGAA |
| | | | GGGGCTGGAGTGGGTCTCATCCATTACTAGTAGTAGTAGTTAC |
| | | | ATGTTCTACGCAGACTCAGTGATGGGCCGATTCACCATCTCCA |
| | | | GAGACAACGCCAAGAACTCACTGTACCTACAAATGAACAGCC |
| | | | TGAGAGCCGAGGACACAGCTGATTATTACTGTGCGAGAGATTC |
| | | | TAATTGGGGGGAGGCTTTTGATATCTGGGGACAAGGGACAAT |
| | | | GGTCACCGTCTCTTCA |
| 221 | ATX-P-430 | VH-nucleotide | GAGGTGCAGCTGGTGGAGTCTGGGGGAGGCTTGGTACAGCCT |
| | | | GGGGGGTCCCTGAGACTCTCCTGTGCAGCCTCTGGATTCACCT |
| | | | TTAGCAGCTATGCCATGAGCTGGGTCCGCCAGGCTCCAGGGAA |
| | | | GGGGCTGGAGTGGGTCTCAGCTATTAGTGGTAGTGGTGGTAGC |
| | | | ACATACTACGCAGACTCCGTGAAGGGCCGGTTCACCATCTCCA |
| | | | GAGACAATTCCAAGAACACGCTGTATCTGCAAATGAACAGCC |
| | | | TGAGAGCCGAGGACACGGCCGTATATTACTGTGCGAAAGACG |
| | | | GTGACTACTACTACTACTACATGGACGTCTGGGGCAAAGGGAC |
| | | | CACGGTCACCGTCTCCTCA |
| 222 | ATX-P-439 | VH-nucleotide | GAGGTGCAGCTGGTGGAGTCTGGGGGAGGCTTGGTACAGCCT |
| | | | GGGGGGTCCCTGAGACTCTCCTGTGCAGCCTCTGGATTCACCT |
| | | | TTAGCAGCTATGCCATGAGCTGGGTCCGCCAGGCTCCAGGGAA |
| | | | GGGGCTGGAGTGGGTCTCAGCTATTAGTGGTAGTGGTGGTAGC |
| | | | ACATACTACGCAGACTCCGTGAAGGGCCGGTTCACCATCTCCA |
| | | | GAGACAATTCCAAGAACACGCTGTATCTGCAAATGAACAGCC |
| | | | TGAGAGCCGAGGACACGGCCGTATATTACTGTGCGAAAGTGG |
| | | | AAAGGGATATGGTTCGGGGCCACTACTACTACTACATGGACGT |
| | | | CTGGGGCAAAGGGACCACGGTCACCGTCTCTTCA |
| 223 | ATX-P-423 | VH-nucleotide | CAGGTGCAGCTGGTGGAGTCTGGGGGAGGCGTGGTCCAGCCT |
| | | | GGGAGGTCCCTGAGACTCTCCTGTGCAGCCTCTGGATTCACCT |
| | | | TCAGTAGCTATGGCATGCACTGGGTCCGCCAGGCTCCAGGCAA |
| | | | GGGGCTGGAGTGGGTGTCACATATAACATATGATGGAAGTGA |
| | | | TAAATACTATGGAGACTCCGTGAAGGGCCGATTCACCATCTCC |
| | | | AGAGACAATTCCAAGAATACGCTGTATCTGCAAATGAACAGC |
| | | | CTGAGAGCTGAGGACACGGCTGTGTATTACTGTGCGAAAGAT |
| | | | GGGTATAGCAGTGGCTGGTGGTACTTTGACTACTGGGGCCAGG |
| | | | GAACCCTGGTCACCGTCTCCTCA |
| 224 | ATX-P-438 | VH--nucleotide | CAGGTGCAGCTGGTGGAGTCCGGGGGAGGCGTGGTCCAGCCT |
| | | | GGGAGGTCCCTGAGACTCTCCTGTGCAGCCTCTGGATTCACCT |
| | | | TCAGTAGCTATGGCATGCACTGGGTCCGCCAGGCTCCAGGCAA |
| | | | GGGGCTGGAGTGGGTGGCAATTATATCATATGATGGAAATAA |
| | | | TAAATACTATGCAGACTCCGTGAAGGGCCGATTCACCATCTCC |
| | | | AGAGACAATTCCAAGAACACGCTGTATCTGCAAATGAACAGC |
| | | | CTGAGAGATGAGGACACGGCTGTTTATTATTGTGCGAAAGAG |
| | | | AGGGGGTATAGCAGCTCGTCCGGCTACTACTACTACTACATGG |
| | | | ACGTCTGGGGCAAAGGGACCACGGTCACCGTCTCCTCA |
| 225 | ATX-P-448 | VH-nucleotide | CAAGTGCAATTGGTGGAGTCCGGTGGAGGAGTAGTACAGCCG |
| | | | GGAAGGTCACTGAGACTTTCCTGCGCTGCTAGTGGATTCCCAT |
| | | | TTAGCTCCTACGGGATGCACTGGGTTCGCCAAGCGCCAGGTAA |
| | | | GGGACTGGAATGGGTGGCTGTCATGAGTTACGATGGAATTAA |
| | | | CGGCTATTATGCCGATAGCGTCAAAGGCCGGTTCACGATCTCA |
| | | | AGAGACAACTCTAAGAACACGCTCTACTTGCAGATGAACAGT |
| | | | CTCCGCGCAGAAGATACGGCCGTGTATTACTGTGCGAGATTGG |
| | | | TGTATGCCGATGATGCTTTTGATATCTGGGGACAAGGGACCAC |
| | | | GGTCACCGTCTCCTCA |
| 226 | ATX-P-449 | VH-nucleotide | CAAGTGCAATTGGTGGAGTCCGGTGGAGGAGTAGTACAGCCG |
| | | | GGAAGGTCACTGAGACTTTCCTGCGCTGCTAGTGGATTCGCCT |
| | | | TTAGTTCCTACGGCATGCACTGGGTTCGCCAAGCGCCAGGTAA |
| | | | GGGACTGGAATGGGTGGCTGTCATCTCCTACTACGGATCTAAT |
| | | | AAATATTATGCCGATAGCGTCAAAGGCCGGTTCACGATCTCAA |
| | | | GAGACAACTCTAAGAACACGCTCTACTTGCAGATGAACAGTCT |
| | | | CCGCGCAGAAGATACGGCCGTGTATCACTGTGCGAGAGGGAT |
| | | | TACTATGGACGTCTGGGGCAAAGGGACCACGGTCACCGTCTCC |
| | | | TCA |
| 227 | ATX-P-433 | VH-nucleotide | GAAGTGCAGCTGGTGGAGTCTGGGGGAGGCTTGGTACAGCCT |
| | | | GGCAGGTCCCTGAGACTCTCCTGTGCAGCCTCTGGATTCACCT |
| | | | TTGATGATTTTGCCATGCACTGGGTCCGGCAAGGTCCAGGGAA |
| | | | GGGCCTGGAGTGGGTCTCAGGTATTGGTTGGAATAGTGGTAGC |
| | | | ATCGGCTATGCGGACTCTGTGAAGGGCCGATTCACCATCTCCA |
| | | | GAGACAACGCCAAGAACTCCCTGTATCTGCAAATGAACAGTCT |
| | | | GAGAGCTGAGGACACGGCCTTATATTACTGTGCAAAAGCCCCT |
| | | | GACTACGGTGACTACTACTTTGACTACTGGGGCCAGGGAACCC |
| | | | TGGTCACCGTCTCCTCA |
| 228 | ATX-P-444 | VL | DVVMTQSPLSLPVTLGQPASISCRSSQSLVYSDGNTYLNWFQQRP |
| | | | GQSPRRLIYKVSNRDSGVPDRFSGSGSGTDFTLKISKVEAEDVGIY |
| | | | YCMQGTHWPWTFGQGTKVDIK |
| 229 | ATX-P-445 | VL | DVVMTQSPLSLPVTLGQPASISCRSSQSLVYSDGNTYLNWFQQRP |
| | | | GQSPRRLIHEVSNRDSGVPDRFSGSGSGTDFTLKISRVEAEDVGV |
| | | | YYCMQGTHWPWTFGQGTKVDIK |
| 230 | ATX-P-446 | VL | DVVMTQSPLSLPVTLGQPASISCRSSQSLVFSDGNTYLNWFQQRP |
| | | | GQSPRRLIYKVSNRDSGVPDRFSGSGSGTDFTLKISRVEAEDVGV |
| | | | YYCMQGTHWPWTFGQGTKVDIK |
| 231 | ATX-P-447 | VL | DVVMTQSPLSLPVTLGQPASISCRSSQSLVYSDGNTYLNWFQQRP |
| | | | GQSPRRLIHEVSNRDSGVPDRFSGSGSGTDFTLKISRVEAEDVGV |
| | | | YYCMQGTHWPWTFGQGTKVDIK |
| 232 | ATX-P-427 | VL | AIQMTQSPSSLSASVGDRVTITCRASQGIRNDLGWYQQKPGKAPK |
| | | | LLIYAASSLQSGVPSRFSGSGSGTDFTLTISSLQPEDFATYYCLQDY |
| | | | NYPYTFGQGTKLEIK |
| 233 | ATX-P-440 | VL | DIQMTQSPSTLSASVGDRVTITCRASQSISSWLAWYQQKPGKAPK |
| | | | LLIYATSSLQSGVPSRFSGSGSGTDFTLTISSLQPEDFATYYCQQSY |
| | | | STPITFGQGTRLEIK |
| 234 | ATX-P-424 | VL | DIQMTQSPSSLSASVGDRVTITCRASQGIRNDLGWYQQKPGKAPK |
| | | | RLIYAASSLPSGVPSRFSGSGSGTEFTLTISSLQPEDFAIYYCLQHN |
| | | | SYPWTFGQGTKVEIK |
| 235 | ATX-P-425 | VL | DIQMTQSPSSLSASVGDRVTITCRASQGIRIDLGWFQQKPGKAPK |
| | | | RLIYAASSLPSGVPSRFSGSGSGTEFTLTISSLQPEDFATYYCLQHN |
| | | | SYPWTFGQGTKVEIK |
| 236 | ATX-P-430 | VL | EIVLTQSPGTLSLSPGERATLSCRASQSVSSSYLAWYQQKPGQAP |
| | | | RLLIYGASSRATGIPDRFSGSGSGTDFTLTISRLEPEDFAVYYCQQ |
| | | | YGSSPPTFGGGTKVEIK |
| 237 | ATX-P-439 | VL | DIQMTQSPSSVSASVGDRVTITCRASQGISSWLAWYQQKPGKAPK |
| | | | LLIYAASSLQSGVPSRFSGSGSGTDFTLTISSLQPEDFATYYCQQA |
| | | | NSFPWTFGQGTKVDIK |
| 238 | ATX-P-423 | VL | DIQMTQSPSSLSASVGDRVTITCRASQGIRNDLGWYQQKPGKAPK |
| | | | RLIYAASSLQSGVPSRFSGSGSGTEFALTISSLQPEDFATYYCLQH |
| | | | NSYPLTFGGGTKLEIK |
| 239 | ATX-P-438 | VL | EIVLTQSPGTLSLSPGERATLSCRASQSVSRSYLAWYQQKPGQAP |
| | | | RLLIIGASTRATGIPDRFSGSGSGTDFTLTISRLEPEDFAVYYCQQG |
| | | | QVIPPTFGQGTKVEIK |
| 240 | ATX-P-448 | VL | EIVLTQSPATLSLSPGERATLSCRASQSVSSYLAWYQQKPGQAPR |
| | | | LLIYDASNRATGIPARFSGSGSGTDFTLTISSLEPEDFAVYYCQQRS |
| | | | NWPLTFGGGTKVEIK |
| 241 | ATX-P-449 | VL | EIVLTQSPATLSLSPGERATLSCRASQSVSSYLAWYQQKPGQAPR |
| | | | LLIYDASNRATGIPARFSGSGSGTDFTLTISSLEPEDFAVYYCQQRS |
| | | | NWPLTFGGGTKVEIK |
| 242 | ATX-P-433 | VL | DIQMTQSPSSLSASVGDRVTITCRASQSISIYLNWYQQKPGTAPKL |
| | | | LIYAASSLQSGVPSRFSGSGSGTDFTLTISSLQPEDFAAYYCQQSY |
| | | | TTPLTFGGGTKVEIK |
| 243 | ATX-P-444 | VL--nucleotide | GATGTTGTGATGACTCAGTCTCCACTCTCCCTGCCCGTCACCCT |
| | | | TGGACAGCCGGCCTCCATCTCCTGCAGGTCTAGTCAAAGCCTC |
| | | | GTATACAGTGATGGAAACACCTACTTGAATTGGTTTCAGCAGA |
| | | | GGCCAGGCCAATCTCCAAGGCGCCTAATTTATAAGGTTTCTAA |
| | | | CCGGGACTCTGGGGTCCCAGACAGATTCAGCGGCAGTGGGTC |
| | | | AGGCACTGATTTCACACTGAAAATCAGCAAGGTGGAGGCTGA |
| | | | GGATGTTGGGATTTATTACTGCATGCAAGGTACACACTGGCCG |
| | | | TGGACGTTCGGCCAAGGGACCAAAGTGGATATCAAA |
| 244 | ATX-P-445 | VL-nucleotide | GATGTTGTGATGACTCAGTCTCCACTCTCCCTGCCCGTCACCCT |
| | | | TGGACAGCCGGCCTCCATCTCCTGCAGGTCTAGTCAAAGCCTC |
| | | | GTATACAGTGATGGAAACACCTACTTGAATTGGTTTCAGCAGA |
| | | | GGCCAGGCCAATCTCCAAGGCGCCTAATTCATGAGGTTTCTAA |
| | | | CCGGGACTCTGGGGTCCCAGACAGATTCAGCGGCAGTGGGTC |
| | | | AGGCACTGATTTCACACTGAAAATCAGCAGGGTGGAGGCTGA |
| | | | GGATGTTGGGGTTTATTACTGCATGCAAGGTACACACTGGCCG |
| | | | TGGACGTTCGGCCAAGGGACCAAAGTGGATATCAAA |
| 245 | ATX-P-446 | VL-nucleotide | GATGTTGTGATGACTCAGTCTCCACTCTCCCTGCCCGTCACCCT |
| | | | TGGACAGCCGGCCTCCATCTCCTGCAGGTCTAGTCAAAGCCTC |
| | | | GTATTCAGTGATGGAAACACCTACTTGAATTGGTTTCAGCAGA |
| | | | GGCCAGGCCAATCTCCAAGGCGCCTAATTTATAAGGTTTCTAA |
| | | | CCGGGACTCTGGGGTCCCAGACAGATTCAGCGGCAGTGGGTC |
| | | | AGGCACTGATTTCACACTGAAAATCAGCAGGGTGGAGGCTGA |
| | | | GGATGTTGGGGTTTATTACTGCATGCAAGGTACACACTGGCCG |
| | | | TGGACGTTCGGCCAAGGGACCAAAGTGGATATCAAA |
| 246 | ATX-P-447 | VL-nucleotide | GATGTTGTGATGACTCAGTCTCCACTCTCCCTGCCCGTCACCCT |
| | | | TGGACAGCCGGCCTCCATCTCCTGCAGGTCTAGTCAAAGCCTC |
| | | | GTATACAGTGATGGAAACACCTACTTGAATTGGTTTCAGCAGA |
| | | | GGCCAGGCCAATCTCCAAGGCGCCTAATTCATGAGGTTTCTAA |
| | | | CCGGGACTCTGGGGTCCCAGACAGATTCAGCGGCAGTGGGTC |
| | | | AGGCACTGATTTCACACTGAAAATCAGCAGGGTGGAGGCTGA |
| | | | GGATGTTGGGGTTTATTACTGCATGCAAGGTACACACTGGCCG |
| | | | TGGACGTTCGGCCAAGGGACCAAAGTGGATATCAAA |
| 247 | ATX-P-427 | VL-nucleotide | GCCATCCAGATGACCCAGTCTCCATCCTCCCTGTCTGCATCTGT |
| | | | AGGAGACAGAGTCACCATCACTTGCCGGGCAAGTCAGGGCAT |
| | | | TAGAAATGATTTAGGCTGGTATCAGCAGAAACCAGGGAAAGC |
| | | | CCCTAAGCTCCTGATCTATGCTGCATCCAGTTTACAAAGTGGG |
| | | | GTCCCATCAAGGTTCAGCGGCAGTGGATCTGGCACAGATTTCA |
| | | | CTCTCACCATCAGCAGCCTGCAGCCTGAAGATTTTGCAACTTA |
| | | | TTACTGTCTACAAGATTACAATTACCCGTACACTTTTGGCCAG |
| | | | GGGACCAAGCTGGAGATCAAA |
| 248 | ATX-P-440 | VL-nucleotide | GACATCCAGATGACCCAGTCTCCTTCCACCCTGTCTGCATCTGT |
| | | | AGGAGACAGAGTCACCATCACTTGCCGGGCCAGTCAGAGTAT |
| | | | TAGTAGCTGGTTGGCCTGGTATCAGCAGAAACCAGGGAAAGC |
| | | | CCCTAAGCTCCTGATCTATGCTACATCCAGTTTGCAAAGTGGG |
| | | | GTCCCATCAAGGTTCAGTGGCAGTGGATCTGGGACAGATTTCA |
| | | | CTCTCACCATCAGCAGTCTGCAACCTGAAGATTTTGCAACTTA |
| | | | CTACTGTCAACAGAGTTACAGTACCCCGATCACCTTCGGCCAA |
| | | | GGGACACGACTGGAGATTAAA |
| 249 | ATX-P-424 | VL-nucleotide | GACATCCAGATGACCCAGTCTCCATCCTCCCTGTCTGCATCTGT |
| | | | AGGAGACAGAGTCACCATCACTTGCCGGGCAAGTCAGGGCAT |
| | | | TAGAAATGATTTAGGCTGGTATCAGCAGAAACCAGGGAAAGC |
| | | | CCCTAAGCGCCTGATCTATGCTGCATCCAGTTTGCCAAGTGGG |
| | | | GTCCCATCAAGGTTCAGCGGCAGTGGATCTGGGACAGAATTCA |
| | | | CTCTCACAATCAGCAGCCTGCAGCCTGAAGATTTTGCAATTTA |
| | | | TTACTGTCTACAGCATAATAGTTACCCGTGGACGTTCGGCCAA |
| | | | GGGACCAAGGTGGAAATCAAA |
| 250 | ATX-P-425 | VL-nucleotide | GACATCCAGATGACCCAGTCTCCATCCTCCCTGTCCGCATCTG |
| | | | TAGGAGACAGAGTCACCATCACTTGCCGGGCAAGTCAGGGCA |
| | | | TTAGAATTGATTTAGGCTGGTTTCAGCAGAAACCAGGGAAAGC |
| | | | CCCTAAGCGCCTCATCTATGCTGCATCCAGTTTGCCAAGTGGG |
| | | | GTCCCATCAAGGTTCAGCGGCAGTGGATCTGGGACAGAATTCA |
| | | | CTCTCACAATCAGCAGCCTGCAGCCTGAAGATTTTGCAACTTA |
| | | | TTACTGTCTACAGCATAATAGTTACCCGTGGACGTTCGGCCAA |
| | | | GGGACCAAGGTGGAGATCAAA |
| 251 | ATX-P-430 | VL-nucleotide | GAAATTGTGTTGACGCAGTCTCCAGGCACCCTGTCTTTGTCTCC |
| | | | AGGGGAAAGAGCCACCCTCTCCTGCAGGGCCAGTCAGAGTGT |
| | | | TAGCAGCAGCTACTTAGCCTGGTACCAGCAGAAACCTGGCCA |
| | | | GGCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCACTCTCACCATCAGCAGACTGGAGCCTGAAGATTTTGCAG |
| | | | TGTATTACTGTCAGCAGTATGGTAGCTCACCTCCCACTTTCGGC |
| | | | GGAGGGACCAAGGTGGAAATCAAA |
| 252 | ATX-P-439 | VL-nucleotide | GACATCCAGATGACCCAGTCTCCATCTTCCGTGTCTGCATCTGT |
| | | | AGGAGACAGAGTCACCATCACTTGTCGGGCGAGTCAGGGTAT |
| | | | TAGCAGCTGGTTAGCCTGGTATCAGCAGAAACCAGGGAAAGC |
| | | | CCCTAAGCTCCTGATCTATGCTGCATCCAGTTTGCAAAGTGGG |
| | | | GTCCCATCAAGGTTCAGCGGCAGTGGATCTGGGACAGATTTCA |
| | | | CTCTCACCATCAGCAGCCTGCAGCCTGAAGATTTTGCAACTTA |
| | | | CTATTGTCAACAGGCTAACAGTTTCCCGTGGACGTTCGGCCAA |
| | | | GGGACCAAAGTGGATATCAAA |
| 253 | ATX-P-423 | VL--nucleotide | GACATCCAGATGACCCAGTCTCCATCCTCCCTGTCTGCATCTGT |
| | | | AGGAGACAGAGTCACCATCACTTGCCGGGCAAGTCAGGGCAT |
| | | | TAGAAATGATTTAGGCTGGTATCAGCAGAAACCAGGGAAAGC |
| | | | CCCTAAGCGCCTGATCTATGCTGCATCCAGTTTGCAAAGTGGG |
| | | | GTCCCATCAAGGTTCAGCGGCAGTGGATCTGGGACAGAATTCG |
| | | | CTCTCACAATCAGCAGCCTGCAGCCTGAAGATTTTGCAACTTA |
| | | | TTACTGTCTACAGCATAATAGTTACCCTCTCACTTTCGGCGGA |
| | | | GGGACCAAGCTGGAGATCAAA |
| 254 | ATX-P-438 | VL-nucleotide | GAAATTGTGCTGACTCAGTCTCCCGGCACACTGTCTCTTAGCC |
| | | | CTGGCGAAAGAGCCACACTGAGCTGTAGAGCCAGCCAGAGCG |
| | | | TGTCCAGAAGCTACCTGGCTTGGTATCAGCAGAAGCCCGGACA |
| | | | GGCTCCCAGACTGCTGATCATCGGAGCCTCTACAAGAGCCACC |
| | | | GGCATTCCCGATAGATTCAGCGGCTCTGGCAGCGGCACCGATT |
| | | | TCACCCTGACAATCAGCAGACTGGAACCCGAGGACTTCGCCGT |
| | | | GTACTACTGTCAGCAGGGCCAAGTGATCCCTCCTACCTTTGGC |
| | | | CAGGGCACCAAGGTGGAAATCAAA |
| 255 | ATX-P-448 | VL-nucleotide | GAAATTGTGTTGACGCAGTCTCCAGCCACCCTGTCTTTGTCTCC |
| | | | AGGGGAAAGAGCCACCCTCTCCTGCAGGGCCAGTCAGAGTGT |
| | | | TAGCAGCTACTTAGCCTGGTACCAACAGAAACCTGGCCAGGCT |
| | | | CCCAGGCTCCTCATCTATGATGCATCCAACAGGGCCACTGGCA |
| | | | TCCCAGCCAGGTTCAGTGGCAGTGGGTCTGGGACAGACTTCAC |
| | | | TCTCACCATCAGCAGCCTAGAGCCTGAAGATTTTGCAGTTTAT |
| | | | TACTGTCAGCAGCGTAGCAACTGGCCTCTCACTTTCGGCGGAG |
| | | | GGACCAAGGTGGAGATCAAA |
| 256 | ATX-P-449 | VL-nucleotide | GAAATTGTGTTGACGCAGTCTCCAGCCACCCTGTCTTTGTCTCC |
| | | | AGGGGAAAGAGCCACCCTCTCCTGCAGGGCCAGTCAGAGTGT |
| | | | TAGCAGCTACTTAGCCTGGTACCAACAGAAACCTGGCCAGGCT |
| | | | CCCAGGCTCCTCATCTATGATGCATCCAACAGGGCCACTGGCA |
| | | | TCCCAGCCAGGTTCAGTGGCAGTGGGTCTGGGACAGACTTCAC |
| | | | TCTCACCATCAGCAGCCTAGAGCCTGAAGATTTTGCAGTTTAT |
| | | | TACTGTCAGCAGCGTAGCAACTGGCCTCTCACTTTCGGCGGAG |
| | | | GGACCAAGGTGGAGATCAAA |
| 257 | ATX-P-433 | VL-nucleotide | GACATCCAGATGACCCAGTCTCCATCCTCCCTGTCTGCATCTGT |
| | | | AGGAGACAGAGTCACCATCACTTGCCGGGCAAGTCAGAGCAT |
| | | | TAGCATCTATTTAAATTGGTATCAGCAGAAACCAGGGACAGCC |
| | | | CCTAAGCTCCTGATCTATGCTGCATCCAGTTTGCAAAGTGGGG |
| | | | TCCCATCAAGGTTCAGTGGCAGTGGATCTGGGACAGATTTCAC |
| | | | TCTCACCATCAGCAGTCTGCAACCTGAAGATTTTGCAGCTTAC |
| | | | TACTGTCAACAGAGTTACACTACCCCGCTCACTTTCGGCGGAG |
| | | | GGACCAAGGTGGAAATCAAA |
| 258 | ATX-P-435 | VH | QLQLQESGPGLVKPSETLSLTCTVSGGSISSSSSYWGWIRQPPGKG |
| | | | LEWIGSIYYSGSTYSNPSLKSRVTISVDTSKNQFSLKLSSVTAADT |
| | | | AVYYCARQYISGTEYFQYWGQGTLVTVSS |
| 259 | ATX-P-436 | VH | QLQLQESGPGLVKPSETLSLTCTVSGGSISSSSSYWGWIRQPPGKG |
| | | | LEWIGSIYYSGSTYSNPSLKSRVTISVDTSKNQFSLKLSSVTAADT |
| | | | AVYYCARQYISGTEYFQYWGQGTLVTVSS |
| 260 | ATX-P-434 | VH | QVQLQESGPGLVKPSETLSLTCTVSGGSVSSGSYYWSWIRQPPGK |
| | | | GLEWIGYIYYSGSTSYNPSLKSRVSISVDTSKNQFSLKLSSVTAAD |
| | | | TAVFYCARAKWEGDYFDYWGQGTLVTVSS |
| 261 | ATX-P-435 | VH-nucleotide | CAGCTGCAGCTGCAGGAGTCGGGCCCAGGACTGGTGAAGCCT |
| | | | TCGGAGACCCTGTCCCTCACCTGCACTGTCTCTGGTGGTTCCAT |
| | | | CAGCAGTAGTAGTTCCTACTGGGGCTGGATCCGCCAGCCCCCA |
| | | | GGGAAGGGACTGGAGTGGATTGGGAGTATCTATTATAGTGGG |
| | | | AGCACCTACTCCAACCCGTCCCTCAAGAGTCGAGTCACCATAT |
| | | | CCGTAGACACGTCCAAGAATCAGTTCTCCCTGAAGCTGAGCTC |
| | | | TGTGACCGCCGCAGACACGGCTGTGTATTACTGTGCGAGACAG |
| | | | TATATAAGTGGAACTGAATACTTCCAGTACTGGGGCCAGGGCA |
| | | | CCCTGGTCACCGTCTCCTCA |
| 262 | ATX-P-436 | VH-nucleotide | CAGCTGCAGCTGCAGGAGTCGGGCCCAGGACTGGTGAAGCCT |
| | | | TCGGAGACCCTGTCCCTCACCTGCACTGTCTCTGGTGGTTCCAT |
| | | | CAGCAGTAGTAGTTCCTACTGGGGCTGGATCCGCCAGCCCCCA |
| | | | GGGAAGGGACTGGAGTGGATTGGGAGTATCTATTATAGTGGG |
| | | | AGCACCTACTCCAACCCGTCCCTCAAGAGTCGAGTCACCATAT |
| | | | CCGTAGACACGTCCAAGAATCAGTTCTCCCTGAAGCTGAGCTC |
| | | | TGTGACCGCCGCAGACACGGCTGTGTATTACTGTGCGAGACAG |
| | | | TATATAAGTGGAACTGAATACTTCCAGTACTGGGGCCAGGGCA |
| | | | CCCTGGTCACCGTCTCCTCA |
| 263 | ATX-P-434 | VH-nucleotide | CAGGTGCAGCTGCAGGAGTCGGGCCCAGGACTGGTGAAGCCT |
| | | | TCGGAGACCCTGTCCCTCACCTGCACTGTCTCTGGTGGCTCCGT |
| | | | CAGCAGTGGTAGTTACTACTGGAGCTGGATCCGGCAGCCCCCA |
| | | | GGGAAGGGACTGGAGTGGATTGGGTATATCTATTACAGTGGG |
| | | | AGCACCAGCTACAACCCCTCCCTCAAGAGTCGAGTCTCCATAT |
| | | | CAGTAGACACGTCCAAGAACCAGTTCTCCCTGAAGCTGAGCTC |
| | | | TGTGACCGCTGCGGACACGGCCGTGTTTTACTGTGCGAGAGCT |
| | | | AAGTGGGAAGGGGACTACTTTGACTACTGGGGCCAGGGAACC |
| | | | CTGGTCACCGTCTCCTCA |
| 264 | ATX-P-435 | VL | EIVLTQSPATLSLSPGERATLSCRASQSVSNYLAWYQQKPGQAPR |
| | | | LLIYDASNRATGIPARFSGSGSGTDFTLTISSLEPEDFAVYYCQQRS |
| | | | NWPSFGQGTRLEIK |
| 265 | ATX-P-436 | VL | DIQMTQSPSSLSASVGDRVTITCRASQSISSYLNWYQQKPGKAPK |
| | | | LLIYAASSLQSGVPSRFSGSGSGTDFTLTISSLQPEDFATYYCQQSY |
| | | | STPLTFGGGTKVEIK |
| 266 | ATX-P-434 | VL | DIQMTQSPSSLSASVGDRVTITCRASQSIYSYLNWYQQKPGKAPN |
| | | | HLIYAASSLQSGVPSRFSGSGSGTDFTLTISSLHPEDFATYYCQQS |
| | | | YSTPPYTFGQGTKVEIK |
| 267 | ATX-P-435 | VL--nucleotide | GAAATTGTGTTGACACAGTCTCCAGCCACCCTGTCTTTGTCTCC |
| | | | AGGGGAAAGAGCCACCCTCTCCTGCAGGGCCAGTCAGAGTGT |
| | | | TAGCAACTACTTAGCCTGGTACCAACAGAAACCTGGCCAGGCT |
| | | | CCCAGGCTCCTCATCTATGATGCATCCAACAGGGCCACTGGCA |
| | | | TCCCAGCCAGATTCAGTGGCAGTGGGTCTGGGACAGACTTCAC |
| | | | TCTCACCATCAGCAGCCTGGAGCCTGAAGATTTTGCAGTTTAT |
| | | | TACTGTCAGCAGCGTAGCAACTGGCCCTCCTTCGGCCAAGGGA |
| | | | CACGACTGGAGATTAAA |
| 268 | ATX-P-436 | VL-nucleotide | GACATCCAGATGACCCAGTCTCCATCCTCCCTGTCTGCATCTGT |
| | | | AGGAGACAGAGTCACCATCACTTGCCGGGCAAGTCAGAGCAT |
| | | | TAGCAGCTATTTAAATTGGTATCAGCAGAAACCAGGGAAAGC |
| | | | CCCTAAGCTCCTGATCTATGCTGCATCCAGTTTGCAAAGTGGG |
| | | | GTCCCATCAAGGTTCAGTGGCAGTGGATCTGGGACAGATTTCA |
| | | | CTCTCACCATCAGCAGTCTGCAACCTGAAGATTTTGCAACTTA |
| | | | CTACTGTCAACAGAGTTACAGTACCCCGCTCACTTTCGGCGGA |
| | | | GGGACCAAGGGGAAATCAAA |
| 269 | ATX-P-434 | VL-nucleotide | GACATCCAGATGACCCAGTCTCCATCCTCCCTGTCTGCATCTGT |
| | | | AGGAGACAGAGTCACCATCACTTGCCGGGCAAGTCAGAGCAT |
| | | | TTACAGCTATTTAAATTGGTATCAGCAGAAACCAGGGAAAGCC |
| | | | CCTAATCACCTGATCTATGCTGCATCCAGTTTGCAAAGTGGGG |
| | | | TCCCATCAAGGTTCAGTGGCAGTGGATCTGGGACAGATTTCAC |
| | | | TCTCACCATCAGCAGTCTGCACCCTGAAGATTTTGCAACTTAC |
| | | | TACTGTCAACAGAGTTACAGTACCCCTCCGTACACTTTTGGCC |
| | | | AGGGGACCAAGGTGGAAATCAAA |

TABLE 10
Anti-ANGPTL7 antibodies (HC IgG1 Fc and LC Kappa sequences)

| SEQ | | | |
|---|---|---|---|
| ID | Antibody | | |
| NO: | Name: | Descriptor: | Sequence: |
| 270 | ATX-P-428 | Variable HC + Constant | QVQLVQSGAEVKKPGASVKVSCKASGYTFSSYGISWVRQA |
| | | hIgG1-Agly-Fc | PGQGLEWMGWISAYNGNTNYAQNLRGRVAMTTDTSTTTAY |
| | | | MELRSLRSDDTAVYYCARVSLVWFGELPRGFDYWGQGTLV |
| | | | TVSSASTKGPSVFPLAPSSKSTSGGTAALGCLVKDYFPEP |
| | | | VTVSWNSGALTSGVHTFPAVLQSSGLYSLSSVVTVPSSSL |
| | | | GTQTYICNVNHKPSNTKVDKRVEPKSCDKTHTCPPCPAPE |
| | | | LLGGPSVFLFPPKPKDTLMISRTPEVTCVVVDVSHEDPEV |
| | | | KFNWYVDGVEVHNAKTKPREEQYASTYRVVSVLTVLHQDW |
| | | | LNGKEYKCKVSNKALPAPIEKTISKAKGQPREPQVYTLPP |
| | | | SREEMTKNQVSLTCLVKGFYPSDIAVEWESNGQPENNYKT |
| | | | TPPVLDSDGSFFLYSKLTVDKSRWQQGNVFSCSVMHEALH |
| | | | NHYTQKSLSLSPG |
| 271 | ATX-P-443 | Variable HC + Constant | QVQLVQSGAEVKKPGASVKVSCKASGYTFTSYGISWVRQA |
| | | hIgG1-Agly-Fc | PGQGLEWMGWIIAYNGNTNYAQKLQGRITMTTNTSTNTAY |
| | | | MELRSLRSDDTAVYYCGRDTGFSFDYWGQGTLVTVSSAST |
| | | | KGPSVFPLAPSSKSTSGGTAALGCLVKDYFPEPVTVSWNS |
| | | | GALTSGVHTFPAVLQSSGLYSLSSVVTVPSSSLGTQTYIC |
| | | | NVNHKPSNTKVDKRVEPKSCDKTHTCPPCPAPELLGGPSV |
| | | | FLFPPKPKDTLMISRTPEVTCVVVDVSHEDPEVKFNWYVD |
| | | | GVEVHNAKTKPREEQYASTYRVVSVLTVLHQDWLNGKEYK |
| | | | CKVSNKALPAPIEKTISKAKGQPREPQVYTLPPSREEMTK |
| | | | NQVSLTCLVKGFYPSDIAVEWESNGQPENNYKTTPPVLDS |
| | | | DGSFFLYSKLTVDKSRWQQGNVFSCSVMHEALHNHYTQKS |
| | | | LSLSPG |
| 272 | ATX-P-429 | Variable HC + Constant | QVQLVQSGAEVKKPGASVKVSCKASGYTFSSYGISWVRQA |
| | | hIgG1-Agly-Fc | PGQGLEWMGWISAYNGNTNYAQNLRGRVAMTTDTSTTTAY |
| | | | MELRSLRSDDTAVYYCARVSLVWFGELPRGFDYWGQGTLV |
| | | | TVSSASTKGPSVFPLAPSSKSTSGGTAALGCLVKDYFPEP |
| | | | VTVSWNSGALTSGVHTFPAVLQSSGLYSLSSVVTVPSSSL |
| | | | GTQTYICNVNHKPSNTKVDKRVEPKSCDKTHTCPPCPAPE |
| | | | LLGGPSVFLFPPKPKDTLMISRTPEVTCVVVDVSHEDPEV |
| | | | KFNWYVDGVEVHNAKTKPREEQYASTYRVVSVLTVLHQDW |
| | | | LNGKEYKCKVSNKALPAPIEKTISKAKGQPREPQVYTLPP |
| | | | SREEMTKNQVSLTCLVKGFYPSDIAVEWESNGQPENNYKT |
| | | | TPPVLDSDGSFFLYSKLTVDKSRWQQGNVFSCSVMHEALH |
| | | | NHYTQKSLSLSPG |
| 273 | ATX-P-431 | Variable HC + Constant | QVQLVQSGAEVKKPGASVKVSCKASGYTFTDYYIHWVRQA |
| | | hIgG1-Agly-Fc | PGPGLEWLGWINPNTGATKYAQKFQGRVTMTRDTSISTAY |
| | | | MELSRLRSDDTAVYYWAREDNGNYGDVFDIWGQGTMVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKEN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHNHY |
| | | | TQKSLSLSPG |
| 274 | ATX-P-432 | Variable HC + Constant | QVQLVQSGAEVKKPGASVKVSCKASGYTFTDYYIHWVRQA |
| | | hIgG1-Agly-Fc | PGPGLEWLGWINPNTGATKYAQKFQGRVTMTRDTSISTAY |
| | | | MELSRLRSDDTAVYYCAREDNGNYGDVFDIWGQGTMVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKEN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHNHY |
| | | | TQKSLSLSPG |
| 275 | ATX-P-428 | Variable HC + Constant | CAGGTTCAGCTGGTGCAGTCTGGAGCTGAGGTGAAGAAGC |
| | | hIgG1-Agly-Fc | CTGGGGCCTCAGTGAAGGTCTCCTGCAAGGCTTCTGGTTA |
| | | (nucleotide) | CACCTTTTCCAGCTATGGTATCAGCTGGGTGCGACAGGCC |
| | | | CCTGGACAAGGGCTTGAGTGGATGGGATGGATCAGCGCTT |
| | | | ACAATGGTAACACAAACTATGCACAGAACCTCCGGGGCAG |
| | | | AGTCGCCATGACCACAGACACATCCACGACCACAGCCTAC |
| | | | ATGGAGTTGAGGAGCCTGAGATCTGACGACACGGCCGTGT |
| | | | ATTACTGTGCGAGAGTTTCATTAGTATGGTTTGGGGAATT |
| | | | ACCCAGGGGTTTTGACTACTGGGGCCAGGGAACCCTGGTC |
| | | | ACCGTCTCCTCAGCTAGCACTAAAGGGCCTTCTGTATTTC |
| | | | CCTTGGCCCCGTCCAGCAAATCGACCTCGGGAGGGACAGC |
| | | | CGCCCTGGGTTGCCTTGTGAAAGATTATTTCCCTGAGCCA |
| | | | GTTACCGTAAGTTGGAACAGTGGGGCGCTGACAAGTGGTG |
| | | | TGCACACGTTTCCTGCCGTCCTGCAATCATCGGGCTTGTA |
| | | | TAGCCTCAGCTCTGTGGTCACTGTCCCAAGTTCATCGCTG |
| | | | GGCACTCAGACGTATATTTGCAATGTGAACCACAAACCTT |
| | | | CAAATACAAAAGTGGATAAACGCGTAGAACCGAAATCGTG |
| | | | TGATAAAACTCACACATGCCCGCCATGCCCGGCACCTGAA |
| | | | CTGCTTGGTGGTCCCAGCGTGTTCCTGTTCCCGCCGAAGC |
| | | | CTAAAGATACTCTAATGATCAGCCGTACGCCAGAGGTGAC |
| | | | ATGTGTCGTGGTTGACGTGTCCCACGAAGATCCCGAAGTT |
| | | | AAGTTCAATTGGTATGTTGATGGTGTAGAGGTACACAATG |
| | | | CTAAGACTAAACCTCGCGAGGAGCAGTACGCCTCGACCTA |
| | | | TCGTGTCGTGAGCGTTCTGACCGTCCTTCACCAAGATTGG |
| | | | CTTAACGGCAAAGAATATAAGTGCAAGGTAAGCAATAAAG |
| | | | CACTTCCGGCCCCAATCGAGAAAACCATTICCAAGGCCAA |
| | | | AGGTCAACCAAGAGAACCCCAGGTGTATACTCTTCCGCCT |
| | | | TCTCGTGAGGAAATGACTAAAAATCAAGTATCCCTTACGT |
| | | | GTCTGGTTAAAGGTTTTTATCCTAGCGATATTGCTGTTGA |
| | | | ATGGGAATCGAACGGTCAGCCGGAGAATAATTATAAAACA |
| | | | ACGCCACCCGTCCTGGATAGCGACGGCTCATTTTTTCTGT |
| | | | ATAGCAAACTGACTGTAGATAAATCACGGTGGCAGCAGGG |
| | | | CAATGTATTCAGTTGCTCCGTTATGCATGAAGCGTTACAT |
| | | | AATCACTACACGCAGAAATCTCTTAGTCTTTCACCCGGT |
| 276 | ATX-P-443 | Variable HC + Constant | CAGGTTCAGCTGGTGCAGTCTGGAGCTGAGGTGAAGAAGC |
| | | hIgG1-Agly-Fc | CTGGGGCCTCAGTGAAGGTCTCCTGCAAGGCTTCTGGTTA |
| | | (nucleotide) | CACCTTTACCAGCTATGGTATCAGCTGGGTGCGACAGGCC |
| | | | CCTGGACAAGGGCTTGAGTGGATGGGATGGATCATCGCTT |
| | | | ACAATGGTAACACAAACTATGCACAGAAGCTCCAGGGCAG |
| | | | AATCACCATGACCACAAACACATCCACGAACACAGCCTAC |
| | | | ATGGAGCTGAGGAGCCTGAGATCTGACGACACGGCCGTGT |
| | | | ATTACTGTGGGAGAGATACGGGGTTTTCTTTTGACTACTG |
| | | | GGGCCAGGGAACCCTGGTCACCGTCTCCTCAGCTAGCACT |
| | | | AAAGGGCCTTCTGTATTTCCCTTGGCCCCGTCCAGCAAAT |
| | | | CGACCTCGGGAGGGACAGCCGCCCTGGGTTGCCTTGTGAA |
| | | | AGATTATTTCCCTGAGCCAGTTACCGTAAGTTGGAACAGT |
| | | | GGGGCGCTGACAAGTGGTGTGCACACGTTTCCTGCCGTCC |
| | | | TGCAATCATQGGGCTTGTATAGCCTCAGCTCTGTGGTCAC |
| | | | TGTCCCAAGTTCATCGCTGGGCACTCAGACGTATATTTGC |
| | | | AATGTGAACCACAAACCTTCAAATACAAAAGTGGATAAAC |
| | | | GCGTAGAACCGAAATCGTGTGATAAAACTCACACATGCCC |
| | | | GCCATGCCCGGCACCTGAACTGCTTGGTGGTCCCAGCGTG |
| | | | TTCCTGTTCCCGCCGAAGCCTAAAGATACTCTAATGATCA |
| | | | GCCGTACGCCAGAGGTGACATGTGTCGTGGTTGACGTGTC |
| | | | CCACGAAGATCCCGAAGTTAAGTTCAATTGGTATGTTGAT |
| | | | GGTGTAGAGGTACACAATGCTAAGACTAAACCTCGCGAGG |
| | | | AGCAGTACGCCTCGACCTATCGTGTCGTGAGQGTTCTGAC |
| | | | CGTCCTTCACCAAGATTGGCTTAACGGCAAAGAATATAAG |
| | | | TGCAAGGTAAGCAATAAAGCACTTCCGGCCCCAATCGAGA |
| | | | AAACCATTTCCAAGGCCAAAGGTCAACCAAGAGAACCCCA |
| | | | GGTGTATACTCTTCCGCCTTCTCGTGAGGAAATGACTAAA |
| | | | AATCAAGTATCCCTTACGTGTCTGGTTAAAGGTTTTTATC |
| | | | CTAGCGATATTGCTGTTGAATGGGAATCGAACGGTCAGCC |
| | | | GGAGAATAATTATAAAACAACGCCACCCGTCCTGGATAGC |
| | | | GACGGCTCATTTTTTCTGTATAGCAAACTGACTGTAGATA |
| | | | AATCACGGTGGCAGCAGGGCAATGTATTCAGTTGCTCCGT |
| | | | TATGCATGAAGCGTTACATAATCACTACACGCAGAAATCT |
| | | | CTTAGTCTTTCACCCGGT |
| 277 | ATX-P-429 | Variable HC + Constant | CAGGTTCAGCTGGTGCAGTCTGGAGCTGAGGTGAAGAAGC |
| | | hIgG1-Agly-Fc | CTGGGGCCTCAGTGAAGGTCTCCTGCAAGGCTTCTGGTTA |
| | | (nucleotide) | CACCTTTTCCAGCTATGGTATCAGCTGGGTGCGACAGGCC |
| | | | CCTGGACAAGGGCTTGAGTGGATGGGATGGATCAGCGCTT |
| | | | ACAATGGTAACACAAACTATGCACAGAACCTCCGGGGCAG |
| | | | AGTCGCCATGACCACAGACACATCCACGACCACAGCCTAC |
| | | | ATGGAGTTGAGGAGCCTGAGATCTGACGACACGGCCGTGT |
| | | | ATTACTGTGCGAGAGTTTCATTAGTATGGTTTGGGGAATT |
| | | | ACCCAGGGGTTTTGACTACTGGGGCCAGGGAACCCTGGTC |
| | | | ACCGTCTCCTCAGCTAGCACTAAAGGGCCTTCTGTATTTC |
| | | | CCTTGGCCCCGTCCAGCAAATCGACCTCGGGAGGGACAGC |
| | | | CGCCCTGGGTTGCCTTGTGAAAGATTATTTCCCTGAGCCA |
| | | | GTTACCGTAAGITGGAACAGTGGGGCGCTGACAAGTGGTG |
| | | | TGCACACGTTTCCTGCCGTCCTGCAATCATCGGGCTTGTA |
| | | | TAGCCTCAGCTCTGTGGTCACTGTCCCAAGTTCATCGCTG |
| | | | GGCACTCAGACGTATATTTGCAATGTGAACCACAAACCTT |
| | | | CAAATACAAAAGTGGATAAACGCGTAGAACCGAAATCGTG |
| | | | TGATAAAACTCACACATGCCCGCCATGCCCGGCACCTGAA |
| | | | CTGCTTGGTGGTCCCAGCGTGTTCCTGTTCCCGCCGAAGC |
| | | | CTAAAGATACTCTAATGATCAGCCGTACGCCAGAGGTGAC |
| | | | ATGTGTCGTGGTTGACGTGTCCCACGAAGATCCCGAAGTT |
| | | | AAGTTCAATTGGTATGTTGATGGTGTAGAGGTACACAATG |
| | | | CTAAGACTAAACCTCGCGAGGAGCAGTACGCCTCGACCTA |
| | | | TQGTGTCGTGAGCGTTCTGACCGTCCTTCACCAAGATTGG |
| | | | CTTAACGGCAAAGAATATAAGTGCAAGGTAAGCAATAAAG |
| | | | CACTTCCGGCCCCAATCGAGAAAACCATTICCAAGGCCAA |
| | | | AGGTCAACCAAGAGAACCCCAGGTGTATACTCTTCCGCCT |
| | | | TCTCGTGAGGAAATGACTAAAAATCAAGTATCCCTTACGT |
| | | | GTCTGGTTAAAGGTTTTTATCCTAGCGATATTGCTGTTGA |
| | | | ATGGGAATCGAACGGTCAGCCGGAGAATAATTATAAAACA |
| | | | ACGCCACCCGTCCTGGATAGCGACGGCTCATTTTTTCTGT |
| | | | ATAGCAAACTGACTGTAGATAAATCACGGTGGCAGCAGGG |
| | | | CAATGTATTCAGTTGCTCCGTTATGCATGAAGCGTTACAT |
| | | | AATCACTACACGCAGAAATCTCTTAGTCTTTCACCCGGT |
| 278 | ATX-P-431 | Variable HC + Constant | CAGGTGCAGCTGGTGCAGTCTGGGGCTGAGGTGAAGAAGC |
| | | hIgG1-Agly-Fc | CTGGGGCCTCAGTGAAGGTCTCCTGCAAGGCTTCTGGATA |
| | | (nucleotide) | CACCTTCACCGACTACTATATACACTGGGTGCGACAGGCC |
| | | | CCTGGGCCAGGGCTTGAGTGGTTGGGCTGGATCAACCCTA |
| | | | ACACTGGTGCCACAAAGTATGCACAGAAGTTTCAGGGCAG |
| | | | GGTCACCATGACCAGGGACACGTCCATCAGCACAGCCTAT |
| | | | ATGGAGCTGAGCAGGCTGAGATCTGACGACACGGCCGTAT |
| | | | ATTATTGGGCGAGAGAAGATAATGGGAACTACGGGGATGT |
| | | | TTTTGATATCTGGGGACAAGGGACAATGGTCACCGTCTCT |
| | | | TCAGCTAGCACTAAAGGGCCTTCTGTATTTCCCTTGGCCC |
| | | | CGTCCAGCAAATCGACCTCGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTICCAAGGCCAAAGGTCAACC |
| | | | AAGAGAACCCCAGGTGTATACTCTTCCGCCTTCTCGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGCGTTACATAATCACTAC |
| | | | ACGCAGAAATCTCTTAGTCTTTCACCCGGT |
| 279 | ATX-P-432 | Variable HC + Constant | CAGGTGCAGCTGGTGCAGTCTGGGGCTGAGGTGAAGAAGC |
| | | hIgG1-Agly-Fc | CTGGGGCCTCAGTGAAGGTCTCCTGCAAGGCTTCTGGATA |
| | | (nucleotide) | CACCTTCACCGACTACTATATACACTGGGTGCGACAGGCC |
| | | | CCTGGGCCAGGGCTTGAGTGGTTGGGCTGGATCAACCCTA |
| | | | ACACTGGTGCCACAAAGTATGCACAGAAGTTTCAGGGCAG |
| | | | GGTCACCATGACCAGGGACACGTCCATCAGCACAGCCTAT |
| | | | ATGGAGCTGAGCAGGCTGAGATCTGACGACACGGCCGTAT |
| | | | ATTATTGTGCGAGAGAAGATAATGGGAACTACGGGGATGT |
| | | | TTTTGATATCTGGGGACAAGGGACAATGGTCACCGTCTCT |
| | | | TCAGCTAGCACTAAAGGGCCTTCTGTATTTCCCTTGGCCC |
| | | | CGTCCAGCAAATCGACCTCGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTTCCAAGGCCAAAGGTCAACC |
| | | | AAGAGAACCCCAGGTGTATACTCTTCCGCCTTCTCGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGCGTTACATAATCACTAC |
| | | | ACGCAGAAATCTCTTAGTCTTTCACCCGGT |
| 280 | ATX-P-428 | Variable LC + Kappa | EIVLTQSPGTLSLSPGERATLSCRASQSVSSSYLAWYQQK |
| | | Constant | PGQAPRLLIYGASSRATGIPDRFSGSGSGTDFTLTISRLE |
| | | | PEDFAVYYCQQYGSSPPTFGGGTKVEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 281 | ATX-P-443 | Variable LC + Kappa | EIVLTQSPGILSLSPGERATLSCRASQSVSSSYLAWYQQK |
| | | Constant | PGQAPRLLIYGASSRATGIPDRFSGSGSGTDFTLTISRLE |
| | | | PEDFAVYYCQQYGSSLTFGGGTKVDIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 282 | ATX-P-429 | Variable LC + Kappa | DIVMTQSPLSLPVTPGEPASISCRSSQSLLHSNRYNYLDW |
| | | Constant | YLQKPGQSPQLLIYLGSNRASGVPDRFSGSGSGTDFTLQI |
| | | | SRVEAEDVGVYYCMQTLQTPYTFGQGTKLEIKRTVAAPSV |
| | | | FIFPPSDEQLKSGTASVVCLLNNFYPREAKVQWKVDNALQ |
| | | | SGNSQESVTEQDSKDSTYSLSSTLTLSKADYEKHKVYACE |
| | | | VTHQGLSSPVTKSFNRGEC |
| 283 | ATX-P-431 | Variable LC + Kappa | EIVLTQSPGTLSLSPGERATLSCRASQSVSSSYLAWYQQK |
| | | Constant | PGQAPRLLIYGASSRATGIPDRFSGSGSGTDFTLTISRLE |
| | | | PEDFAVYYCQQYGSSPPTFGGGTKVEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 284 | ATX-P-432 | Variable LC + Kappa | EIVLTQSPGTLSLSPGERATLSCRASQSVSSSYLAWYQQK |
| | | Constant | PGQAPRLLIYGASSRATGIPDRFSGSGSGTDFTLTISRLE |
| | | | PEDFAVYYCQQYGSSPPTFGGGTKVEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 285 | ATX-P-428 | Variable LC + Kappa | GAAATTGTGTTGACGCAGTCTCCAGGCACCCTGTCTTTGT |
| | | Constant (nucleotide) | CTCCAGGGGAAAGAGCCACCCTCTCCTGCAGGGCCAGTCA |
| | | | GAGTGTTAGCAGCAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACCTCCCACTTTCGGCGGAGGGACCAAGGTGGAGAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 286 | ATX-P-443 | Variable LC + Kappa | GAAATTGTGTTGACGCAGTCTCCAGGCACCCTGTCTTTGT |
| | | Constant (nucleotide) | CTCCAGGGGAAAGAGCCACCCTCTCCTGCAGGGCCAGTCA |
| | | | GAGTGTTAGCAGCAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACTCACTTTCGGCGGAGGGACCAAAGTGGATATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 287 | ATX-P-429 | Variable LC + Kappa | GATATTGTGATGACTCAGTCTCCACTCTCCCTGCCCGTCA |
| | | Constant (nucleotide) | CCCCTGGAGAGCCGGCCTCCATCTCCTGCAGGTCTAGTCA |
| | | | GAGCCTCCTGCATAGTAATAGATACAACTATTTGGATTGG |
| | | | TACCTGCAGAAGCCAGGGCAGTCTCCACAGCTCCTGATCT |
| | | | ATTTGGGTTCTAATCGGGCCTCCGGGGTCCCTGACAGGTT |
| | | | CAGTGGCAGTGGATCAGGCACAGATTTTACACTGCAAATC |
| | | | AGCAGAGTGGAGGCTGAGGATGTTGGGGTTTATTACTGCA |
| | | | TGCAAACTCTACAAACTCCGTACACTTTTGGCCAGGGGAC |
| | | | CAAGCTGGAGATCAAACGTACGGTAGCTGCCCCTTCAGTT |
| | | | TTTATCTTTCCGCCGTCTGACGAGCAGTTAAAATCCGGGA |
| | | | CCGCTTCTGTAGITTGCCTGCTGAATAATTTTTATCCGCG |
| | | | TGAGGCTAAAGTACAATGGAAAGTCGACAATGCTTTGCAG |
| | | | TCGGGAAATTCACAGGAAAGTGTTACGGAGCAGGATTCTA |
| | | | AAGATTCCACATATTCACTCAGCTCCACCCTTACACTGAG |
| | | | CAAAGCCGACTATGAAAAACATAAAGITTACGCATGTGAG |
| | | | GTGACGCACCAAGGATTATCCAGTCCGGTCACAAAATCGT |
| | | | TTAACCGCGGTGAGTGT |
| 288 | ATX-P-431 | Variable LC + Kappa | GAAATTGTGTTGACGCAGTCTCCAGGCACCCTGTCTTTGT |
| | | Constant (nucleotide) | CTCCAGGGGAAAGAGCCACCCTCTCCTGCAGGGCCAGTCA |
| | | | GAGTGTTAGCAGCAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACCTCCCACTTTCGGCGGAGGGACCAAGGTGGAAAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 289 | ATX-P-432 | Variable LC + Kappa | GAAATTGTGTTGACGCAGTCTCCAGGCACCCTGTCTTTGT |
| | | Constant (nucleotide) | CTCCAGGGGAAAGAGCCACCCTCTCCTGCAGGGCCAGTCA |
| | | | GAGTGTTAGCAGCAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACCTCCCACTTTCGGCGGAGGGACCAAGGTGGAAAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 290 | ATX-P-437 | Variable HC + Constant | QITLKESGPTLVKPTQTLTLTCTFSGFSLSTSGVGVGWIR |
| | | hIgG1-Agly-Fc | QPPGKALEWLALIYWNDDKQYSPSLKSRLTITKDTSKNQV |
| | | | VLTMTNMDPMDTATYYCAHNPDYGDFFDYWGQGTLVTVSS |
| | | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKRVEPKSCDKTHTCPPCPAPELLGG |
| | | | PSVFLFPPKPKDTLMISRTPEVTCVVVDVSHEDPEVKFNW |
| | | | YVDGVEVHNAKTKPREEQYASTYRVVSVLTVLHQDWLNGK |
| | | | EYKCKVSNKALPAPIEKTISKAKGQPREPQVYTLPPSREE |
| | | | MTKNQVSLTCLVKGFYPSDIAVEWESNGQPENNYKTTPPV |
| | | | LDSDGSFFLYSKLTVDKSRWQQGNVFSCSVMHEALHNHYT |
| | | | QKSLSLSPG |
| 291 | ATX-P-442 | Variable HC + Constant | QITLKESGPTLVKPTQTLTLTCTFSGFSLSTSGVGVGWIR |
| | | hIgG1-Agly-Fc | QPPGKALEWLALIYWNDDKRYSPSLKSRLTITKDTSKNLV |
| | | | VLTMTNMDPVDTATYYCARDYGDYWFFDLWGRGTPVTVSS |
| | | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKRVEPKSCDKTHTCPPCPAPELLGG |
| | | | PSVFLFPPKPKDTLMISRTPEVTCVVVDVSHEDPEVKFNW |
| | | | YVDGVEVHNAKTKPREEQYASTYRVVSVLTVLHQDWLNGK |
| | | | EYKCKVSNKALPAPIEKTISKAKGQPREPQVYTLPPSREE |
| | | | MTKNQVSLTCLVKGFYPSDIAVEWESNGQPENNYKTTPPV |
| | | | LDSDGSFFLYSKLTVDKSRWQQGNVFSCSVMHEALHNHYT |
| | | | QKSLSLSPG |
| 292 | ATX-P-437 | Variable HC + Constant | CAGATCACCTTGAAGGAGTCTGGTCCTACGCTGGTGAAAC |
| | | hIgG1-Agly-Fc | CCACACAGACCCTCACGTTGACCTGCACCTTCTCTGGGTT |
| | | (nucleotide) | CTCACTCAGCACTAGTGGCGTGGGTGTGGGCTGGATCCGT |
| | | | CAGCCCCCAGGAAAGGCCCTGGAGTGGCTTGCACTCATTT |
| | | | ATTGGAATGATGATAAACAGTACAGCCCTTCTCTGAAGAG |
| | | | CAGGCTCACCATCACCAAGGACACCTCCAAAAACCAGGTG |
| | | | GTCCTTACAATGACCAACATGGACCCTATGGACACAGCCA |
| | | | CATATTACTGTGCACACAACCCTGACTACGGTGACTTCTT |
| | | | TGACTACTGGGGCCAGGGAACCCTGGTCACCGTCTCCTCA |
| | | | GCTAGCACTAAAGGGCCTTCTGTATTTCCCTTGGCCCCGT |
| | | | CCAGCAAATCGACCTCGGGAGGGACAGCCGCCCTGGGTTG |
| | | | CCTTGTGAAAGATTATTTCCCTGAGCCAGTTACCGTAAGT |
| | | | TGGAACAGTGGGGCGCTGACAAGTGGTGTGCACACGTTTC |
| | | | CTGCCGTCCTGCAATCATCGGGCTTGTATAGCCTCAGCTC |
| | | | TGTGGTCACTGTCCCAAGTTCATCGCTGGGCACTCAGACG |
| | | | TATATTTGCAATGTGAACCACAAACCTTCAAATACAAAAG |
| | | | TGGATAAACGCGTAGAACCGAAATCGTGTGATAAAACTCA |
| | | | CACATGCCCGCCATGCCCGGCACCTGAACTGCTTGGTGGT |
| | | | CCCAGCGTGTTCCTGTTCCCGCCGAAGCCTAAAGATACTC |
| | | | TAATGATCAGCCGTACGCCAGAGGTGACATGTGTCGTGGT |
| | | | TGACGTGTCCCACGAAGATCCCGAAGTTAAGTTCAATTGG |
| | | | TATGTTGATGGTGTAGAGGTACACAATGCTAAGACTAAAC |
| | | | CTCGCGAGGAGCAGTACGCCTCGACCTATCGTGTCGTGAG |
| | | | CGTTCTGACCGTCCTTCACCAAGATTGGCTTAACGGCAAA |
| | | | GAATATAAGTGCAAGGTAAGCAATAAAGCACTTCCGGCCC |
| | | | CAATCGAGAAAACCATTICCAAGGCCAAAGGTCAACCAAG |
| | | | AGAACCCCAGGTGTATACTCTTCCGCCTTCTCGTGAGGAA |
| | | | ATGACTAAAAATCAAGTATCCCTTACGTGTCTGGTTAAAG |
| | | | GTTTTTATCCTAGCGATATTGCTGTTGAATGGGAATCGAA |
| | | | CGGTCAGCCGGAGAATAATTATAAAACAACGCCACCCGTC |
| | | | CTGGATAGCGACGGCTCATTTTTTCTGTATAGCAAACTGA |
| | | | CTGTAGATAAATCACGGTGGCAGCAGGGCAATGTATTCAG |
| | | | TTGCTCCGTTATGCATGAAGCGTTACATAATCACTACACG |
| | | | CAGAAATCTCTTAGTCTTTCACCCGGT |
| 293 | ATX-P-442 | Variable HC + Constant | CAGATCACCTTGAAGGAGTCTGGTCCTACGCTGGTGAAAC |
| | | hIgG1-Agly-Fc | CCACACAGACCCTCACGCTGACCTGCACCTTCTCTGGGTT |
| | | (nucleotide) | CTCACTCAGCACTAGTGGAGTGGGTGTGGGCTGGATCCGT |
| | | | CAGCCCCCAGGAAAGGCCCTGGAGTGGCTTGCACTCATTT |
| | | | ATTGGAATGATGATAAGCGCTACAGCCCATCTCTGAAGAG |
| | | | CAGGCTCACCATCACCAAGGACACCTCCAAAAACCTGGTG |
| | | | GTCCTTACAATGACCAACATGGACCCTGTGGACACAGCCA |
| | | | CATATTACTGTGCACGTGACTACGGTGACTACTGGTTCTT |
| | | | CGATCTCTGGGGCCGTGGCACCCCGGTCACCGTCTCCTCA |
| | | | GCTAGCACTAAAGGGCCTTCTGTATTTCCCTTGGCCCCGT |
| | | | CCAGCAAATCGACCTQGGGAGGGACAGCCGCCCTGGGTTG |
| | | | CCTTGTGAAAGATTATTTCCCTGAGCCAGTTACCGTAAGT |
| | | | TGGAACAGTGGGGCGCTGACAAGTGGTGTGCACACGTTTC |
| | | | CTGCCGTCCTGCAATCATCGGGCTTGTATAGCCTCAGCTC |
| | | | TGTGGTCACTGTCCCAAGTTCATCGCTGGGCACTCAGACG |
| | | | TATATTTGCAATGTGAACCACAAACCTTCAAATACAAAAG |
| | | | TGGATAAACGCGTAGAACCGAAATCGTGTGATAAAACTCA |
| | | | CACATGCCCGCCATGCCCGGCACCTGAACTGCTTGGTGGT |
| | | | CCCAGCGTGTTCCTGTTCCCGCCGAAGCCTAAAGATACTC |
| | | | TAATGATCAGCCGTACGCCAGAGGTGACATGTGTCGTGGT |
| | | | TGACGTGTCCCACGAAGATCCCGAAGTTAAGTTCAATTGG |
| | | | TATGTTGATGGTGTAGAGGTACACAATGCTAAGACTAAAC |
| | | | CTCGCGAGGAGCAGTACGCCTCGACCTATCGTGTCGTGAG |
| | | | CGTTCTGACCGTCCTTCACCAAGATTGGCTTAACGGCAAA |
| | | | GAATATAAGTGCAAGGTAAGCAATAAAGCACTTCCGGCCC |
| | | | CAATCGAGAAAACCATTTCCAAGGCCAAAGGTCAACCAAG |
| | | | AGAACCCCAGGTGTATACTCTTCCGCCTTCTCGTGAGGAA |
| | | | ATGACTAAAAATCAAGTATCCCTTACGTGTCTGGTTAAAG |
| | | | GTTTTTATCCTAGCGATATTGCTGTTGAATGGGAATCGAA |
| | | | CGGTCAGCCGGAGAATAATTATAAAACAACGCCACCCGTC |
| | | | CTGGATAGCGACGGCTCATTTTTTCTGTATAGCAAACTGA |
| | | | CTGTAGATAAATCACGGTGGCAGCAGGGCAATGTATTCAG |
| | | | TTGCTCCGTTATGCATGAAGCGTTACATAATCACTACACG |
| | | | CAGAAATCTCTTAGTCTTTCACCCGGT |
| 294 | ATX-P-437 | Variable LC + Kappa | DIQMTQSPSSLSASVGDRVTITCRASQSISSYLNWYQQKP |
| | | Constant | GKAPKLLIYAASSLQSGVPSRFSGSGSGTDFILTISSLQP |
| | | | EDFATYYCQQSFSTPLTFGGGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 295 | ATX-P-442 | Variable LC + Kappa | DIQMTQSPSSLSASVGDRVTITCRASQGISSYLAWYQQKP |
| | | Constant | GKAPKLLIYAASSLQSGVPSRFSGSGSGTDFTLTISSLQP |
| | | | EDFATYYCQQSFSTPLTFGGGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 296 | ATX-P-437 | Variable LC + Kappa | GACATCCAGATGACCCAGTCTCCATCCTCCCTGTCTGCAT |
| | | Constant (nucleotide) | CTGTAGGAGACAGAGTCACCATCACTTGCCGGGCAAGTCA |
| | | | GAGCATTAGCAGCTATTTAAATTGGTATCAGCAGAAACCA |
| | | | GGGAAAGCCCCTAAGCTCCTGATCTATGCTGCATCCAGTT |
| | | | TGCAAAGTGGGGTCCCATCAAGGTTCAGTGGCAGTGGATC |
| | | | TGGGACAGATTTCATTCTCACCATCAGCAGTCTGCAACCT |
| | | | GAAGATTTTGCAACTTACTACTGTCAACAGAGTTTCAGTA |
| | | | CCCCGCTCACTTTCGGCGGAGGGACCAAGCTGGAAATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 297 | ATX-P-442 | Variable LC + Kappa | GACATCCAGATGACCCAGTCTCCATCCTCCCTGTCTGCAT |
| | | Constant (nucleotide) | CTGTAGGAGACAGAGTCACCATCACTTGCCGGGCCAGTCA |
| | | | GGGCATTAGCAGTTATTTAGCCTGGTATCAGCAAAAACCA |
| | | | GGGAAAGCCCCTAAGCTCCTGATCTATGCTGCATCCAGTT |
| | | | TGCAAAGTGGGGTCCCATCAAGGTTCAGTGGCAGTGGATC |
| | | | TGGGACAGATTTCACTCTCACCATCAGCAGTCTGCAACCT |
| | | | GAAGATTTTGCAACTTACTACTGTCAACAGAGTTTCAGTA |
| | | | CCCCGCTCACTTTCGGCGGAGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 298 | ATX-P-444 | Variable HC + Constant | EVQLVESGGGLVQPGGSLRLSCAASGFTLSVYDMHWVRQV |
| | | hIgG1-Agly-Fc | SGKGLEWVSGIDPDGDTYYPGSVKGRFTISRENAKNSLYL |
| | | | QMNSLRVGDTAVYYCLRGEESNSSEDGFDIWGQGTMVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHNHY |
| | | | TQKSLSLSPG |
| 299 | ATX-P-445 | Variable HC + Constant | EVQLVESGGGLVQPGGSLRLSCAASGFTFSSHDMHWVRQV |
| | | hIgG1-Agly-Fc | TGKGLEWVSGIDPAGDTYYPDSVKGRFTISRENAKNSLYL |
| | | | QMNSLRAGDTAVYYCTRGEASSSSEDAFDIWGQGTMVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKEN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHNHY |
| | | | TQKSLSLSPG |
| 300 | ATX-P-446 | Variable HC + Constant | EVQLVESGGGLVQPGGSLRLSCAASGFTLSSYDMHWVRQV |
| | | higG1-Agly-Fc | IGKGLEWVSGIDPDGDTYYPGSVKGRFTISREDAKNSLYL |
| | | | QMNSLRAGDTAVYYCTRGEDNSSSEDAFDIWGQGTMVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKEN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHNHY |
| | | | TQKSLSLSPG |
| 301 | ATX-P-447 | Variable HC + Constant | EVQLVESGGGLVQPGGSLRLSCAASGFTISDYDMHWVRQT |
| | | hIgG1-Agly-Fc | TGKGLEWVSGIDPDGDTYYPGSLKGRFTISRENAKNSLYL |
| | | | QMNSLRAGDTAVYYCTRGEDSSSSEDAFDIWGQGTMVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHINH |
| | | | YTQKSLSLSPG |
| 302 | ATX-P-427 | Variable HC + Constant | EVQLVESGGGVVRPGGSLRLSCAASGFSFDDYGMSWVRQV |
| | | hIgG1-Agly-Fc | PGKGLEWVSGINWNGGSTGYADSVKGRFTISRDNAKNSLY |
| | | | LQMSSLRAEDTALYYCASYSGSFRDAFDIWGQGTMVTVSS |
| | | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKRVEPKSCDKTHTCPPCPAPELLGG |
| | | | PSVFLFPPKPKDTLMISRTPEVTCVVVDVSHEDPEVKFNW |
| | | | YVDGVEVHNAKTKPREEQYASTYRVVSVLTVLHQDWLNGK |
| | | | EYKCKVSNKALPAPIEKTISKAKGQPREPQVYTLPPSREE |
| | | | MTKNQVSLTCLVKGFYPSDIAVEWESNGQPENNYKTTPPV |
| | | | LDSDGSFFLYSKLTVDKSRWQQGNVFSCSVMHEALHNHYT |
| | | | QKSLSLSPG |
| 303 | ATX-P-440 | Variable HC + Constant | EVQLVESGGGVVRPGGSLRLSCAASGFTFDDYGMSWVRQA |
| | | hIgG1-Agly-Fc | PGKGLEWVSTINWNGGNTGYADSVKGRFTISRDNAKNSLY |
| | | | LQMNSLRAEDTALYYCARDEGAAGLGYYFDYWGQGTLVTV |
| | | | SSASTKGPSVFPLAPSSKSTSGGTAALGCLVKDYFPEPVT |
| | | | VSWNSGALTSGVHTFPAVLQSSGLYSLSSVVTVPSSSLGT |
| | | | QTYICNVNHKPSNTKVDKRVEPKSCDKTHTCPPCPAPELL |
| | | | GGPSVFLFPPKPKDTLMISRTPEVTCVVVDVSHEDPEVKF |
| | | | NWYVDGVEVHNAKTKPREEQYASTYRVVSVLTVLHQDWLN |
| | | | GKEYKCKVSNKALPAPIEKTISKAKGQPREPQVYTLPPSR |
| | | | EEMTKNQVSLTCLVKGFYPSDIAVEWESNGQPENNYKTTP |
| | | | PVLDSDGSFFLYSKLTVDKSRWQQGNVFSCSVMHEALHNH |
| | | | YTQKSLSLSPG |
| 304 | ATX-P-424 | Variable HC + Constant | EVQLVESGGGLVKPGGSLRLSCAASGFTFSSYSMNWVRQA |
| | | higG1-Agly-Fc | PGKGLEWVSSITSSSYIFYADSLKGRFTISRDNAKNSLFL |
| | | | QMNSLRAEDTAVYYCARDSNWGEAFDIWGQGTMVTVSSAS |
| | | | TKGPSVFPLAPSSKSTSGGTAALGCLVKDYFPEPVTVSWN |
| | | | SGALTSGVHTFPAVLQSSGLYSLSSVVTVPSSSLGTQTYI |
| | | | CNVNHKPSNTKVDKRVEPKSCDKTHTCPPCPAPELLGGPS |
| | | | VFLFPPKPKDTLMISRTPEVTCVVVDVSHEDPEVKFNWYV |
| | | | DGVEVHNAKTKPREEQYASTYRVVSVLTVLHQDWLNGKEY |
| | | | KCKVSNKALPAPIEKTISKAKGQPREPQVYTLPPSREEMT |
| | | | KNQVSLTCLVKGFYPSDIAVEWESNGQPENNYKTTPPVLD |
| | | | SDGSFFLYSKLTVDKSRWQQGNVFSCSVMHEALHNHYTQK |
| | | | SLSLSPG |
| 305 | ATX-P-425 | Variable HC + Constant | EVQLVESGGGLVKPGGSLRLSCAASGFTFSTYSMNWVRQA |
| | | hIgG1-Agly~Fc | PGKGLEWVSSITSSSSYMFYADSVMGRFTISRDNAKNSLY |
| | | | LQMNSLRAEDTADYYCARDSNWGEAFDIWGQGTMVTVSSA |
| | | | STKGPSVFPLAPSSKSTSGGTAALGCLVKDYFPEPVTVSW |
| | | | NSGALTSGVHTFPAVLQSSGLYSLSSVVTVPSSSLGTQTY |
| | | | ICNVNHKPSNTKVDKRVEPKSCDKTHTCPPCPAPELLGGP |
| | | | SVFLFPPKPKDTLMISRTPEVTCVVVDVSHEDPEVKFNWY |
| | | | VDGVEVHNAKTKPREEQYASTYRVVSVLTVLHQDWLNGKE |
| | | | YKCKVSNKALPAPIEKTISKAKGQPREPQVYTLPPSREEM |
| | | | TKNQVSLTCLVKGFYPSDIAVEWESNGQPENNYKTTPPVL |
| | | | DSDGSFFLYSKLTVDKSRWQQGNVFSCSVMHEALHNHYTQ |
| | | | KSLSLSPG |
| 306 | ATX-P-430 | Variable HC + Constant | EVQLVESGGGLVQPGGSLRLSCAASGFTFSSYAMSWVRQA |
| | | hIgG1-Agly-Fc | PGKGLEWVSAISGSGGSTYYADSVKGRFTISRDNSKNTLY |
| | | | LQMNSLRAEDTAVYYCAKDGDYYYYYMDVWGKGTTVTVSS |
| | | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKRVEPKSCDKTHTCPPCPAPELLGG |
| | | | PSVFLFPPKPKDTLMISRTPEVTCVVVDVSHEDPEVKENW |
| | | | YVDGVEVHNAKTKPREEQYASTYRVVSVLTVLHQDWLNGK |
| | | | EYKCKVSNKALPAPIEKTISKAKGQPREPQVYTLPPSREE |
| | | | MTKNQVSLTCLVKGFYPSDIAVEWESNGQPENNYKTTPPV |
| | | | LDSDGSFFLYSKLTVDKSRWQQGNVFSCSVMHEALHNHYT |
| | | | QKSLSLSPG |
| 307 | ATX-P-439 | Variable HC + Constant | EVQLVESGGGLVQPGGSLRLSCAASGFTFSSYAMSWVRQA |
| | | hIgG1-Agly-Fc | PGKGLEWVSAISGSGGSTYYADSVKGRFTISRDNSKNTLY |
| | | | LQMNSLRAEDTAVYYCAKVERDMVRGHYYYYMDVWGKGTT |
| | | | VTVSSASTKGPSVFPLAPSSKSTSGGTAALGCLVKDYFPE |
| | | | PVTVSWNSGALTSGVHTFPAVLQSSGLYSLSSVVTVPSSS |
| | | | LGTQTYICNVNHKPSNTKVDKRVEPKSCDKTHTCPPCPAP |
| | | | ELLGGPSVFLFPPKPKDTLMISRTPEVTCVVVDVSHEDPE |
| | | | VKFNWYVDGVEVHNAKTKPREEQYASTYRVVSVLTVLHQD |
| | | | WLNGKEYKCKVSNKALPAPIEKTISKAKGQPREPQVYTLP |
| | | | PSREEMTKNQVSLTCLVKGFYPSDIAVEWESNGQPENNYK |
| | | | TTPPVLDSDGSFFLYSKLTVDKSRWQQGNVFSCSVMHEAL |
| | | | HNHYTQKSLSLSPG |
| 308 | ATX-P-423 | Variable HC + Constant | QVQLVESGGGVVQPGRSLRLSCAASGFTFSSYGMHWVRQA |
| | | hIgG1-Agly-Fc | PGKGLEWVSHITYDGSDKYYGDSVKGRFTISRDNSKNTLY |
| | | | LQMNSLRAEDTAVYYCAKDGYSSGWWYFDYWGQGTLVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHNHY |
| | | | TQKSLSLSPG |
| 309 | ATX-P-438 | Variable HC + Constant | QVQLVESGGGVVQPGRSLRLSCAASGFTFSSYGMHWVRQA |
| | | bigG1-Agly-Fc | PGKGLEWVAIISYDGNNKYYADSVKGRFTISRDNSKNTLY |
| | | | LQMNSLRDEDTAVYYCAKERGYSSSSGYYYYYMDVWGKGT |
| | | | TVTVSSASTKGPSVFPLAPSSKSTSGGTAALGCLVKDYFP |
| | | | EPVTVSWNSGALTSGVHTFPAVLQSSGLYSLSSVVTVPSS |
| | | | SLGTQTYICNVNHKPSNTKVDKRVEPKSCDKTHTCPPCPA |
| | | | PELLGGPSVFLFPPKPKDTLMISRTPEVTCVVVDVSHEDP |
| | | | EVKFNWYVDGVEVHNAKTKPREEQYASTYRVVSVLTVLHQ |
| | | | DWLNGKEYKCKVSNKALPAPIEKTISKAKGQPREPQVYTL |
| | | | PPSREEMTKNQVSLTCLVKGFYPSDIAVEWESNGQPENNY |
| | | | KTTPPVLDSDGSFFLYSKLTVDKSRWQQGNVFSCSVMHEA |
| | | | LHNHYTQKSLSLSPG |
| 310 | ATX-P-448 | Variable HC + Constant | QVQLVESGGGVVQPGRSLRLSCAASGFPFSSYGMHWVRQA |
| | | hIgG1-Agly-Fc | PGKGLEWVAVMSYDGINGYYADSVKGRFTISRDNSKNTLY |
| | | | LQMNSLRAEDTAVYYCARLVYADDAFDIWGQGTTVTVSSA |
| | | | STKGPSVFPLAPSSKSTSGGTAALGCLVKDYFPEPVTVSW |
| | | | NSGALTSGVHTFPAVLQSSGLYSLSSVVTVPSSSLGTQTY |
| | | | ICNVNHKPSNTKVDKRVEPKSCDKTHTCPPCPAPELLGGP |
| | | | SVFLFPPKPKDTLMISRTPEVTCVVVDVSHEDPEVKFNWY |
| | | | VDGVEVHNAKTKPREEQYASTYRVVSVLTVLHQDWLNGKE |
| | | | YKCKVSNKALPAPIEKTISKAKGQPREPQVYTLPPSREEM |
| | | | TKNQVSLTCLVKGFYPSDIAVEWESNGQPENNYKTTPPVL |
| | | | DSDGSFFLYSKLTVDKSRWQQGNVFSCSVMHEALHNHYTQ |
| | | | KSLSLSPG |
| 311 | ATX-P-449 | Variable HC + Constant | QVQLVESGGGVVQPGRSLRLSCAASGFAFSSYGMHWVRQA |
| | | hIgG1-Agly-Fc | PGKGLEWVAVISYYGSNKYYADSVKGRFTISRDNSKNTLY |
| | | | LQMNSLRAEDTAVYHCARGITMDVWGKGTTVTVSSASTKG |
| | | | PSVFPLAPSSKSTSGGTAALGCLVKDYFPEPVTVSWNSGA |
| | | | LTSGVHTFPAVLQSSGLYSLSSVVTVPSSSLGTQTYICNV |
| | | | NHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKCK |
| | | | VSNKALPAPIEKTISKAKGQPREPQVYTLPPSREEMTKNQ |
| | | | VSLTCLVKGFYPSDIAVEWESNGQPENNYKTTPPVLDSDG |
| | | | SFFLYSKLTVDKSRWQQGNVFSCSVMHEALHNHYTQKSLS |
| | | | LSPG |
| 312 | ATX-P-433 | Variable HC + Constant | EVQLVESGGGLVQPGRSLRLSCAASGFTFDDFAMHWVRQG |
| | | hIgG1-Agly-Fc | PGKGLEWVSGIGWNSGSIGYADSVKGRFTISRDNAKNSLY |
| | | | LQMNSLRAEDTALYYCAKAPDYGDYYFDYWGQGTLVTVSS |
| | | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKRVEPKSCDKTHTCPPCPAPELLGG |
| | | | PSVFLFPPKPKDTLMISRTPEVTCVVVDVSHEDPEVKFNW |
| | | | YVDGVEVHNAKTKPREEQYASTYRVVSVLTVLHQDWLNGK |
| | | | EYKCKVSNKALPAPIEKTISKAKGQPREPQVYTLPPSREE |
| | | | MTKNQVSLTCLVKGFYPSDIAVEWESNGQPENNYKTTPPV |
| | | | LDSDGSFFLYSKLTVDKSRWQQGNVFSCSVMHEALHNHYT |
| | | | QKSLSLSPG |
| 313 | ATX-P-444 | Variable HC + Constant | GAGGTGCAGCTGGTGGAGTCGGGGGGAGGCTTGGTACAGC |
| | | hIgG1-Agly-Fc | CTGGGGGGTCCCTGAGACTCTCCTGTGCAGCCTCTGGATT |
| | | (nucleotide) | CACCCTCAGTGTCTACGACATGCACTGGGTCCGCCAAGTT |
| | | | TCAGGAAAAGGTCTGGAGTGGGTCTCAGGTATTGATCCTG |
| | | | ATGGTGACACATACTATCCCGGCTCCGTGAAGGGCCGATT |
| | | | CACCATCTCCAGAGAAAATGCCAAGAACTCCTTGTATCTT |
| | | | CAAATGAACAGCCTGAGAGTCGGGGACACGGCTGTGTATT |
| | | | ATTGTCTAAGAGGGGAGGAAAGCAACTCGTCCGAGGATGG |
| | | | TTTTGATATATGGGGACAAGGGACAATGGTCACCGTCTCC |
| | | | TCAGCTAGCACTAAAGGGCCTTCTGTATTTCCCTTGGCCC |
| | | | CGTCCAGCAAATCGACCTCGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTICCAAGGCCAAAGGTCAACC |
| | | | AAGAGAACCCCAGGTGTATACTCTTCCGCCTTCTCGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGCGTTACATAATCACTAC |
| | | | ACGCAGAAATCTCTTAGTCTTTCACCCGGT |
| 314 | ATX-P-445 | Variable HC + Constant | GAGGTGCAGCTGGTGGAGTCTGGGGGAGGCTTGGTACAGC |
| | | hIgG1-Agly-Fc | CTGGGGGGTCCCTGAGACTCTCCTGTGCAGCCTCTGGATT |
| | | (nucleotide) | CACCTTCAGTAGCCACGACATGCACTGGGTCCGCCAAGTT |
| | | | ACAGGAAAAGGTCTGGAGTGGGTCTCAGGTATTGATCCTG |
| | | | CTGGTGACACATACTATCCAGACTCCGTGAAGGGCCGATT |
| | | | CACCATCTCTAGAGAAAATGCCAAGAACTCCTTGTATCTT |
| | | | CAAATGAACAGCCTGAGAGCCGGGGACACGGCTGTGTATT |
| | | | ACTGTACAAGAGGAGAGGCTAGTAGTTCGTCCGAGGATGC |
| | | | TTTTGATATCTGGGGACAAGGGACAATGGTCACTGTCTCC |
| | | | TCAGCTAGCACTAAAGGGCCTTCTGTATTTCCCTTGGCCC |
| | | | CGTCCAGCAAATCGACCTCGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTTCCAAGGCCAAAGGTCAACC |
| | | | AAGAGAACCCCAGGTGTATACTCTTCCGCCTTCTQGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGCGTTACATAATCACTAC |
| | | | ACGCAGAAATCTCTTAGTCTTTCACCCGGT |
| 315 | ATX-P-446 | Variable HC + Constant | GAGGTGCAGCTGGTGGAGTCTGGGGGAGGCTTGGTACAGC |
| | | hIgG1-Agly-Fc | CTGGGGGGTCCCTGAGACTCTCCTGTGCAGCCTCTGGATT |
| | | (nucleotide) | CACCCTCAGTAGCTACGACATGCACTGGGTCCGCCAAGTT |
| | | | ATAGGAAAAGGTCTGGAGTGGGTCTCAGGTATTGATCCTG |
| | | | ATGGTGACACATACTATCCAGGCTCCGTGAAGGGCCGATT |
| | | | CACCATCTCCAGAGAAGATGCCAAGAACTCCTTGTATCTT |
| | | | CAAATGAACAGCCTGAGAGCCGGGGACACGGCTGTGTATT |
| | | | ACTGTACAAGAGGAGAGGATAACAGCTCGTCCGAGGATGC |
| | | | TTTTGATATCTGGGGACAAGGGACAATGGTCACTGTCTCC |
| | | | TCAGCTAGCACTAAAGGGCCTTCTGTATTTCCCTTGGCCC |
| | | | CGTCCAGCAAATCGACCTCGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTICCAAGGCCAAAGGTCAACC |
| | | | AAGAGAACCCCAGGTGTATACTCTTCCGCCTTCTCGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGCGTTACATAATCACTAC |
| | | | ACGCAGAAATCTCTTAGTCTTTCACCCGGT |
| 316 | ATX-P-447 | Variable HC + Constant | GAGGTCCAGCTGGTGGAGTCTGGGGGAGGCTTGGTACAGC |
| | | hIgG1-Agly-Fc | CGGGGGGGTCCCTGAGACTCTCCTGTGCAGCCTCTGGATT |
| | | (nucleotide) | CACCATCAGTGACTACGACATGCACTGGGTCCGCCAAACT |
| | | | ACAGGAAAAGGTCTAGAATGGGTCTCAGGTATTGATCCTG |
| | | | ATGGCGACACATATTATCCAGGCTCCTTGAAGGGGCGATT |
| | | | CACCATCTCCAGAGAAAATGCCAAGAATTCTTTGTATCTT |
| | | | CAAATGAACAGTCTGAGAGCCGGGGACACGGCTGTGTATT |
| | | | ACTGTACAAGAGGAGAGGATAGTAGTTCGTCCGAGGATGC |
| | | | TTTTGATATCTGGGGACAAGGGACAATGGTCACCGTCTCC |
| | | | TCAGCTAGCACTAAAGGGCCTTCTGTATTTCCCTTGGCCC |
| | | | CGTCCAGCAAATCGACCTQGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTTCCAAGGCCAAAGGTCAACC |
| | | | AAGAGAACCCCAGGTGTATACTCTTCCGCCTTCTCGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGCGTTACATAATCACTAC |
| | | | ACGCAGAAATCTCTTAGTCTTTCACCCGGT |
| 317 | ATX-P-427 | Variable HC + Constant | GAGGTGCAGCTGGTGGAGTCTGGGGGAGGTGTGGTACGGC |
| | | hIgG1-Agly-Fc | CTGGGGGGTCCCTGAGACTCTCCTGTGCAGCCTCTGGATT |
| | | (nucleotide) | CAGCTTTGATGATTATGGCATGAGCTGGGTCCGCCAAGTT |
| | | | CCAGGGAAGGGGCTGGAGTGGGTCTCTGGTATTAATTGGA |
| | | | ATGGTGGTAGTACAGGTTATGCAGACTCTGTGAAGGGCCG |
| | | | ATTCACCATCTCCAGAGACAACGCCAAGAACTCCCTGTAT |
| | | | CTGCAAATGAGCAGTCTGAGAGCCGAGGACACGGCCTTGT |
| | | | ATTACTGTGCGAGCTATAGTGGGAGCTTCCGTGATGCTTT |
| | | | TGATATCTGGGGACAAGGGACAATGGTCACCGTCTCTTCA |
| | | | GCTAGCACTAAAGGGCCTTCTGTATTTCCCTTGGCCCCGT |
| | | | CCAGCAAATCGACCTCGGGAGGGACAGCCGCCCTGGGTTG |
| | | | CCTTGTGAAAGATTATTTCCCTGAGCCAGTTACCGTAAGT |
| | | | TGGAACAGTGGGGCGCTGACAAGTGGTGTGCACACGTTTC |
| | | | CTGCCGTCCTGCAATCATCGGGCTTGTATAGCCTCAGCTC |
| | | | TGTGGTCACTGTCCCAAGTTCATCGCTGGGCACTCAGACG |
| | | | TATATTTGCAATGTGAACCACAAACCTTCAAATACAAAAG |
| | | | TGGATAAACGCGTAGAACCGAAATCGTGTGATAAAACTCA |
| | | | CACATGCCCGCCATGCCCGGCACCTGAACTGCTTGGTGGT |
| | | | CCCAGCGTGTTCCTGTTCCCGCCGAAGCCTAAAGATACTC |
| | | | TAATGATCAGCCGTACGCCAGAGGTGACATGTGTCGTGGT |
| | | | TGACGTGTCCCACGAAGATCCCGAAGTTAAGTTCAATTGG |
| | | | TATGTTGATGGTGTAGAGGTACACAATGCTAAGACTAAAC |
| | | | CTCGCGAGGAGCAGTACGCCTCGACCTATCGTGTCGTGAG |
| | | | CGTTCTGACCGTCCTTCACCAAGATTGGCTTAACGGCAAA |
| | | | GAATATAAGTGCAAGGTAAGCAATAAAGCACTTCCGGCCC |
| | | | CAATCGAGAAAACCATTTCCAAGGCCAAAGGTCAACCAAG |
| | | | AGAACCCCAGGTGTATACTCTTCCGCCTTCTCGTGAGGAA |
| | | | ATGACTAAAAATCAAGTATCCCTTACGTGTCTGGTTAAAG |
| | | | GTTTTTATCCTAGCGATATTGCTGTTGAATGGGAATCGAA |
| | | | CGGTCAGCCGGAGAATAATTATAAAACAACGCCACCCGTC |
| | | | CTGGATAGCGACGGCTCATTTTTTCTGTATAGCAAACTGA |
| | | | CTGTAGATAAATCACGGTGGCAGCAGGGCAATGTATTCAG |
| | | | TTGCTCCGTTATGCATGAAGCGTTACATAATCACTACACG |
| | | | CAGAAATCTCTTAGTCTTTCACCCGGT |
| 318 | ATX-P-440 | Variable HC + Constant | GAGGTGCAGCTGGTGGAGTCTGGGGGAGGTGTGGTACGGC |
| | | hIgG1-Agly-Fc | CTGGGGGGTCCCTGAGACTCTCCTGTGCAGCCTCTGGATT |
| | | (nucleotide) | CACCTTTGATGATTATGGCATGAGCTGGGTCCGCCAAGCT |
| | | | CCAGGGAAGGGGCTGGAGTGGGTCTCTACTATTAATTGGA |
| | | | ATGGTGGTAACACAGGTTATGCAGACTCTGTGAAGGGCCG |
| | | | ATTCACCATCTCCAGAGACAACGCCAAGAACTCCCTGTAT |
| | | | CTGCAAATGAACAGTCTGAGAGCCGAGGACACGGCCTTGT |
| | | | ATTACTGTGCGAGAGATGAAGGAGCAGCTGGACTTGGATA |
| | | | CTACTTTGACTACTGGGGCCAGGGAACCCTGGTCACCGTC |
| | | | TCCTCAGCTAGCACTAAAGGGCCTTCTGTATTTCCCTTGG |
| | | | CCCCGTCCAGCAAATCGACCTCGGGAGGGACAGCCGCCCT |
| | | | GGGTTGCCTTGTGAAAGATTATTTCCCTGAGCCAGTTACC |
| | | | GTAAGTTGGAACAGTGGGGCGCTGACAAGTGGTGTGCACA |
| | | | CGTTTCCTGCCGTCCTGCAATCATCGGGCTTGTATAGCCT |
| | | | CAGCTCTGTGGTCACTGTCCCAAGTTCATCGCTGGGCACT |
| | | | CAGACGTATATTTGCAATGTGAACCACAAACCTTCAAATA |
| | | | CAAAAGTGGATAAACGCGTAGAACCGAAATCGTGTGATAA |
| | | | AACTCACACATGCCCGCCATGCCCGGCACCTGAACTGCTT |
| | | | GGTGGTCCCAGCGTGTTCCTGTTCCCGCCGAAGCCTAAAG |
| | | | ATACTCTAATGATCAGCCGTACGCCAGAGGTGACATGTGT |
| | | | CGTGGTTGACGTGTCCCACGAAGATCCCGAAGTTAAGTTC |
| | | | AATTGGTATGTTGATGGTGTAGAGGTACACAATGCTAAGA |
| | | | CTAAACCTCGCGAGGAGCAGTACGCCTCGACCTATCGTGT |
| | | | CGTGAGCGTTCTGACCGTCCTTCACCAAGATTGGCTTAAC |
| | | | GGCAAAGAATATAAGTGCAAGGTAAGCAATAAAGCACTTC |
| | | | CGGCCCCAATCGAGAAAACCATTICCAAGGCCAAAGGTCA |
| | | | ACCAAGAGAACCCCAGGTGTATACTCTTCCGCCTTCTCGT |
| | | | GAGGAAATGACTAAAAATCAAGTATCCCTTACGTGTCTGG |
| | | | TTAAAGGTTTTTATCCTAGCGATATTGCTGTTGAATGGGA |
| | | | ATCGAACGGTCAGCCGGAGAATAATTATAAAACAACGCCA |
| | | | CCCGTCCTGGATAGCGACGGCTCATTTTTTCTGTATAGCA |
| | | | AACTGACTGTAGATAAATCACGGTGGCAGCAGGGCAATGT |
| | | | ATTCAGTTGCTCCGTTATGCATGAAGCGTTACATAATCAC |
| | | | TACACGCAGAAATCTCTTAGTCTTTCACCCGGT |
| 319 | ATX-P-424 | Variable HC + Constant | GAGGTGCAGCTGGTGGAGTCTGGGGGAGGCCTGGTCAAGC |
| | | hIgG1-Agly-Fc | CTGGGGGGTCCCTGAGACTCTCCTGTGCAGCCTCTGGATT |
| | | (nucleotide) | CACCTTCAGTAGCTATAGCATGAACTGGGTCCGCCAGGCT |
| | | | CCAGGGAAGGGGCTGGAGTGGGTCTCATCCATTACTAGTA |
| | | | GTAGTTACATATTCTACGCAGACTCATTGAAGGGCCGATT |
| | | | CACCATCTCCAGAGACAACGCCAAGAACTCACTGTTTCTG |
| | | | CAAATGAACAGCCTGAGAGCCGAGGACACAGCTGTTTATT |
| | | | ACTGTGCGAGAGATTCTAACTGGGGCGAGGCTTTTGATAT |
| | | | CTGGGGACAAGGGACAATGGTCACCGTCTCTTCAGCTAGC |
| | | | ACTAAAGGGCCTTCTGTATTTCCCTTGGCCCCGTCCAGCA |
| | | | AATCGACCTCGGGAGGGACAGCCGCCCTGGGTTGCCTTGT |
| | | | GAAAGATTATTTCCCTGAGCCAGTTACCGTAAGTTGGAAC |
| | | | AGTGGGGCGCTGACAAGTGGTGTGCACACGTTTCCTGCCG |
| | | | TCCTGCAATCATCGGGCTTGTATAGCCTCAGCTCTGTGGT |
| | | | CACTGTCCCAAGTTCATCGCTGGGCACTCAGACGTATATT |
| | | | TGCAATGTGAACCACAAACCTTCAAATACAAAAGTGGATA |
| | | | AACGCGTAGAACCGAAATCGTGTGATAAAACTCACACATG |
| | | | CCCGCCATGCCCGGCACCTGAACTGCTTGGTGGTCCCAGC |
| | | | GTGTTCCTGTTCCCGCCGAAGCCTAAAGATACTCTAATGA |
| | | | TCAGCCGTACGCCAGAGGTGACATGTGTCGTGGTTGACGT |
| | | | GTCCCACGAAGATCCCGAAGTTAAGTTCAATTGGTATGTT |
| | | | GATGGTGTAGAGGTACACAATGCTAAGACTAAACCTQGCG |
| | | | AGGAGCAGTACGCCTCGACCTATCGTGTCGTGAGCGTTCT |
| | | | GACCGTCCTTCACCAAGATTGGCTTAACGGCAAAGAATAT |
| | | | AAGTGCAAGGTAAGCAATAAAGCACTTCCGGCCCCAATCG |
| | | | AGAAAACCATTTCCAAGGCCAAAGGTCAACCAAGAGAACC |
| | | | CCAGGTGTATACTCTTCCGCCTTCTCGTGAGGAAATGACT |
| | | | AAAAATCAAGTATCCCTTACGTGTCTGGTTAAAGGTTTTT |
| | | | ATCCTAGCGATATTGCTGTTGAATGGGAATCGAACGGTCA |
| | | | GCCGGAGAATAATTATAAAACAACGCCACCCGTCCTGGAT |
| | | | AGCGACGGCTCATTTTTTCTGTATAGCAAACTGACTGTAG |
| | | | ATAAATCACGGTGGCAGCAGGGCAATGTATTCAGTTGCTC |
| | | | CGTTATGCATGAAGCGTTACATAATCACTACACGCAGAAA |
| | | | TCTCTTAGTCTTTCACCCGGT |
| 320 | ATX-P-425 | Variable HC + Constant | GAGGTGCAGCTGGTGGAGTCTGGGGGAGGCCTGGTCAAGC |
| | | hIgG1-Agly-Fc | CTGGGGGGTCCCTGAGACTCTCCTGTGCAGCCTCTGGTTT |
| | | (nucleotide) | CACCTTCAGTACTTATAGCATGAACTGGGTCCGCCAGGCT |
| | | | CCAGGGAAGGGGCTGGAGTGGGTCTCATCCATTACTAGTA |
| | | | GTAGTAGTTACATGTTCTACGCAGACTCAGTGATGGGCCG |
| | | | ATTCACCATCTCCAGAGACAACGCCAAGAACTCACTGTAC |
| | | | CTACAAATGAACAGCCTGAGAGCCGAGGACACAGCTGATT |
| | | | ATTACTGTGCGAGAGATTCTAATTGGGGGGAGGCTTTTGA |
| | | | TATCTGGGGACAAGGGACAATGGTCACCGTCTCTTCAGCT |
| | | | AGCACTAAAGGGCCTTCTGTATTTCCCTTGGCCCCGTCCA |
| | | | GCAAATCGACCTCGGGAGGGACAGCCGCCCTGGGTTGCCT |
| | | | TGTGAAAGATTATTTCCCTGAGCCAGTTACCGTAAGTTGG |
| | | | AACAGTGGGGCGCTGACAAGTGGTGTGCACACGTTTCCTG |
| | | | CCGTCCTGCAATCATCGGGCTTGTATAGCCTCAGCTCTGT |
| | | | GGTCACTGTCCCAAGTTCATCGCTGGGCACTCAGACGTAT |
| | | | ATTTGCAATGTGAACCACAAACCTTCAAATACAAAAGTGG |
| | | | ATAAACGCGTAGAACCGAAATCGTGTGATAAAACTCACAC |
| | | | ATGCCCGCCATGCCCGGCACCTGAACTGCTTGGTGGTCCC |
| | | | AGCGTGTTCCTGTTCCCGCCGAAGCCTAAAGATACTCTAA |
| | | | TGATCAGCCGTACGCCAGAGGTGACATGTGTCGTGGTTGA |
| | | | CGTGTCCCACGAAGATCCCGAAGTTAAGTTCAATTGGTAT |
| | | | GTTGATGGTGTAGAGGTACACAATGCTAAGACTAAACCTC |
| | | | GCGAGGAGCAGTACGCCTCGACCTATCGTGTCGTGAGCGT |
| | | | TCTGACCGTCCTTCACCAAGATTGGCTTAACGGCAAAGAA |
| | | | TATAAGTGCAAGGTAAGCAATAAAGCACTTCCGGCCCCAA |
| | | | TCGAGAAAACCATTICCAAGGCCAAAGGTCAACCAAGAGA |
| | | | ACCCCAGGTGTATACTCTTCCGCCTTCTCGTGAGGAAATG |
| | | | ACTAAAAATCAAGTATCCCTTACGTGTCTGGTTAAAGGTT |
| | | | TTTATCCTAGCGATATTGCTGTTGAATGGGAATCGAACGG |
| | | | TCAGCCGGAGAATAATTATAAAACAACGCCACCQGTCCTG |
| | | | GATAGCGACGGCTCATTTTTTCTGTATAGCAAACTGACTG |
| | | | TAGATAAATCACGGTGGCAGCAGGGCAATGTATTCAGTTG |
| | | | CTCCGTTATGCATGAAGCGTTACATAATCACTACACGCAG |
| | | | AAATCTCTTAGTCTTTCACCCGGT |
| 321 | ATX-P-430 | Variable HC + Constant | GAGGTGCAGCTGGTGGAGTCTGGGGGAGGCTTGGTACAGC |
| | | higG1-Agly-Fc | CTGGGGGGTCCCTGAGACTCTCCTGTGCAGCCTCTGGATT |
| | | (nucleotide) | CACCTTTAGCAGCTATGCCATGAGCTGGGTCCGCCAGGCT |
| | | | CCAGGGAAGGGGCTGGAGTGGGTCTCAGCTATTAGTGGTA |
| | | | GTGGTGGTAGCACATACTACGCAGACTCCGTGAAGGGCCG |
| | | | GTTCACCATCTCCAGAGACAATTCCAAGAACACGCTGTAT |
| | | | CTGCAAATGAACAGCCTGAGAGCCGAGGACACGGCCGTAT |
| | | | ATTACTGTGCGAAAGACGGTGACTACTACTACTACTACAT |
| | | | GGACGTCTGGGGCAAAGGGACCACGGTCACCGTCTCCTCA |
| | | | GCTAGCACTAAAGGGCCTTCTGTATTTCCCTTGGCCCCGT |
| | | | CCAGCAAATCGACCTCGGGAGGGACAGCCGCCCTGGGTTG |
| | | | CCTTGTGAAAGATTATTTCCCTGAGCCAGTTACCGTAAGT |
| | | | TGGAACAGTGGGGCGCTGACAAGTGGTGTGCACACGTTTC |
| | | | CTGCCGTCCTGCAATCATCGGGCTTGTATAGCCTCAGCTC |
| | | | TGTGGTCACTGTCCCAAGTTCATCGCTGGGCACTCAGACG |
| | | | TATATTTGCAATGTGAACCACAAACCTTCAAATACAAAAG |
| | | | TGGATAAACGCGTAGAACCGAAATCGTGTGATAAAACTCA |
| | | | CACATGCCCGCCATGCCCGGCACCTGAACTGCTTGGTGGT |
| | | | CCCAGCGTGTTCCTGTTCCCGCCGAAGCCTAAAGATACTC |
| | | | TAATGATCAGCCGTACGCCAGAGGTGACATGTGTCGTGGT |
| | | | TGACGTGTCCCACGAAGATCCCGAAGTTAAGTTCAATTGG |
| | | | TATGTTGATGGTGTAGAGGTACACAATGCTAAGACTAAAC |
| | | | CTCGCGAGGAGCAGTACGCCTCGACCTATCGTGTCGTGAG |
| | | | CGTTCTGACCGTCCTTCACCAAGATTGGCTTAACGGCAAA |
| | | | GAATATAAGTGCAAGGTAAGCAATAAAGCACTTCCGGCCC |
| | | | CAATCGAGAAAACCATTTCCAAGGCCAAAGGTCAACCAAG |
| | | | AGAACCCCAGGTGTATACTCTTCCGCCTTCTCGTGAGGAA |
| | | | ATGACTAAAAATCAAGTATCCCTTACGTGTCTGGTTAAAG |
| | | | GTTTTTATCCTAGCGATATTGCTGTTGAATGGGAATCGAA |
| | | | CGGTCAGCCGGAGAATAATTATAAAACAACGCCACCCGTC |
| | | | CTGGATAGCGACGGCTCATTTTTTCTGTATAGCAAACTGA |
| | | | CTGTAGATAAATCACGGTGGCAGCAGGGCAATGTATTCAG |
| | | | TTGCTCCGTTATGCATGAAGCGTTACATAATCACTACACG |
| | | | CAGAAATCTCTTAGTCTTTCACCCGGT |
| 322 | ATX-P-439 | Variable HC + Constant | GAGGTGCAGCTGGTGGAGTCTGGGGGAGGCTTGGTACAGC |
| | | higG1-Agly-Fc | CTGGGGGGTCCCTGAGACTCTCCTGTGCAGCCTCTGGATT |
| | | (nucleotide) | CACCTTTAGCAGCTATGCCATGAGCTGGGTCCGCCAGGCT |
| | | | CCAGGGAAGGGGCTGGAGTGGGTCTCAGCTATTAGTGGTA |
| | | | GTGGTGGTAGCACATACTACGCAGACTCCGTGAAGGGCCG |
| | | | GTTCACCATCTCCAGAGACAATTCCAAGAACACGCTGTAT |
| | | | CTGCAAATGAACAGCCTGAGAGCCGAGGACACGGCCGTAT |
| | | | ATTACTGTGCGAAAGTGGAAAGGGATATGGTTCGGGGCCA |
| | | | CTACTACTACTACATGGACGTCTGGGGCAAAGGGACCACG |
| | | | GTCACCGTCTCTTCAGCTAGCACTAAAGGGCCTTCTGTAT |
| | | | TTCCCTTGGCCCCGTCCAGCAAATCGACCTCGGGAGGGAC |
| | | | AGCCGCCCTGGGTTGCCTTGTGAAAGATTATTTCCCTGAG |
| | | | CCAGTTACCGTAAGTTGGAACAGTGGGGCGCTGACAAGTG |
| | | | GTGTGCACACGTTTCCTGCCGTCCTGCAATCATCGGGCTT |
| | | | GTATAGCCTCAGCTCTGTGGTCACTGTCCCAAGTTCATCG |
| | | | CTGGGCACTCAGACGTATATTTGCAATGTGAACCACAAAC |
| | | | CTTCAAATACAAAAGTGGATAAACGCGTAGAACCGAAATC |
| | | | GTGTGATAAAACTCACACATGCCCGCCATGCCCGGCACCT |
| | | | GAACTGCTTGGTGGTCCCAGCGTGTTCCTGTTCCCGCCGA |
| | | | AGCCTAAAGATACTCTAATGATCAGCCGTACGCCAGAGGT |
| | | | GACATGTGTCGTGGTTGACGTGTCCCACGAAGATCCCGAA |
| | | | GTTAAGTTCAATTGGTATGTTGATGGTGTAGAGGTACACA |
| | | | ATGCTAAGACTAAACCTCGCGAGGAGCAGTACGCCTCGAC |
| | | | CTATCGTGTCGTGAGCGTTCTGACCGTCCTTCACCAAGAT |
| | | | TGGCTTAACGGCAAAGAATATAAGTGCAAGGTAAGCAATA |
| | | | AAGCACTTCCGGCCCCAATCGAGAAAACCATTICCAAGGC |
| | | | CAAAGGTCAACCAAGAGAACCCCAGGTGTATACTCTTCCG |
| | | | CCTTCTCGTGAGGAAATGACTAAAAATCAAGTATCCCTTA |
| | | | CGTGTCTGGTTAAAGGTTTTTATCCTAGCGATATTGCTGT |
| | | | TGAATGGGAATCGAACGGTCAGCCGGAGAATAATTATAAA |
| | | | ACAACGCCACCCGTCCTGGATAGCGACGGCTCATTTTTTC |
| | | | TGTATAGCAAACTGACTGTAGATAAATCACGGTGGCAGCA |
| | | | GGGCAATGTATTCAGTTGCTCCGTTATGCATGAAGCGTTA |
| | | | CATAATCACTACACGCAGAAATCTCTTAGTCTTTCACCCG |
| | | | GT |
| 323 | ATX-P-423 | Variable HC + Constant | CAGGTGCAGCTGGTGGAGTCTGGGGGAGGCGTGGTCCAGC |
| | | hIgG1-Agly-Fc | CTGGGAGGTCCCTGAGACTCTCCTGTGCAGCCTCTGGATT |
| | | (nucleotide) | CACCTTCAGTAGCTATGGCATGCACTGGGTCCGCCAGGCT |
| | | | CCAGGCAAGGGGCTGGAGTGGGTGTCACATATAACATATG |
| | | | ATGGAAGTGATAAATACTATGGAGACTCCGTGAAGGGCCG |
| | | | ATTCACCATCTCCAGAGACAATTCCAAGAATACGCTGTAT |
| | | | CTGCAAATGAACAGCCTGAGAGCTGAGGACACGGCTGTGT |
| | | | ATTACTGTGCGAAAGATGGGTATAGCAGTGGCTGGTGGTA |
| | | | CTTTGACTACTGGGGCCAGGGAACCCTGGTCACCGTCTCC |
| | | | TCAGCTAGCACTAAAGGGCCTTCTGTATTTCCCTTGGCCC |
| | | | CGTCCAGCAAATCGACCTCGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTICCAAGGCCAAAGGTCAACC |
| | | | AAGAGAACCCCAGGTGTATACTCTTCCGCCTTCTCGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGQGTTACATAATCACTAC |
| | | | ACGCAGAAATCTCTTAGTCTTTCACCCGGT |
| 324 | ATX-P-438 | Variable HC + Constant | CAGGTGCAGCTGGTGGAGTCCGGGGGAGGCGTGGTCCAGC |
| | | higG1-Agly-Fc | CTGGGAGGTCCCTGAGACTCTCCTGTGCAGCCTCTGGATT |
| | | (nucleotide) | CACCTTCAGTAGCTATGGCATGCACTGGGTCCGCCAGGCT |
| | | | CCAGGCAAGGGGCTGGAGTGGGTGGCAATTATATCATATG |
| | | | ATGGAAATAATAAATACTATGCAGACTCCGTGAAGGGCCG |
| | | | ATTCACCATCTCCAGAGACAATTCCAAGAACACGCTGTAT |
| | | | CTGCAAATGAACAGCCTGAGAGATGAGGACACGGCTGTTT |
| | | | ATTATTGTGCGAAAGAGAGGGGGTATAGCAGCTCGTCCGG |
| | | | CTACTACTACTACTACATGGACGTCTGGGGCAAAGGGACC |
| | | | ACGGTCACCGTCTCCTCAGCTAGCACTAAAGGGCCTTCTG |
| | | | TATTTCCCTTGGCCCCGTCCAGCAAATCGACCTCGGGAGG |
| | | | GACAGCCGCCCTGGGTTGCCTTGTGAAAGATTATTTCCCT |
| | | | GAGCCAGTTACCGTAAGTTGGAACAGTGGGGCGCTGACAA |
| | | | GTGGTGTGCACACGTTTCCTGCCGTCCTGCAATCATCGGG |
| | | | CTTGTATAGCCTCAGCTCTGTGGTCACTGTCCCAAGTTCA |
| | | | TCGCTGGGCACTCAGACGTATATTTGCAATGTGAACCACA |
| | | | AACCTTCAAATACAAAAGTGGATAAACGCGTAGAACCGAA |
| | | | ATCGTGTGATAAAACTCACACATGCCCGCCATGCCCGGCA |
| | | | CCTGAACTGCTTGGTGGTCCCAGCGTGTTCCTGTTCCCGC |
| | | | CGAAGCCTAAAGATACTCTAATGATCAGCCGTACGCCAGA |
| | | | GGTGACATGTGTCGTGGTTGACGTGTCCCACGAAGATCCC |
| | | | GAAGTTAAGTTCAATTGGTATGTTGATGGTGTAGAGGTAC |
| | | | ACAATGCTAAGACTAAACCTCGCGAGGAGCAGTACGCCTC |
| | | | GACCTATCGTGTCGTGAGCGTTCTGACCGTCCTTCACCAA |
| | | | GATTGGCTTAACGGCAAAGAATATAAGTGCAAGGTAAGCA |
| | | | ATAAAGCACTTCCGGCCCCAATCGAGAAAACCATTTCCAA |
| | | | GGCCAAAGGTCAACCAAGAGAACCCCAGGTGTATACTCTT |
| | | | CCGCCTTCTCGTGAGGAAATGACTAAAAATCAAGTATCCC |
| | | | TTACGTGTCTGGTTAAAGGTTTTTATCCTAGCGATATTGC |
| | | | TGTTGAATGGGAATCGAACGGTCAGCCGGAGAATAATTAT |
| | | | AAAACAACGCCACCCGTCCTGGATAGCGACGGCTCATTTT |
| | | | TTCTGTATAGCAAACTGACTGTAGATAAATCACGGTGGCA |
| | | | GCAGGGCAATGTATTCAGTTGCTCCGTTATGCATGAAGCG |
| | | | TTACATAATCACTACACGCAGAAATCTCTTAGTCTTTCAC |
| | | | CCGGT |
| 325 | ATX-P-448 | Variable HC + Constant | CAAGTGCAATTGGTGGAGTCCGGTGGAGGAGTAGTACAGC |
| | | higG1-Agly-Fc | CGGGAAGGTCACTGAGACTTTCCTGCGCTGCTAGTGGATT |
| | | (nucleotide) | CCCATTTAGCTCCTACGGGATGCACTGGGTTCGCCAAGCG |
| | | | CCAGGTAAGGGACTGGAATGGGTGGCTGTCATGAGTTACG |
| | | | ATGGAATTAACGGCTATTATGCCGATAGCGTCAAAGGCCG |
| | | | GTTCACGATCTCAAGAGACAACTCTAAGAACACGCTCTAC |
| | | | TTGCAGATGAACAGTCTCCGCGCAGAAGATACGGCCGTGT |
| | | | ATTACTGTGCGAGATTGGTGTATGCCGATGATGCTTTTGA |
| | | | TATCTGGGGACAAGGGACCACGGTCACCGTCTCCTCAGCT |
| | | | AGCACTAAAGGGCCTTCTGTATTTCCCTTGGCCCCGTCCA |
| | | | GCAAATCGACCTCGGGAGGGACAGCCGCCCTGGGTTGCCT |
| | | | TGTGAAAGATTATTTCCCTGAGCCAGTTACCGTAAGTTGG |
| | | | AACAGTGGGGCGCTGACAAGTGGTGTGCACACGTTTCCTG |
| | | | CCGTCCTGCAATCATCGGGCTTGTATAGCCTCAGCTCTGT |
| | | | GGTCACTGTCCCAAGTTCATCGCTGGGCACTCAGACGTAT |
| | | | ATTTGCAATGTGAACCACAAACCTTCAAATACAAAAGTGG |
| | | | ATAAACGCGTAGAACCGAAATCGTGTGATAAAACTCACAC |
| | | | ATGCCCGCCATGCCCGGCACCTGAACTGCTTGGTGGTCCC |
| | | | AGCGTGTTCCTGTTCCCGCCGAAGCCTAAAGATACTCTAA |
| | | | TGATCAGCCGTACGCCAGAGGTGACATGTGTCGTGGTTGA |
| | | | CGTGTCCCACGAAGATCCCGAAGTTAAGTTCAATTGGTAT |
| | | | GTTGATGGTGTAGAGGTACACAATGCTAAGACTAAACCTC |
| | | | GCGAGGAGCAGTACGCCTCGACCTATCGTGTCGTGAGCGT |
| | | | TCTGACCGTCCTTCACCAAGATTGGCTTAACGGCAAAGAA |
| | | | TATAAGTGCAAGGTAAGCAATAAAGCACTTCCGGCCCCAA |
| | | | TCGAGAAAACCATTICCAAGGCCAAAGGTCAACCAAGAGA |
| | | | ACCCCAGGTGTATACTCTTCCGCCTTCTCGTGAGGAAATG |
| | | | ACTAAAAATCAAGTATCCCTTACGTGTCTGGTTAAAGGTT |
| | | | TTTATCCTAGCGATATTGCTGTTGAATGGGAATCGAACGG |
| | | | TCAGCCGGAGAATAATTATAAAACAACGCCACCCGTCCTG |
| | | | GATAGCGACGGCTCATTTTTTCTGTATAGCAAACTGACTG |
| | | | TAGATAAATCACGGTGGCAGCAGGGCAATGTATTCAGTTG |
| | | | CTCCGTTATGCATGAAGCGTTACATAATCACTACACGCAG |
| | | | AAATCTCTTAGTCTTTCACCCGGT |
| 326 | ATX-P-449 | Variable HC + Constant | CAAGTGCAATTGGTGGAGTCCGGTGGAGGAGTAGTACAGC |
| | | hIgG1-Agly-Fc | CGGGAAGGTCACTGAGACTTTCCTGCGCTGCTAGTGGATT |
| | | (nucleotide) | CGCCTTTAGTTCCTACGGCATGCACTGGGTTCGCCAAGCG |
| | | | CCAGGTAAGGGACTGGAATGGGTGGCTGTCATCTCCTACT |
| | | | ACGGATCTAATAAATATTATGCCGATAGCGTCAAAGGCCG |
| | | | GTTCACGATCTCAAGAGACAACTCTAAGAACACGCTCTAC |
| | | | TTGCAGATGAACAGTCTCQGCGCAGAAGATACGGCCGTGT |
| | | | ATCACTGTGCGAGAGGGATTACTATGGACGTCTGGGGCAA |
| | | | AGGGACCACGGTCACCGTCTCCTCAGCTAGCACTAAAGGG |
| | | | CCTTCTGTATTTCCCTTGGCCCCGTCCAGCAAATCGACCT |
| | | | CGGGAGGGACAGCCGCCCTGGGTTGCCTTGTGAAAGATTA |
| | | | TTTCCCTGAGCCAGTTACCGTAAGTTGGAACAGTGGGGCG |
| | | | CTGACAAGTGGTGTGCACACGTTTCCTGCCGTCCTGCAAT |
| | | | CATCGGGCTTGTATAGCCTCAGCTCTGTGGTCACTGTCCC |
| | | | AAGTTCATCGCTGGGCACTCAGACGTATATTTGCAATGTG |
| | | | AACCACAAACCTTCAAATACAAAAGTGGATAAACGCGTAG |
| | | | AACCGAAATCGTGTGATAAAACTCACACATGCCCGCCATG |
| | | | CCCGGCACCTGAACTGCTTGGTGGTCCCAGCGTGTTCCTG |
| | | | TTCCCGCCGAAGCCTAAAGATACTCTAATGATCAGCCGTA |
| | | | CGCCAGAGGTGACATGTGTCGTGGTTGACGTGTCCCACGA |
| | | | AGATCCCGAAGTTAAGTTCAATTGGTATGTTGATGGTGTA |
| | | | GAGGTACACAATGCTAAGACTAAACCTCGCGAGGAGCAGT |
| | | | ACGCCTCGACCTATCGTGTCGTGAGCGTTCTGACCGTCCT |
| | | | TCACCAAGATTGGCTTAACGGCAAAGAATATAAGTGCAAG |
| | | | GTAAGCAATAAAGCACTTCCGGCCCCAATCGAGAAAACCA |
| | | | TTTCCAAGGCCAAAGGTCAACCAAGAGAACCCCAGGTGTA |
| | | | TACTCTTCCGCCTTCTCGTGAGGAAATGACTAAAAATCAA |
| | | | GTATCCCTTACGTGTCTGGTTAAAGGTTTTTATCCT\AGC |
| | | | GATATTGCTGTTGAATGGGAATCGAACGGTCAGCCGGAGA |
| | | | ATAATTATAAAACAACGCCACCCGTCCTGGATAGCGACGG |
| | | | CTCATTTTTTCTGTATAGCAAACTGACTGTAGATAAATCA |
| | | | CGGTGGCAGCAGGGCAATGTATTCAGTTGCTCCGTTATGC |
| | | | ATGAAGCGTTACATAATCACTACACGCAGAAATCTCTTAG |
| | | | TCTTTCACCCGGT |
| 327 | ATX-P-433 | Variable HC + Constant | GAAGTGCAGCTGGTGGAGTCTGGGGGAGGCTTGGTACAGC |
| | | hIgG1-Agly-Fc | CTGGCAGGTCCCTGAGACTCTCCTGTGCAGCCTCTGGATT |
| | | (nucleotide) | CACCTTTGATGATTTTGCCATGCACTGGGTCCGGCAAGGT |
| | | | CCAGGGAAGGGCCTGGAGTGGGTCTCAGGTATTGGTTGGA |
| | | | ATAGTGGTAGCATCGGCTATGCGGACTCTGTGAAGGGCCG |
| | | | ATTCACCATCTCCAGAGACAACGCCAAGAACTCCCTGTAT |
| | | | CTGCAAATGAACAGTCTGAGAGCTGAGGACACGGCCTTAT |
| | | | ATTACTGTGCAAAAGCCCCTGACTACGGTGACTACTACTT |
| | | | TGACTACTGGGGCCAGGGAACCCTGGTCACCGTCTCCTCA |
| | | | GCTAGCACTAAAGGGCCTTCTGTATTTCCCTTGGCCCCGT |
| | | | CCAGCAAATCGACCTCGGGAGGGACAGCCGCCCTGGGTTG |
| | | | CCTTGTGAAAGATTATTTCCCTGAGCCAGTTACCGTAAGT |
| | | | TGGAACAGTGGGGCGCTGACAAGTGGTGTGCACACGTTTC |
| | | | CTGCCGTCCTGCAATCATCGGGCTTGTATAGCCTCAGCTC |
| | | | TGTGGTCACTGTCCCAAGTTCATCGCTGGGCACTCAGACG |
| | | | TATATTTGCAATGTGAACCACAAACCTTCAAATACAAAAG |
| | | | TGGATAAACGCGTAGAACCGAAATCGTGTGATAAAACTCA |
| | | | CACATGCCCGCCATGCCCGGCACCTGAACTGCTTGGTGGT |
| | | | CCCAGCGTGTTCCTGTTCCCGCCGAAGCCTAAAGATACTC |
| | | | TAATGATCAGCCGTACGCCAGAGGTGACATGTGTCGTGGT |
| | | | TGACGTGTCCCACGAAGATCCCGAAGTTAAGTTCAATTGG |
| | | | TATGTTGATGGTGTAGAGGTACACAATGCTAAGACTAAAC |
| | | | CTCGCGAGGAGCAGTACGCCTCGACCTATCGTGTCGTGAG |
| | | | CGTTCTGACCGTCCTTCACCAAGATTGGCTTAACGGCAAA |
| | | | GAATATAAGTGCAAGGTAAGCAATAAAGCACTTCCGGCCC |
| | | | CAATCGAGAAAACCATTICCAAGGCCAAAGGTCAACCAAG |
| | | | AGAACCCCAGGTGTATACTCTTCCGCCTTCTCGTGAGGAA |
| | | | ATGACTAAAAATCAAGTATCCCTTACGTGTCTGGTTAAAG |
| | | | GTTTTTATCCTAGCGATATTGCTGTTGAATGGGAATCGAA |
| | | | CGGTCAGCCGGAGAATAATTATAAAACAACGCCACCCGTC |
| | | | CTGGATAGCGACGGCTCATTTTTTCTGTATAGCAAACTGA |
| | | | CTGTAGATAAATCACGGTGGCAGCAGGGCAATGTATTCAG |
| | | | TTGCTCCGTTATGCATGAAGCGTTACATAATCACTACACG |
| | | | CAGAAATCTCTTAGTCTTTCACCCGGT |
| 328 | ATX-P-444 | Variable LC + Kappa | DVVMTQSPLSLPVTLGQPASISCRSSQSLVYSDGNTYLNW |
| | | Constant | FQQRPGQSPRRLIYKVSNRDSGVPDRFSGSGSGTDFTLKI |
| | | | SKVEAEDVGIYYCMQGTHWPWTFGQGTKVDIKRTVAAPSV |
| | | | FIFPPSDEQLKSGTASVVCLLNNFYPREAKVQWKVDNALQ |
| | | | SGNSQESVTEQDSKDSTYSLSSTLTLSKADYEKHKVYACE |
| | | | VTHQGLSSPVIKSFNRGEC |
| 329 | ATX-P-445 | Variable LC + Kappa | DVVMTQSPLSLPVTLGQPASISCRSSQSLVYSDGNTYLNW |
| | | Constant | FQQRPGQSPRRLIHEVSNRDSGVPDRFSGSGSGTDFTLKI |
| | | | SRVEAEDVGVYYCMQGTHWPWTFGQGTKVDIKRTVAAPSV |
| | | | FIFPPSDEQLKSGTASVVCLLNNFYPREAKVQWKVDNALQ |
| | | | SGNSQESVTEQDSKDSTYSLSSTLTLSKADYEKHKVYACE |
| | | | VTHQGLSSPVTKSFNRGEC |
| 330 | ATX-P-446 | Variable LC + Kappa | DVVMTQSPLSLPVTLGQPASISCRSSQSLVFSDGNTYLNW |
| | | Constant | FQQRPGQSPRRLIYKVSNRDSGVPDRFSGSGSGTDFTLKI |
| | | | SRVEAEDVGVYYCMQGTHWPWTFGQGTKVDIKRTVAAPSV |
| | | | FIFPPSDEQLKSGTASVVCLLNNFYPREAKVQWKVDNALQ |
| | | | SGNSQESVTEQDSKDSTYSLSSTLTLSKADYEKHKVYACE |
| | | | VTHQGLSSPVTKSFNRGEC |
| 331 | ATX-P-447 | Variable LC + Kappa | DVVMTQSPLSLPVTLGQPASISCRSSQSLVYSDGNTYLNW |
| | | Constant | FQQRPGQSPRRLIHEVSNRDSGVPDRFSGSGSGTDFTLKI |
| | | | SRVEAEDVGVYYCMQGTHWPWTFGQGTKVDIKRTVAAPSV |
| | | | FIFPPSDEQLKSGTASVVCLLNNFYPREAKVQWKVDNALQ |
| | | | SGNSQESVTEQDSKDSTYSLSSTLTLSKADYEKHKVYACE |
| | | | VTHQGLSSPVTKSENRGEC |
| 332 | ATX-P-427 | Variable LC + Kappa | AIQMTQSPSSLSASVGDRVTITCRASQGIRNDLGWYQQKP |
| | | Constant | GKAPKLLIYAASSLQSGVPSRFSGSGSGTDFTLTISSLQP |
| | | | EDFATYYCLQDYNYPYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 333 | ATX-P-440 | Variable LC + Kappa | DIQMTQSPSTLSASVGDRVTITCRASQSISSWLAWYQQKP |
| | | Constant | GKAPKLLIYATSSLQSGVPSRFSGSGSGTDFTLTISSLQP |
| | | | EDFATYYCQQSYSTPITFGQGTRLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 334 | ATX-P-424 | Variable LC + Kappa | DIQMTQSPSSLSASVGDRVTITCRASQGIRNDLGWYQQKP |
| | | Constant | GKAPKRLIYAASSLPSGVPSRFSGSGSGTEFTLTISSLQP |
| | | | EDFAIYYCLQHNSYPWTFGQGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 335 | ATX-P-425 | Variable LC + Kappa | DIQMTQSPSSLSASVGDRVTITCRASQGIRIDLGWFQQKP |
| | | Constant | GKAPKRLIYAASSLPSGVPSRFSGSGSGTEFTLTISSLQP |
| | | | EDFATYYCLQHNSYPWTFGQGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 336 | ATX-P-430 | Variable LC + Kappa | EIVLTQSPGTLSLSPGERATLSCRASQSVSSSYLAWYQQK |
| | | Constant | PGQAPRLLIYGASSRATGIPDRFSGSGSGTDFTLTISRLE |
| | | | PEDFAVYYCQQYGSSPPTFGGGTKVEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 337 | ATX-P-439 | Variable LC + Kappa | DIQMTQSPSSVSASVGDRVTITCRASQGISSWLAWYQQKP |
| | | Constant | GKAPKLLIYAASSLQSGVPSRFSGSGSGTDFTLTISSLQP |
| | | | EDFATYYCQQANSFPWTFGQGTKVDIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 338 | ATX-P-423 | Variable LC + Kappa | DIQMTQSPSSLSASVGDRVTITCRASQGIRNDLGWYQQKP |
| | | Constant | GKAPKRLIYAASSLQSGVPSRFSGSGSGTEFALTISSLQP |
| | | | EDFATYYCLQHNSYPLTFGGGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 339 | ATX-P-438 | Variable LC + Kappa | EIVLTQSPGTLSLSPGERATLSCRASQSVSRSYLAWYQQK |
| | | Constant | PGQAPRLLIIGASTRATGIPDRFSGSGSGTDFTLTISRLE |
| | | | PEDFAVYYCQQGQVIPPTFGQGTKVEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 340 | ATX-P-448 | Variable LC + Kappa | EIVLTQSPATLSLSPGERATLSCRASQSVSSYLAWYQQKP |
| | | Constant | GQAPRLLIYDASNRATGIPARFSGSGSGTDFTLTISSLEP |
| | | | EDFAVYYCQQRSNWPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 341 | ATX-P-449 | Variable LC + Kappa | EIVLTQSPATLSLSPGERATLSCRASQSVSSYLAWYQQKP |
| | | Constant | GQAPRLLIYDASNRATGIPARFSGSGSGTDFTLTISSLEP |
| | | | EDFAVYYCQQRSNWPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 342 | ATX-P-433 | Variable LC + Kappa | DIQMTQSPSSLSASVGDRVTITCRASQSISIYLNWYQQKP |
| | | Constant | GTAPKLLIYAASSLQSGVPSRFSGSGSGTDFTLTISSLQP |
| | | | EDFAAYYCQQSYTTPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 343 | ATX-P-444 | Variable LC + Kappa | GATGTTGTGATGACTCAGTCTCCACTCTCCCTGCCCGTCA |
| | | Constant (nucleotide) | CCCTTGGACAGCCGGCCTCCATCTCCTGCAGGTCTAGTCA |
| | | | AAGCCTCGTATACAGTGATGGAAACACCTACTTGAATTGG |
| | | | TTTCAGCAGAGGCCAGGCCAATCTCCAAGGCGCCTAATTT |
| | | | ATAAGGTTTCTAACCGGGACTCTGGGGTCCCAGACAGATT |
| | | | CAGCGGCAGTGGGTCAGGCACTGATTTCACACTGAAAATC |
| | | | AGCAAGGTGGAGGCTGAGGATGTTGGGATTTATTACTGCA |
| | | | TGCAAGGTACACACTGGCCGTGGACGTTCGGCCAAGGGAC |
| | | | CAAAGTGGATATCAAACGTACGGTAGCTGCCCCTTCAGTT |
| | | | TTTATCTTTCCGCCGTCTGACGAGCAGTTAAAATCCGGGA |
| | | | CCGCTTCTGTAGTTTGCCTGCTGAATAATTTTTATCCGCG |
| | | | TGAGGCTAAAGTACAATGGAAAGTCGACAATGCTTTGCAG |
| | | | TCGGGAAATTCACAGGAAAGTGTTACGGAGCAGGATTCTA |
| | | | AAGATTCCACATATTCACTCAGCTCCACCCTTACACTGAG |
| | | | CAAAGCCGACTATGAAAAACATAAAGTTTACGCATGTGAG |
| | | | GTGACGCACCAAGGATTATCCAGTCCGGTCACAAAATCGT |
| | | | TTAACCGCGGTGAGTGT |
| 344 | ATX-P-445 | Variable LC + Kappa | GATGTTGTGATGACTCAGTCTCCACTCTCCCTGCCCGTCA |
| | | Constant (nucleotide) | CCCTTGGACAGCCGGCCTCCATCTCCTGCAGGTCTAGTCA |
| | | | AAGCCTCGTATACAGTGATGGAAACACCTACTTGAATTGG |
| | | | TTTCAGCAGAGGCCAGGCCAATCTCCAAGGCGCCTAATTC |
| | | | ATGAGGTTTCTAACCGGGACTCTGGGGTCCCAGACAGATT |
| | | | CAGCGGCAGTGGGTCAGGCACTGATTTCACACTGAAAATC |
| | | | AGCAGGGTGGAGGCTGAGGATGTTGGGGTTTATTACTGCA |
| | | | TGCAAGGTACACACTGGCCGTGGACGTTCGGCCAAGGGAC |
| | | | CAAAGTGGATATCAAACGTACGGTAGCTGCCCCTTCAGTT |
| | | | TTTATCTTTCCGCCGTCTGACGAGCAGTTAAAATCCGGGA |
| | | | CCGCTTCTGTAGTTTGCCTGCTGAATAATTTTTATCCGCG |
| | | | TGAGGCTAAAGTACAATGGAAAGTCGACAATGCTTTGCAG |
| | | | TCGGGAAATTCACAGGAAAGTGTTACGGAGCAGGATTCTA |
| | | | AAGATTCCACATATTCACTCAGCTCCACCCTTACACTGAG |
| | | | CAAAGCCGACTATGAAAAACATAAAGTTTACGCATGTGAG |
| | | | GTGACGCACCAAGGATTATCCAGTCCGGTCACAAAATCGT |
| | | | TTAACCGCGGTGAGTGT |
| 345 | ATX-P-446 | Variable LC + Kappa | GATGTTGTGATGACTCAGTCTCCACTCTCCCTGCCCGTCA |
| | | Constant (nucleotide) | CCCTTGGACAGCCGGCCTCCATCTCCTGCAGGTCTAGTCA |
| | | | AAGCCTCGTATTCAGTGATGGAAACACCTACTTGAATTGG |
| | | | TTTCAGCAGAGGCCAGGCCAATCTCCAAGGCGCCTAATTT |
| | | | ATAAGGTTTCTAACCGGGACTCTGGGGTCCCAGACAGATT |
| | | | CAGCGGCAGTGGGTCAGGCACTGATTTCACACTGAAAATC |
| | | | AGCAGGGGGAGGCTGAGGATGTTGGGGTTTATTACTGCAT |
| | | | GCAAGGTACACACTGGCCGTGGACGTTCGGCCAAGGGACC |
| | | | AAAGTGGATATCAAACGTACGGTAGCTGCCCCTTCAGTTT |
| | | | TTATCTTTCCGCCGTCTGACGAGCAGTTAAAATCCGGGAC |
| | | | CGCTTCTGTAGTTTGCCTGCTGAATAATTTTTATCCGCGT |
| | | | GAGGCTAAAGTACAATGGAAAGTCGACAATGCTTTGCAGT |
| | | | CGGGAAATTCACAGGAAAGTGTTACGGAGCAGGATTCTAA |
| | | | AGATTCCACATATTCACTCAGCTCCACCCTTACACTGAGC |
| | | | AAAGCCGACTATGAAAAACATAAAGTTTACGCATGTGAGG |
| | | | TGACGCACCAAGGATTATCCAGTCCGGTCACAAAATCGTT |
| | | | TAACCGCGGTGAGTGT |
| 346 | ATX-P-447 | Variable LC + Kappa | GATGTTGTGATGACTCAGTCTCCACTCTCCCTGCCCGTCA |
| | | Constant (nucleotide) | CCCTTGGACAGCCGGCCTCCATCTCCTGCAGGTCTAGTCA |
| | | | AAGCCTCGTATACAGTGATGGAAACACCTACTTGAATTGG |
| | | | TTTCAGCAGAGGCCAGGCCAATCTCCAAGGCGCCTAATTC |
| | | | ATGAGGTTTCTAACCGGGACTCTGGGGTCCCAGACAGATT |
| | | | CAGCGGCAGTGGGTCAGGCACTGATTTCACACTGAAAATC |
| | | | AGCAGGGTGGAGGCTGAGGATGTTGGGGTTTATTACTGCA |
| | | | TGCAAGGTACACACTGGCCGTGGACGTTCGGCCAAGGGAC |
| | | | CAAAGTGGATATCAAACGTACGGTAGCTGCCCCTTCAGTT |
| | | | TTTATCTTTCCGCCGTCTGACGAGCAGTTAAAATCCGGGA |
| | | | CCGCTTCTGTAGTTTGCCTGCTGAATAATTTTTATCCGCG |
| | | | TGAGGCTAAAGTACAATGGAAAGTCGACAATGCTTTGCAG |
| | | | TCGGGAAATTCACAGGAAAGTGTTACGGAGCAGGATTCTA |
| | | | AAGATTCCACATATTCACTCAGCTCCACCCTTACACTGAG |
| | | | CAAAGCCGACTATGAAAAACATAAAGITTACGCATGTGAG |
| | | | GTGACGCACCAAGGATTATCCAGTCCGGTCACAAAATCGT |
| | | | TTAACCGCGGTGAGTGT |
| 347 | ATX-P-427 | Variable LC + Kappa | GCCATCCAGATGACCCAGTCTCCATCCTCCCTGTCTGCAT |
| | | Constant (nucleotide) | CTGTAGGAGACAGAGTCACCATCACTTGCCGGGCAAGTCA |
| | | | GGGCATTAGAAATGATTTAGGCTGGTATCAGCAGAAACCA |
| | | | GGGAAAGCCCCTAAGCTCCTGATCTATGCTGCATCCAGTT |
| | | | TACAAAGTGGGGTCCCATCAAGGTTCAGCGGCAGTGGATC |
| | | | TGGCACAGATTTCACTCTCACCATCAGCAGCCTGCAGCCT |
| | | | GAAGATTTTGCAACTTATTACTGTCTACAAGATTACAATT |
| | | | ACCCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 348 | ATX-P-440 | Variable LC + Kappa | GACATCCAGATGACCCAGTCTCCTTCCACCCTGTCTGCAT |
| | | Constant (nucleotide) | CTGTAGGAGACAGAGTCACCATCACTTGCCGGGCCAGTCA |
| | | | GAGTATTAGTAGCTGGTTGGCCTGGTATCAGCAGAAACCA |
| | | | GGGAAAGCCCCTAAGCTCCTGATCTATGCTACATCCAGTT |
| | | | TGCAAAGTGGGGTCCCATCAAGGTTCAGTGGCAGTGGATC |
| | | | TGGGACAGATTTCACTCTCACCATCAGCAGTCTGCAACCT |
| | | | GAAGATTTTGCAACTTACTACTGTCAACAGAGTTACAGTA |
| | | | CCCCGATCACCTTCGGCCAAGGGACACGACTGGAGATTAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 349 | ATX-P-424 | Variable LC + Kappa | GACATCCAGATGACCCAGTCTCCATCCTCCCTGTCTGCAT |
| | | Constant (nucleotide) | CTGTAGGAGACAGAGTCACCATCACTTGCCGGGCAAGTCA |
| | | | GGGCATTAGAAATGATTTAGGCTGGTATCAGCAGAAACCA |
| | | | GGGAAAGCCCCTAAGCGCCTGATCTATGCTGCATCCAGTT |
| | | | TGCCAAGTGGGGTCCCATCAAGGTTCAGCGGCAGTGGATC |
| | | | TGGGACAGAATTCACTCTCACAATCAGCAGCCTGCAGCCT |
| | | | GAAGATTTTGCAATTTATTACTGTCTACAGCATAATAGTT |
| | | | ACCCGTGGACGTTQGGCCAAGGGACCAAGGTGGAAATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 350 | ATX-P-425 | Variable LC + Kappa | GACATCCAGATGACCCAGTCTCCATCCTCCCTGTQCGCAT |
| | | Constant (nucleotide) | CTGTAGGAGACAGAGTCACCATCACTTGCCGGGCAAGTCA |
| | | | GGGCATTAGAATTGATTTAGGCTGGTTTCAGCAGAAACCA |
| | | | GGGAAAGCCCCTAAGCGCCTCATCTATGCTGCATCCAGTT |
| | | | TGCCAAGTGGGGTCCCATCAAGGTTCAGCGGCAGTGGATC |
| | | | TGGGACAGAATTCACTCTCACAATCAGCAGCCTGCAGCCT |
| | | | GAAGATTTTGCAACTTATTACTGTCTACAGCATAATAGTT |
| | | | ACCCGTGGACGTTCGGCCAAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 351 | ATX-P-430 | Variable LC + Kappa | GAAATTGTGTTGACGCAGTCTCCAGGCACCCTGTCTTTGT |
| | | Constant (nucleotide) | CTCCAGGGGAAAGAGCCACCCTCTCCTGCAGGGCCAGTCA |
| | | | GAGTGTTAGCAGCAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACCTCCCACTTTCGGCGGAGGGACCAAGGTGGAAAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 352 | ATX-P-439 | Variable LC + Kappa | GACATCCAGATGACCCAGTCTCCATCTTCCGTGTCTGCAT |
| | | Constant (nucleotide) | CTGTAGGAGACAGAGTCACCATCACTTGTCGGGCGAGTCA |
| | | | GGGTATTAGCAGCTGGTTAGCCTGGTATCAGCAGAAACCA |
| | | | GGGAAAGCCCCTAAGCTCCTGATCTATGCTGCATCCAGTT |
| | | | TGCAAAGTGGGGTCCCATCAAGGTTCAGCGGCAGTGGATC |
| | | | TGGGACAGATTTCACTCTCACCATCAGCAGCCTGCAGCCT |
| | | | GAAGATTTTGCAACTTACTATTGTCAACAGGCTAACAGTT |
| | | | TCCCGTGGACGTTCGGCCAAGGGACCAAAGTGGATATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 353 | ATX-P-423 | Variable LC + Kappa | GACATCCAGATGACCCAGTCTCCATCCTCCCTGTCTGCAT |
| | | Constant (nucleotide) | CTGTAGGAGACAGAGTCACCATCACTTGCCGGGCAAGTCA |
| | | | GGGCATTAGAAATGATTTAGGCTGGTATCAGCAGAAACCA |
| | | | GGGAAAGCCCCTAAGCGCCTGATCTATGCTGCATCCAGTT |
| | | | TGCAAAGTGGGGTCCCATCAAGGTTCAGCGGCAGTGGATC |
| | | | TGGGACAGAATTCGCTCTCACAATCAGCAGCCTGCAGCCT |
| | | | GAAGATTTTGCAACTTATTACTGTCTACAGCATAATAGTT |
| | | | ACCCTCTCACTTTCGGQGGAGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 354 | ATX-P-438 | Variable LC + Kappa | GAAATTGTGCTGACTCAGTCTCCCGGCACACTGTCTCTTA |
| | | Constant (nucleotide) | GCCCTGGCGAAAGAGCCACACTGAGCTGTAGAGCCAGCCA |
| | | | GAGCGTGTCCAGAAGCTACCTGGCTTGGTATCAGCAGAAG |
| | | | CCCGGACAGGCTCCCAGACTGCTGATCATQGGAGCCTCTA |
| | | | CAAGAGCCACCGGCATTCCCGATAGATTCAGCGGCTCTGG |
| | | | CAGCGGCACCGATTTCACCCTGACAATCAGCAGACTGGAA |
| | | | CCCGAGGACTTCGCCGTGTACTACTGTCAGCAGGGCCAAG |
| | | | TGATCCCTCCTACCTTTGGCCAGGGCACCAAGGTGGAAAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 355 | ATX-P-448 | Variable LC + Kappa | GAAATTGTGTTGACGCAGTCTCCAGCCACCCTGTCTTTGT |
| | | Constant (nucleotide) | CTCCAGGGGAAAGAGCCACCCTCTCCTGCAGGGCCAGTCA |
| | | | GAGTGTTAGCAGCTACTTAGCCTGGTACCAACAGAAACCT |
| | | | GGCCAGGCTCCCAGGCTCCTCATCTATGATGCATCCAACA |
| | | | GGGCCACTGGCATCCCAGCCAGGTTCAGTGGCAGTGGGTC |
| | | | TGGGACAGACTTCACTCTCACCATCAGCAGCCTAGAGCCT |
| | | | GAAGATTTTGCAGTTTATTACTGTCAGCAGCGTAGCAACT |
| | | | GGCCTCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCQGGTCACAAAATCGTTTAACCGCGGTGAGT |
| | | | GT |
| 356 | ATX-P-449 | Variable LC + Kappa | GAAATTGTGTTGACGCAGTCTCCAGCCACCCTGTCTTTGT |
| | | Constant (nucleotide) | CTCCAGGGGAAAGAGCCACCCTCTCCTGCAGGGCCAGTCA |
| | | | GAGTGTTAGCAGCTACTTAGCCTGGTACCAACAGAAACCT |
| | | | GGCCAGGCTCCCAGGCTCCTCATCTATGATGCATCCAACA |
| | | | GGGCCACTGGCATCCCAGCCAGGTTCAGTGGCAGTGGGTC |
| | | | TGGGACAGACTTCACTCTCACCATCAGCAGCCTAGAGCCT |
| | | | GAAGATTTTGCAGTTTATTACTGTCAGCAGCGTAGCAACT |
| | | | GGCCTCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 357 | ATX-P-433 | Variable LC + Kappa | GACATCCAGATGACCCAGTCTCCATCCTCCCTGTCTGCAT |
| | | Constant (nucleotide) | CTGTAGGAGACAGAGTCACCATCACTTGCCGGGCAAGTCA |
| | | | GAGCATTAGCATCTATTTAAATTGGTATCAGCAGAAACCA |
| | | | GGGACAGCCCCTAAGCTCCTGATCTATGCTGCATCCAGTT |
| | | | TGCAAAGTGGGGTCCCATCAAGGTTCAGTGGCAGTGGATC |
| | | | TGGGACAGATTTCACTCTCACCATCAGCAGTCTGCAACCT |
| | | | GAAGATTTTGCAGCTTACTACTGTCAACAGAGTTACACTA |
| | | | CCCCGCTCACTTTCGGCGGAGGGACCAAGGTGGAAATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 358 | ATX-P-435 | Variable HC + Constant | QLQLQESGPGLVKPSETLSLTCTVSGGSISSSSSYWGWIR |
| | | hIgG1-Agly-Fc | QPPGKGLEWIGSIYYSGSTYSNPSLKSRVTISVDTSKNQF |
| | | | SLKLSSVTAADTAVYYCARQYISGTEYFQYWGQGTLVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHNHY |
| | | | TQKSLSLSPG |
| 359 | ATX-P-436 | Variable HC + Constant | QLQLQESGPGLVKPSETLSLTCTVSGGSISSSSSYWGWIR |
| | | hIgG1-Agly-Fc | QPPGKGLEWIGSIYYSGSTYSNPSLKSRVTISVDTSKNQF |
| | | | SLKLSSVTAADTAVYYCARQYISGTEYFQYWGQGTLVTVS |
| | | | SASTKGPSVFPLAPSSKSTSGGTAALGCLVKDYFPEPVTV |
| | | | SWNSGALTSGVHTFPAVLQSSGLYSLSSVVTVPSSSLGTQ |
| | | | TYICNVNHKPSNTKVDKRVEPKSCDKTHTCPPCPAPELLG |
| | | | GPSVFLFPPKPKDTLMISRTPEVTCVVVDVSHEDPEVKFN |
| | | | WYVDGVEVHNAKTKPREEQYASTYRVVSVLTVLHQDWLNG |
| | | | KEYKCKVSNKALPAPIEKTISKAKGQPREPQVYTLPPSRE |
| | | | EMTKNQVSLTCLVKGFYPSDIAVEWESNGQPENNYKTTPP |
| | | | VLDSDGSFFLYSKLTVDKSRWQQGNVFSCSVMHEALHNHY |
| | | | TQKSLSLSPG |
| 360 | ATX-P-434 | Variable HC + Constant | QVQLQESGPGLVKPSETLSLTCTVSGGSVSSGSYYWSWIR |
| | | hIgG1-Agly-Fc | QPPGKGLEWIGYIYYSGSTSYNPSLKSRVSISVDTSKNQF |
| | | | SLKLSSVTAADTAVFYCARAKWEGDYFDYWGQGTLVTVSS |
| | | | ASTKGPSVFPLAPSSKSTSGGTAALGCLVKDYFPEPVTVS |
| | | | WNSGALTSGVHTFPAVLQSSGLYSLSSVVTVPSSSLGTQT |
| | | | YICNVNHKPSNTKVDKRVEPKSCDKTHTCPPCPAPELLGG |
| | | | PSVFLFPPKPKDTLMISRTPEVTCVVVDVSHEDPEVKFNW |
| | | | YVDGVEVHNAKTKPREEQYASTYRVVSVLTVLHQDWLNGK |
| | | | EYKCKVSNKALPAPIEKTISKAKGQPREPQVYTLPPSREE |
| | | | MTKNQVSLTCLVKGFYPSDIAVEWESNGQPENNYKTTPPV |
| | | | LDSDGSFFLYSKLTVDKSRWQQGNVFSCSVMHEALHNHYT |
| | | | QKSLSLSPG |
| 361 | ATX-P-435 | Variable HC + Constant | CAGCTGCAGCTGCAGGAGTCGGGCCCAGGACTGGTGAAGC |
| | | hIgG1-Agly-Fc | CTTCGGAGACCCTGTCCCTCACCTGCACTGTCTCTGGTGG |
| | | (nucleotide) | TTCCATCAGCAGTAGTAGTTCCTACTGGGGCTGGATCCGC |
| | | | CAGCCCCCAGGGAAGGGACTGGAGTGGATTGGGAGTATCT |
| | | | ATTATAGTGGGAGCACCTACTCCAACCCGTCCCTCAAGAG |
| | | | TCGAGTCACCATATCCGTAGACACGTCCAAGAATCAGTTC |
| | | | TCCCTGAAGCTGAGCTCTGTGACCGCCGCAGACACGGCTG |
| | | | TGTATTACTGTGCGAGACAGTATATAAGTGGAACTGAATA |
| | | | CTTCCAGTACTGGGGCCAGGGCACCCTGGTCACCGTCTCC |
| | | | TCAGCTAGCACTAAAGGGCCTTCTGTATTTCCCTTGGCCC |
| | | | CGTCCAGCAAATCGACCTCGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTTCCAAGGCCAAAGGTCAACC |
| | | | AAGAGAACCCCAGGTGTATACTCTTCCGCCTTCTCGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGCGTTACATAATCACTAC |
| | | | ACGCAGAAATCTCTTAGTCTTTCACCCGGT |
| 362 | ATX-P-436 | Variable HC + Constant | CAGCTGCAGCTGCAGGAGTCGGGCCCAGGACTGGTGAAGC |
| | | hIgG1-Agly-Fc | CTTCGGAGACCCTGTCCCTCACCTGCACTGTCTCTGGTGG |
| | | (nucleotide) | TTCCATCAGCAGTAGTAGTTCCTACTGGGGCTGGATCCGC |
| | | | CAGCCCCCAGGGAAGGGACTGGAGTGGATTGGGAGTATCT |
| | | | ATTATAGTGGGAGCACCTACTCCAACCCGTCCCTCAAGAG |
| | | | TCGAGTCACCATATCCGTAGACACGTCCAAGAATCAGTTC |
| | | | TCCCTGAAGCTGAGCTCTGTGACCGCCGCAGACACGGCTG |
| | | | TGTATTACTGTGCGAGACAGTATATAAGTGGAACTGAATA |
| | | | CTTCCAGTACTGGGGCCAGGGCACCCTGGTCACCGTCTCC |
| | | | TCAGCTAGCACTAAAGGGCCTTCTGTATTTCCCTTGGCCC |
| | | | CGTCCAGCAAATCGACCTCGGGAGGGACAGCCGCCCTGGG |
| | | | TTGCCTTGTGAAAGATTATTTCCCTGAGCCAGTTACCGTA |
| | | | AGTTGGAACAGTGGGGCGCTGACAAGTGGTGTGCACACGT |
| | | | TTCCTGCCGTCCTGCAATCATCGGGCTTGTATAGCCTCAG |
| | | | CTCTGTGGTCACTGTCCCAAGTTCATCGCTGGGCACTCAG |
| | | | ACGTATATTTGCAATGTGAACCACAAACCTTCAAATACAA |
| | | | AAGTGGATAAACGCGTAGAACCGAAATCGTGTGATAAAAC |
| | | | TCACACATGCCCGCCATGCCCGGCACCTGAACTGCTTGGT |
| | | | GGTCCCAGCGTGTTCCTGTTCCCGCCGAAGCCTAAAGATA |
| | | | CTCTAATGATCAGCCGTACGCCAGAGGTGACATGTGTCGT |
| | | | GGTTGACGTGTCCCACGAAGATCCCGAAGTTAAGTTCAAT |
| | | | TGGTATGTTGATGGTGTAGAGGTACACAATGCTAAGACTA |
| | | | AACCTCGCGAGGAGCAGTACGCCTCGACCTATCGTGTCGT |
| | | | GAGCGTTCTGACCGTCCTTCACCAAGATTGGCTTAACGGC |
| | | | AAAGAATATAAGTGCAAGGTAAGCAATAAAGCACTTCCGG |
| | | | CCCCAATCGAGAAAACCATTTCCAAGGCCAAAGGTCAACC |
| | | | AAGAGAACCCCAGGTGTATACTCTTCCGCCTTCTCGTGAG |
| | | | GAAATGACTAAAAATCAAGTATCCCTTACGTGTCTGGTTA |
| | | | AAGGTTTTTATCCTAGCGATATTGCTGTTGAATGGGAATC |
| | | | GAACGGTCAGCCGGAGAATAATTATAAAACAACGCCACCC |
| | | | GTCCTGGATAGCGACGGCTCATTTTTTCTGTATAGCAAAC |
| | | | TGACTGTAGATAAATCACGGTGGCAGCAGGGCAATGTATT |
| | | | CAGTTGCTCCGTTATGCATGAAGCGTTACATAATCACTAC |
| | | | ACGCAGAAATCTCTTAGTCTTTCACCCGGT |
| 363 | ATX-P-434 | Variable HC + Constant | CAGGTGCAGCTGCAGGAGTCGGGCCCAGGACTGGTGAAGC |
| | | hIgG1-Agly-Fc | CTTCGGAGACCCTGTCCCTCACCTGCACTGTCTCTGGTGG |
| | | (nucleotide) | CTCCGTCAGCAGTGGTAGTTACTACTGGAGCTGGATCCGG |
| | | | CAGCCCCCAGGGAAGGGACTGGAGTGGATTGGGTATATCT |
| | | | ATTACAGTGGGAGCACCAGCTACAACCCCTCCCTCAAGAG |
| | | | TCGAGTCTCCATATCAGTAGACACGTCCAAGAACCAGTTC |
| | | | TCCCTGAAGCTGAGCTCTGTGACCGCTGCGGACACGGCCG |
| | | | TGTTTTACTGTGCGAGAGCTAAGTGGGAAGGGGACTACTT |
| | | | TGACTACTGGGGCCAGGGAACCCTGGTCACCGTCTCCTCA |
| | | | GCTAGCACTAAAGGGCCTTCTGTATTTCCCTTGGCCCCGT |
| | | | CCAGCAAATCGACCTCGGGAGGGACAGCCGCCCTGGGTTG |
| | | | CCTTGTGAAAGATTATTTCCCTGAGCCAGTTACCGTAAGT |
| | | | TGGAACAGTGGGGCGCTGACAAGTGGTGTGCACACGTTTC |
| | | | CTGCCGTCCTGCAATCATCGGGCTTGTATAGCCTCAGCTC |
| | | | TGTGGTCACTGTCCCAAGTTCATCGCTGGGCACTCAGACG |
| | | | TATATTTGCAATGTGAACCACAAACCTTCAAATACAAAAG |
| | | | TGGATAAACGCGTAGAACCGAAATCGTGTGATAAAACTCA |
| | | | CACATGCCCGCCATGCCCGGCACCTGAACTGCTTGGTGGT |
| | | | CCCAGCGTGTTCCTGTTCCCGCCGAAGCCTAAAGATACTC |
| | | | TAATGATCAGCCGTACGCCAGAGGTGACATGTGTCGTGGT |
| | | | TGACGTGTCCCACGAAGATCCCGAAGTTAAGTTCAATTGG |
| | | | TATGTTGATGGTGTAGAGGTACACAATGCTAAGACTAAAC |
| | | | CTCGCGAGGAGCAGTACGCCTCGACCTATCGTGTCGTGAG |
| | | | CGTTCTGACCGTCCTTCACCAAGATTGGCTTAACGGCAAA |
| | | | GAATATAAGTGCAAGGTAAGCAATAAAGCACTTCCGGCCC |
| | | | CAATCGAGAAAACCATTTCCAAGGCCAAAGGTCAACCAAG |
| | | | AGAACCCCAGGTGTATACTCTTCCGCCTTCTCGTGAGGAA |
| | | | ATGACTAAAAATCAAGTATCCCTTACGTGTCTGGTTAAAG |
| | | | GTTTTTATCCTAGCGATATTGCTGTTGAATGGGAATCGAA |
| | | | CGGTCAGCCGGAGAATAATTATAAAACAACGCCACCCGTC |
| | | | CTGGATAGCGACGGCTCATTTTTTCTGTATAGCAAACTGA |
| | | | CTGTAGATAAATCACGGTGGCAGCAGGGCAATGTATTCAG |
| | | | TTGCTCCGTTATGCATGAAGCGTTACATAATCACTACACG |
| | | | CAGAAATCTCTTAGTCTTTCACCCGGT |
| 364 | ATX-P-435 | Variable LC + Kappa | EIVLTQSPATLSLSPGERATLSCRASQSVSNYLAWYQQKP |
| | | Constant | GQAPRLLIYDASNRATGIPARFSGSGSGTDFTLTISSLEP |
| | | | EDFAVYYCQQRSNWPSFGQGTRLEIKRTVAAPSVFIFPPS |
| | | | DEQLKSGTASVVCLLNNFYPREAKVQWKVDNALQSGNSQE |
| | | | SVTEQDSKDSTYSLSSTLTLSKADYEKHKVYACEVTHQGL |
| | | | SSPVTKSFNRGEC |
| 365 | ATX-P-436 | Variable LC + Kappa | DIQMTQSPSSLSASVGDRVTITCRASQSISSYLNWYQQKP |
| | | Constant | GKAPKLLIYAASSLQSGVPSRFSGSGSGTDFTLTISSLQP |
| | | | EDFATYYCQQSYSTPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 366 | ATX-P-434 | Variable LC + Kappa | DIQMTQSPSSLSASVGDRVTITCRASQSIYSYLNWYQQKP |
| | | Constant | GKAPNHLIYAASSLQSGVPSRFSGSGSGTDFTLTISSLHP |
| | | | EDFATYYCQQSYSTPPYTFGQGTKVEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVIKSFNRGEC |
| 367 | ATX-P-435 | Variable LC + Kappa | GAAATTGTGTTGACACAGTCTCCAGCCACCCTGTCTTTGT |
| | | Constant (nucleotide) | CTCCAGGGGAAAGAGCCACCCTCTCCTGCAGGGCCAGTCA |
| | | | GAGTGTTAGCAACTACTTAGCCTGGTACCAACAGAAACCT |
| | | | GGCCAGGCTCCCAGGCTCCTCATCTATGATGCATCCAACA |
| | | | GGGCCACTGGCATCCCAGCCAGATTCAGTGGCAGTGGGTC |
| | | | TGGGACAGACTTCACTCTCACCATCAGCAGCCTGGAGCCT |
| | | | GAAGATTTTGCAGTTTATTACTGTCAGCAGCGTAGCAACT |
| | | | GGCCCTCCTTCGGCCAAGGGACACGACTGGAGATTAAACG |
| | | | TACGGTAGCTGCCCCTTCAGTTTTTATCTTTCCGCCGTCT |
| | | | GACGAGCAGTTAAAATCCGGGACCGCTTCTGTAGTTTGCC |
| | | | TGCTGAATAATTTTTATCCGCGTGAGGCTAAAGTACAATG |
| | | | GAAAGTCGACAATGCTTTGCAGTCGGGAAATTCACAGGAA |
| | | | AGTGTTACGGAGCAGGATTCTAAAGATTCCACATATTCAC |
| | | | TCAGCTCCACCCTTACACTGAGCAAAGCCGACTATGAAAA |
| | | | ACATAAAGTTTACGCATGTGAGGTGACGCACCAAGGATTA |
| | | | TCCAGTCCGGTCACAAAATCGTTTAACCGCGGTGAGTGT |
| 368 | ATX-P-436 | Variable LC + Kappa | GACATCCAGATGACCCAGTCTCCATCCTCCCTGTCTGCAT |
| | | Constant (nucleotide) | CTGTAGGAGACAGAGTCACCATCACTTGCCGGGCAAGTCA |
| | | | GAGCATTAGCAGCTATTTAAATTGGTATCAGCAGAAACCA |
| | | | GGGAAAGCCCCTAAGCTCCTGATCTATGCTGCATCCAGTT |
| | | | TGCAAAGTGGGGTCCCATCAAGGTTCAGTGGCAGTGGATC |
| | | | TGGGACAGATTTCACTCTCACCATCAGCAGTCTGCAACCT |
| | | | GAAGATTTTGCAACTTACTACTGTCAACAGAGTTACAGTA |
| | | | CCCCGCTCACTTTCGGCGGAGGGACCAAGGTGGAAATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 369 | ATX-P-434 | Variable LC + Kappa | GACATCCAGATGACCCAGTCTCCATCCTCCCTGTCTGCAT |
| | | Constant (nucleotide) | CTGTAGGAGACAGAGTCACCATCACTTGCCGGGCAAGTCA |
| | | | GAGCATTTACAGCTATTTAAATTGGTATCAGCAGAAACCA |
| | | | GGGAAAGCCCCTAATCACCTGATCTATGCTGCATCCAGTT |
| | | | TGCAAAGTGGGGTCCCATCAAGGTTCAGTGGCAGTGGATC |
| | | | TGGGACAGATTTCACTCTCACCATCAGCAGTCTGCACCCT |
| | | | GAAGATTTTGCAACTTACTACTGTCAACAGAGTTACAGTA |
| | | | CCCCTCCGTACACTTTTGGCCAGGGGACCAAGGTGGAAAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |

Human Angiopoietin-related
protein 7 (UniProt Accession No. O43827):
(SEQ ID NO: 370)
MLKKPLSAVTWLCIFIVAFVSHPAWLOKLSKHKTPAQPQLKAANC
CEEVKELKAQVANLSSLLSELNKKQERDWVSVVMQVMELESNSKR
MESRLTDAESKYSEMNNQIDIMQLQAAQTVTQTSADAIYDCSSLY
QKNYRISGVYKLPPDDFLGSPELEVFCDMETSGGGWTIIQRRKSG
LVSFYRDWKQYKQGFGSIRGDFWLGNEHIHRLSRQPTRLRVEMED
WEGNLRYAEYSHFVLGNELNSYRLFLGNYTGNVGNDALQYHNNTA
FSTKDKDNDNCLDKCAQLRKGGYWYNCCTDSNLNGVYYRLGEHNK
HLDGITWYGWHGSTYSLKRVEMKIRPEDFKP.
Human Angiopoietin-related protein 7
L59P L84P variant (ATX-P-62):
(SEQ ID NO: 371)
MLKKPLSAVTWLCIFIVAFVSHPAWLQKLSKHKTPAQPQLKAANC
CEEVKELKAQVANPSSLLSELNKKQERDWVSVVMQVMEPESNSKR
MESRLTDAESKYSEMNNQIDIMQLQAAQTVTQTSADAIYDCSSLY
QKNYRISGVYKLPPDDFLGSPELEVFCDMETSGGGWTIIQRRKSG
LVSFYRDWKQYKQGFGSIRGDFWLGNEHIHRLSRQPTRLRVEMED
WEGNLRYAEYSHFVLGNELNSYRLFLGNYTGNVGNDALQYHNNTA
FSTKDKDNDNCLDKCAQLRKGGYWYNCCTDSNLNGVYYRLGEHNK
HLDGITWYGWHGSTYSLKRVEMKIRPEDFKP.
Human Angiopoietin-related protein 7
L59 GGPGG variant (ATX-P-63):
(SEQ ID NO: 372)
MLKKPLSAVTWLCIFIVAFVSHPAWLQKLSKHKTPAQPQLKAANC
CEEVKELKAQVANGGPGGSSLLSELNKKQERDWVSVVMQVMELES
NSKRMESRLTDAESKYSEMNNQIDIMQLQAAQTVTQTSADAIYDC
SSLYQKNYRISGVYKLPPDDFLGSPELEVFCDMETSGGGWTIIQR
RKSGLVSFYRDWKQYKQGFGSIRGDFWLGNEHIHRLSRQPTRLRV
EMEDWEGNLRYAEYSHFVLGNELNSYRLFLGNYTGNVGNDALQYH
NNTAFSTKDKDNDNCLDKCAQLRKGGYWYNCCTDSNLNGVYYRLG
EHNKHLDGITWYGWHGSTYSLKRVEMKIRPEDFKP.
Human Angiopoietin-related protein 7
fibrinogen domain (ATX-P-60):
(SEQ ID NO: 373)
YDCSSLYQKNYRISGVYKLPPDDFLGSPELEVFCDMETSGGGWTI
IQRRKSGLVSFYRDWKQYKQGFGSIRGDFWLGNEHIHRLSRQPTR
LRVEMEDWEGNLRYAEYSHFVLGNELNSYRLFLGNYTGNVGNDAL
QYHNNTAFSTKDKDNDNCLDKCAQLRKGGYWYNCCTDSNLNGVYY
RLGEHNKHLDGITWYGWHGSTYSLKRVEMKIRPED.
Human Angiopoietin-related protein 7 wild
type multimer (PExt-1):
(SEQ ID NO: 374)
MLKKPLSAVTWLCIFIVAFVSHPAWLQKLSKHKTPAQPQLKAANC
CEEVKELKAQVANLSSLLSELNKKQERDWVSVVMQVMELESNSKR
MESRLTDAESKYSEMNNQIDIMQLQAAQTVTQTSADAIYDCSSLY
QKNYRISGVYKLPPDDFLGSPELEVFCDMETSGGGWTIIQRRKSG
LVSFYRDWKQYKQGFGSIRGDFWLGNEHIHRLSRQPTRLRVEMED
WEGNLRYAEYSHFVLGNELNSYRLFLGNYTGNVGNDALQYHNNTA
FSTKDKDNDNCLDKCAQLRKGGYWYNCCTDSNLNGVYYRLGEHNK
HLDGITWYGWHGSTYSLKRVEMKIRPEDFKP.

TABLE 11
Summary of SEQ ID NOs for individual exemplary antibodies of the present disclosure.

| | | | | | | | VH + | VH + | VL + | VL + |
|---|---|---|---|---|---|---|---|---|---|---|
| Antibody | HCDRs 1, | LCDRs 1, | VH | VH | VL | VL | constant | constant | constant | constant |
| Name | 2, 3 (aa) | 2, 3 (aa) | (aa) | (nucleotide) | (aa) | (nucleotide) | (aa) | (nucleotide) | (aa) | (nucleotide) |
| ATX-P-423 | 40, 56, 71 | 90, 102, 114 | 208 | 223 | 238 | 253 | 308 | 323 | 338 | 353 |
| ATX-P-424 | 34, 50, 65 | 88, 100, 112 | 204 | 219 | 234 | 249 | 304 | 319 | 333 | 349 |
| ATX-P-425 | 35, 51, 66 | 89, 101, 113 | 205 | 220 | 235 | 250 | 305 | 320 | 335 | 350 |
| ATX-P-427 | 32, 48, 63 | 97, 109, 121 | 202 | 217 | 232 | 247 | 302 | 317 | 332 | 347 |
| ATX-P-428 | 2, 8, 13 | 144, 154, 164 | 170 | 175 | 180 | 185 | 270 | 275 | 280 | 285 |
| ATX-P-429 | 3, 9, 14 | 123, 129, 135 | 172 | 177 | 182 | 187 | 272 | 277 | 282 | 287 |
| ATX-P-430 | 36, 52, 67 | 148, 158, 168 | 206 | 221 | 236 | 251 | 306 | 321 | 336 | 351 |
| ATX-P-431 | 5, 11, 16 | 146, 156, 166 | 173 | 178 | 183 | 188 | 273 | 278 | 283 | 188 |
| ATX-P-432 | 6, 12, 17 | 147, 157, 167 | 174 | 179 | 184 | 189 | 274 | 279 | 284 | 289 |
| ATX-P-433 | 42, 58, 73 | 94, 106, 118 | 212 | 227 | 242 | 257 | 312 | 327 | 342 | 357 |
| ATX-P-434 | 75, 79, 83 | 96, 108, 120 | 260 | 263 | 266 | 269 | 360 | 363 | 366 | 369 |
| ATX-P-435 | 76, 80, 84 | 143, 153, 163 | 258 | 261 | 264 | 267 | 358 | 361 | 364 | 367 |
| ATX-P-436 | 77, 81, 85 | 95, 107, 119 | 259 | 262 | 265 | 268 | 359 | 362 | 365 | 368 |
| ATX-P-437 | 20, 23, 26 | 92, 104, 116 | 190 | 192 | 194 | 196 | 290 | 292 | 294 | 296 |
| ATX-P-438 | 41, 57, 72 | 149, 159, 169 | 209 | 224 | 239 | 254 | 309 | 324 | 339 | 354 |
| ATX-P-439 | 37, 53, 68 | 87, 99, 111 | 207 | 222 | 237 | 252 | 307 | 322 | 337 | 352 |
| ATX-P-440 | 33, 49, 64 | 93, 105, 117 | 203 | 218 | 233 | 248 | 303 | 318 | 333 | 348 |
| ATX-P-442 | 19, 22, 25 | 91, 103, 115 | 191 | 193 | 195 | 197 | 291 | 293 | 295 | 297 |
| ATX-P-443 | 4, 10, 15 | 145, 155, 165 | 171 | 176 | 181 | 186 | 271 | 276 | 281 | 286 |
| ATX-P-444 | 28, 44, 59 | 124, 130, 136 | 198 | 213 | 228 | 243 | 298 | 313 | 328 | 343 |
| ATX-P-445 | 29, 45, 60 | 125, 131, 137 | 199 | 214 | 229 | 244 | 299 | 314 | 329 | 344 |
| ATX-P-446 | 30, 46, 61 | 126, 132, 138 | 200 | 215 | 230 | 245 | 300 | 315 | 330 | 345 |
| ATX-P-447 | 31, 47, 62 | 127, 133, 139 | 201 | 216 | 231 | 246 | 301 | 316 | 331 | 346 |
| ATX-P-448 | 39, 55, 70 | 142, 152, 162 | 210 | 225 | 240 | 255 | 310 | 325 | 340 | 355 |
| ATX-P-449 | 38, 54, 69 | 141, 151, 161 | 211 | 226 | 241 | 256 | 311 | 326 | 341 | 356 |

KEY: aa = amino acid; HCDR 1, 2, 3 = heavy chain CDRs 1, 2, and 3 (in numerical order); LCDRs 1, 2, 3 = light chain CDRs 1, 2, and 3 (in numerical order); VH = heavy chain variable region; VL = light chain variable region; VH + constant = heavy chain variable region sequence with an exemplary human heavy chain constant region sequence (typically IgG1); VL + constant = light chain variable region sequence with an exemplary human light chain constant region sequence (typically kappa). All numbers except in “Antibody Name” correspond to SEQ ID NOs in the sequence listing and Tables 8, 9, and 10.

Various embodiments of the present disclosure are described herein. Variations of those embodiments may become apparent to those of ordinary skill in the art upon reading the foregoing description. The inventors expect skilled artisans to employ such variations as appropriate, and the inventors intend for the various embodiments of the present disclosure to be practiced otherwise than as specifically described herein. Accordingly, embodiments of the present disclosure include all modifications and equivalents of the subject matter recited in the claims appended hereto as permitted by applicable law. Moreover, any combination of the above-described elements in all possible variations thereof is encompassed by the various embodiments of the present disclosure unless otherwise indicated herein or otherwise clearly contradicted by context.

What is claimed is:

1. An antibody, or an antigen binding fragment thereof, which specifically binds human Angiopoietin-Like Protein 7 (ANGPTL7), optionally wherein said human ANGPTL7 is a polypeptide which comprises or consists of the amino acid sequence of any one of SEQ ID NOs: 370 to 374.

2. The antibody, or an antigen binding fragment thereof, of claim 1, which exhibits any one or more the following functional characteristics:
a. increases outflow facility compared to a control when administered to the eye of a subject, optionally wherein the control is vehicle treatment, dexamethasone treatment, ANGPTL7 protein treatment, or ANGPTL7 protein with an isotype control antibody treatment; and/or
b. binds to ANGPTL7 with a K_{D} of about 100 nM or lower; and/or
c. binds to the same epitope on ANGPTL7 as an antibody comprising the VH and VL sequences of any one of the exemplary antibodies the sequences of which are provided in Table 11; and/or
d. competes for binding to ANGPTL7 with an antibody comprising the VH and VL sequences of any one of the exemplary antibodies the sequences of which are provided in Table 11.

3. The antibody, or an antigen binding fragment thereof, of claim 1 or claim 2, which is monoclonal, optionally recombinant.

4. The antibody, or an antigen binding fragment thereof, of any one of claims 1 to 3, which is human, humanized or chimeric.

5. The antibody, or an antigen binding fragment thereof, of any one of claims 1 to 4, which is a full length antibody, a single chain antibody, a single chain variable fragment (scFv), a variable fragment (Fv), a fragment antigen-binding region (Fab), a Fab-C, a Fab′-SH, a (Fab′)2, a single-domain antibody (sdAb), a VHH antibody, a nanobody, a camelid-derived single-domain antibody, a shark IgNAR-derived single-domain antibody fragment (VNAR), a diabody, a triabody, an anticalin or an aptamer, optionally wherein the antibody is a full length antibody comprising an Fc region such as a human IgG1, IgG2, IgG3 or IgG4 region.

6. The antibody, or an antigen binding fragment thereof, of any one of claims 1 to 5, which is conjugated to at least one additional moiety, optionally selected from:
a. an antigen binding moiety, such as an antibody or antigen-binding fragment thereof, which is capable of specific binding to a target which is not human ANGPTL7, preferably wherein said target is expressed in the human eye;
b. a therapeutic or cytotoxic moiety;
c. a detection moiety;
d. a purification moiety;
e. a half-life extension moiety, optionally a polypeptide that is at least 20 amino acids in length and comprises any combination of G, A, S T, E, and P residue, which polypeptide is conjugated to the C- or N-terminus of the antibody.

7. The antibody, or an antigen binding fragment thereof, of any one of claims 1 to 6, which is a polypeptide comprising:
a. one, two or all three HCDRs of any one of the exemplary antibodies the sequences of which are provided in Table 11, and optionally also one, two or all three of the corresponding LCDRs of said exemplary antibody; and/or
b. a VH sequence having at least 90% identity to the VH sequence of any one of the exemplary antibodies the sequences of which are provided in Table 11, and optionally also a VL sequence having at least 90% identity to the corresponding VL sequence of said exemplary antibody, preferably wherein variation is not permitted in the HCDRs or LCDRs; and/or
c. all six CDRs of any one of the exemplary antibodies the sequences of which are provided in Table 11; and/or
d. the VH and VL sequences of any one of the exemplary antibodies the sequences of which are provided in Table 11; and/or
e. the full length heavy chain (VH+constant) sequence of any one of the exemplary antibodies the sequences of which are provided in Table 11, and optionally also the corresponding full length light chain (VL+constant) sequence of said exemplary antibody.

8. A polynucleotide encoding an antibody, or an antigen binding fragment thereof, of any one of claims 1 to 7, optionally wherein said polynucleotide comprises or consists of a nucleic acid sequence having at least 70%, 80%, 90% or 100% identity to a nucleic acid sequence of any one of the exemplary antibodies the sequences of which are provided in Table 11.

9. An expression vector comprising the polynucleotide of claim 8, which is optionally an adeno-associated virus (AAV) vector, a lentiviral (LV) vector, a herpes simplex virus (HSV) vector, or a retrovirus vector.

10. A pharmaceutical composition comprising an antibody, or an antigen binding fragment thereof, a polynucleotide, or a vector according to any one of the preceding claims, and optionally:
a. at least one pharmaceutically acceptable carrier, diluent or preservative; and/or
b. at least one additional active ingredient.

11. The pharmaceutical composition of claim 10, which is suitable for ocular administration to a subject, optionally by delivery using a conjunctival insert, a contact lens, a gel, a nanoparticle, a mucoadhesive polymer, an ointment, a solution, a suspension, eye drops, and/or an implant, preferably by injection into the vitreous fluid.

12. The antibody, or an antigen binding fragment thereof, according to any one of claims 1 to 7, the polynucleotide of claim 8, the vector of claim 9, or the composition of claim 10 or 11, for use as a medicament, optionally for use in a method of treating a disease of the eye in a subject.

13. The antibody, fragment, polynucleotide, vector, or composition for use according to claim 12, wherein said disease is characterized by increased intraocular pressure and/or reduced outflow facility in the eye of the subject.

14. The antibody, fragment, polynucleotide, vector or composition for use according to claim 12 or 13, wherein the method comprises ocular administration of the antibody, preferably by injection into the vitreous fluid, and wherein said administration preferably relieves at least one symptom in the subject selected from eye pain, eye pressure, headaches, rainbow-colored halos around lights, low vision, blurred vision, narrowed vision, impaired peripheral vision, blind spots, nausea, vomiting, and red eyes.

15. The antibody, fragment, polynucleotide, vector or composition for use according to any one of claims 12 to 14, where the disease is glaucoma and/or a disease affecting the optic nerve or retinal ganglion cells, optionally wherein said glaucoma is primary or glucocorticoid-induced glaucoma.

16. An antibody directed against Angiopoietin-Like Protein 7 (ANGPTL7) peptides, or an antigen-binding fragment thereof, comprising a heavy chain variable region (VH) comprising complementarity determining regions (CDRs) HCDR1, HCDR2, and HCDR3, and a light chain variable region (VL) comprising complementarity determining regions (CDRs) LCDR1, LCDR2, and LCDR3, wherein:
the HCDR1 comprises one of the following amino acid sequences:
(a) X₁YX₂IX₃ (SEQ ID NO: 1), wherein X₁ is S or D; X₂ is G or Y; X₃ is S or H;
(b) TSGVGVG (SEQ ID NO: 18);
(c) X₁X₂X₃MX₄ (SEQ ID NO: 27), wherein X₁ is V, S, D, or T; X₂ is Y, H, or F; X₃ is D, G, S, or A; X₄ is H, S, or N; or
(d) SX₁SX₂YWX₃ (SEQ ID NO: 74), wherein X₁ is S or G; X₂ is S or Y; X₃ is G or S;
wherein the HCDR2 comprises one of the following amino acid sequences:
(a) WIX₁X₂X₃X₄GX₅TX₆YAQX₇X₈X₉G (SEQ ID NO: 7), wherein X₁ is S, 1, or N; X₂ is A or P; X₃ is Y or N; X₄ is N or T; X₅ is N or A; X₆ is N or K; X₇ is N or K; X₈ is L or F; X₉ is R or Q;
(b) LIYWNDDKX₁YSPSLKS (SEQ ID NO: 21), wherein X₁ is R or Q;
(c) X₁X₂X₃X₄X₅X₆X₇X₈X₉X₁₀X₁₁X₁₂X₁₃X₁₄X₁₅G (SEQ ID NO: 43), wherein X₁ is G, T, S, A, V, H, or I; X₂ is I or M X₃ is D, N, T, S, or G; X₄ is P, W, S, G, or Y; X₅ is D, A, N, S, or Y; X₆ is G or S; X₇ is D, G, Y, S, I, or N; X₈ is T, S, N, I, Y, or D; X₉ is Y, T, F, M, K, G, or I; X₁₀ is Y, G, or F; X₁₁ is P, Y, or A; X₁₂ is G, D, or A; X₁₃ is S or D; X₁₄ is V, L, or S; X₁₅ is K or M; or
(d) X₁IYYSGSTX₂SNPSLKS (SEQ ID NO: 78) wherein X₁ is S, or Y; X₂ is Y or S;
wherein the HCDR3 comprises one of the following amino acid sequences:
(a) SEQ ID NOs: 13-17;
(b) X₁X₂X₃X₄X₅X₆FFDX₇ (SEQ ID NO: 24) wherein X₁ is S, D, or N; X₂ is Y or P; X₃ is G or D; X₄ is D or Y; X₅ is Y or G; X₆ is W or D; X₇ is L or Y;
(c) SEQ ID NOs: 59-73; or
(d) X₁X₂X₃X₄GX₅X₆X₇X₈X₉Y (SEQ ID NO: 82) wherein X₁ is Q or A; X₂ is Y or K; X₃ is I or W; X₄ is S or E; X₅ is T or D; X₆ is E or Y; X₇ is Y or F; X₈ is F or D; X₉ is Q or Y; and
wherein the LCDR1 comprises an amino acid sequences of any of SEQ ID NOs: 87-97, SEQ ID NOs: 123-127, or SEQ ID NOs: 141-149; the LCDR2 comprises an amino acid sequence of any of SEQ ID NOs: 99-109, SEQ ID NOs: 129-133, or SEQ ID NOs 151-159; and the LCDR3 comprises an amino acid sequence of any of SEQ ID NOs: 111-121, SEQ ID NOs: 135-139, or SEQ ID NOs: 161-169.

17. An antibody directed against Angiopoietin-Like Protein 7 (ANGPTL7) peptides, or an antigen-binding fragment thereof, comprising a heavy chain variable region (VH) comprising complementarity determining regions (CDRs) HCDR1, HCDR2, and HCDR3, and a light chain variable region (VL) comprising complementarity determining regions (CDRs) LCDR1, LCDR2, and LCDR3, wherein:
the LCDR1 comprises one of the following amino acid sequences:
(a) RASQX₁IX₂X₃X₄LX₅ (SEQ ID NO: 86), wherein X₁ is G or S; X₂ is S, R, or Y; X₃ is S, N, or I; X₄ is W, D, or Y; X₅ is A, G, or N;
(b) RSSQSLX₁X₂SX₃X₄X₅X₆YLX₇ (SEQ ID NO: 122), wherein X₁ is L or V; X₂ is H, Y, or F; X₃ is N or D; X₄ is R or G; X₅ is Y or N; X₆ is N or T; X₇ is D or N; or
(c) RASQSVSX₁X₂X₃X₄A (SEQ ID NO: 140), wherein X₁ is S, N, or R; X₂ is Y or S; X₃ is L or Y; X₄ is A or L;
wherein the LCDR2 comprises one of the following amino acid sequences:
(a) AX₁SSLX₂S (SEQ ID NO: 98), wherein X₁ is A or T; X₂ is Q or P;
(b) X₁X₂SNRX₃S (SEQ ID NO: 128), wherein X₁ is L, K, or E; X₂ is G or V; X₃ is A or D; or
(c) X₁ASX₂RAT (SEQ ID NO: 150), wherein X₁ is D or G; X₂ is N, S, or T;
wherein the LCDR3 comprises one of the following amino acid sequences:
(a) X₁QX₂X₃X₄X₅PX₆X₇ (SEQ ID NO: 110), wherein X₁ is L or Q; X₂ is A, H, S, or D; X₃ is N, F, or Y; X₄ is S, T, or N; X₅ is F, Y, or T; X₆ is W, L, I, P, or Y; X₇ is T or Y;
(b) MQX₁X₂X₃X₄PX₅T (SEQ ID NO: 134), wherein X₁ is T or G; X₂ is L or T; X₃ is Q or H; X₄ is T or W; X₅ is Y or W; or
(c) QQX₁X₂X₃X₄X₅X₆T (SEQ ID NO: 160), wherein X₁ is R, Y, or G; X₂ is S, G, or Q; X₃ is N, S, or V; X₄ is W, S, or I; X₅ is P or L; X₆ is L, S, P, or T; and
wherein the HCDR1 comprises an amino acid sequences of any of SEQ ID NOs: 2-6, SEQ ID NOs: 19-20, SEQ ID NOs: 28-42, or SEQ ID NOs: 75-77; the HCDR2 comprises an amino acid sequence of any of SEQ ID NOs: 8-12, SEQ ID NOs: 22-23, SEQ ID NOs 44-58, or SEQ ID NOs: 79-81; and the HCDR3 comprises an amino acid sequence of any of SEQ ID NOs: 13-17, SEQ ID NOs: 25-26, SEQ ID NOs: 59-73, or SEQ ID NOs: 83-85.

18. The antibody of claim 16 or claim 17, wherein the HCDR1 comprises the amino acid sequence of SEQ ID NO: 2; the HCDR2 comprises the amino acid sequence of SEQ ID NO: 8; and the HCDR3 comprises the amino acid sequence of SEQ ID NO: 13.

19. The antibody of claim 16 or claim 17, wherein the HCDR1 comprises the amino acid sequence of SEQ ID NO: 3; the HCDR2 comprises the amino acid sequence of SEQ ID NO: 9; and the HCDR3 comprises the amino acid sequence of SEQ ID NO: 14.

20. The antibody of claim 16 or claim 17, wherein the HCDR1 comprises the amino acid sequence of SEQ ID NO: 4; the HCDR2 comprises the amino acid sequence of SEQ ID NO: 10; and the HCDR3 comprises the amino acid sequence of SEQ ID NO: 15.

21. The antibody of claim 16 or claim 17, wherein the HCDR1 comprises the amino acid sequence of SEQ ID NO: 5; the HCDR2 comprises the amino acid sequence of SEQ ID NO: 11; and the HCDR3 comprises the amino acid sequence of SEQ ID NO: 16.

22. The antibody of claim 16 or claim 17, wherein the HCDR1 comprises the amino acid sequence of SEQ ID NO: 6; the HCDR2 comprises the amino acid sequence of SEQ ID NO: 12; and the HCDR3 comprises the amino acid sequence of SEQ ID NO: 17.

23. The antibody of claim 16 or claim 17, wherein the HCDR1 comprises the amino acid sequence of SEQ ID NO: 19; the HCDR2 comprises the amino acid sequence of SEQ ID NO: 22; and the HCDR3 comprises the amino acid sequence of SEQ ID NO: 25.

24. The antibody of claim 16 or claim 17, wherein the HCDR1 comprises the amino acid sequence of SEQ ID NO: 20; the HCDR2 comprises the amino acid sequence of SEQ ID NO: 23; and the HCDR3 comprises the amino acid sequence of SEQ ID NO: 26.

25. The antibody of claim 16 or claim 17, wherein the HCDR1 comprises the amino acid sequence of SEQ ID NO: 28; the HCDR2 comprises the amino acid sequence of SEQ ID NO: 44; and the HCDR3 comprises the amino acid sequence of SEQ ID NO: 59.

26. The antibody of claim 16 or claim 17, wherein the HCDR1 comprises the amino acid sequence of SEQ ID NO: 29; the HCDR2 comprises the amino acid sequence of SEQ ID NO: 45; and the HCDR3 comprises the amino acid sequence of SEQ ID NO: 60.

27. The antibody of claim 16 or claim 17, wherein the HCDR1 comprises the amino acid sequence of SEQ ID NO: 30: the HCDR2 comprises the amino acid sequence of SEQ ID NO: 46; and the HCDR3 comprises the amino acid sequence of SEQ ID NO: 61.

28. The antibody of claim 16 or claim 17, wherein the HCDR1 comprises the amino acid sequence of SEQ ID NO: 31; the HCDR2 comprises the amino acid sequence of SEQ ID NO: 47; and the HCDR3 comprises the amino acid sequence of SEQ ID NO: 62.

29. The antibody of claim 16 or claim 17, wherein the HCDR1 comprises the amino acid sequence of SEQ ID NO: 32; the HCDR2 comprises the amino acid sequence of SEQ ID NO: 48; and the HCDR3 comprises the amino acid sequence of SEQ ID NO: 63.

30. The antibody of claim 16 or claim 17, wherein the HCDR1 comprises the amino acid sequence of SEQ ID NO: 33; the HCDR2 comprises the amino acid sequence of SEQ ID NO: 49; and the HCDR3 comprises the amino acid sequence of SEQ ID NO: 64.

31. The antibody of claim 16 or claim 17, wherein the HCDR1 comprises the amino acid sequence of SEQ ID NO: 34; the HCDR2 comprises the amino acid sequence of SEQ ID NO: 50; and the HCDR3 comprises the amino acid sequence of SEQ ID NO: 65.

32. The antibody of claim 16 or claim 17, wherein the HCDR1 comprises the amino acid sequence of SEQ ID NO: 35; the HCDR2 comprises the amino acid sequence of SEQ ID NO: 51; and the HCDR3 comprises the amino acid sequence of SEQ ID NO: 66.

33. The antibody of claim 16 or claim 17, wherein the HCDR1 comprises the amino acid sequence of SEQ ID NO: 36; the HCDR2 comprises the amino acid sequence of SEQ ID NO: 52; and the HCDR3 comprises the amino acid sequence of SEQ ID NO: 67.

34. The antibody of claim 16 or claim 17, wherein the HCDR1 comprises the amino acid sequence of SEQ ID NO: 37; the HCDR2 comprises the amino acid sequence of SEQ ID NO: 53; and the HCDR3 comprises the amino acid sequence of SEQ ID NO: 68.

35. The antibody of claim 16 or claim 17, wherein the HCDR1 comprises the amino acid sequence of SEQ ID NO: 38; the HCDR2 comprises the amino acid sequence of SEQ ID NO: 54; and the HCDR3 comprises the amino acid sequence of SEQ ID NO: 69.

36. The antibody of claim 16 or claim 17, wherein the HCDR1 comprises the amino acid sequence of SEQ ID NO: 39; the HCDR2 comprises the amino acid sequence of SEQ ID NO: 55; and the HCDR3 comprises the amino acid sequence of SEQ ID NO: 70.

37. The antibody of claim 16 or claim 17, wherein the HCDR1 comprises the amino acid sequence of SEQ ID NO: 40; the HCDR2 comprises the amino acid sequence of SEQ ID NO: 56; and the HCDR3 comprises the amino acid sequence of SEQ ID NO: 71.

38. The antibody of claim 16 or claim 17, wherein the HCDR1 comprises the amino acid sequence of SEQ ID NO: 41; the HCDR2 comprises the amino acid sequence of SEQ ID NO: 57; and the HCDR3 comprises the amino acid sequence of SEQ ID NO: 72.

39. The antibody of claim 16 or claim 17, wherein the HCDR1 comprises the amino acid sequence of SEQ ID NO: 42; the HCDR2 comprises the amino acid sequence of SEQ ID NO: 58; and the HCDR3 comprises the amino acid sequence of SEQ ID NO: 73.

40. The antibody of claim 16 or claim 17, wherein the HCDR1 comprises the amino acid sequence of SEQ ID NO: 75; the HCDR2 comprises the amino acid sequence of SEQ ID NO: 79; and the HCDR3 comprises the amino acid sequence of SEQ ID NO: 83.

41. The antibody of claim 16 or claim 17, wherein the HCDR1 comprises the amino acid sequence of SEQ ID NO: 76: the HCDR2 comprises the amino acid sequence of SEQ ID NO: 80; and the HCDR3 comprises the amino acid sequence of SEQ ID NO: 84.

42. The antibody of claim 16 or claim 17, wherein the HCDR1 comprises the amino acid sequence of SEQ ID NO: 77: the HCDR2 comprises the amino acid sequence of SEQ ID NO: 81; and the HCDR3 comprises the amino acid sequence of SEQ ID NO: 85.

43. The antibody of claim 16 or claim 17, wherein the LCDR1 comprises the amino acid sequence of SEQ ID NO: 87; the LCDR2 comprises the amino acid sequence of SEQ ID NO: 99; and the LCDR3 comprises the amino acid sequence of SEQ ID NO: 111.

44. The antibody of claim 16 or claim 17, wherein the LCDR1 comprises the amino acid sequence of SEQ ID NO: 88; the LCDR2 comprises the amino acid sequence of SEQ ID NO: 100; and the LCDR3 comprises the amino acid sequence of SEQ ID NO: 112.

45. The antibody of claim 16 or claim 17, wherein the LCDR1 comprises the amino acid sequence of SEQ ID NO: 89; the LCDR2 comprises the amino acid sequence of SEQ ID NO: 101; and the LCDR3 comprises the amino acid sequence of SEQ ID NO: 113.

46. The antibody of claim 16 or claim 17, wherein the LCDR1 comprises the amino acid sequence of SEQ ID NO: 90; the LCDR2 comprises the amino acid sequence of SEQ ID NO: 102; and the LCDR3 comprises the amino acid sequence of SEQ ID NO: 114.

47. The antibody of claim 16 or claim 17, wherein the LCDR1 comprises the amino acid sequence of SEQ ID NO: 91; the LCDR2 comprises the amino acid sequence of SEQ ID NO: 103; and the LCDR3 comprises the amino acid sequence of SEQ ID NO: 115.

48. The antibody of claim 16 or claim 17, wherein the LCDR1 comprises the amino acid sequence of SEQ ID NO: 92; the LCDR2 comprises the amino acid sequence of SEQ ID NO: 104; and the LCDR3 comprises the amino acid sequence of SEQ ID NO: 116.

49. The antibody of claim 16 or claim 17, wherein the LCDR1 comprises the amino acid sequence of SEQ ID NO: 93; the LCDR2 comprises the amino acid sequence of SEQ ID NO: 105; and the LCDR3 comprises the amino acid sequence of SEQ ID NO: 117.

50. The antibody of claim 16 or claim 17, wherein the LCDR1 comprises the amino acid sequence of SEQ ID NO: 94; the LCDR2 comprises the amino acid sequence of SEQ ID NO: 106; and the LCDR3 comprises the amino acid sequence of SEQ ID NO: 118.

51. The antibody of claim 16 or claim 17, wherein the LCDR1 comprises the amino acid sequence of SEQ ID NO: 95; the LCDR2 comprises the amino acid sequence of SEQ ID NO: 107; and the LCDR3 comprises the amino acid sequence of SEQ ID NO: 119.

52. The antibody of claim 16 or claim 17, wherein the LCDR1 comprises the amino acid sequence of SEQ ID NO: 96; the LCDR2 comprises the amino acid sequence of SEQ ID NO: 108; and the LCDR3 comprises the amino acid sequence of SEQ ID NO: 120.

53. The antibody of claim 16 or claim 17, wherein the LCDR1 comprises the amino acid sequence of SEQ ID NO: 97; the LCDR2 comprises the amino acid sequence of SEQ ID NO: 109; and the LCDR3 comprises the amino acid sequence of SEQ ID NO: 121.

54. The antibody of claim 16 or claim 17, wherein the LCDR1 comprises the amino acid sequence of SEQ ID NO: 123; the LCDR2 comprises the amino acid sequence of SEQ ID NO: 129; and the LCDR3 comprises the amino acid sequence of SEQ ID NO: 135.

55. The antibody of claim 16 or claim 17, wherein the LCDR1 comprises the amino acid sequence of SEQ ID NO: 124; the LCDR2 comprises the amino acid sequence of SEQ ID NO: 130; and the LCDR3 comprises the amino acid sequence of SEQ ID NO: 136.

56. The antibody of claim 16 or claim 17, wherein the LCDR1 comprises the amino acid sequence of SEQ ID NO: 125: the LCDR2 comprises the amino acid sequence of SEQ ID NO: 131; and the LCDR3 comprises the amino acid sequence of SEQ ID NO: 137.

57. The antibody of claim 16 or claim 17, wherein the LCDR1 comprises the amino acid sequence of SEQ ID NO: 126; the LCDR2 comprises the amino acid sequence of SEQ ID NO: 132; and the LCDR3 comprises the amino acid sequence of SEQ ID NO: 138.

58. The antibody of claim 16 or claim 17, wherein the LCDR1 comprises the amino acid sequence of SEQ ID NO: 127; the LCDR2 comprises the amino acid sequence of SEQ ID NO: 133; and the LCDR3 comprises the amino acid sequence of SEQ ID NO: 139.

59. The antibody of claim 16 or claim 17, wherein the LCDR1 comprises the amino acid sequence of SEQ ID NO: 141; the LCDR2 comprises the amino acid sequence of SEQ ID NO: 151; and the LCDR3 comprises the amino acid sequence of SEQ ID NO: 161.

60. The antibody of claim 16 or claim 17, wherein the LCDR1 comprises the amino acid sequence of SEQ ID NO: 142; the LCDR2 comprises the amino acid sequence of SEQ ID NO: 152; and the LCDR3 comprises the amino acid sequence of SEQ ID NO: 162.

61. The antibody of claim 16 or claim 17, wherein the LCDR1 comprises the amino acid sequence of SEQ ID NO: 143; the LCDR2 comprises the amino acid sequence of SEQ ID NO: 153; and the LCDR3 comprises the amino acid sequence of SEQ ID NO: 163.

62. The antibody of claim 16 or claim 17, wherein the LCDR1 comprises the amino acid sequence of SEQ ID NO: 144; the LCDR2 comprises the amino acid sequence of SEQ ID NO: 154; and the LCDR3 comprises the amino acid sequence of SEQ ID NO: 164.

63. The antibody of claim 16 or claim 17, wherein the LCDR1 comprises the amino acid sequence of SEQ ID NO: 145; the LCDR2 comprises the amino acid sequence of SEQ ID NO: 155; and the LCDR3 comprises the amino acid sequence of SEQ ID NO: 165.

64. The antibody of claim 16 or claim 17, wherein the LCDR1 comprises the amino acid sequence of SEQ ID NO: 146; the LCDR2 comprises the amino acid sequence of SEQ ID NO: 156; and the LCDR3 comprises the amino acid sequence of SEQ ID NO: 166.

65. The antibody of claim 16 or claim 17, wherein the LCDR1 comprises the amino acid sequence of SEQ ID NO: 147; the LCDR2 comprises the amino acid sequence of SEQ ID NO: 157; and the LCDR3 comprises the amino acid sequence of SEQ ID NO: 167.

66. The antibody of claim 16 or claim 17, wherein the LCDR1 comprises the amino acid sequence of SEQ ID NO: 148; the LCDR2 comprises the amino acid sequence of SEQ ID NO: 158; and the LCDR3 comprises the amino acid sequence of SEQ ID NO: 168.

67. The antibody of claim 16 or claim 17, wherein the LCDR1 comprises the amino acid sequence of SEQ ID NO: 149; the LCDR2 comprises the amino acid sequence of SEQ ID NO: 159; and the LCDR3 comprises the amino acid sequence of SEQ ID NO: 169.

68. The antibody of claim 16 or claim 17, wherein the VH comprises an amino acid sequence that is at least 90% identical to any of:
(a) SEQ ID NOs: 170-174;
(b) SEQ ID NOs: 190-191;
(c) SEQ ID NOs: 198-212; or
(d) SEQ ID NOs: 258-260.

69. The antibody of claim 16 or claim 17, wherein the VL comprises an amino acid sequence that is at least 90% identical to any of:
(a) SEQ ID NOs: 180-184;
(b) SEQ ID NOs: 194-195;
(c) SEQ ID NOs: 228-242; or
(d) SEQ ID NOs: 264-266.

70. The antibody of claim 16 or claim 17, wherein the VH comprises an amino acid sequence that is at least 90% identical to SEQ ID NO: 170 and the VL comprises an amino acid sequence that is at least 90% identical to SEQ ID NO: 180.

71. The antibody of claim 16 or claim 17, wherein the VH comprises an amino acid sequence that is at least 90% identical to SEQ ID NO: 171 and the VL comprises an amino acid sequence that is at least 90% identical to SEQ ID NO: 181.

72. The antibody of claim 16 or claim 17, wherein the VH comprises an amino acid sequence that is at least 90% identical to SEQ ID NO: 172 and the VL comprises an amino acid sequence that is at least 90% identical to SEQ ID NO: 182.

73. The antibody of claim 16 or claim 17, wherein the VH comprises an amino acid sequence that is at least 90% identical to SEQ ID NO: 173 and the VL comprises an amino acid sequence that is at least 90% identical to SEQ ID NO: 183.

74. The antibody of claim 16 or claim 17, wherein the VH comprises an amino acid sequence that is at least 90% identical to SEQ ID NO: 174 and the VL comprises an amino acid sequence that is at least 90% identical to SEQ ID NO: 184.

75. The antibody of claim 16 or claim 17, wherein the VH comprises an amino acid sequence that is at least 90% identical to SEQ ID NO: 190 and the VL comprises an amino acid sequence that is at least 90% identical to SEQ ID NO: 194.

76. The antibody of claim 16 or claim 17, wherein the VH comprises an amino acid sequence that is at least 90% identical to SEQ ID NO: 191 and the VL comprises an amino acid sequence that is at least 90% identical to SEQ ID NO: 195.

77. The antibody of claim 16 or claim 17, wherein the VH comprises an amino acid sequence that is at least 90% identical to SEQ ID NO: 198 and the VL comprises an amino acid sequence that is at least 90% identical to SEQ ID NO: 228.

78. The antibody of claim 16 or claim 17, wherein the VH comprises an amino acid sequence that is at least 90% identical to SEQ ID NO: 199 and the VL comprises an amino acid sequence that is at least 90% identical to SEQ ID NO: 229.

79. The antibody of claim 16 or claim 17, wherein the VH comprises an amino acid sequence that is at least 90% identical to SEQ ID NO: 200 and the VL comprises an amino acid sequence that is at least 90% identical to SEQ ID NO: 230.

80. The antibody of claim 16 or claim 17, wherein the VH comprises an amino acid sequence that is at least 90% identical to SEQ ID NO: 201 and the VL comprises an amino acid sequence that is at least 90% identical to SEQ ID NO: 231.

81. The antibody of claim 16 or claim 17, wherein the VH comprises an amino acid sequence that is at least 90% identical to SEQ ID NO: 202 and the VL comprises an amino acid sequence that is at least 90% identical to SEQ ID NO: 232.

82. The antibody of claim 16 or claim 17, wherein the VH comprises an amino acid sequence that is at least 90% identical to SEQ ID NO: 203 and the VL comprises an amino acid sequence that is at least 90% identical to SEQ ID NO: 233.

83. The antibody of claim 16 or claim 17, wherein the VH comprises an amino acid sequence that is at least 90% identical to SEQ ID NO: 204 and the VL comprises an amino acid sequence that is at least 90% identical to SEQ ID NO: 234.

84. The antibody of claim 16 or claim 17, wherein the VH comprises an amino acid sequence that is at least 90% identical to SEQ ID NO: 205 and the VL comprises an amino acid sequence that is at least 90% identical to SEQ ID NO: 235.

85. The antibody of claim 16 or claim 17, wherein the VH comprises an amino acid sequence that is at least 90% identical to SEQ ID NO: 206 and the VL comprises an amino acid sequence that is at least 90% identical to SEQ ID NO: 236.

86. The antibody of claim 16 or claim 17, wherein the VH comprises an amino acid sequence that is at least 90% identical to SEQ ID NO: 207 and the VL comprises an amino acid sequence that is at least 90% identical to SEQ ID NO: 237.

87. The antibody of claim 16 or claim 17, wherein the VH comprises an amino acid sequence that is at least 90% identical to SEQ ID NO: 208 and the VL comprises an amino acid sequence that is at least 90% identical to SEQ ID NO: 238.

88. The antibody of claim 16 or claim 17, wherein the VH comprises an amino acid sequence that is at least 90% identical to SEQ ID NO: 209 and the VL comprises an amino acid sequence that is at least 90% identical to SEQ ID NO: 239.

89. The antibody of claim 16 or claim 17, wherein the VH comprises an amino acid sequence that is at least 90% identical to SEQ ID NO: 210 and the VL comprises an amino acid sequence that is at least 90% identical to SEQ ID NO: 240.

90. The antibody of claim 16 or claim 17, wherein the VH comprises an amino acid sequence that is at least 90% identical to SEQ ID NO: 211 and the VL comprises an amino acid sequence that is at least 90% identical to SEQ ID NO: 241.

91. The antibody of claim 16 or claim 17, wherein the VH comprises an amino acid sequence that is at least 90% identical to SEQ ID NO: 212 and the VL comprises an amino acid sequence that is at least 90% identical to SEQ ID NO: 242.

92. The antibody of claim 16 or claim 17, wherein the VH comprises an amino acid sequence that is at least 90% identical to SEQ ID NO: 258 and the VL comprises an amino acid sequence that is at least 90% identical to SEQ ID NO: 264.

93. The antibody of claim 16 or claim 17, wherein the VH comprises an amino acid sequence that is at least 90% identical to SEQ ID NO: 259 and the VL comprises an amino acid sequence that is at least 90% identical to SEQ ID NO: 265.

94. The antibody of claim 16 or claim 17, wherein the VH comprises an amino acid sequence that is at least 90% identical to SEQ ID NO: 260 and the VL comprises an amino acid sequence that is at least 90% identical to SEQ ID NO: 266.

95. The antibody of any one of claims 16 to 94, wherein the antibody binds ANGPTL7 and increases outflow facility compared to a control.

96. The antibody of 95, wherein the control is selected from the group consisting of vehicle treatment, dexamethasone treatment, ANGPTL7 protein treatment, and ANGPTL7 protein with an isotype control antibody treatment.

97. The antibody of claim 95 or claim 96, wherein;
(a) the VH comprises an amino acid sequence that is at least 90% identical to SEQ ID NO: 210 and the VL comprises an amino acid sequence that is at least 90% identical to SEQ ID NO: 240;
(b) the VH comprises an amino acid sequence that is at least 90% identical to SEQ ID NO: 200 and the VL comprises an amino acid sequence that is at least 90% identical to SEQ ID NO: 230;
(c) the VH comprises an amino acid sequence that is at least 90% identical to SEQ ID NO: 258 and the VL comprises an amino acid sequence that is at least 90% identical to SEQ ID NO: 264;
(d) the VH comprises an amino acid sequence that is at least 90% identical to SEQ ID NO: 207 and the VL comprises an amino acid sequence that is at least 90% identical to SEQ ID NO: 237;
(e) the VH comprises an amino acid sequence that is at least 90% identical to SEQ ID NO: 204 and the VL comprises an amino acid sequence that is at least 90% identical to SEQ ID NO: 234;
(f) the VH comprises an amino acid sequence that is at least 90% identical to SEQ ID NO: 260 and the VL comprises an amino acid sequence that is at least 90% identical to SEQ ID NO: 266;
(g) the VH comprises an amino acid sequence that is at least 90% identical to SEQ ID NO: 205 and the VL comprises an amino acid sequence that is at least 90% identical to SEQ ID NO: 235;
(h) the VH comprises an amino acid sequence that is at least 90% identical to SEQ ID NO: 206 and the VL comprises an amino acid sequence that is at least 90% identical to SEQ ID NO: 236
(i) the VH comprises an amino acid sequence that is at least 90% identical to SEQ ID NO: 208 and the VL comprises an amino acid sequence that is at least 90% identical to SEQ ID NO: 238;
(j) the VH comprises an amino acid sequence that is at least 90% identical to SEQ ID NO: 191 and the VL comprises an amino acid sequence that is at least 90% identical to SEQ ID NO: 195;
(k) the VH comprises an amino acid sequence that is at least 90% identical to SEQ ID NO: 203 and the VL comprises an amino acid sequence that is at least 90% identical to SEQ ID NO: 233;
(l) the VH comprises an amino acid sequence that is at least 90% identical to SEQ ID NO: 212 and the VL comprises an amino acid sequence that is at least 90% identical to SEQ ID NO: 242;
(m) the VH comprises an amino acid sequence that is at least 90% identical to SEQ ID NO: 198 and the VL comprises an amino acid sequence that is at least 90% identical to SEQ ID NO: 228;
(n) the VH comprises an amino acid sequence that is at least 90% identical to SEQ ID NO: 190 and the VL comprises an amino acid sequence that is at least 90% identical to SEQ ID NO: 194;
(o) the VH comprises an amino acid sequence that is at least 90% identical to SEQ ID NO: 202 and the VL comprises an amino acid sequence that is at least 90% identical to SEQ ID NO: 232;
(p) the VH comprises an amino acid sequence that is at least 90% identical to SEQ ID NO: 211 and the VL comprises an amino acid sequence that is at least 90% identical to SEQ ID NO: 241; or
(q) the VH comprises an amino acid sequence that is at least 90% identical to SEQ ID NO: 199 and the VL comprises an amino acid sequence that is at least 90% identical to SEQ ID NO: 229.

98. The antibody of claim 81 or claim 82, wherein;
(a) the VH comprises an amino acid sequence that is at least 90% identical to SEQ ID NO: 210 and the VL comprises an amino acid sequence that is at least 90% identical to SEQ ID NO: 240;
(b) the VH comprises an amino acid sequence that is at least 90% identical to SEQ ID NO: 200 and the VL comprises an amino acid sequence that is at least 90% identical to SEQ ID NO: 230; or
(c) the VH comprises an amino acid sequence that is at least 90% identical to SEQ ID NO: 258 and the VL comprises an amino acid sequence that is at least 90% identical to SEQ ID NO: 264.

99. The antibody of claim 95 or claim 96, wherein;
(a) the VH comprises an amino acid sequence that is at least 90% identical to SEQ ID NO: 207 and the VL comprises an amino acid sequence that is at least 90% identical to SEQ ID NO: 237;
(b) the VH comprises an amino acid sequence that is at least 90% identical to SEQ ID NO: 204 and the VL comprises an amino acid sequence that is at least 90% identical to SEQ ID NO: 234;
(c) the VH comprises an amino acid sequence that is at least 90% identical to SEQ ID NO: 260 and the VL comprises an amino acid sequence that is at least 90% identical to SEQ ID NO: 266;
(d) the VH comprises an amino acid sequence that is at least 90% identical to SEQ ID NO: 205 and the VL comprises an amino acid sequence that is at least 90% identical to SEQ ID NO: 235;
(e) the VH comprises an amino acid sequence that is at least 90% identical to SEQ ID NO: 206 and the VL comprises an amino acid sequence that is at least 90% identical to SEQ ID NO: 236
(f) the VH comprises an amino acid sequence that is at least 90% identical to SEQ ID NO: 208 and the VL comprises an amino acid sequence that is at least 90% identical to SEQ ID NO: 238;
(g) the VH comprises an amino acid sequence that is at least 90% identical to SEQ ID NO: 191 and the VL comprises an amino acid sequence that is at least 90% identical to SEQ ID NO: 195;
(h) the VH comprises an amino acid sequence that is at least 90% identical to SEQ ID NO: 203 and the VL comprises an amino acid sequence that is at least 90% identical to SEQ ID NO: 233;
(i) the VH comprises an amino acid sequence that is at least 90% identical to SEQ ID NO: 212 and the VL comprises an amino acid sequence that is at least 90% identical to SEQ ID NO: 242;
(j) the VH comprises an amino acid sequence that is at least 90% identical to SEQ ID NO: 198 and the VL comprises an amino acid sequence that is at least 90% identical to SEQ ID NO: 228; or
(k) the VH comprises an amino acid sequence that is at least 90% identical to SEQ ID NO: 190 and the VL comprises an amino acid sequence that is at least 90% identical to SEQ ID NO: 194.

100. The antibody of claim 95 or claim 96, wherein;
(a) the VH comprises an amino acid sequence that is at least 90% identical to SEQ ID NO: 202 and the VL comprises an amino acid sequence that is at least 90% identical to SEQ ID NO: 232;
(b) the VH comprises an amino acid sequence that is at least 90% identical to SEQ ID NO: 211 and the VL comprises an amino acid sequence that is at least 90% identical to SEQ ID NO: 241; or
(c) the VH comprises an amino acid sequence that is at least 90% identical to SEQ ID NO: 199 and the VL comprises an amino acid sequence that is at least 90% identical to SEQ ID NO: 229.

101. The antibody of any one of claims 16 to 100, wherein the antibody is a monoclonal antibody, a human antibody, a humanized antibody, and/or a chimeric antibody.

102. The antibody of any one of claims 16 to 101, wherein the antibody is a fragment selected from the group consisting of Fab, Fab-C, Fab′-SH, Fv, scFv, and (Fab′)₂ fragments.

103. The antibody of any one of claims 16 to 102, wherein the antibody is a monospecific antibody.

104. The antibody of any one of claims 16 to 103, wherein the antibody is a bispecific antibody.

105. The antibody of any one of claims 16 to 104, wherein the antibody comprises a detection moiety.

106. The antibody of any one of claims 16 to 105, wherein the antibody comprises a purification moiety.

107. The antibody of any one of claims 16 to 106, wherein the antibody comprises a half-life extension moiety.

108. The antibody of claim 107, wherein the half-life extension moiety comprises a polypeptide that is at least 20 amino acids in length and comprises any combination of G, A, S T, E, and P residues.

109. The antibody of claim 108, wherein the half-life extension polypeptide is attached to the C-terminus or N-terminus of the antibody.

110. A pharmaceutical composition comprising any of the antibodies of claims 1 to 109.

111. The composition of claim 110, wherein the composition is suitable for ocular administration.

112. The composition of claim 111, wherein the ocular administration comprises injection into vitreous fluid.

113. The composition of claim 111, wherein the ocular administration comprises delivering the antibody using a conjunctival insert, a contact lens, a gel, a nanoparticle, a mucoadhesive polymer, an ointment, a solution, a suspension, eye drops, and/or an implant.

114. A method of treating glaucoma and/or a disease affecting the optic nerve or retinal ganglion cells comprising administering a pharmaceutical composition comprising an effective amount of the antibody of claim 16 or claim 17, to a subject in need thereof.

115. The method of claim 114, wherein the pharmaceutical composition is administered ocularly and treats at least one symptom associated with glaucoma and/or a disease affecting the optic nerve or retinal ganglion cells.

116. The method of claim 114 or claim 115, wherein the at least one symptom associated with glaucoma and/or a disease affecting the optic nerve or retinal ganglion cells comprises eye pain, eye pressure, headaches, rainbow-colored halos around lights, low vision, blurred vision, narrowed vision, impaired peripheral vision, blind spots, nausea, vomiting, and red eyes.

117. The method of any of claims 114 to 116, wherein administering the pharmaceutical composition attenuates intraocular pressure and/or increases outflow facility in the subject's eye.

118. The method of any of claims 114 to 117, wherein the pharmaceutical composition is administered at a dose ranging from about 0.0001 mg/dose to about 100 mg/dose.

119. The method of any of claims 114 to 117, wherein the pharmaceutical composition is administered at a dose ranging from about 0.0001 mg/ml to about 100 mg/ml.

120. A polynucleotide having at least 70% identity to any of the following nucleic acid sequences:
(a) SEQ ID NOs: 175-179;
(b) SEQ ID NOs: 192-193;
(c) SEQ ID NOs: 213-227; or
(d) SEQ ID NOs: 261-263.

121. A polynucleotide having at least 70% identity to any of the following nucleic acid sequences:
(a) SEQ ID NOs: 185-189;
(b) SEQ ID NOs: 196-197;
(c) SEQ ID NOs: 243-257; or
(d) SEQ ID NOs: 267-269.

122. A polynucleotide having at least 80% identity to any of the following nucleic acid sequences:
(a) SEQ ID NOs: 175-179;
(b) SEQ ID NOs: 192-193;
(c) SEQ ID NOs: 213-227; or
(d) SEQ ID NOs: 261-263.

123. A polynucleotide having at least 80% identity to any of the following nucleic acid sequences:
(a) SEQ ID NOs: 185-189;
(b) SEQ ID NOs: 196-197;
(c) SEQ ID NOs: 243-257; or
(d) SEQ ID NOs: 267-269.

124. A polynucleotide comprising:
(a) a nucleic acid sequence that is at least 70% identical to SEQ ID NO: 175 and a nucleic acid sequence that is at least 70% identical to SEQ ID NO: 185;
(b) a nucleic acid sequence that is at least 70% identical to SEQ ID NO: 176 and a nucleic acid sequence that is at least 70% identical to SEQ ID NO: 186;
(c) a nucleic acid sequence that is at least 70% identical to SEQ ID NO: 177 and a nucleic acid sequence that is at least 70% identical to SEQ ID NO: 187;
(d) a nucleic acid sequence that is at least 70% identical to SEQ ID NO: 178 and a nucleic acid sequence that is at least 70% identical to SEQ ID NO: 188; or
(e) a nucleic acid sequence that is at least 70% identical to SEQ ID NO: 179 and a nucleic acid sequence that is at least 70% identical to SEQ ID NO: 189.

125. A polynucleotide comprising:
(a) a nucleic acid sequence that is at least 70% identical to SEQ ID NO: 192 and a nucleic acid sequence that is at least 70% identical to SEQ ID NO: 196; or
(b) a nucleic acid sequence that is at least 70% identical to SEQ ID NO: 193 and a nucleic acid sequence that is at least 70% identical to SEQ ID NO: 197.

126. A polynucleotide comprising:
(a) a nucleic acid sequence that is at least 70% identical to SEQ ID NO: 213 and a nucleic acid sequence that is at least 70% identical to SEQ ID NO: 243;
(b) a nucleic acid sequence that is at least 70% identical to SEQ ID NO: 214 and a nucleic acid sequence that is at least 70% identical to SEQ ID NO: 244;
(c) a nucleic acid sequence that is at least 70% identical to SEQ ID NO: 215 and a nucleic acid sequence that is at least 70% identical to SEQ ID NO: 245;
(d) a nucleic acid sequence that is at least 70% identical to SEQ ID NO: 216 and a nucleic acid sequence that is at least 70% identical to SEQ ID NO: 246;
(e) a nucleic acid sequence that is at least 70% identical to SEQ ID NO: 217 and a nucleic acid sequence that is at least 70% identical to SEQ ID NO: 247;
(f) a nucleic acid sequence that is at least 70% identical to SEQ ID NO: 218 and a nucleic acid sequence that is at least 70% identical to SEQ ID NO: 248;
(g) a nucleic acid sequence that is at least 70% identical to SEQ ID NO: 219 and a nucleic acid sequence that is at least 70% identical to SEQ ID NO: 249;
(h) a nucleic acid sequence that is at least 70% identical to SEQ ID NO: 220 and a nucleic acid sequence that is at least 70% identical to SEQ ID NO: 250;
(i) a nucleic acid sequence that is at least 70% identical to SEQ ID NO: 221 and a nucleic acid sequence that is at least 70% identical to SEQ ID NO: 251;
(j) a nucleic acid sequence that is at least 70% identical to SEQ ID NO: 222 and a nucleic acid sequence that is at least 70% identical to SEQ ID NO: 252; or
(k) a nucleic acid sequence that is at least 70% identical to SEQ ID NO: 223 and a nucleic acid sequence that is at least 70% identical to SEQ ID NO: 253;
(l) a nucleic acid sequence that is at least 70% identical to SEQ ID NO: 224 and a nucleic acid sequence that is at least 70% identical to SEQ ID NO: 254;
(m) a nucleic acid sequence that is at least 70% identical to SEQ ID NO: 225 and a nucleic acid sequence that is at least 70% identical to SEQ ID NO: 255;
(n) a nucleic acid sequence that is at least 70% identical to SEQ ID NO: 226 and a nucleic acid sequence that is at least 70% identical to SEQ ID NO: 256; or
(o) a nucleic acid sequence that is at least 70% identical to SEQ ID NO: 227 and a nucleic acid sequence that is at least 70% identical to SEQ ID NO: 257.

127. A polynucleotide comprising:
(a) a nucleic acid sequence that is at least 70% identical to SEQ ID NO: 261 and a nucleic acid sequence that is at least 70% identical to SEQ ID NO: 267;
(b) a nucleic acid sequence that is at least 70% identical to SEQ ID NO: 262 and a nucleic acid sequence that is at least 70% A identical to SEQ ID NO: 268; or
(c) a nucleic acid sequence that is at least 70% identical to SEQ ID NO: 263 and a nucleic acid sequence that is at least 70% A identical to SEQ ID NO: 269.

128. An expression vector comprising any of the polynucleotides of claims 120 to 127.

129. The expression vector of claim 128, wherein the expression vector is at least one of: an adeno-associated virus (AAV) vector, a lentiviral (LV) vector, a herpes simplex virus (HSV) vector, and a retrovirus vector.

130. A method of administering ocular gene therapy to a subject in need thereof comprising injecting a pharmaceutical composition comprising an effective amount of the expression vector of claim 128 or claim 129.

131. A method of treating glaucoma and/or a disease affecting the optic nerve or retinal ganglion cells comprising administering a pharmaceutical composition comprising an effective amount of the expression vector of claim 128 or claim 129, wherein administering the pharmaceutical composition treats at least one symptom of glaucoma and/or a disease affecting the optic nerve or retinal ganglion cells.

132. The antibody of claim 16 or claim 17, wherein the antibody binds an epitope from an ANGPTL7 polypeptide having any amino acid sequence of SEQ ID NOs: 370-374.

133. The antibody of claim 16 or claim 17, wherein the antibody binds an epitope from an ANGPTL7 polypeptide with a K_{D} of about 100 nM or lower.
